SEQUENCE LISTING (6 sequences)

SEQ ID NO: 1 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 2 - protein, 523 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEED

SEQ ID NO: 3 - protein, 699 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTTASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 4 - protein, 558 aa, Homo sapiens
MELRARGWWLLCAAAALVACARGDPASKSRSCGEVRQIYGAKGFSLSDVPQAEISGEHLRICPQGYTCCTSEMEENLANRSHAELETALRDSSRVLQAMLATQLRSFDDHFQHLLNDSERTLQATFPGAFGELYTQNARAFRDLYSELRLYYRGANLHLEETLAEFWARLLERLFKQLHPQLLLPDDYLDCLGKQAEALRPFGEAPRELRLRATRAFVAARSFVQGLGVASDVVRKVAQVPLGPECSRAVMKLVYCAHCLGVPGARPCPDYCRNVLKGCLANQADLDAEWRNLLDSMVLITDKFWGTSGVESVIGSVHTWLAEAINALQDNRDTLTAKVIQGCGNPKVNPQGPGPEEKRRRGKLAPRERPPSGTLEKLVSEAKAQLRDVQDFWISLPGTLCSEKMALSTASDDRCWNGMARGRYLPEVMGDGLANQINNPEVEVDITKPDMTIRQQIMQLKIMTNRLRSAYNGNDVDFQDASDDGSGSGSGDGCLDDLCSRKVSRKSSSSRTPLTHALPGLSEQEGQKTSAASCPQPPTFLLPLLLFLALTVARPRWR

SEQ ID NO: 5 - protein, 788 aa, Homo sapiens
MGIELLCLFFLFLGRNDHVQGGCALGGAETCEDCLLIGPQCAWCAQENFTHPSGVGERCDTPANLLAKGCQLNFIENPVSQVEILKNKPLSVGRQKNSSDIVQIAPQSLILKLRPGGAQTLQVHVRQTEDYPVDLYYLMDLSASMDDDLNTIKELGSRLSKEMSKLTSNFRLGFGSFVEKPVSPFVKTTPEEIANPCSSIPYFCLPTFGFKHILPLTNDAERFNEIVKNQKISANIDTPEGGFDAIMQAAVCKEKIGWRNDSLHLLVFVSDADSHFGMDSKLAGIVIPNDGLCHLDSKNEYSMSTVLEYPTIGQLIDKLVQNNVLLIFAVTQEQVHLYENYAKLIPGATVGLLQKDSGNILQLIISAYEELRSEVELEVLGDTEGLNLSFTAICNNGTLFQHQKKCSHMKVGDTASFSVTVNIPHCERRSRHIIIKPVGLGDALELLVSPECNCDCQKEVEVNSSKCHHGNGSFQCGVCACHPGHMGPRCECGEDMLSTDSCKEAPDHPSCSGRGDCYCGQCICHLSPYGNIYGPYCQCDNFSCVRHKGLLCGGNGDCDCGECVCRSGWTGEYCNCTTSTDSCVSEDGVLCSGRGDCVCGKCVCTNPGASGPTCERCPTCGDPCNSKRSCIECHLSAAGQAREECVDKCKLAGATISEEEDFSKDGSVSCSLQGENECLITFLITTDNEGKTIIHSINEKDCPKPPNIPMIMLGVSLAILLIGVVLLCIWKLLVSFHDRKEVAKFEAERSKAKWQTGTNPLYRGSTSTFKNVTYKHREKQKVDLSTDC

SEQ ID NO: 6 - protein, 788 aa, Homo sapiens
MGIELLCLFFLFLGRNDHVQGGCALGGAETCEDCLLIGPQCAWCAQENFTHPSGVGERCDTPANLLAKGCQLNFIENPVSQVEILKNKPLSVGRQKNSSDIVQIAPQSLILKLRPGGAQTLQVHVRQTEDYPVDLYYLMDLSASMDDDLNTIKELGSRLSKEMSKLTSNFRLGFGSFVEKPVSPFVKTTPEEIANPCSSIPYFCLPTFGFKHILPLTNDAERFNEIVKNQKISANIDTPEGGFDAIMQAAVCKEKIGWRNDSLHLLVFVSDADSHFGMDSKLAGIVIPNDGLCHLDSKNEYSMSTVLEYPTIGQLIDKLVQNNVLLIFAVTQEQVHLYENYAKLIPGATVGLLQKDSGNILQLIISAYEELRSEVELEVLGDTEGLNLSFTAICNNGTLFQHQKKCSHMKVGDTASFSVTVNIPHCERRSRHIIIKPVGLGDALELLVSPECNCDCQKEVEVNSSKCHHGNGSFQCGVCACHPGHMGPRCECGEDMLSTDSCKEAPDHPSCSGRGDCYCGQCICHLSPYGNIYGPYCQCDNFSCVRHKGLLCGGNGDCDCGECVCRSGWTGEYCNCITSTDSCVSEDGVLCSGRGDCVCGKCVCTNPGASGPTCERCPTCGDPCNSKRSCIECHLSAAGQAREECVDKCKLAGATISEEEDFSKDGSVSCSLQGENECLITFLITTDNEGKTIIHSINEKDCPKPPNIPMIMLGVSLAILLIGVVLLCIWKLLVSFHDRKEVAKFEAERSKAKWQTGTNPLYRGSTSTFKNVTYKHREKQKVDLSTDC

(19) United States
(12) Patent Application Publication
(10) Pub. No.: US 2023/0046007 A1
(43) Pub. Date: Feb. 16, 2023

(54) TUBULAR SHAPED ELONGATED CATHETER DEVICE ASSEMBLIES FOR INTERACTING WITH COMPONENTS OF BODILY FLUIDS, METHOD FOR RECOVERING CELLS, CELL AGGREGATES AND EXOSOMES FROM A TUBULAR SHAPED ELONGATED CATHETER DEVICE AND SMART TUBULAR SHAPED ELONGATED CATHETER DEVICE ASSEMBLIES FOR MONITORING INTERACTION WITH COMPONENTS OF BODILY FLUIDS

(71) Applicant: CIRLO GMBH, Wolfratshausen (DE)

(72) Inventors: Sabine Bauer, Wolfratshausen (DE); Thomas Ischinger, Munich (DE)

(73) Assignee: CIRLO GMBH, Wolfratshausen (DE)

(21) Appl. No.: 17/787,121

(22) PCT Filed: Dec. 16, 2020
(86) PCT No.: PCT/EP2020/086502

§ 371 Date: Jun. 17, 2022

(30) Foreign Application Priority Data
Dec. 17, 2019 (GB) 1918668.3
Dec. 17, 2019 (GB) 1918669.1
Dec. 17, 2019 (GB) 1918671.7

(51) Int. Cl. A61F 2/01

(52) U.S. Cl. CPC A61F 2/011; A61F 2/012; A61F 2250/0098; A61M 2025/1095

(57) ABSTRACT

A tubular shaped elongated catheter device assembly with a distal and a proximal end, comprising an intraluminal distal segment and an extraluminal proximal segment, comprising one or more chemical and/or biological agents, for interaction with bodily fluids of luminal organs, wherein the intraluminal segment comprises at least one expandable cross-sectional area which in its expanded state is smaller than the cross-sectional area of the luminal target site and wherein at least the expandable portion of the intraluminal segment is capable of interacting with at least one component of the bodily fluid via an interactive contact surface.

### FIELD OF THE INVENTION

The present invention relates to a tubular shaped elongated catheter device assembly comprising with a distal and a proximal end, comprising an intraluminal distal segment and an extraluminal proximal segment, comprising one or more chemical and/or biological agents, for interaction with bodily fluids of luminal organs, wherein the intraluminal segment comprises at least one expandable cross-sectional area which in its expanded state is smaller than the cross-sectional area of the luminal target site, and wherein at least the expandable portion of the intraluminal segment is capable of interacting with at least one component of the bodily fluid via an interactive contact surface, as well as corresponding methods of treatment. Also envisaged is a method of manufacturing the device assembly. The present invention further relates to a method for recovering cells, aggregates of cells and/or tumor cell derived exosomes and/or proteins and/or nucleic acids from a tubular shaped elongated catheter device assembly comprising one or more chemical and/or biological agents wherein said device being capable of recruiting said elements and thereby removes them from circulation, was implanted in a blood or lymphatic vessel, as well as corresponding methods of treatment. Also envisaged are cells, proteins or nucleic acids obtained from the method, methods of diagnosing cancer, a method of identifying a target cell or protein and a method of monitoring the effect of a disease treatment. In addition, the present invention relates to a tubular shaped elongated catheter device assembly comprising one or more chemical and/or biological agents wherein the device is capable of interacting with certain components of bodily fluids such as circulating cells, cell aggregates, exosomes or immunologic cells, wherein said device is designed to allow a read out and/or monitoring of the device with respect to the recruiting of or interaction with said components. Further envisaged is such a tubular shaped elongated catheter device assembly use in diagnosing and/or monitoring a disease and a corresponding method of monitoring the effect of a disease treatment.

### BACKGROUND OF THE INVENTION

According to the World Health Organisation, cancer represents the second most important cause of death and morbidity in Europe with more than 3.7 million new cases and 1.9 million deaths each year. On a global scale, cancer accounted for 8.2 million deaths (around 13% of the total) in 2012. Tobacco consumption and excessive alcohol consumption cause about 40% of the total cancer burden. Although more than 40% of cancer deaths can theoretically be prevented, cancer still accounts for 20% of deaths in the European Region. While Europe comprises only one eighth of the total world population it has around one quarter of the global total of cancer cases. The most cancer deaths each year are caused by lung, breast, stomach, liver, colon and breast cancer cause. Cancer thus remains a key public health concern and a tremendous burden on EU societies since it is the second largest cause of death in the European Union. According to the US National cancer institute the number of people living beyond a cancer diagnosis reached nearly 14.5 million in 2014 and is expected to rise to almost 19 million by 2024. More importantly, approximately 39.6 percent of men and women will be diagnosed with cancer at some point during their lifetimes, based on 2010-2012 data.

One defining feature of cancer is the rapid creation of abnormal cells that grow beyond their usual boundaries, and which can then invade adjoining parts of the body and spread to other organs, the latter process is referred to as metastasizing. Metastases are a major cause of death from cancer.

Metastasis to distant organs is an ominous feature of most malignant tumors but the natural history of this process varies in different cancers. The cellular origin, intrinsic properties of the tumor, tissue affinities and circulation patterns determine not only the sites of tumor spread, but also the temporal course and severity of metastasis to vital organs. Tumor progression towards metastasis is often depicted as a multistage process in which malignant cells spread from the tumor of origin to colonize distant organs (Christofori, Nature 441, 444-450, 2006). A salient feature of metastasis is the ability of different tumor types to colonize the same or different organ sites. For example, prostate cancer metastasis is largely confined to bone and metastasis by ocular melanoma is almost exclusively confined to the liver (Edlund et al, J. Cell. Biochem, 91, 686-705, 2004; Triozzi et al., Cancer Treat. Rev., 34, 247-258, 2008). Adenocarcinomas of the breast and lung typically relapse within a similar range of organs, including bone, lung, liver and brain. Breast cancer recurrences are often detected following years or decades of remission, whereas lung cancers establish distant macrometastases within months of diagnosis (Nguyen et al., Nature Reviews Cancer, 9, 274-284, 2009).

In contrast to tumor types that rapidly colonize distant organs with a short disease-free interval on initial diagnosis there are tumors that can efficiently infiltrate distant organs at early stages but are unable to promptly grow as macrometastases. In breast cancer, disseminated tumor cells (DTCs) enter a state of metastatic latency, which is defined as the time between primary tumor diagnosis and clinically detectable metastatic relapse. Malignant cells from breast tumors that disseminate early can reside as single cells or as micrometastatic clusters, as shown in studies of bone marrow samples from patients without overt metastatic disease. These DTCs either lack the ability to colonize or are prevented from displaying colonization by the environment. As a result, DTCs can enter a state of proliferative dormancy by exiting the proliferative cycle for an indefinite period (Nguyen et al., Nature Reviews Cancer, 9, 274-284, 2009). It has further been found that the presence of DTCs in patients whose primary tumors have been removed correlates with metastatic relapse, suggesting that these cells are a source of future recurrence (Braun et al., N. Engl. J. Med., 353, 793-802, 2005). DTCs have, for example, been detected primarily in the bone marrow but also in the peripheral blood and lymph nodes. The lack of specific markers and the difficulty of isolating DTCs from other organs renders unclear, whether these cells widely disseminate or the bone marrow preferentially acts as an initial reservoir of DTCs. It is further uncertain whether metastatic outgrowth preferentially occurs from these earliest latent DTCs or initiates from a later seeding of cancer cells that had already become more aggressive in the context of the expanding primary tumor. It could be shown that overt metastases and most aggressive primary tumors share similar gene expression patterns, implying that at least some metastatic traits are common between metastases and their primary tumor of origin at some stage (Nguyen et al., Nature Reviews Cancer, 9, 274-284, 2009).

The treatment options for metastases mainly focus on the provision of antibody or small mole-cute medicaments. For example, WO 2017/023994 discloses small molecule compounds, which are capable of binding to urokinase-type plasminogen activator receptor on the surface of metastatic cells and thereby recruit antibodies to the cancerous cell. The targeted cells are subsequently destroyed.

In WO 2005/014006 a combination of bisphonates and Cathepsin K inhibitors is disclosed for the prevention and treatment of bone metastases is disclosed.

In WO 2017/009837 a micro-RNA based treatment approach for solid tumors and metastases is presented, in which miR-96 and miR-182 RNA molecules are administered to patients.

Document US 2012/0208770 discloses a tumor targeting strategy based on the peptide CHP (Carcinoma Homing Peptide) which was able to inhibit metastatic tumor growth in a lung metastasis model.

However, in the currently used metastatic therapy approaches, early forms of metastatic cells, disseminated tumor cells or circulating cells with metastatic potential typically escape treatment attempts due to mal-focused administration schemes and a relatively low concentration and incidence of these cells in the vascular system due to high unspecific binding.

More importantly, in many cases the presence of infrequently occurring tumorous cells, cell aggregates or derived exosomes is very difficult to detect, thus increasing the probability of missing the opportunity to start treatment approaches at an early stage or in the beginning of relapses.

In addition, in many situations a surveillance system for presence and concentration changes in terms of cellular bodily fluid components or exosomes could provide at the same time an alert, diagnostic and first responding treatment option.

There is thus a need for an alternative therapeutic approach, which allows to trap and eradicate this group of highly dangerous and evasive cells, as well as other disease-associated elements in bodily fluids. Furthermore, it is necessary to reduce the tumor mass in particular when the tumor tissue is of high density as, e.g. in pancreatic cancer since dense ECM typically prevent necessary concentrations of therapeutics at the site of tumor growth and also hinders sufficient infiltration of tumor antigen presenting immune cells. There is an additional need for an efficient and personalized methodological approach, which allows to effectively and suitably trap and analyse this group of highly dangerous and evasive cells in bodily fluids. There is further a need for a physiologically suitable and efficient system, which allows for a rapid and detailed read out and monitoring of component interactions from bodily fluids in luminal organs.

### OBJECTS AND SUMMARY OF THE INVENTION

The present invention addresses these needs and provides in a first main aspect a tubular shaped elongated catheter device assembly with a distal and a proximal end, comprising an intraluminal distal segment and an extraluminal proximal segment, comprising one or more chemical and/or biological agents, for interaction with bodily fluids of luminal organs, wherein the intraluminal segment comprises at least one expandable cross-sectional area which in its expanded state is smaller than the cross-sectional area of the luminal target site and wherein at least the expandable portion of the intraluminal segment is capable of interacting with at least one component of the bodily fluid via an interactive contact surface. The present inventors have surprisingly found that the use of a device which is capable of interacting with at least one component of a bodily fluid such as a circulating tumor cell, a circulating metastatic cell, a motile parts thereof, an exosome or tumor cell aggregates, as well as cells of the immune system allows to capture and thus remove these components from the bodily fluids, in particular from the bloodstream or lymphatic fluids, and/or to facilitate their destruction subsequent to the interaction with the device. The thus bound cells or particles are removed from circulation and are therefore no longer capable of depositing at downstream organs or tissues. The cells' presence in the device further allows for their dis-enablement by interaction with immune cells, and/or toxins or pro-apoptotic elements. In addition, the cells can be modified by interaction with molecules present in or on the device leading to the presentation of signs of apoptosis by the cells which results in immune cell attacks. This innovative concept thus facilitates a therapeutic, as well as preventive advancement against circulating components in the bodily fluids such as tumor cells, exosomes, or cell aggregates, in particular circulating metastatic cells, which are otherwise hardly detectable. An intratumor device according to the invention is further able to generate comparable biological anti-tumor effects. In this context it is advantageous that the device is biodegradable.

An additional advantage of the inventive device is its perioperative and post-operative usability for an efficient capturing and elimination of circulating cells or cell aggregates, which are detached or loosened during tumor resections. By capturing these cells, a new settling of such cells can be effectively prevented, thus significantly increasing the success rate of the surgery or enables surgery by reducing the tumor mass, or circumvent the generation of resistance mechanisms (e.g. gemcitabine resistance). Moreover, the local and selective capture of circulating cells and motile parts thereof in a chosen target vessel advantageously allows the device to be inserted into the body by a routine minimally invasive catheter procedure, for example after puncture of the target vessel or a vessel leading to the target. Alternatively, an intratumoral or near tumor scaffold device can be placed inside the tumor tissue or in close vicinity of the tumor, also applying, for example, minimal invasive methods. Furthermore, the replacement of certain components of the device, in particular the filter membranes, allows for a very efficient use over a prolonged period of time. The present invention's approach thus significantly improves the therapy and prognosis of patients, in particular of cancer patients with risk of metastatic disease or manifest metastatic disease. An intratumor scaffold device reduces the tumor mass and the tumor cell debris can be taken up by immune cells. This in turn is assumed to lead to an enhanced immune response towards the tumor. Immune checkpoints and immune checkpoint inhibitors may locally be applied at a slow and continuous kinetic trying to beneficially influence an immune quiet ECM of the tumor.

Vascular catheter-, stent- or scaffold-filter technology, which resembles the presently claimed approach on a mechanistic level, is well established. A stent or scaffold usually is a permanent implant into a diseased and obstructed artery or vein in order to improve blood flow and maintain patency of the formerly obstructed vessel. Stents may, for example, be balloon expandable or self-expandable. Balloon expandable stents are typically made of plastically deformable material such as 316 L steel or cobalt-chrome alloys, and are mounted on a deflated balloon, positioned in the target zone and expanded by inflation of the balloon. Self-expandable stents are usually, but not exclusively, made of a nitinol mesh, i.e. an elastically deformable, memory metal, which is held constraint on a catheter by an outer constraining mechanism and is released in the target organ by withdrawal of the constraining tube. The self-expanding stent then takes its predetermined shape via the memory metal effect.

In addition, there are examples of capturing devices such as the Filterwire (Boston Scientific) or Spider FX (Covidien) device (see also FIG. 54), which are essentially filters resembling umbrellas on a wire with uniform pores of typically 80 μm-110 μm in diameter. Their purpose is the capture of macroscopically visible, large size debris during an interventional procedure. At termination of the procedure the filters are withdrawn. Also known in the art is the so called vena cava filter (Cook, Crux Biomedical), which is a crude filter device that is released in the Vena cava in patients with extremely high risk of spontaneous venous thromboembolism. The purpose of this filter is to trap large masses of thrombotic material in order to avoid pulmonary embolism. While it is intended for permanent implantation, it may be retrieved by lasso or other known catheter techniques as long as it is not overgrown by tissue or not perforated into the vessel wall. Recently, a non-permanent vena cava embolic filter has been introduced for the prevention of pulmonary embolism in high risk medical situations in critically ill patients, i.e. during trauma surgery and when anticoagulation is contraindicated (Angel catheter, Bio2Medical).

There is, however, no prior art disclosure of a dedicated catheter device assembly, which is specially designed for non-permanent use and for being capable of recruiting a circulating tumor or circulating metastatic cell or motile parts of tumor cells and thereby removes said cell or motile part thereof from circulation, featuring, for example, inter alia self-expansion and antimigratory mechanical properties, clogging resistance according to the present invention. Thus, while resembling some aspects of traditional stents and conventional embolic filters, the device of the present invention advantageously transforms some of the physical features of traditional stent and emboli (blood clots) preventing filter technology into pharmaceutical composition-like devices which are capable of fulfilling completely different purposes such as tumor cell scavenging.

Another advantage and a primary goal of the presently envisaged device is that its filter component is aiming at the capture of cells, or of parts of cells such as exosomes and not the capture of emboli or debris, thus featuring significantly smaller filter structures or pore sizes. The device is accordingly designed to maintain blood flow even through the microscopically small filter structures by creating areas with unhindered flow within or adjacent to the filter membranes in order to minimize reduction or stasis of flow or thrombosis. This approach is in clear contrast to filters known in the art, which are typically intended to completely cover the cross-section of a vessel in order to provide full embolic protection.

In a further, second main aspect the present invention provides a method for recovering cells, aggregates of cells and/or tumor cell derived exosomes and/or proteins and/or nucleic acids from a tubular shaped elongated catheter device assembly comprising one or more chemical and/or biological agents wherein said device, which is capable of recruiting a circulating tumor and/or circulating metastatic cell and/or motile parts of tumor cells and/or an aggregate of tumor cells and/or a tumor cell derived exosome and/or immune cells and thereby removes said cell or motile part thereof from circulation, was implanted in a blood or lymphatic vessel downstream of an existing cancer site or close to a site of potential metastasis formation in a subject, wherein said device is retrievable or partially retrievable, preferably by catheter means and/or in a minimal invasive manner. The present inventors have surprisingly found that a tubular shaped elongated catheter device assembly comprising one or more chemical and/or biological agents wherein said device, which is capable of recruiting a circulating tumor and/or circulating metastatic cell and/or motile parts of tumor cells and/or an aggregate of tumor cells and/or a tumor cell derived exosome, can very effectively be used for the recovery of aggregates of cells and/or tumor cell derived exosomes and/or proteins and/or nucleic acids and/or recruited immune cells from a tubular shaped elongated catheter device since these components are removed from circulation after the device was implanted in a blood or lymphatic vessel downstream of an existing cancer site or close to a site of potential metastasis formation in a subject and, subsequently, was retrieved from its implantation site.

The thus bound cells or particles are removed from circulation and can be analyzed ex vivo, e.g. after having been removed from the device and transferred to cell cultures or after their molecular identity has been determined, e.g. by nucleic acid sequencing, amino acid analysis, signal pathway mapping, FACS or ELISA. This innovative concept thus facilitates a personalized diagnostic approach which helps understanding the molecular situation at certain locations within the vascular system or in specific luminal organs via fingerprinting the interactive components of the bodily fluids passing said system or said organs. Corresponding insights can advantageously be used for several important purposes such as the adapting and fine-tuning the treatment strategy for diseases, in particular cancer treatments, monitoring of treatment effects, basic research of underlying processes in cellular movements within the vascular system, e.g. after development of primary tumors or prognosticating disease developments. The envisaged approach further allows for the potential discovery of new biomarkers, for disease and therapy monitoring, the development of new personalized treatment regimens and patient tailored therapies. A further advantageous effect is that new therapeutic windows are opened for existing therapies such as immune therapies.

It further becomes possible to develop a tumor tissue depositary or bank which can be used to deposit patients' tumor samples or cells and tissues recovered before, during and after a treatment.

In another, third main aspect the present invention provides a tubular shaped elongated catheter device assembly comprising one or more chemical and/or biological agents wherein the device is capable of interacting with a circulating tumor cell and/or a circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein within bodily fluids of luminal organs, wherein said device is designed to allow a read out and/or monitoring of the device with respect to the recruiting of or interaction with a circulating tumor and/or circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein.

The present inventors have further found that the use of a device which is capable of interacting with at least one component of a bodily fluid such as a circulating tumor cell, a circulating metastatic cell, a motile parts thereof, an exosome or tumor cell aggregates, as well as cells of the immune system allows a read out and/or monitoring of the device with respect to the recruiting of or interaction with said components. Accordingly, by using smart technology such as electro-active polymeric (EAP) materials and connectivity modules the presence and increase/decrease in concentration of the components can advantageously be sensed, allowing for (i) a rapid (online) diagnosis of the patient's health state, (ii) an adjustment of possible therapy or surgery options, or (iii) a first pharmacological response directly from the device, e.g. by releasing suitable drugs or drug combinations. This innovative device, which is, in some embodiments, designed as a combination of an online in vivo monitoring system and an external data receiver advantageously allows individual patient data transfer to the clinic or a laboratory or the doctor's practice. This option drastically reduces costs of the health care system and allows for personalized therapy monitoring and personalized treatment regimens. A further advantage is that the captured components are removed from the bodily fluids, in particular from the bloodstream or lymphatic fluids subsequent to the interaction with the device. The thus bound cells or particles are removed from circulation and are therefore no longer capable of depositing at downstream organs or tissues. This innovative concept thus facilitates a diagnostic, as well as preventive advancement against circulating components in the bodily fluids such as tumor cells, exosomes, or cell aggregates, in particular circulating metastatic cells, which are otherwise hardly detectable.

Moreover, the device of the present invention can be used for neurological monitoring, e.g. in the context of Parkinson's disease. The device allows to measure in vivo, for example, the amount of SIRT protein expression allowing for a differential diagnose of Parkinson's disease vs. atypical Parkinson syndrome. Furthermore, the approach allows for the detection of antibody complex formation during autoimmune diseases, such as SLE and RA. Also, the replacement of certain components of the device, in particular the filter membranes, allows for a very efficient use over a prolonged period of time. The present invention's approach thus significantly improves the diagnosis of patients, in particular of cancer patients with risk of metastatic disease or manifest metastatic disease. According the present concept, there is further no need for frequent clinic visits, and personalized treatment regimens, thus contributing to an overall cost reduction.

Further advantages include facilitated retrieving due to less contact to the walls of the vessel, easy handling by interventional radiologists, and enlargement of contact surface. Moreover, the described compartments are valuable for different drugs, e.g. cytokines, small molecules, cytostatics, space for cell and debris, recruiting and storage.

In a preferred embodiment of the first main aspect of the invention the tubular shaped elongated catheter device assembly is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

In a further embodiment, the at least one expandable portion of the intraluminal segment is an inflatable balloon, wherein said expandable portion is designed to bind a tumor cell, a tumor cell exosome, and/or a tumor cell aggregate and/or a pathogen.

In a further preferred embodiment, the cross-sectional area of the intraluminal segment is at least about 50% smaller than the cross-sectional area of the luminal target site.

In a further preferred embodiment, the intraluminal segment comprises at least one expandable portion, preferably a reversibly expandable portion.

In yet another embodiment, the expandable portion is capable of increasing the interactive contact surface with the bodily fluid.

A further preferred embodiment relates to a device as defined above, wherein the expandable portion at maximal expansion comprises a cross-sectional area of equal or less than about two thirds of the cross-sectional area of the luminal target site.

In yet another preferred embodiment, the expandable portions only partially covering the cross-sectional area of the luminal target are arranged sequentially along the longitudinal extension in a clock-wise orientation, preferably with more than 90° differences between the sequential positions.

In a further embodiment, the device additionally comprises at least two localization markers, preferably opposite to each other.

It is particularly preferred that said localization markers are located at the starting and end point of a segment comprising chemical and/or biological agents.

In a further preferred embodiment said localization marker is a radiopaque marker, an ultra-sound marker, an MRT marker or a CT marker, preferably further comprising at least one sensor such as an optical sensor, an analyte detecting sensor, a thermal sensor or a flow sensor.

In another preferred embodiment, the device comprises sections permitting free flow of bodily fluids without any obstructing structural elements at any given point along the longitudinal extension of the device.

In yet another preferred embodiment, the device comprises sections permitting at least 30%, preferably 50% cross-sectional unhindered flow of bodily fluids.

In an additional embodiment, the device according to the present invention comprises sections which permit a free flow of bodily fluids, interrupted by one or more sections permitting an interaction with components of the bodily fluids, preferably a filtering by means of a membrane like component, wherein preferably the interactive sections spare cross-sectional areas of free flow.

It is particularly preferred that the tubular shaped elongated catheter device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is anchorable in a target vessel; (v) it is designed to fit into and be connected to a permanent device present in a target vessel as a shuttle docking to a receiving site.

The present invention also envisages that the proximal end of the extraluminal segment is connected to an extraluminal fixation element for temporary implantation and for fixation of the intraluminal device and prevention of intraluminal migration of the free-floating device.

In yet another embodiment, the device comprises a through lumen housing of the expandable portions of the device.

It is further preferred that the device is characterized by at least one wire lumen within at least the interluminal segment of the device.

In yet another embodiment, the reversibly expandable segment of the device is comprised of at least one expandable balloon surface having a capability for interaction with at least one component of the bodily fluid, wherein the expandable balloon is preferably arranged with the distal end of the intraluminal portion of the device.

In another embodiment, the elongated tubular shape is provided by a memory shaped wire, preferably a flexible nitinol wire.

In a further embodiment, the device comprises extensions of the wire in the form of circular loops or ellipsoids or non-straight longitudinal extensions.

Further envisaged is a device as defined above, wherein the expandable portion comprises a porous membranous surface.

In a further embodiment, said expandable portion comprises a filter membrane.

It is preferred the filter membrane has one or more of the following properties: (i) it comprises pores, (ii) it is expandable; (iii) it is retrievable; (iv) it is disposed within or around an expandable body; (v) it is characterized by a memory shaped metallic or memory shaped polymer structure; (vi) it comprises a detachment mechanism cooperating between the expandable portion and an intraluminal segment of the device or (vi) it is self-expandable.

In yet another embodiment, the device additionally comprises longitudinally extending free floating microfilaments; or comprises an outer sheath concentrically disposed about the device, wherein the outer sheath and the device are movable relative to one another.

Also envisaged is a device as defined above, which additionally comprises a downstream embolic debris filter, wherein the filter is preferably retrievable and/or has a pore diameter of >100 μm.

In yet another preferred embodiment, the device as defined above comprises a longitudinally extending catheter.

Further envisaged by the present invention are devices as defined above, wherein said filter membrane has at least one of following properties: (i) the filter membrane is attached to and arranged with the expandable portion of the device between its proximal and distal end; (ii) the filter membrane is a non-permanent, retrievable filter membrane.

In particularly preferred embodiments, said pores have a pore diameter which ranges from about 25 nm to about 100 μm, more preferably in a differential manner such as comprising differential ranges of 25 nm to 100 nm, 100 nm to 10 μm, 10 μm to 25 μm, or 25 μm to 100 μm.

It is also preferred that said expandable portions are at least partially coated with said one or more chemical and/or biological agents, wherein preferably said agents are permanently fixed or releasable.

In another embodiment, said device is provided in a tubular, onion like, pearl-chain-like, or a birds-nest like shape, or in any mixture of these shapes.

In a preferred embodiment, said tubular shape is provided by a memory shaped spiraling wire, which forms a tubular spiral.

In yet another preferred embodiment, the memory shaped spiraling wire is modified into a single spiral-like wire structure, characterized by incomplete wire circles and an interdigiting structure.

In a further preferred embodiment, said onion like or pearl-chain-like shape is provided by an elastic memory shape meshwork.

In a further aspect in the context of the first main aspect of the invention, the invention relates to a tubular shaped device assembly for intraluminal use with a distal and a proximal end, comprising an intraluminal segment which comprises one or more chemical and/or biological agents, for interaction with bodily fluids of luminal organs, wherein at least one portion of the intraluminal segment is expandable and capable of interacting with at least one component of the surrounding bodily fluids via an interactive expandable contact surface, wherein the maximal increase in interactive surface area is at least 3 fold, wherein the interactive segment in its expanded state leaves at least 50% of the surrounding continuous cross-sectional luminal plane void of any structural or interactive component of the expansive device at any level of the longitudinal extension of the interactive segment of the device.

In a preferred embodiment of the tubular shaped device assembly for intraluminal use said device assembly comprises an intraluminal distal segment and an extraluminal proximal segment, further comprising at least two channels within at least a portion of the longitudinal extension of the device, wherein at least one channel is a through channel for use as wire or infusion channel, and wherein at least one channel is a balloon inflation channel, wherein at least one balloon inflation channel is in fluid connection with at least one flow directable compliant expandable balloon, wherein the balloon is arranged around the distal end section of the intraluminal device, wherein expanded balloon diameters are smaller than the surrounding tubular organ diameter, and wherein at least the expandable portions of the intraluminal segment are interactive with elements of bodily fluids in the target area.

In a further preferred embodiment of the tubular shaped device assembly for intraluminal use said at least one expandable portion of the intraluminal segment is an inflatable balloon, wherein said expandable portion is designed to bind a tumor cell, a tumor cell exosome, and/or a tumor cell aggregate, and/or a pathogen.

In a further preferred embodiment of the tubular shaped device assembly for intraluminal use any expanded and interactive portion at any level of the longitudinal extension of the intraluminal segment spares at least about 50% of the surrounding cross-sectional area from any flow obstructive device components or interactive device elements in favor of more than 50% cross-sectional area void of any device elements adjacent or within the interactive site of the device.

In a further preferred embodiment of the tubular shaped device assembly for intraluminal use said intraluminal segment comprises at least two reversibly expandable interactive portions arranged in a tandem like order along the longitudinal axis of the intraluminal segment and sequentially arranged eccentrically and spaced to each other on the circumference of the tubular intraluminal segment, with an offset in a clockwise orientation with at least 90° differences or opposite to each other, preferably with 180° difference in circumferential position.

In a further preferred embodiment of the tubular shaped device assembly for intraluminal use said at least one expandable portion of the intraluminal segment is extending over any length ranging from about 10 mm to more than 50% of the intraluminal length of the device assembly and is capable of increasing the interactive contact surface with the bodily fluid.

In a further preferred embodiment of the tubular shaped device assembly for intraluminal use said at least a portion of the intraluminal segment of the device provides structural elements as reservoirs for drug or biological agents or their compounds, wherein release of these agents is controlled by the release kinetics of the drug compound or is activated by expansive forces such as balloon or self expansion.

In a further preferred embodiment of the tubular shaped device assembly for intraluminal use said device additionally comprises at least two localization markers on the intraluminal segment, identifying the proximal and distal end of interactive sites, localized eccentrically and opposite to each other on the circumference of the tubular shaped device, the markers being characterized by different configurations as visualizable by medical imaging, wherein the eccentric markers are designed to permit visualization of longitudinal and rotational positioning.

In a further preferred embodiment of the tubular shaped device assembly for intraluminal use said localization markers for medical imaging comprise contrast deposits visible in medical imaging.

In a further preferred embodiment of the tubular shaped device assembly for intraluminal use said contrast deposits visible in medical imaging are MRI visible, preferably comprising Gadolinium.

In a further preferred embodiment of the tubular shaped device assembly for intraluminal use said contrast deposits are arranged within balloon like cavities along a non-through lumen such as the inflation lumen.

In a further preferred embodiment of the tubular shaped device assembly for intraluminal use the specific device design maintains continuous cross-sectional areas of more than 60% void of any structural or interactive device components adjacent or along its interactive expandable sites at any given point along the longitudinal extension of the device assembly.

In a further preferred embodiment of the tubular shaped device assembly for intraluminal use the device assembly comprises inactive sections which permit a free flow of bodily fluids along or within the device, interrupted by one or more sections comprising interactive sites which are designed to permit an interaction with components of the bodily fluids, preferably a filtering by means of a membrane like component, wherein preferably the interactive sites spare cross-sectional areas of more than 50% of the cross-sectional surrounding area for free flow void of any device assembly elements.

In a further preferred embodiment of the tubular shaped device assembly for intraluminal use, said device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is atraumatically anchorable in a target vessel; (v) it is designed to fit into and be connected to a permanent device present in a target vessel as a shuttle docking to a receiving site.

In a further preferred embodiment of the tubular shaped device assembly for intraluminal use, said device assembly is characterized by at least one wire lumen within at least the intraluminal segment of the device and at least one inflation lumen.

In a further preferred embodiment of the tubular shaped device assembly for intraluminal use a through lumen within the device assembly houses an optical fiber for light application or a probe for energy application or transmission of sensor activity, preferably an ultrasound probe.

In a further preferred embodiment of the tubular shaped device assembly for intraluminal use said at least one expandable interactive site comprises magnetically active components for scavenging magnetically marked antigen/antibody complexes

In a further preferred embodiment of the tubular shaped device assembly for intraluminal use the device assembly further comprises one or more of the features of the tubular shaped elongated catheter device assembly as defined above.

In yet another aspect in the context of the first main aspect of the invention, the invention relates a multilumen tubular device, characterized by a multilumen design and by a flow-directable balloon design, comprising reversibly expandable interactive sites which are characterized by interactive surfaces for physical and biochemical or molecular interaction with elements of bodily fluids, wherein at least two interactive sites are arranged in a longitudinally extended tandem position and in a circumferentially clockwise offset position of at least 90° around the tubular device, wherein a continuous cross-sectional area of at least >50% of total surrounding luminal cross-section is void of any device components at any point along the longitudinal extension of the device, wherein the device is designed for non permanent use as free floating intraluminal device with extracorporeal fixation unit, wherein at least the beginning and end of an interactive site of the device is marked by markers readable by any medical imaging technique and wherein eccentric markers of different configurations on the tubular device permit appreciation of rotational position by medical imaging techniques.

In a preferred embodiment of the multilumen tubular device said further comprises one or more of the features of the tubular shaped elongated catheter device assembly as defined above.

In another embodiment of the present invention the tubular shaped elongated catheter device assembly, the tubular shaped device assembly for intraluminal use or the multilumen tubular device as defined herein above is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques such as X ray.

In yet another embodiment, said tubular shaped elongated catheter device assembly, said tubular shaped device assembly for intraluminal use or said multilumen tubular device is self-expandable.

In a further preferred embodiment, at least a portion of said tubular shaped elongated catheter device assembly, said tubular shaped device assembly for intraluminal use or said multilumen tubular device can be activated by balloon inflation.

In an additional preferred embodiment, said tubular shaped elongated catheter device assembly, said tubular shaped device assembly for intraluminal use or said multilumen tubular device comprises at least one docking element at least one end, preferably at the proximal end, for retrieval.

It is particularly preferred that said expandable portion is composed of elastic, foldable polymer material such as polyurethane, or of micromeshes comprising ultrathin wires, metallic or polymeric material.

In specific embodiments, at least one cross-sectional area of the tubular device body is at least partially covered by the filter membrane.

It is further preferred that the plane of the filter membrane as defined herein above is arranged perpendicular to the direction of the longitudinal axis of the device body. Alternatively, the plane of the filter membrane is in an angle which is non perpendicular to the longitudinal axis of the device body.

In yet another embodiment, said tubular shaped elongated catheter device assembly, said tubular shaped device assembly for intraluminal use or said multilumen tubular device as defined herein above comprises at least two filter membranes, each of which incompletely covers the cross-sectional area, and which are arranged in tandem position along the longitudinal axis of the device body, preferably opposite to each other within the circumference of the device body or shifted in clockwise orientation in case of more than two filter membranes in tandem position.

In yet another embodiment, said tubular shaped elongated catheter device assembly, said tubular shaped device assembly for intraluminal use or said multilumen tubular device comprises alternating non-completely covering filter membranes, preferably in association with a tubular scaffold like and/or in a pearl-chain, onion type or birds-nest like or bi- or trifoil like shape.

In a further preferred embodiment, the filter membranes as mentioned herein have differential pore diameters and/or differential pattern. In a particularly preferred embodiment they range from about 25 nm to about 100 μm. In further embodiments two or more filter membranes have differential pore diameters and/or differential pattern, preferably ranging from about 25 nm to about 100 μm such as comprising differential pore diameter ranges of 25 nm to 100 nm, 100 nm to 10 urn, 10 μm to 25 μm, or 25 μm to 100 μm.

In a further embodiment, at least one filter membrane is fully or partially coated on its interior side; or on its exterior side; or on both sides with said one or more chemical and/or biological agents; or wherein said coating differs between different filter membranes.

In another embodiment, said coating as mentioned above is a passive coating with one or more polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) or linear aliphatic polyesters.

It is further preferred that said passive coating adheres to the structural support material via an adhesive layer, preferably of sugar, starch, polyvinylalcohol or degradable products of these materials.

In a particularly preferred embodiment, said one or more chemical and/or biological agents constitute an extracellular matrix-like structure. It is further preferred that said extracellular matrix-like structure is covalently or non-covalently bound to the passive coating and/or the device material.

The present invention also relates to an embodiment, wherein said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof; preferably a protein mixture secreted by Engelbreth-Holm-Swarm (EHS) mouse sarcoma cells, Matrigel, BioCoat or GelTrex. It is particularly preferred that protein mixtures with human proteins are employed.

In another preferred embodiment, the tubular shaped elongated catheter device assembly, said tubular shaped device assembly for intraluminal use or said multilumen tubular device as mentioned herein provides an environment for circulating metastatic cells.

It is further preferred that said tubular shaped elongated catheter device assembly, said tubular shaped device assembly for intraluminal use or said multilumen tubular device comprises a biological agent as an active coating, which is capable of binding to a tumor marker; or to a cell of the immune system or a corresponding immune cell marker. Particularly preferred are tumor markers which are specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma.

In a further particularly preferred embodiment, said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I).

In yet another embodiment, said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

It is particularly preferred that said homing factor is Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

Another embodiment of the present invention relates to a tubular shaped elongated catheter device assembly, said tubular shaped device assembly for intraluminal use or said multilumen tubular device as defined herein above, wherein said cell of the immune system is a CD8⁺ cell, a dendritic cell, a T cell, an engineered T cell, a B cell or an NK cell.

It is further preferred that said biological agent is linked to the passive coating of the device or to the structural support material via a spacer element.

In another preferred embodiment, said linkage to the structural support material is binding to a metal ion resin, such as ion-NTA or ion-agarose.

In yet another preferred embodiment, said spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

In another embodiment, said spacer has a length of about 1 to 20 nm.

In yet another embodiment, said spacer elements are provided in a density of 2 to 500 per μm² on the surface of the device.

In a further embodiment, said biological agent comprises, essentially consists of, or consists of a binding domain capable of binding to a tumor marker.

It is further envisaged that said binding domain is a peptide or polypeptide molecule having a length of about 20 to about 250 amino acids, preferably of about 20 to about 120 amino acids. It is particularly preferred that said binding domain has a length of 20, 25, 30, 35, 40, 45, 50, 55, 60, 65, 70, 75, 80, 85, 90, 95, 100, 105, 110, 115 or 120 amino acids.

In another embodiment, said biological agent additionally comprises one or more functional domains.

In an additional embodiment, said further functional domain is or comprises, partially comprises or consists of an apoptosis inducing factor or a functional domain of an apoptosis inducing factor capable of inducing apoptosis, or a domain capable of binding to a cell of the immune system. It is particularly preferred that said apoptosis inducing factor is FasL/CD95L, TNF-alpha, APO3L or APO2L/TRAIL.

In yet another embodiment, said domain is capable of binding to a tumor marker and said do-main capable of inducing apoptosis are provided as fused domains or are linked via a linker element of about 1 to 20 amino acids length.

In a further embodiment, said biological agent is covalently or non-covalently connected to said spacer.

In a further embodiment, said connection is a linker element. It is preferred that said linker element is a peptide, preferably having a length of 1, 2, 3, 4, 5, 6, 7, 8, 9, 10, 11, 12, 13, 14, 15, 16, 17, 18, 19 or 20 amino acids.

In yet another embodiment, said biological agent is provided as linear or circular element or as an element composed of linear and circular parts, preferably as a linear or circular or partially linear/circular peptide or polypeptide, or a protein with secondary or tertiary structure conformation.

In a further embodiment, said circular biological agent has or is part of a structure comprising a loop or a loop and a stem, such as a human Fc part; or of a linear structure, which is linked to said spacer element.

Also envisaged are devices as defined herein above, wherein said loop structure or linear structure comprises said biological agent at an exposed position allowing for the binding to a tumor marker or a tumor cell, a tumor cell exosome, and/or a tumor cell aggregate.

In a preferred embodiment of a tubular shaped elongated catheter device assembly, said tubular shaped device assembly for intraluminal use or said multilumen tubular device as defined herein above, said peptide has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined conjugated with stabilizing components such as PEG or lipids, preferably forming lipoglyco-peptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, essentially comprises or consists of D-amino acids and/or L-amino acids; (xi) it partially comprises, essentially comprises or consists homo-amino acids such as beta-homo-amino acids, N-methyl amino acids, or alpha-methyl-amino acids; (xii) it partially comprises, essentially comprises or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide.

In further embodiments, said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

Also envisaged are tubular shaped elongated catheter device assemblies, tubular shaped device assemblies for intraluminal use or multilumen tubular devices as defined herein above, which further comprise a pharmaceutical agent, preferably selected from the group comprising an antiproliferative agent and an anticoagulant.

In a preferred embodiment, said antiproliferative agent is paclitaxel, sirolimus, or an analogue thereof.

In another preferred embodiment, said anticoagulant is reteplase, heparin, or a peptide such as a bifunctional peptide preventing coagulation.

In a further aspect, the present invention relates to a tubular shaped elongated catheter device assembly, a tubular shaped device assembly for intraluminal use or a multilumen tubular device as defined herein above for use in treating cancer and/or metastasis, preferably in luminal organs such as blood vessels.

In yet another aspect, the present invention relates to a tubular shaped elongated catheter device assembly, a tubular shaped device assembly for intraluminal use or a multilumen tubular device as defined herein above for use in preventing cancer and/or metastasis, preferably in luminal organs such as blood vessels.

In preferred embodiments, said tubular shaped elongated catheter device assembly for use, said tubular shaped device assembly for use or said multilumen tubular device for use is capable of quantitatively capturing circulating tumor and/or circulating metastatic cell and/or motile parts of tumor cells and/or an aggregate of tumor cells and/or a tumor cell derived exosome, preferably circulating metastatic cells, in a subject's body.

It is further preferred that said tubular shaped elongated catheter device assembly for use, said tubular shaped device assembly for use or said multilumen tubular device for use is capable of preventing downstream organs or tissues to be reached by circulating tumor and/or circulating metastatic cell and/or motile parts of tumor cells and/or an aggregate of tumor cells and/or a tumor cell derived exosome, preferably metastatic cells, which are circulating in a subject's body.

In a preferred embodiment, said cancer is colon cancer, breast cancer, lung cancer, melanoma, esophageal cancer, prostate cancer, pancreatic cancer, ovarian cancer, myeloma, lymphoma such as ALL, CLL, AML.

It is particularly preferred that said metastasis is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarian tumor or nephroblastoma such as Wilms tumor.

In a specific set of embodiments, the tubular shaped elongated catheter device assembly, the tubular shaped device assembly or the multilumen tubular device as defined above, or the tubular shaped elongated catheter device assembly for use, the tubular shaped device assembly for use or the multilumen tubular device for use as defined above is designed to be implanted into a luminal organ, preferably a blood vessel such as an artery, an elastic artery, a distributing artery, an arteriole, a capillary, a venule or a vein, preferably into vena cava or a tubular organ, or wherein said device is designed to be free floating or wherein said device is designed to be implanted percutaneously, via a minimal invasive implantation, endoscopically, laparoscopically, or transcutaneously. Further envisaged and preferred is a transvascular implantation. In certain embodiments, the device is designed to be transvascularly advanced and implanted at a target site. The device may accordingly be designed to have adequate dimensions for an advancement in the target tissue or region. It may, in further embodiments, be catheter based and preferably steerable. In a preferred embodiment, said device or device for use is implanted in a blood or lymphatic vessel downstream of an existing cancer site in a subject.

In yet another preferred embodiment, said device or device for use is implanted in close proximity to said existing cancer site.

In yet another preferred embodiment, said device or device for use is implanted in a blood vessel upstream of a tissue with a high risk of developing metastasis.

In yet another preferred embodiment, said device or device for use is implanted during and/or after the treatment of a subject with a therapeutic agent or during and/or after surgery removing a tumor load.

It is preferred that said treatment is an anti-cancer therapy.

In yet another embodiment, said device or device for use is implanted into a healthy subject or a subject showing no symptoms of a disease, preferably symptoms of cancer or metastasis.

It is also preferred that said device or device for use is implanted into a subject being at risk of developing cancer and/or metastasis.

In a further aspect, the present invention relates to a method of treating cancer and/or metastasis, comprising implanting a tubular shaped elongated catheter device assembly as defined herein above into a subject in need thereof.

In yet another aspect, the present invention relates to a method of preventing cancer and/or metastasis, comprising implanting a tubular shaped elongated catheter device assembly, a tubular shaped device assembly for intraluminal use or a multilumen tubular device as defined herein above into a healthy subject or a subject being at risk of developing cancer and/or metastasis.

A further aspect, the invention relates to a method of manufacturing a tubular shaped elongated catheter device assembly, a tubular shaped device assembly for intraluminal use or a multilumen tubular device as defined herein above.

In a preferred embodiment, the method comprises the step of providing a biological agent as defined herein above by expressing said biological agent as polypeptide in a suitable host cell, optionally further modifying the polypeptide by adding one or more elements selected from the group comprising sugar, branched or unbranched multiple sugar structures, alkynes, azides, streptavidin, biotin, amines, carboxylic acids, active esters, epoxides and aziridines.

In preferred embodiment of the invention in the context of the second main aspect the device is an elongated tubular catheter based device assembly, with an intraluminal segment carrying the distal tip of the device and an extraluminal segment carrying the proximal end of the device, comprising a flow directed balloon tipped vascular catheter, wherein the distal end of the intraluminal segment is comprised of a compliant balloon, the balloon being in fluid connection with the proximal end of the extraluminal segment, wherein portions of the intraluminal segment are expandable and interactive with elements of bodily fluids in the target area.

In another embodiment, the at least one expandable portion of the intraluminal segment is an inflatable balloon, wherein said expandable portion is designed to bind a tumor cell, a tumor cell exosome, and/or a tumor cell aggregate and/or an immune cell.

In a further preferred embodiment the cross-sectional area of the intraluminal segment is at least about 50% smaller than the cross-sectional area of the luminal target site.

In a further preferred embodiment the intraluminal segment comprises at least one expandable portion, preferably a reversibly expandable portion.

In yet another embodiment, the expandable portion is capable of increasing the interactive contact surface with the bodily fluid.

A further preferred embodiment relates to a method as described above wherein the expandable portion of the device at maximal expansion comprises a cross-sectional area of equal or less than about two thirds of the cross-sectional area of the luminal target site.

In yet another preferred embodiment, the expandable portions only partially covering the cross-sectional area of the luminal target are arranged sequentially along the longitudinal extension in a clock-wise orientation, preferably with more than 90° differences between the sequential positions.

In a further embodiment, the present invention relates to a method as defined herein above, wherein the device additionally comprises at least two localization markers, preferably opposite to each other.

It is particularly preferred that said localization markers are located at the starting and end point of a segment comprising chemical and/or biological agents.

In a further preferred embodiment said localization marker is a radiopaque marker, an ultra-sound marker, an MRT marker or a CT marker, preferably further comprising at least one sensor such as an optical sensor, an analyte detecting sensor, a thermal sensor or a flow sensor.

In another preferred embodiment the present invention relates to a method wherein the device comprises sections permitting free flow of bodily fluids without any obstructing structural elements at any given point along the longitudinal extension of the device.

In yet another preferred embodiment sections are comprised permitting at least 30%, preferably 50% cross-sectional unhindered flow of bodily fluids.

In an additional embodiment of the method of the present invention the device comprises sections which permit a free flow of bodily fluids, interrupted by one or more sections permitting an interaction with components of the bodily fluids, preferably a filtering by means of a membrane like component, wherein preferably the interactive sections spare cross-sectional areas of free flow.

It is particularly preferred that the tubular shaped elongated catheter device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is anchorable in a target vessel; (v) it is designed to fit into and be connected to a permanent device present in a target vessel as a shuttle docking to a receiving site.

The present invention also envisages that the proximal end of the extraluminal segment is connected to an extraluminal fixation element for temporary implantation and for fixation of the intraluminal device and prevention of intraluminal migration of the free floating device.

In yet another embodiment of the method of the present invention the device comprises a through lumen housing of the expandable portions of the device.

It is further preferred that the device is characterized by at least one wire lumen within at least the intraluminal segment of the device.

In yet another embodiment of the method the reversibly expandable segment of the device as defined herein is comprised of at least one expandable balloon surface having a capability for interaction with at least one component of the bodily fluid, wherein the expandable balloon is preferably arranged with the distal end of the intraluminal portion of the device.

In another embodiment of the method, the elongated tubular shape as defined above is provided by a memory shaped wire, preferably a flexible nitinol wire.

In a further embodiment of the method, the device as defined above comprises extensions of the wire in the form of circular loops or ellipsoids or non-straight longitudinal extensions.

Further envisaged is a method wherein the wherein the expandable portion of the device as defined above comprises a porous membranous surface.

In a further embodiment, said expandable portion comprises a filter membrane. It is preferred the filter membrane has one or more of the following properties: (i) it comprises pores, (ii) it is expandable; (iii) it is retrievable; (iv) it is disposed within or around an expandable body; (v) it is characterized by a memory shaped metallic or memory shaped polymer structure; (vi) it comprises a detachment mechanism cooperating between the expandable portion and an intraluminal segment of the device or (vi) it is self-expandable.

In yet another embodiment of the method, the device as defined above additionally comprises a longitudinally extending free floating microfilaments; or comprises an outer sheath concentrically disposed about the device, wherein the outer sheath and the device are movable relative to one another.

Also envisaged is a method wherein a device as defined above additionally comprises a downstream embolic debris filter, wherein the filter is preferably retrievable and/or has a pore diameter of about >100 μm.

In yet another preferred embodiment, the device as defined above comprises a longitudinally extending catheter.

Further envisaged by the present invention are methods relating to devices as defined above, wherein said filter membrane has at least one of following properties: (i) the filter membrane is attached to and arranged with the expandable portion of the device between its proximal and distal end; (ii) the filter membrane is a non permanent, retrievable filter membrane.

In particularly preferred embodiment, said pores have a pore diameter which ranges from about 25 nm to about 100 μm, more preferably in a differential manner such as comprising differential ranges of 25 nm to 100 nm, 100 nm to 10 μm, 10 μm to 25 μm, or 25 μm to 100 μm.

It is also preferred that said expandable portions are at least partially coated with said one or more chemical and/or biological agents.

In another embodiment, said device is provided in a tubular, onion like, pearl-chain-like, or a birds-nest like shape, or in any mixture of these shapes.

In a preferred embodiment, said tubular shape is provided by a memory shaped spiraling wire, which forms a tubular spiral.

In yet another preferred embodiment, the memory shaped spiraling wire is modified into a single spiral-like wire structure, characterized by incomplete wire circles and an interdigiting structure.

In a further preferred embodiment, said onion like or pearl-chain-like shape is provided by an elastic memory shape meshwork.

In another embodiment of the method according to the present invention, the device as defined herein above is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques such as X ray.

In yet another embodiment, said device is self-expandable.

In a further preferred embodiment, at least a portion of the device can be activated by balloon inflation.

In an additional preferred embodiment, the device comprises at least one docking element at the proximal end for retrieval.

It is particularly preferred that said expandable portion is composed of elastic, foldable polymer material such as polyurethane, or of micromeshes comprising ultrathin wires, metallic or polymeric material.

In specific embodiments of the method according to the present invention, at least one cross-sectional area of the tubular device body is at least partially covered by the filter membrane.

It is further preferred that the plane of the filter membrane as defined herein above is arranged perpendicular to the direction of the longitudinal axis of the device body. Alternatively, the plane of the filter membrane is in an angle which is non perpendicular to the longitudinal axis of the device body.

In yet another embodiment of the method according to the present invention, the device as defined herein above comprises at least two filter membranes, each of which incompletely covers the cross-sectional area, and which are arranged in tandem position along the longitudinal axis of the device body, preferably opposite to each other within the circumference of the device body or shifted in clock-wise orientation in case of more than two filter membranes in tandem position.

In yet another embodiment, said device comprises alternating non-completely covering filter membranes, preferably in a pearl-chain, onion type or birds-nest like shape.

In a further preferred embodiment, the filter membranes as mentioned herein have differential pore diameters and/or differential pattern. In a particularly preferred embodiment they range from about 25 nm to about 100 μm. In further embodiments two or more filter membranes have differential pore diameters and/or differential pattern, preferably ranging from about 25 nm to about 100 μm such as comprising differential pore diameter ranges of 25 nm to 100 nm, 100 nm to 10 μm, 10 μm to 25 μm, or 25 μm to 100 μm.

In a further embodiment of the method of the present invention, at least one filter membrane is fully or partially coated on its interior side; or on its exterior side; or on both sides with said one or more chemical and/or biological agents; or wherein said coating differs between different filter membranes.

In another embodiment said coating as mentioned above is a passive coating with one or more polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) or linear aliphatic polyesters.

It is further preferred that said passive coating adheres to the structural support material via an adhesive layer, preferably of sugar, starch, polyvinylalcohol or degradable products of these materials.

In a particularly preferred embodiment said one or more chemical and/or biological agents constitute an extracellular matrix-like structure. It is further preferred that said extracellular matrix-like structure is covalently or non-covalently bound to the passive coating and/or the device material.

The present invention also relates to an embodiment of the method, wherein said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof; preferably a protein mixture secreted by Engelbreth-Holm-Swarm (EHS) mouse sarcoma cells, Matrigel, BioCoat or GelTrex. It is particularly preferred that protein mixtures with human proteins are employed.

In another preferred embodiment of the claimed method, the device as mentioned herein provides an environment for circulating metastatic cells.

It is further preferred that the method relates to a device comprising a biological agent as an active coating, which is capable of binding to a tumor marker; or to a cell of the immune system or a corresponding immune cell marker. Particularly preferred are tumor markers which are specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma.

In a further particularly preferred embodiment said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I).

In yet another embodiment said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

It is particularly preferred that said homing factor is Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

Another embodiment of the present invention relates to a device as defined herein above, wherein said cell of the immune system is a CD8⁺ cell, a dendritic cell, a T cell, an engineered T cell, a B cell or an NK cell.

It is further preferred that said biological agent is linked to the passive coating of the device or to the structural support material via a spacer element.

In another preferred embodiment said linkage to the structural support material is binding to a metal ion resin, such as ion-NTA or ion-agarose.

In yet another preferred embodiment said spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

In another embodiment said spacer has a length of about 1 to 20 nm.

In yet another embodiment said spacer elements are provided in a density of 1 mg per cm² on the surface of the device.

In a further embodiment said biological agent comprises, essentially consists of, or consists of a binding domain capable of binding to a tumor marker.

It is further envisaged that said binding domain is a peptide or polypeptide molecule having a length of about 20 to about 250 amino acids, preferably of about 20 to about 120 amino acids. It is particularly preferred that said binding domain has a length of 20, 25, 30, 35, 40, 45, 50, 55, 60, 65, 70, 75, 80, 85, 90, 95, 100, 105, 110, 115 or 120 amino acids.

In another embodiment said biological agent additionally comprises one or more functional domains.

In a further embodiment, said biological agent is covalently or non-covalently connected to said spacer.

In a further embodiment, said connection is a linker element. It is preferred that said linker element is a peptide, preferably having a length of 1, 2, 3, 4, 5, 6, 7, 8, 9, 10, 11, 12, 13, 14, 15, 16, 17, 18, 19 or 20 amino acids.

In yet another embodiment of the method of the present invention, said biological agent is provided as linear or circular element or as an element composed of linear and circular parts, preferably as a linear or circular or partially linear/circular peptide or polypeptide, or a protein with secondary or tertiary structure conformation.

In a further embodiment said circular biological agent has or is part of a structure comprising a loop or a loop and a stem, such as a human Fc part; or of a linear structure, which is linked to said spacer element.

Also envisaged are methods, wherein said loop structure or linear structure comprises said biological agent at an exposed position allowing for the binding to a tumor marker or a tumor cell, a tumor cell exosome, and/or a tumor cell aggregate.

In a preferred embodiment of a method as defined herein said peptide has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined conjugated with stabilizing components such as PEG or lipids, preferably forming lipoglycopeptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, essentially comprises or consists of D-amino acids and/or L-amino acids; (xi) it partially comprises, essentially comprises or consists homo-amino acids such as beta-homo-amino acids, N-methyl amino acids, or alpha-methyl-amino acids; (xii) it partially comprises, essentially comprises or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide.

In further embodiments, said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

In a further preferred embodiment of the method of the present invention the part of the device which is retrievable comprises the filter membrane or part of it, or an embolic filter or part of it.

In a further embodiment of the method the recovering of cells and/or proteins from the device is performed ex vivo after 1, 2, 3, 4, 5, 6, 7 days, 2, 3, 4 weeks or 2, 3, 4, 5, 6, 7, 8, 9, 10, 11, 12 months of implantation time, or after a signal indicating a filling state at or beyond a predefined threshold is received or measured.

In yet another preferred embodiment of the method, the recovering of cells and/or proteins is performed subsequent to a tumor treatment, preferably for recovering any circulating tumor and/or circulating metastatic cell and/or motile part of tumor cells and/or aggregates of tumor cells and/or tumor cell derived exosomes which are still present in the circulation.

In a further preferred embodiment, the method comprises the cultivation of recovered cell(s).

In yet another preferred embodiment, the method comprises the molecular, chemical, histological and/or physical analysis of the recovered cell(s).

In yet another preferred embodiment, the method comprises the analysis of proteins and/or nucleic acids present in or on the recovered cell(s).

In a further a aspect the present invention relates to a cell, a cell aggregate or an exosome or part of any of the before mentioned, obtained from the method as defined herein above.

In a further aspect the present invention relates to a protein or nucleic acid obtained from the method of as defined herein above.

In a further aspect the present invention relates to a method of diagnosing cancer and/or metastasis, or of determining an increased likelihood for developing cancer and/or metastasis, and/or of determining the subject's metastatic immune status, preferably near a metastatic site comprising implanting a device as defined herein above into a subject in need thereof and detecting the presence of circulating tumor and/or circulating metastatic cell and/or motile part of tumor cells and/or aggregates of tumor cells and/or tumor cell derived exosomes and or immune cells captured by the device subsequent to the recovery of said device.

In yet another aspect the present invention relates to a method of diagnosing cancer and/or metastasis, or of determining an increased likelihood for developing cancer and/or metastasis, comprising implanting a device as defined herein above into a healthy subject, a subject showing no symptoms of a disease, preferably symptoms of cancer or metastasis, or a subject being at risk of developing cancer and/or metastasis and detecting the presence of circulating tumor and/or circulating metastatic cell and/or motile part of tumor cells and/or aggregates of tumor cells and/or tumor cell derived exosomes captured by the device.

In a further aspect the present invention relates to a method of monitoring the effect of a disease treatment, preferably cancer treatment, comprising implanting a device as defined herein above into a patient currently being treated for a disease, preferably cancer and/or metastasis, or a patients who has finished his disease treatment, preferably cancer and/or metastasis treatment, within a time period of about 1 week to 2 years and subsequently recovering said device.

In yet another aspect the present invention relates to a method of data collection comprising (i) monitoring a device as defined herein above implanted in a subject via ultrasound scanning, tomography, optical, or by determining the device environment for changes indicative of a disease; and (ii) collecting data over time for recognizing changes of the monitored values.

In yet another aspect the present invention relates to a method of identifying a target cell, target protein or target nucleic acid, comprising analyzing a cell, a cell aggregate or an exosome or part of any of the before mentioned as, or a protein or nucleic acid.

In a preferred embodiment of the method, the method comprises a recovering of cells, aggregates of cells and/or tumor cell derived exosomes and/or proteins and/or nucleic acids and/or recruited immune cells from the device after a time period of about 1 day to 2 years.

In another preferred embodiment, said recovered cells are sequenced and/or biochemically analyzed and/or compared with previous data or database values on recovered cells to provide a disease status profile.

It is further envisaged that said method comprises a step of prognosticating the length and/or outcome of the treatment.

Also preferred, in a further embodiment, is the method as defined above comprising an additional step of adjusting a disease treatment strategy to the diagnostic or target identification values obtained, preferably by modifying the type and/or amount of pharmaceutical agent to be administered.

In a preferred embodiment of the invention in the context of the third main aspect the read out and/or monitoring is performed via ultrasound scanning, tomography, optical analysis, and/or by electrochemical measurements, and/or by determining the implant environment for changes indicative of a disease.

In a further embodiment, the device has a distal and a proximal end and comprises an intraluminal distal segment and an extraluminal proximal segment, wherein the intraluminal segment comprises at least one expandable cross-sectional area which in its expanded state is smaller than the cross-sectional area of the luminal target site and wherein at least the expandable portion of the intraluminal segment is capable of interacting with at least one component of the bodily fluid via an interactive contact surface.

In a further preferred embodiment the catheter device is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

In a further preferred embodiment the at least one expandable portion of the intraluminal segment is an inflatable balloon, wherein said expandable portion is designed to bind a tumor cell, a tumor cell exosome, and/or a tumor cell aggregate, and/or an immunologic cell such as a T cell, B cell or dendritic cell, and/or an antibody complex such as an autoantibody complex, and/or a neurologic serum marker protein.

In yet another embodiment, the cross-sectional area of the intraluminal segment is at least about 50% smaller than the cross-sectional area of the luminal target site.

A further preferred embodiment relates to a device as defined above, wherein the intraluminal segment comprises at least one expandable portion, preferably a reversibly expandable portion.

In yet another preferred embodiment, the expandable portion is capable of increasing the interactive contact surface with the bodily fluid.

In a further embodiment, the expandable portion at maximal expansion comprises a cross-sectional area of equal or less than about two thirds of the cross-sectional area of the luminal target site.

In a further preferred embodiment the expandable portions only partially covering the cross-sectional area of the luminal target are arranged sequentially along the longitudinal extension in a clock-wise orientation, preferably with more than 90° differences between the sequential positions.

In another preferred embodiment the device additionally comprises at least two localization markers, preferably opposite to each other.

In a particularly preferred embodiment said localization markers are located at the starting and end point of a segment comprising chemical and/or biological agents.

In an additional embodiment said localization marker is a radiopaque marker, an ultrasound marker, an MRT marker or a CT marker.

In a further preferred embodiment, the device comprises sections permitting free flow of bodily fluids without any obstructing structural elements at any given point along the longitudinal extension of the device.

It is particularly preferred that the device comprises sections permitting at least 30%, preferably 50% cross-sectional unhindered flow of bodily fluids.

In a further embodiment, the device comprises sections which permit a free flow of bodily fluids, interrupted by one or more sections permitting an interaction with components of the bodily fluids, preferably a filtering by means of a membrane like component, wherein preferably the interactive sections spare cross-sectional areas of free flow.

It is particularly preferred that the tubular shaped elongated catheter device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is anchorable in a target vessel; (v) it is designed to fit into and be connected to a permanent device present in a target vessel as a shuttle docking to a receiving site. It is further preferred that the device is atraumatically anchorable in a target vessel.

The present invention also envisages that the proximal end of the extraluminal segment is connected to an extraluminal fixation element for temporary implantation and for fixation of the intraluminal device and prevention of intraluminal migration of the free floating device.

In yet another embodiment the device comprises a through lumen housing of the expandable portions of the device.

It is further preferred that the device is characterized by at least one wire lumen within at least the interluminal segment of the device.

In yet another embodiment the reversibly expandable segment of the device is comprised of at least one interactive expandable balloon surface having a capability for interaction with at least one component of the bodily fluid, wherein the expandable balloon is preferably arranged with the distal end of the intraluminal portion of the device and wherein the interactive balloon may preferably also serve as a flow directing balloon.

In another embodiment, the elongated tubular shape is provided by a memory shaped wire, preferably a flexible nitinol wire.

In a further embodiment, the device comprises extensions of the wire in the form of circular loops or ellipsoids or non-straight longitudinal extensions.

Further envisaged is a device as defined above, wherein the expandable portion comprises a porous membranous surface.

In a further embodiment, said expandable portion comprises a filter membrane.

It is preferred the filter membrane has one or more of the following properties: (i) it comprises pores, (ii) it is expandable; (iii) it is retrievable; (iv) it is disposed within or around an expandable body; (v) it is characterized by a memory shaped metallic or memory shaped polymer structure; (vi) it comprises a detachment mechanism cooperating between the expandable portion and an intraluminal segment of the device or (vi) it is self-expandable.

In yet another embodiment, the device additionally comprises a longitudinally extending free floating microfilaments; or comprises an outer sheath concentrically disposed about the device, wherein the outer sheath and the device are movable relative to one another.

Also envisaged is a device as defined above, which additionally comprises a downstream embolic debris filter, wherein the filter is preferably retrievable and/or has a pore diameter of >100 μm.

In yet another preferred embodiment, the device as defined above comprises a longitudinally extending catheter.

Further envisaged by the present invention are devices as defined above, wherein said filter membrane has at least one of following properties: (i) the filter membrane is attached to and arranged with the expandable portion of the device between its proximal and distal end; (ii) the filter membrane is a non permanent, retrievable filter membrane.

In a particularly preferred embodiment, said pores have a pore diameter which ranges from about 25 nm to about 100 μm, more preferably in a differential manner such as comprising differential ranges of 25 nm to 100 nm, 100 nm to 10 μm, 10 μm to 25 μm, or 25 μm to 100 μm.

It is also preferred that said expandable portions are at least partially coated with said one or more chemical and/or biological agents.

In another embodiment, said device is provided in a tubular, onion like, pearl-chain-like, or a birds-nest like shape, or in any mixture of these shapes.

In a preferred embodiment, said tubular shape is provided by a memory shaped spiraling wire, which forms a tubular spiral.

In yet another preferred embodiment, the memory shaped spiraling wire is modified into a single spiral-like wire structure, characterized by incomplete wire circles and an interdigiting structure.

In a further preferred embodiment, said onion like or pearl-chain-like shape is provided by an elastic memory shape meshwork.

In another embodiment of the present invention, the device as defined herein above is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques such as X ray.

In yet another embodiment, said device is self-expandable.

In a further preferred embodiment, at least a portion of the device can be activated by balloon inflation.

In an additional preferred embodiment, the device comprises at least one docking element at the proximal end for retrieval.

It is particularly preferred that said expandable portion is composed of elastic, foldable polymer material such as polyurethane, or of micromeshes comprising ultrathin wires, metallic or polymeric material.

In specific embodiments, at least one cross-sectional area of the tubular device body is at least partially covered by the filter membrane.

It is further preferred that the plane of the filter membrane as defined herein above is arranged perpendicular to the direction of the longitudinal axis of the device body. Alternatively, the plane of the filter membrane is in an angle which is non perpendicular to the longitudinal axis of the device body.

In yet another embodiment, the device as defined herein above comprises at least two filter membranes, each of which incompletely covers the cross-sectional area, and which are arranged in tandem position along the longitudinal axis of the device body, preferably opposite to each other within the circumference of the device body or shifted in clockwise orientation in case of more than two filter membranes in tandem position.

In yet another embodiment, said device comprises alternating non-completely covering filter membranes, preferably in a pearl-chain, onion type or birds-nest like shape.

In a further preferred embodiment, the filter membranes as mentioned herein have differential pore diameters and/or differential pattern. In a particularly preferred embodiment they range from about 25 nm to about 100 μm. In further embodiments two or more filter membranes have differential pore diameters and/or differential pattern, preferably ranging from about 25 nm to about 100 μm such as comprising differential pore diameter ranges of 25 nm to 100 nm, 100 nm to 10 μm, 10 μm to 25 μm, or 25 μm to 100 μm.

In a further embodiment, at least one filter membrane is fully or partially coated on its interior side; or on its exterior side; or on both sides with said one or more chemical and/or biological agents; or wherein said coating differs between different filter membranes.

In another embodiment said coating is a coating with one or more conductive materials.

It is further preferred that said conductive materials are electro-active polymeric (EAP) materials.

In a particularly preferred embodiment said coating with conductive materials is a partial coating, preferably a coating covering between 5% and 95% of the surface of the device.

In a particularly preferred embodiment said coating is provided in the form of symmetrically distributed areas on the surface of the device, preferably within pores.

The present invention also relates to an embodiment, wherein said EAP material coating comprises one or more areas of insulator (about 10⁻⁹ to 10⁻¹² S cm⁻¹), semi-conductive (about 10 to 10⁻⁹ S cm⁻¹) and/or or purely conductive (about 10⁶ to 10 S cm⁻¹) EAP materials.

It is particularly preferred that said EAP materials comprise poly(acetylene) (PAc), poly (p-vinylene) (PPV), poly (p-phenylene) (PPP), poly (γ-phenylene sulphide) (PPS), polypyrrole (PPy), polyaniline (PANI), polythiophene (PTh), poly (3,4-ethylenedioxythiophene) (PEDOT), emeraldine base polyaniline (EB-PANI), polypyrrole/graphene (PYG), poly(3,4-ethylenedioxythiophene) poly(styrenesulfo-nate) (PE-DOT:PSS) and/or poly(isothianaphtene) (PITN).

In another preferred embodiment, said EAP materials are doped or non-doped.

In another preferred embodiment, the coating is provided on porous filter material, additionally comprising co-porogenes such as NaCl crystals and PEG powder.

It is further preferred that said device additionally comprises one or more substrate electrodes, preferably composed of platinum, glassy carbon, gold, SnO₂, metallized plastics, carbon fibers or TiO₂.

In a further preferred embodiment the device comprises, at least in some areas, additionally a passive coating with one or more non electroactive polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) am linear aliphatic polyesters.

In yet another embodiment said passive coating adheres to the structural support material via an adhesive layer, preferably of sugar, starch, polyethylene or degradable products of these materials.

In yet another preferred embodiment, said one or more chemical and/or biological agents constitute an extracellular matrix-like structure layer, wherein said extracellular matrix-like structure layer is preferably located above or in juxtaposition to said EPA materials or said layer composed of said EPA materials.

In yet another preferred embodiment said extracellular matrix-like structure is covalently or non-covalently bound to the coating and/or the device material, wherein preferably a layer composed of EPA materials is pervaded by elements implementing the covalent or non-covalent binding to the coating and/or the device.

It is further preferred that said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof, or integrins; preferably a protein mixture secreted by Engelbreth-Holm-Swarm (EHS) mouse sarcoma cells, Matrigel, BioCoat or GelTrex. Particularly preferred are protein mixtures with substitutions of human proteins.

Another embodiment of the present invention relates to a device as defined herein above, wherein the device comprises a biological agent as an active coating, which is capable of binding to a tumor marker and/or a biological agent as an active coating, which is capable of binding to an immunologic receptor or interactor or to a cell of the immune system.

In a preferred embodiment, the tumor marker is specific for breast tumors, prostate tumors, pancreas tumors, colon tumors, small cell lung tumors, lymphoma, multiple lymphoma, T-cell tumors, Mycosis fungoides, melanoma, neuroblastoma, sarcoma, fibrosarcoma, Wilms tumor or Squamous cell carcinoma

It is particularly preferred that said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I).

In another preferred embodiment said cell of the immune system is a CD8⁺ cell, a dendritic cell, a T cell, an engineered T cell, a B cell or an NK cell.

In another preferred embodiment said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

In yet another preferred embodiment said homing factor is Osteopontin, Hyaluronate, CXCL12, CCL21, Dipeptidyl Dipeptidase IV, PECAM-1, Bone Sialoprotein, Peripheral Node Addressin (CD34), MAD-CAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

In a further preferred embodiment said biological agent is linked to the coating of the device or to the structural support material via a spacer element.

In yet another preferred embodiment said linkage to the structural support material is binding to a metal ion resin, such as ion-NTA or ion-agarose.

In a further preferred embodiment, said spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

In another embodiment said spacer has a length of about 1 to 20 nm.

In yet another embodiment said spacer elements are provided in a density of 1 mg per cm² on the surface of the device.

In a further embodiment said biological agent comprises, essentially consists of, or consists of a binding domain capable of binding to a tumor marker or a binding domain capable of binding to an immunologic receptor or interactor or to a cell of the immune system.

It is further envisaged that said binding domain is a peptide or polypeptide molecule having a length of about 20 to about 250 amino acids, preferably of about 20 to about 120 amino acids. It is particularly preferred that said binding domain has a length of 20, 25, 30, 35, 40, 45, 50, 55, 60, 65, 70, 75, 80, 85, 90, 95, 100, 105, 110, 115 or 120 amino acids.

In another embodiment said biological agent additionally comprises one or more functional domains.

In a further embodiment, said biological agent is covalently or non-covalently connected to said spacer.

In a further embodiment, said connection is a linker element. It is preferred that said linker element is a peptide, preferably having a length of 1, 2, 3, 4, 5, 6, 7, 8, 9, 10, 11, 12, 13, 14, 15, 16, 17, 18, 19 or 20 amino acids.

In yet another embodiment, said biological agent is provided as linear or circular element or as an element composed of linear and circular parts, preferably as a linear or circular or partially linear/circular peptide or polypeptide.

In a further embodiment said circular biological agent has or is part of a structure comprising a loop or a loop and a stem, such as a human Fc part; or of a linear structure, which is linked to said spacer element.

Also envisaged are devices as defined herein above, wherein said loop structure or linear structure comprises said biological agent at an exposed position allowing for the binding to a tumor marker or a tumor cell, a tumor cell exosome, and/or a tumor cell aggregate or for the binding to an immunologic receptor or interactor or a cell of the immune system, and/or for the binding of an antibody complex such as an autoantibody complex, and/or a neurologic serum marker protein.

In a preferred embodiment of a device as defined herein above, said peptide has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined conjugated with stabilizing components such as PEG or lipids, preferably forming lipoglycopeptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, essentially comprises or consists of D-amino acids and/or L-amino acids; (xi) it partially comprises, essentially comprises or consists homo-amino acids such as beta-homo-amino acids, N-methyl amino acids, or alpha-methyl-amino acids; (xii) it partially comprises, essentially comprises or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide.

In a further preferred embodiment said loop or linear structure comprises additional elements selected from the group comprising a sensor unit or an interaction unit, preferably an external interaction unit.

In a particularly preferred embodiment, said sensor unit is a conformation sensor capable of changing its conformation upon binding of a ligand to the biological agent and of (i) conveying this conformation change to a receiving unit, which is close to the spacer element, the extracellular matrix-like structure, the coating of the device and/or on the surface of the implant, or of (ii) emitting light or heat upon said conformational change.

In a further preferred embodiment, said conformational change is converted into a sensed signal which is transmitted from the device to the environment.

In further embodiments, said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

Also envisaged are devices as defined herein above, are partially composed of or comprises material selected from the group comprising material readable by tomography or suitable for electrochemical or electronic readouts.

In a preferred embodiment, said device further comprises, is combined with or is designed to be combinable with one or more elements selected from (i) an electrical electrode, (ii) a microprocessor or circuit component, (iii) a read-out module, (iv) a digital domain, (v) a controller component and (vi) a communication module.

In yet another preferred embodiment, said read-out module is composed of an analog front-end (AFE) and an analog-to-digital converter (ADC), capable of capturing and processing the sensed signal and converting it to the digital domain.

In yet another preferred embodiment, said sensed signal is processed by a digital signal processor (DSP).

It is also preferred that the communication module provides communication between the device and an outside receiving module.

In a preferred embodiment, said communication module operates with a wireless transceiver, preferably Bluetooth, a radiofrequency (RF) component, a WLAN or WiFi component, or with an optic fiber or a cable connection, or on the basis of electric resistance measurements.

In another preferred embodiment, said electric resistance measurement is based on an electric signal which is provided to the device, preferably from the outside, and which elicits a signal in dependence on the electrical current conduction and resistance in the device, wherein an increased resistance is indicative for the presence of bound cells, cell aggregates, exosomes or parts thereof.

In another preferred embodiment, said device is connected to an optic fiber or a cable allowing for a read-out from outside of a subject's body.

In yet another preferred embodiment, said read-out module is capable of registering device parameters with regard to one or more properties selected from the group comprising oxygen content, sugar content, temperature, ion concentration, impedance, conductivity, pH, pressure, colors, color changes or color pattern, fluorescence, or bioluminescence.

In a further embodiment the outside receiving module is, or is comprised or integrated in, or associated with: a mobile phone, a computer, a tablet, a handheld device, or a network server, or a network cloud computing device, preferably a hand-held physiological signal measurement device or hand-held radio transmission and receiving device.

In a specific embodiment, said hand-held physiological signal measurement device or hand-held radio transmission and receiving device comprises one or more of the following: (i) a housing, (ii) a plurality of electrodes attached to the surface of the housing, (iii) a contact with the skin surface of the subject, obtaining from the user a physiological signal's; (iv) a front-end circuit, preferably located inside the housing and connected to the plurality of electrodes to receive the physiological signal; (v) an analog/digital conversion circuit, preferably located inside the housing and connected to the front end circuit; (vi) a wireless transceiver interface located inside the housing; (vii) a processing unit preferably located in the inner housing, which is connected to the analog/digital conversion circuit and a transceiver.

In a further embodiment said receiving module is connected with a data processing system.

In further preferred embodiment said data processing system comprises a program capable of collecting data sent by said communication module over time and/or of analyzing or processing said data.

In yet another preferred embodiment +said program is additionally capable of representing said data graphically and/or comparing said data with one or more reference values and/or wherein said program is capable of comparing a threshold value, preferably derived from background noise, with a measured electrical current value, thereby monitoring clinically relevant events and transforming them into a signal which is being read from the external signal receiving module.

In a further embodiment said device additionally comprises a drug release module.

In yet another preferred embodiment said release module is controllable by the communication module. It is particularly preferred that said drug release module is controllable by heat, electrical, magnetic or ultrasound stimuli.

According to a further preferred embodiment said drug release module comprises one or more pharmaceutically active compounds such as an anti-cancer drug, an anti-thrombotic drugs, a cardiovascular drug, a drug against a neurologic disease, preferably Alzheimer's disease.

In a further aspect the present invention relates to a tubular shaped elongated catheter device assembly as defined herein above for use in diagnosing and/or monitoring a disease, preferably cancer, tumor metastases, a cardiovascular disease, or for determining an increased likelihood for developing a disease, preferably cancer, metastases, a cardiovascular disease, or a neurologic disease such as Alzheimer's disease in a subject.

In a further aspect the present invention relates to tubular shaped elongated catheter device assembly as defined herein above for use in treating cancer and/or metastasis, preferably in luminal organs such as blood vessels.

In yet another aspect the present invention relates to a tubular shaped elongated catheter device assembly as defined herein above for use in preventing a disease, preferably cancer, metastases, cardiovascular diseases, or neurologic diseases.

In yet another aspect the present invention relates to a tubular shaped elongated catheter device assembly as defined herein above use in treating a disease, preferably cancer, metastases, a cardiovascular disease, or a neurologic disease.

In a preferred embodiment said cancer is colon cancer, breast cancer, lung cancer, melanoma, esophageal cancer, prostate cancer, myeloma, lymphoma such as ALL, CLL, AML, cervix carcinoma, renal carcinoma, urinary bladder carcinoma or brain tumors. In a further preferred embodiment, said metastasis is derived from a colon cancer, breast cancer, lung cancer, or melanoma.

In preferred embodiments, said device is designed to be implanted into a luminal organ, preferably a blood vessel such as an artery, an elastic artery, a distributing artery, an arteriole, a capillary, a venule or a vein or wherein said device is designed to be implanted percutaneously, via a minimal invasive surgical implantation, via a transvascular or transluminal approach, endoscopically or laparoscopically. Further envisaged and preferred is a transvascular implantation. In certain embodiments, the device is designed to be transvascularly advanced and implanted at a target site. The device may accordingly be designed to have adequate dimensions for an advancement in the target tissue or region. It may, in further embodiments, be catheter based and preferably steerable. It is particularly preferred that the device is steerable is by its torque stable structure or by comprising a steerable wire.

In a preferred embodiment, said device is implanted in a blood or lymphatic vessel downstream of an existing cancer site in a subject.

In a further preferred embodiment, said device is implanted in close proximity to said existing cancer site.

In a further preferred embodiment, said device is implanted in a blood vessel upstream of a tissue with a high risk of developing metastasis.

In yet another preferred embodiment, said device is implanted during and/or after the treatment of a subject with a therapeutic agent or during and/or after surgery which removes a tumor load.

It is particularly preferred that said treatment is an anti-cancer therapy.

In yet another preferred embodiment, said device is implanted into a healthy subject or a subject showing no symptoms of a disease, preferably symptoms of cancer or metastasis.

In yet another preferred embodiment said device is implanted into a subject being at risk of developing cancer and/or metastasis.

In a further aspect the present invention relates to a method of diagnosing or monitoring a disease such as cancer and/or metastasis and/or a cardiovascular disease, or of determining an increased likelihood for developing a disease such as cancer and/or metastasis or a cardiovascular disease comprising implanting a tubular shaped elongated catheter device assembly as defined herein above into a subject in need thereof and detecting the presence of cancer and/or metastatic cells captured by the implant.

In a further aspect the present invention relates to a method of diagnosing or monitoring a disease such as cancer and/or metastasis and/or a cardiovascular disease, or of determining an increased likelihood for developing a disease such as cancer and/or metastasis and/or a cardiovascular disease comprising implanting a tubular shaped elongated catheter device assembly as defined herein above into a healthy subject, a subject showing no symptoms of a disease, preferably symptoms of cancer or metastasis or a cardiovascular disease, or a subject being at risk of developing a disease such as cancer and/or metastasis or a cardiovascular disease and detecting the presence of cancer and/or metastatic cells captured by the device

In yet another aspect the present invention relates to a method of monitoring the effect of disease treatment, comprising implanting a tubular shaped elongated catheter device assembly as defined herein above into a patient currently being treated for a disease such as cancer and/or metastasis and or a cardiovascular disease, or a patients who has finished his disease treatment such as cancer and/or metastasis or cardiovascular disease or neurologic disease treatment within a time period of about 1 day to 2 years.

In final aspect the present invention relates to a method of data collection comprising (i) monitoring a tubular shaped elongated catheter device assembly as defined in any one of claims 1 to 106 implanted in a subject via ultrasound scanning, tomography, optical, or electrochemical measurements, or by determining the tubular shaped elongated catheter device assembly environment for changes indicative a disease electrochemical measurements; and (ii) collecting data over time for recognizing changes of the monitored values.

### BRIEF DESCRIPTION OF THE FIGURES

FIGS. 1 to 48 show embodiments of the present invention.

FIG. 49A shows CRL-1918 spheroids without the use of the Taxus Liberté stent. FIG. 49B shows CRL-1918 spheroids after use of Taxus Liberté stent.

FIG. 50 shows a device comprising three onion-like device bodies in tandem positions.

FIG. 51 shows an implant comprising a pearl-chain-like device body with differential pearl size diameters.

FIG. 52 shows an implant comprising a pearl-chain-like device body with tandem filters with different membrane locations.

FIGS. 53A and B show different orientations of a filter membrane vis-à-vis the longitudinal axis of the implant body.

FIG. 54 shows an implant wherein cross-sectional planes of the circular loops or ellipsoids are oriented parallel to each other or may be oriented in an angle to each other.

FIG. 55 shows a Filterwire (Boston Scientific) device comprising filters resembling umbrellas on a wire with uniform pores of 80 um-110 um in diameter.

FIG. 56 shows an implant comprising a retrievable embolic filter.

FIG. 57 shows a bioactive coated implant comprising a microfilament assembly. The assembly is shown in 3 different versions, i.e. an open, closed and spiral version (1, 2 and 3). The figure further indicates the application of the catheter for placement and retrieval of the assembly.

FIG. 58 depicts the human artery system.

FIGS. 69 to 61 show examples of clinical uses of the device according to the present invention.

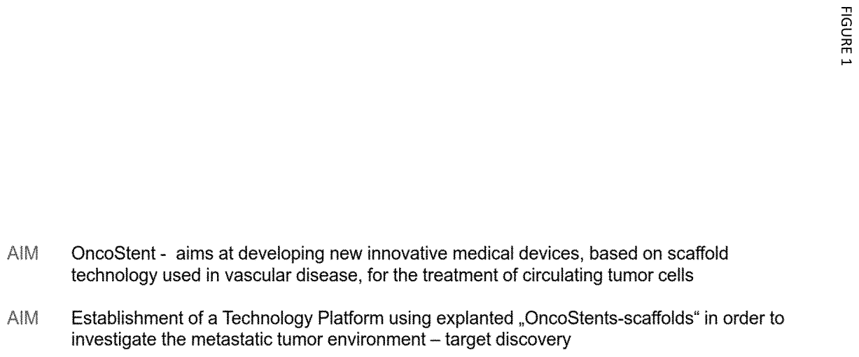

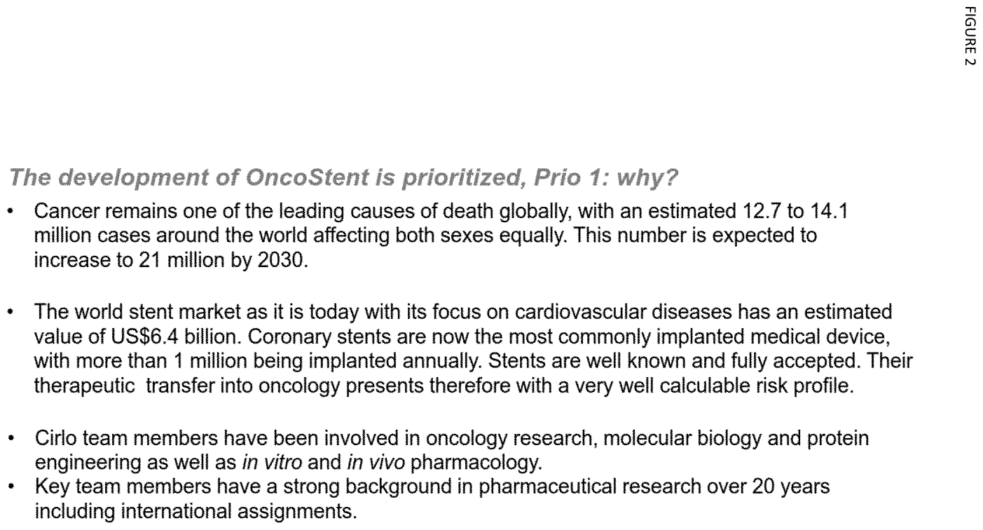

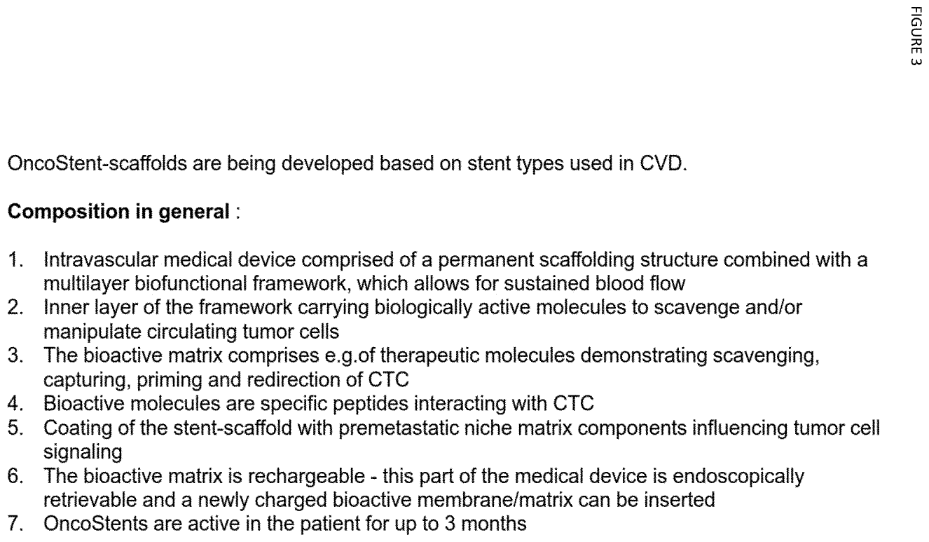

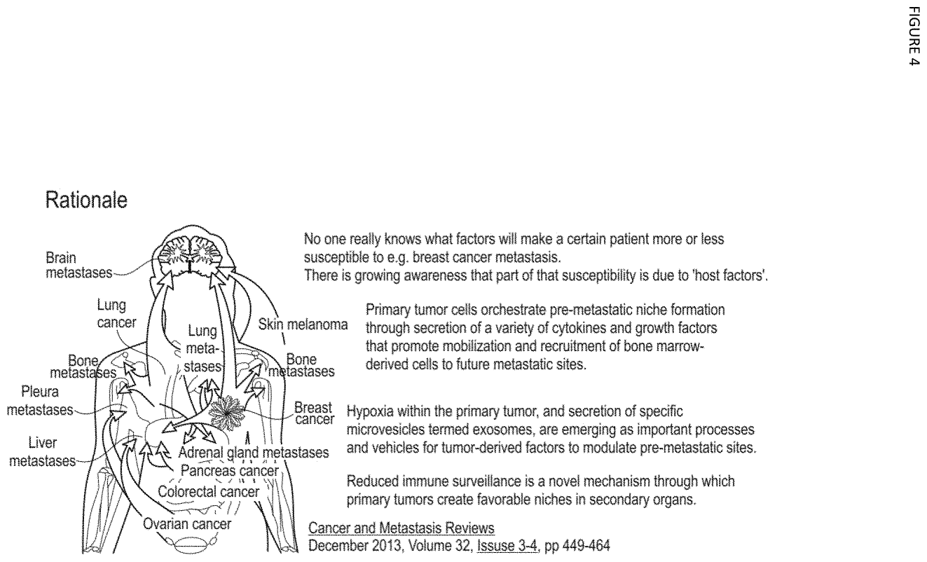

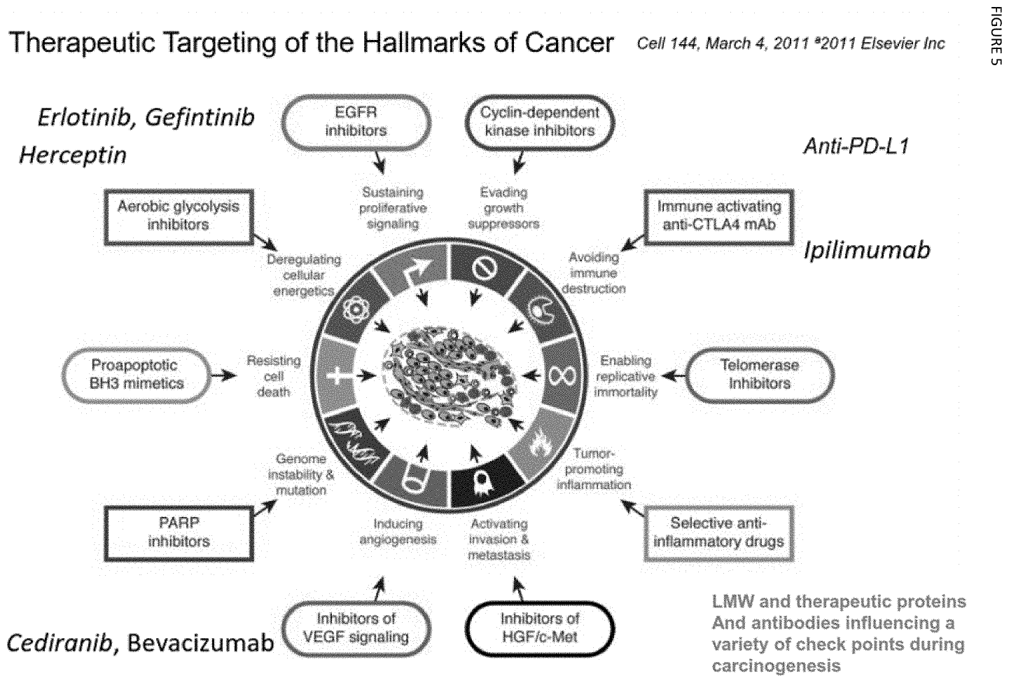

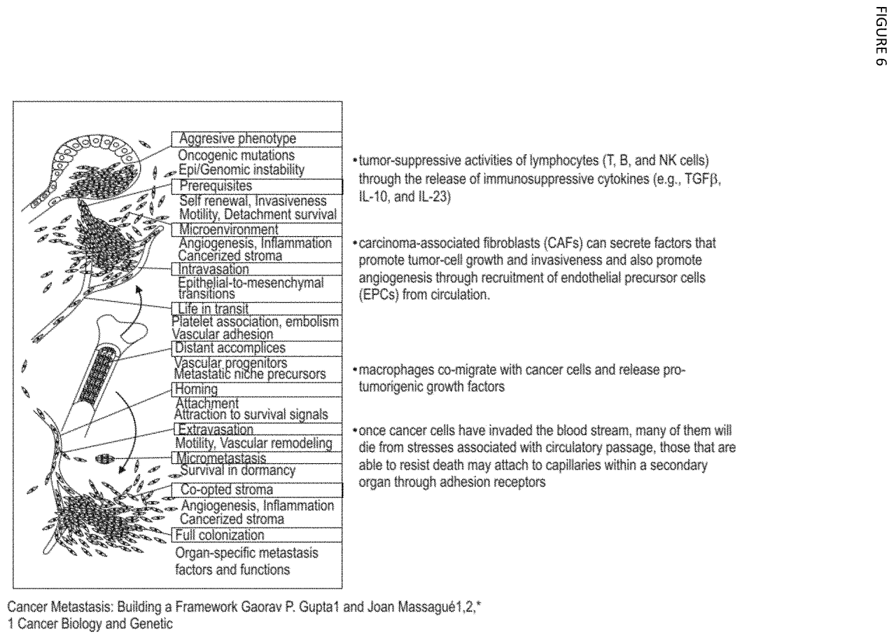

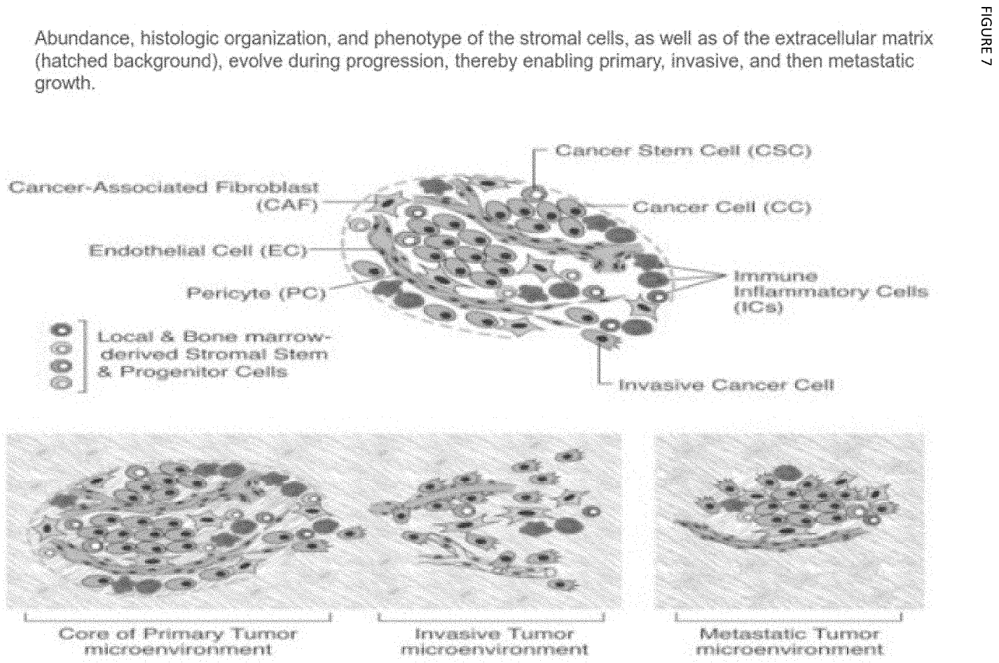

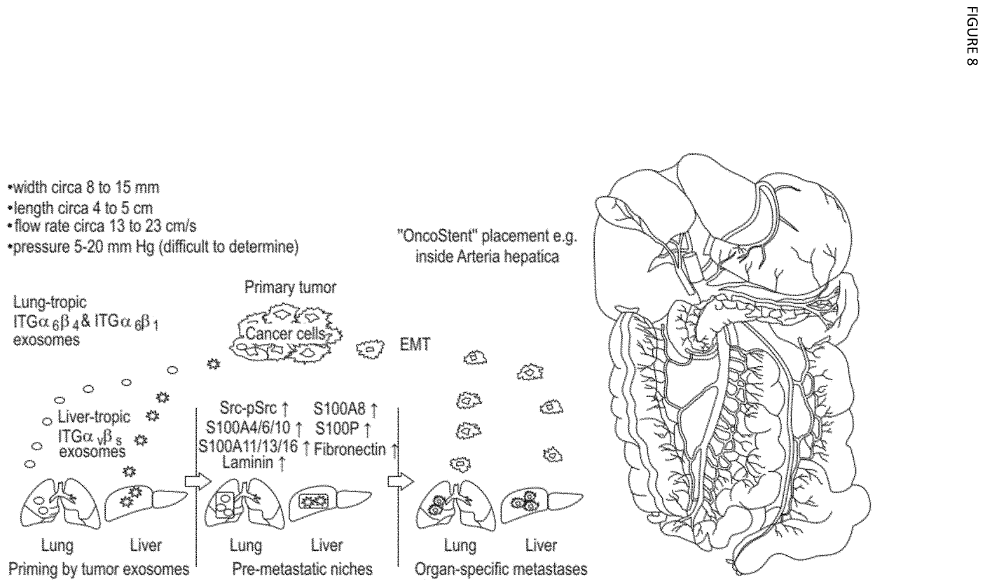

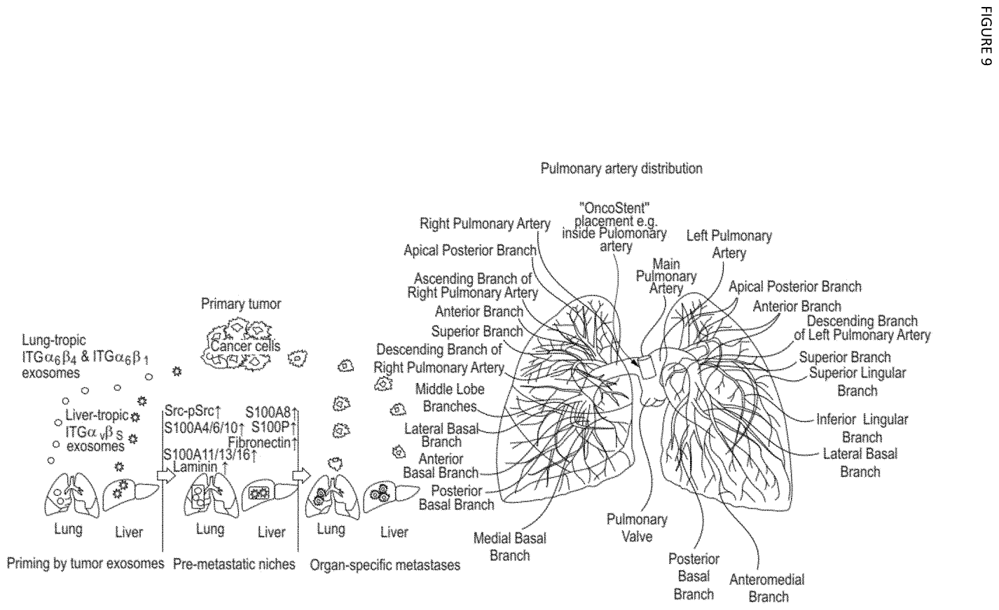

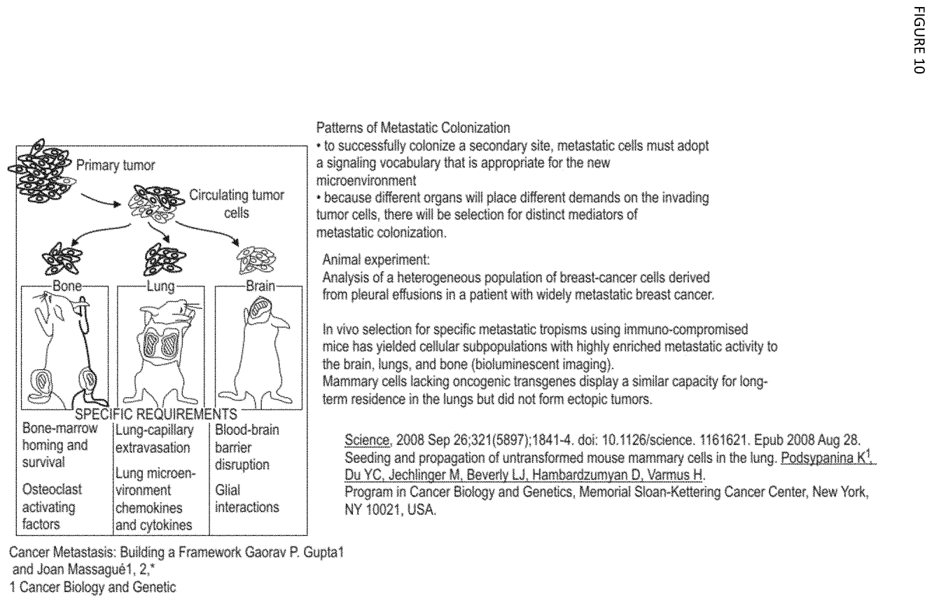

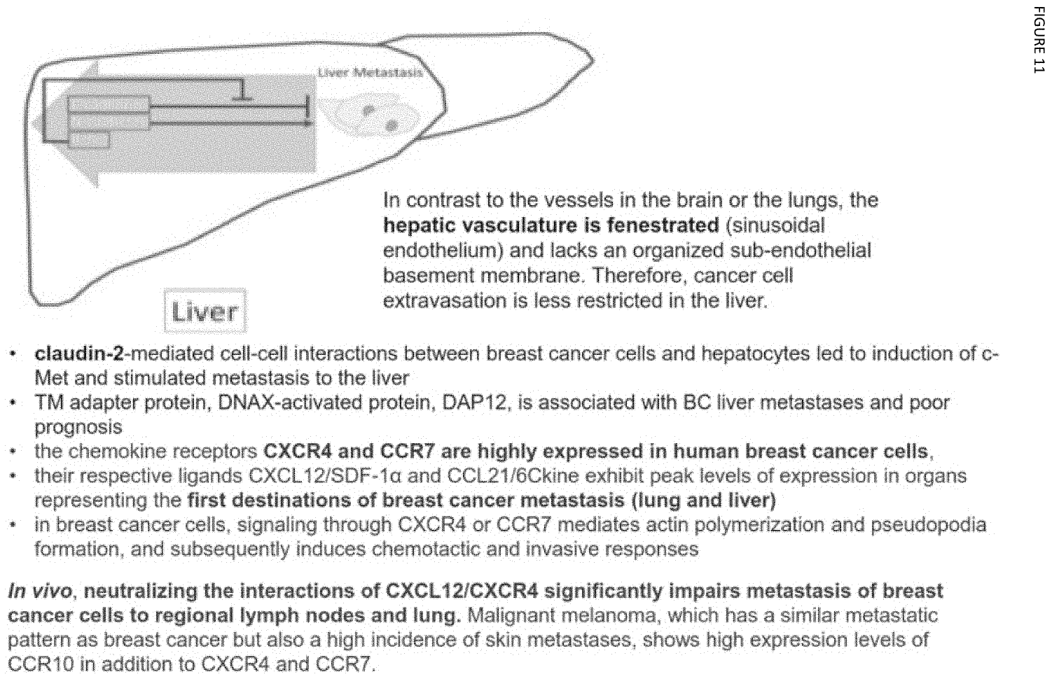

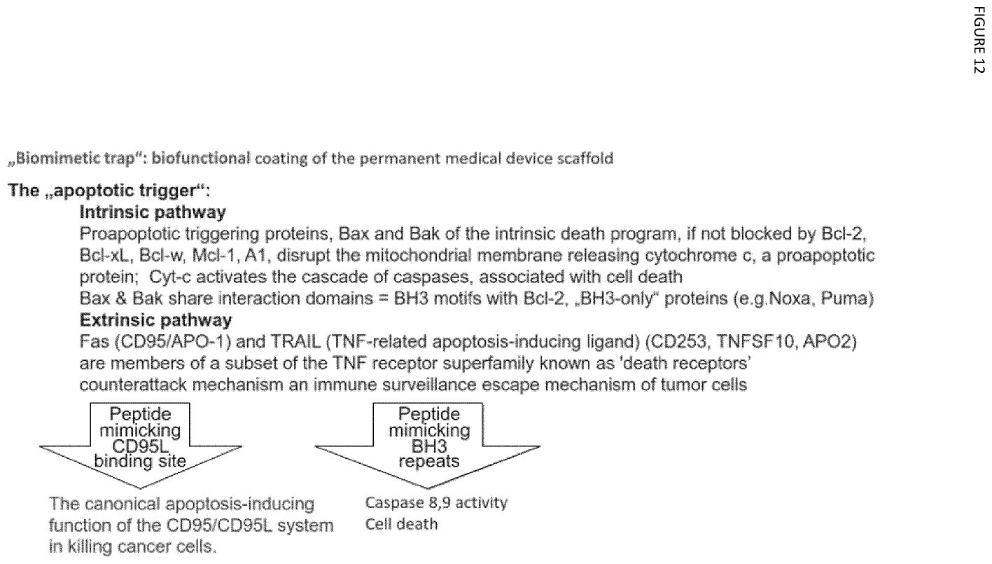

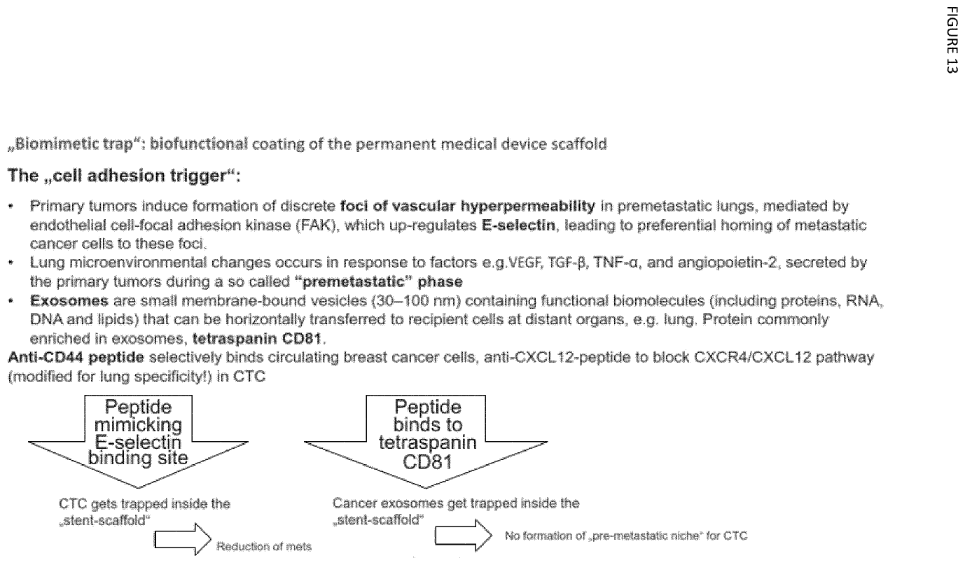

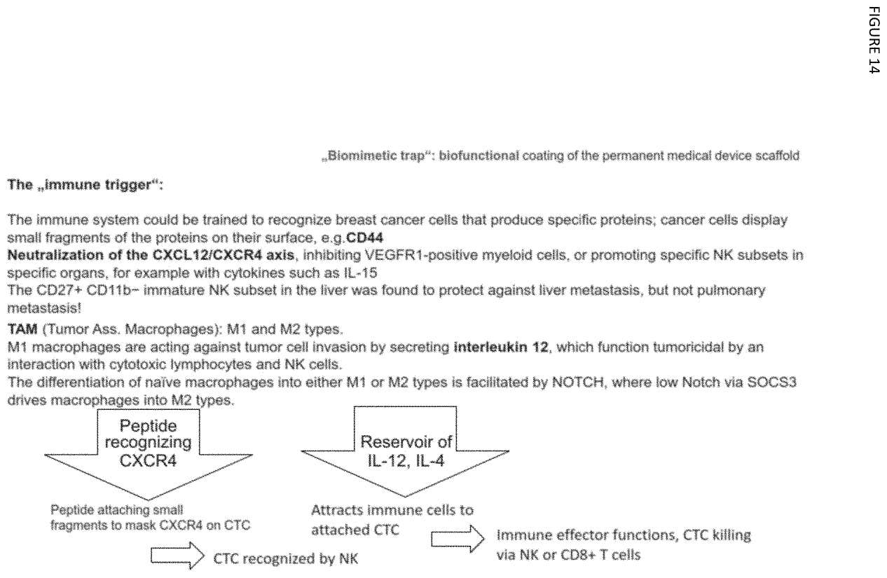

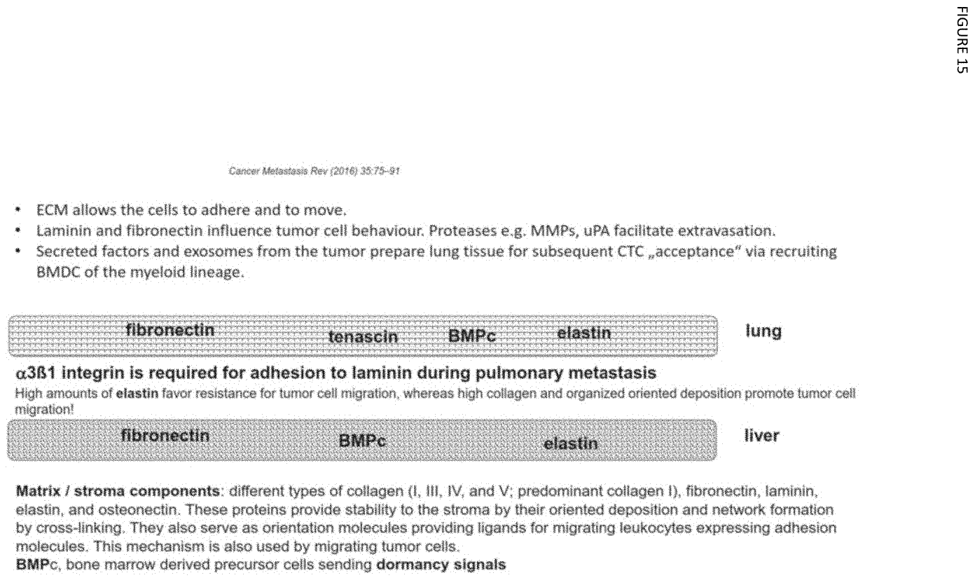

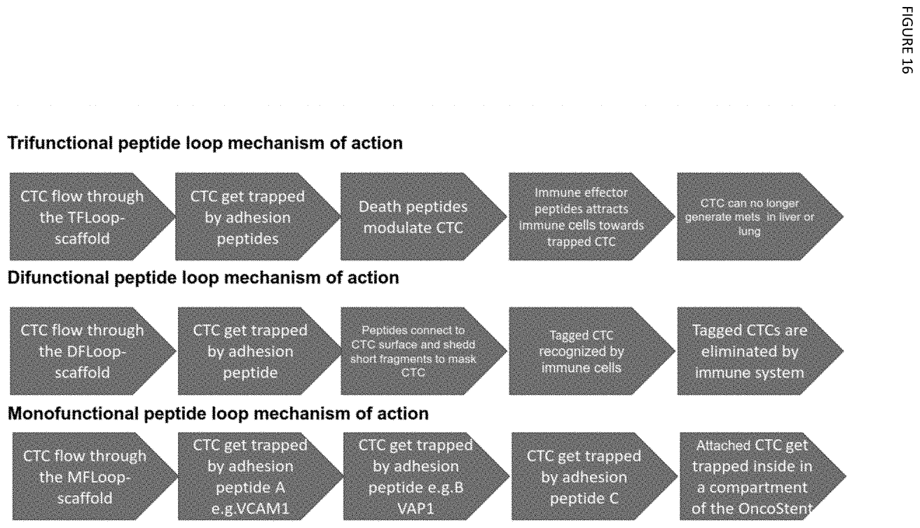

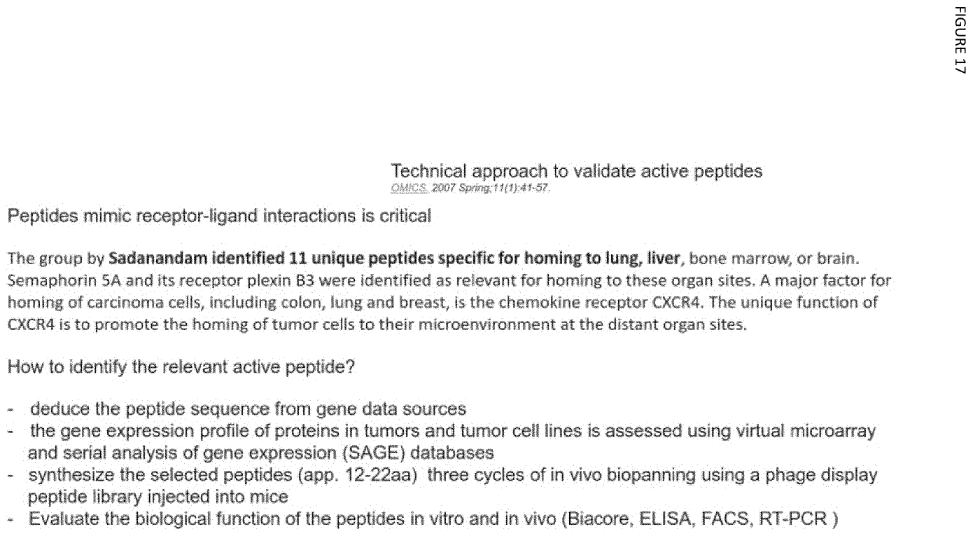

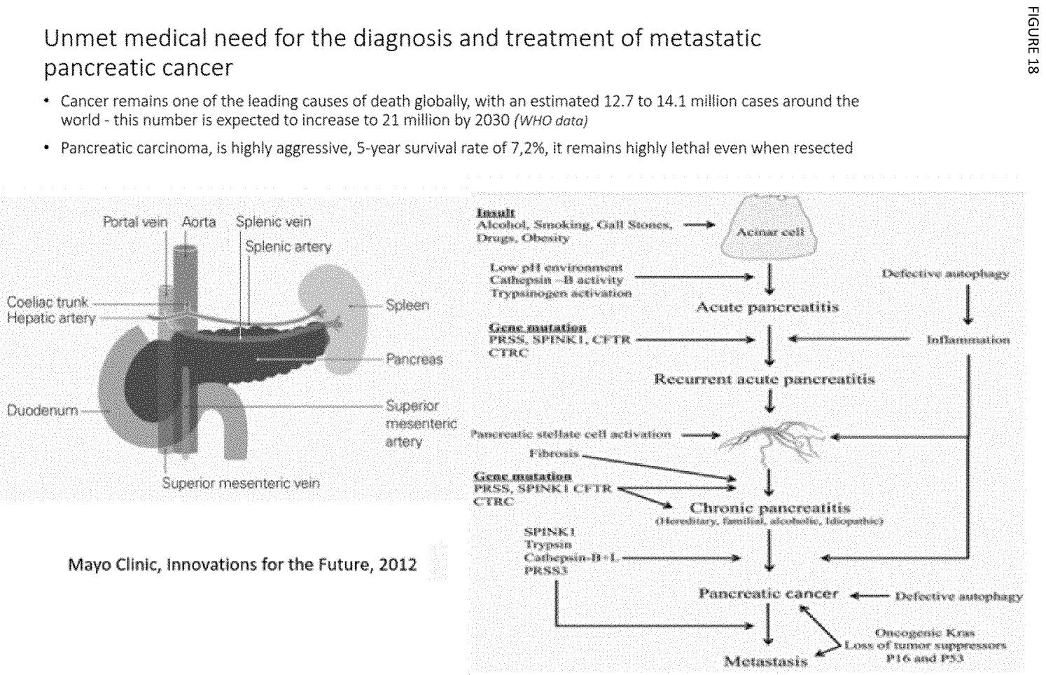

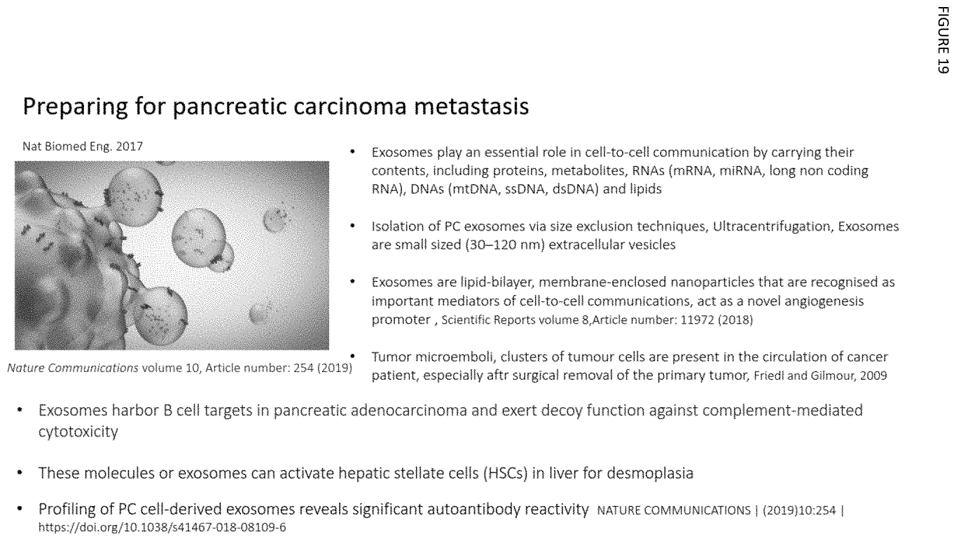

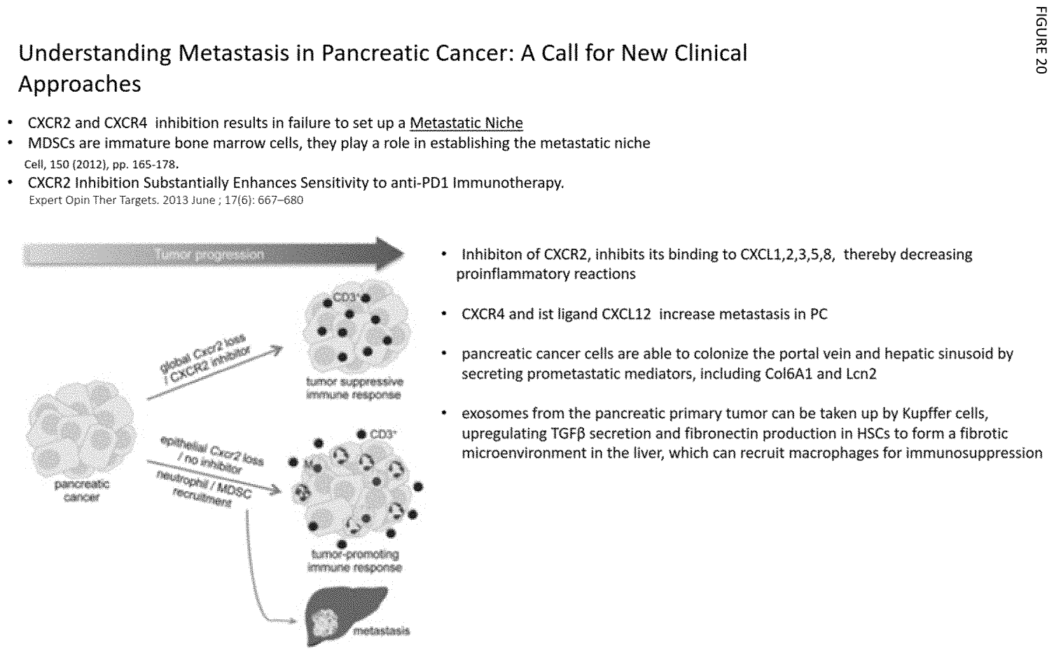

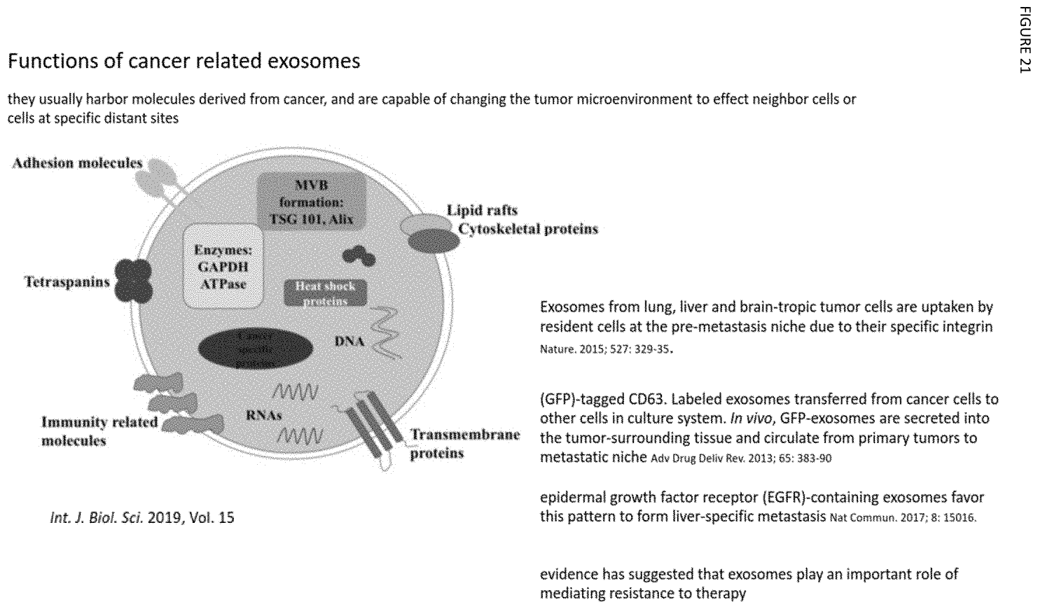

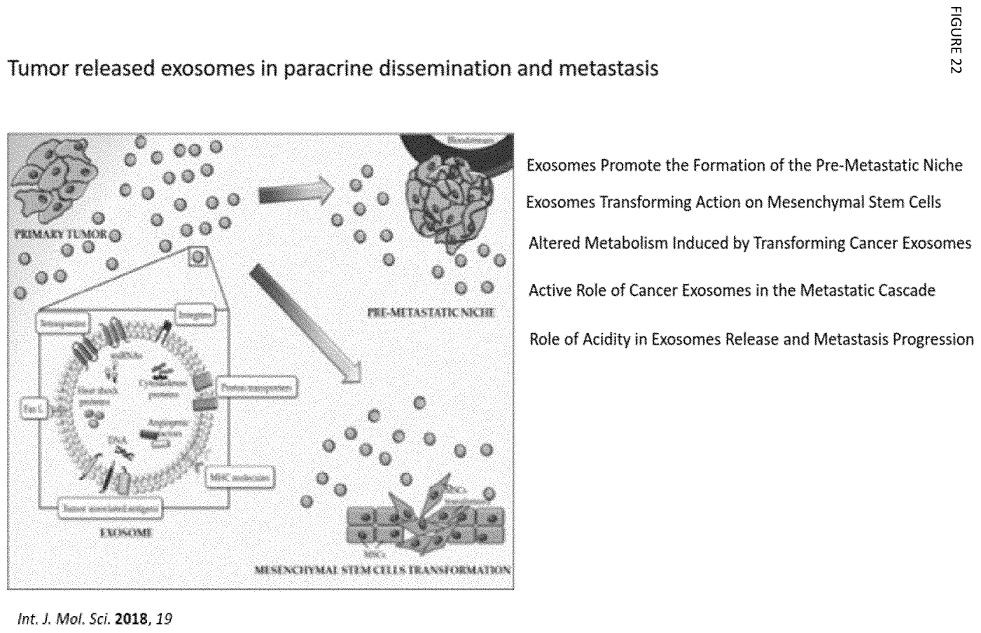

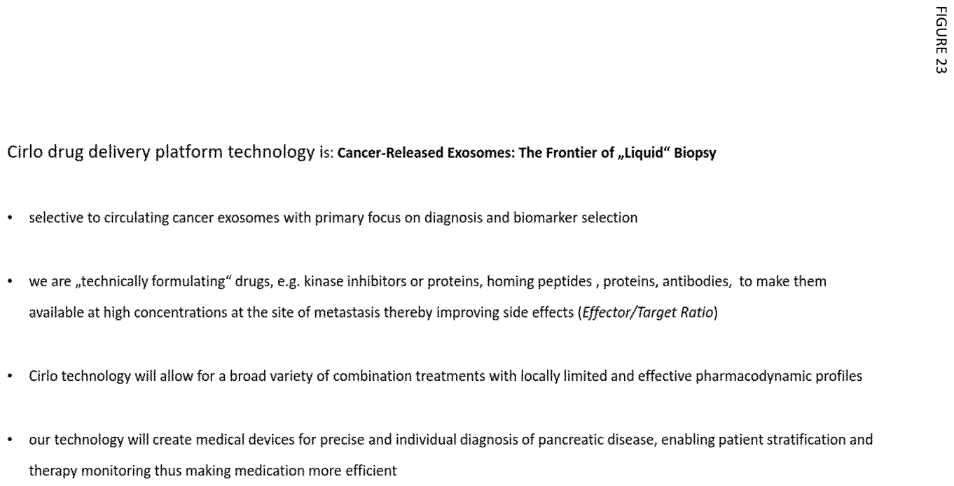

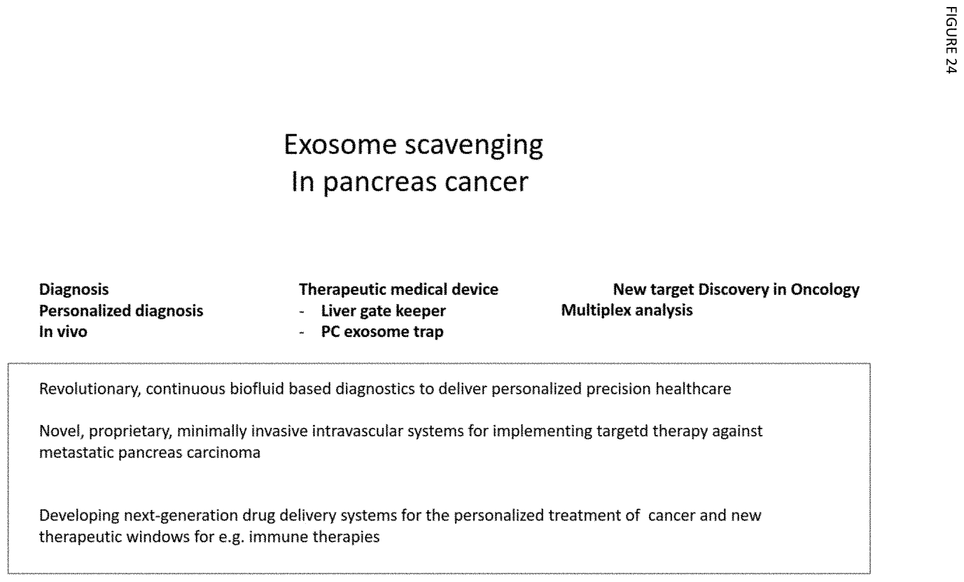

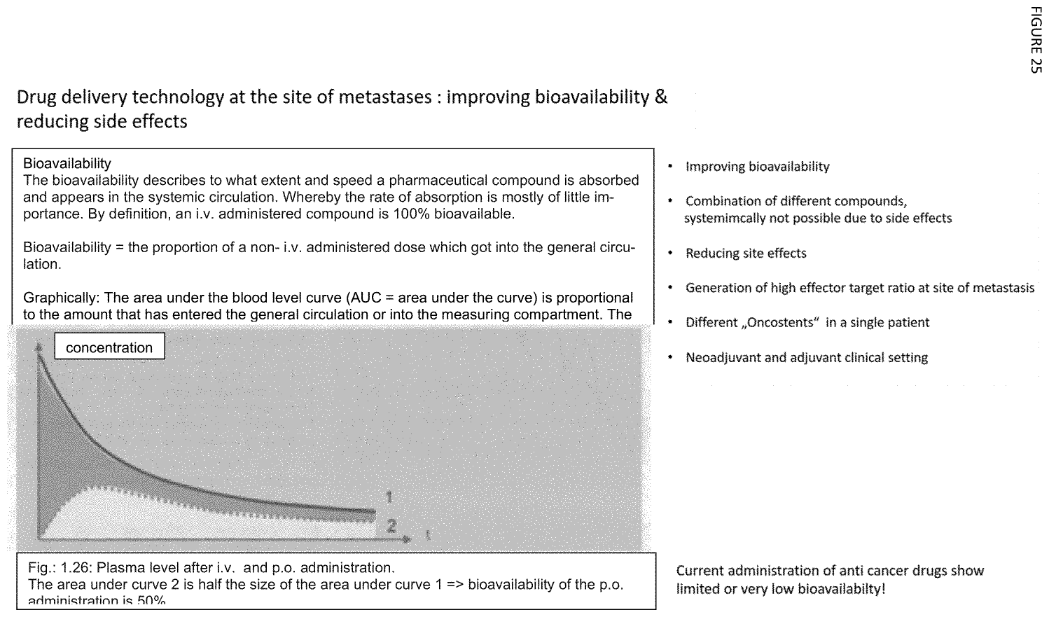

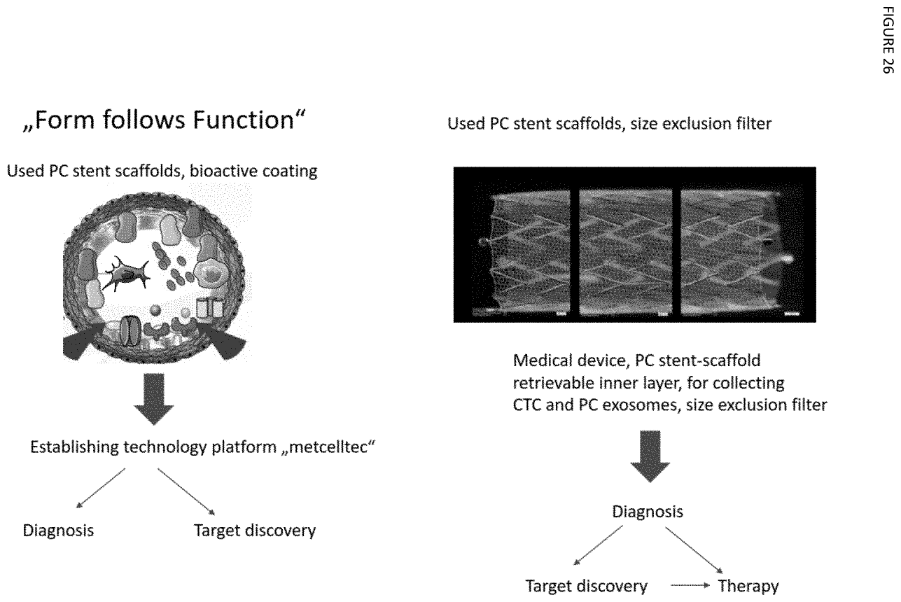

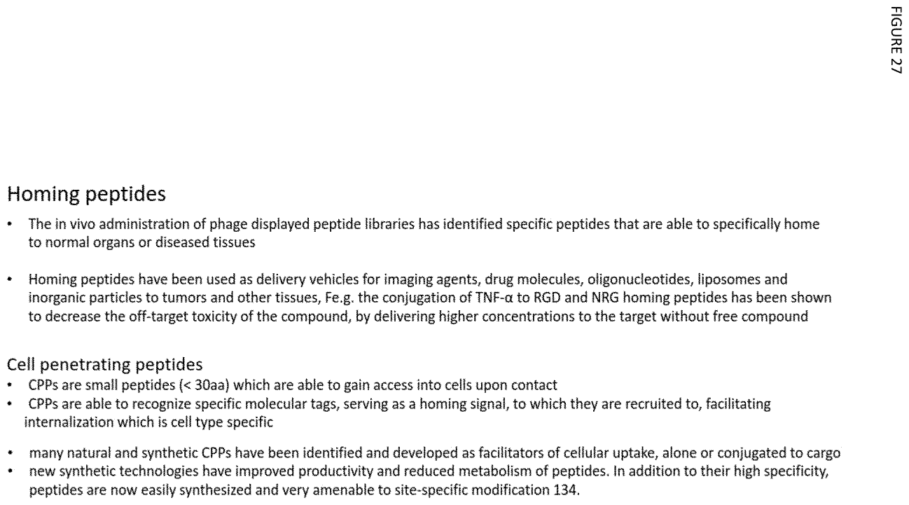

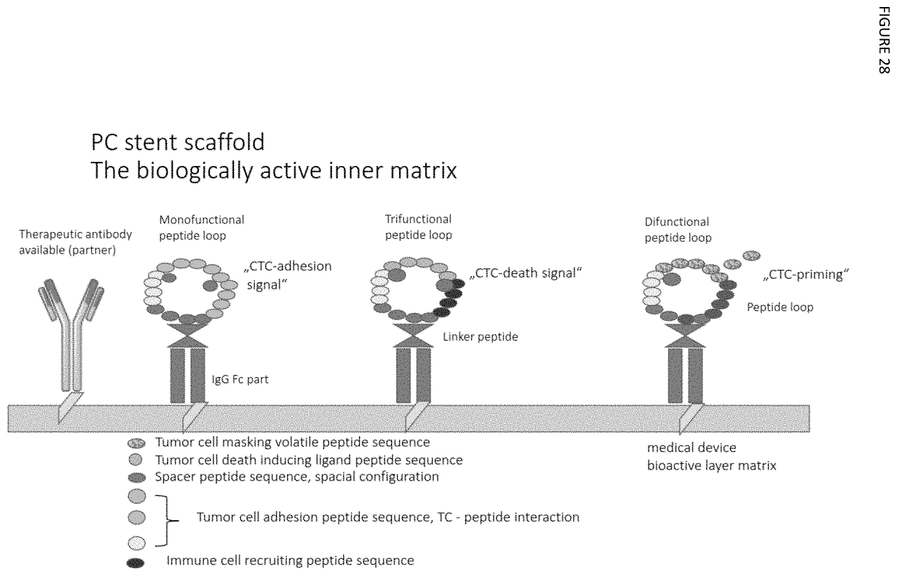

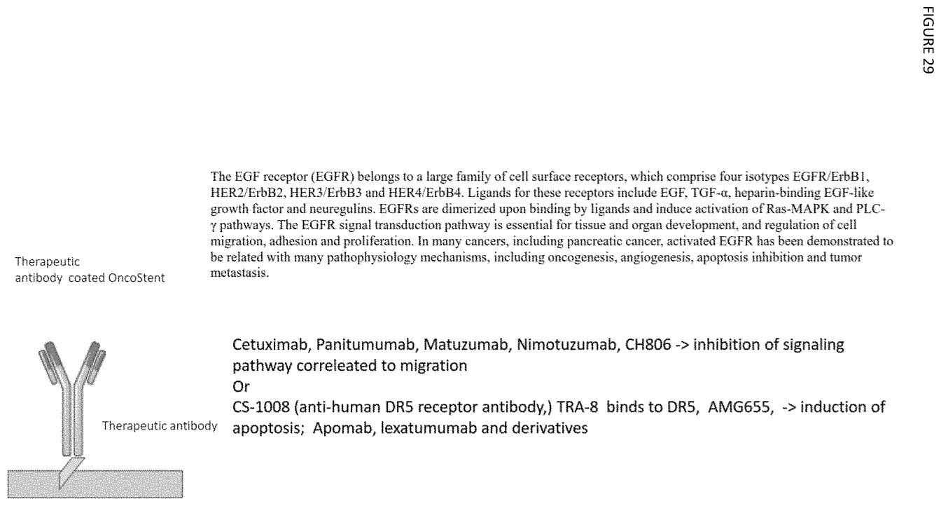

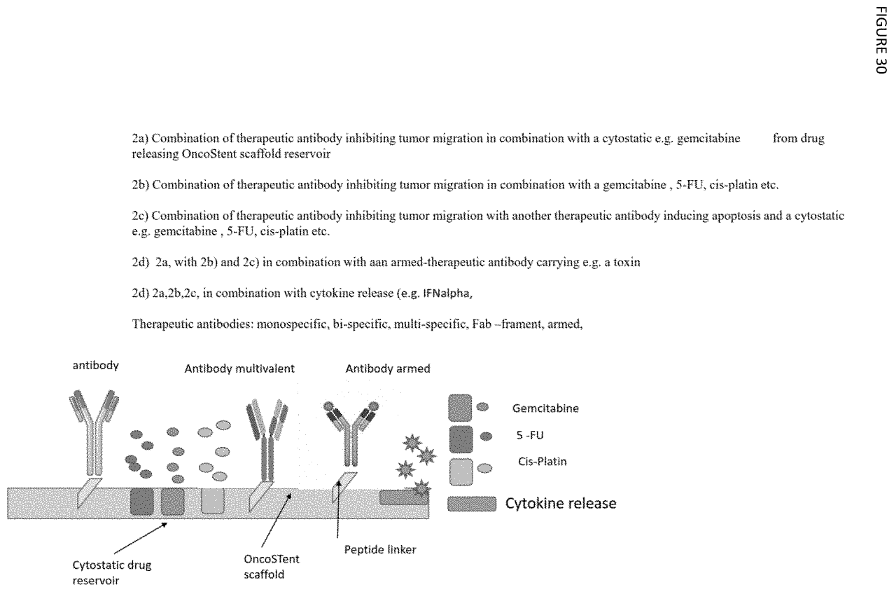

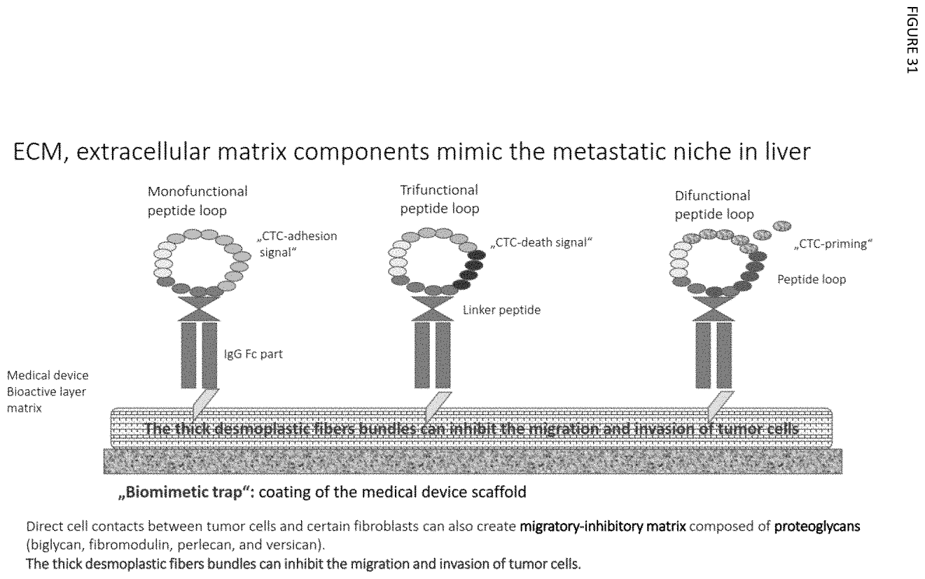

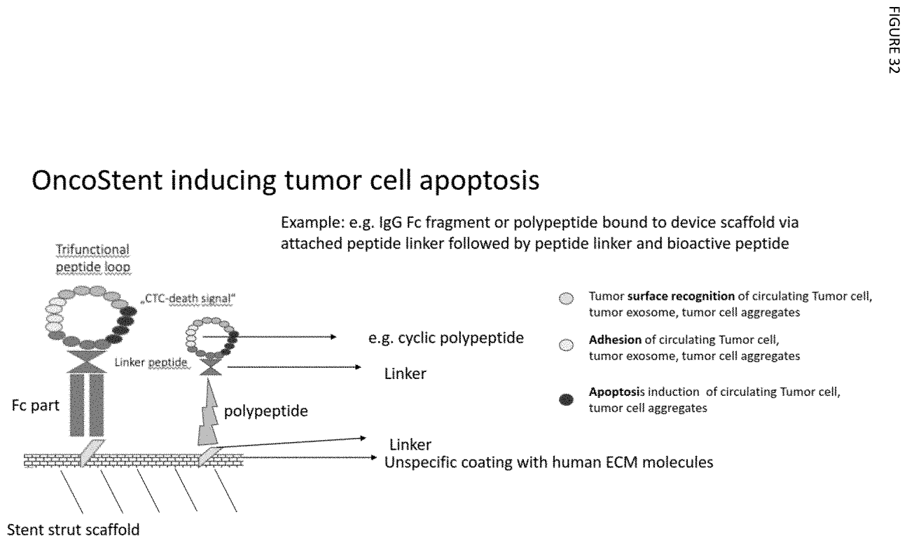

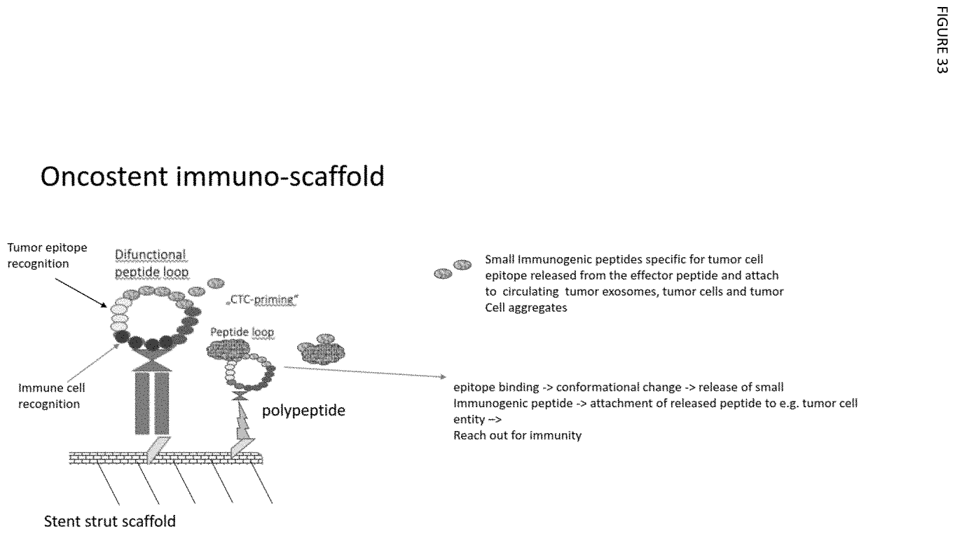

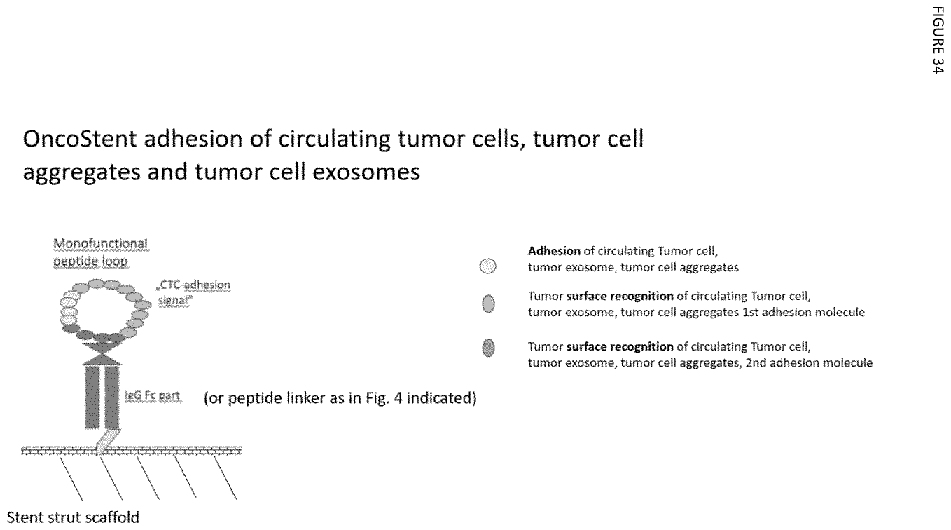

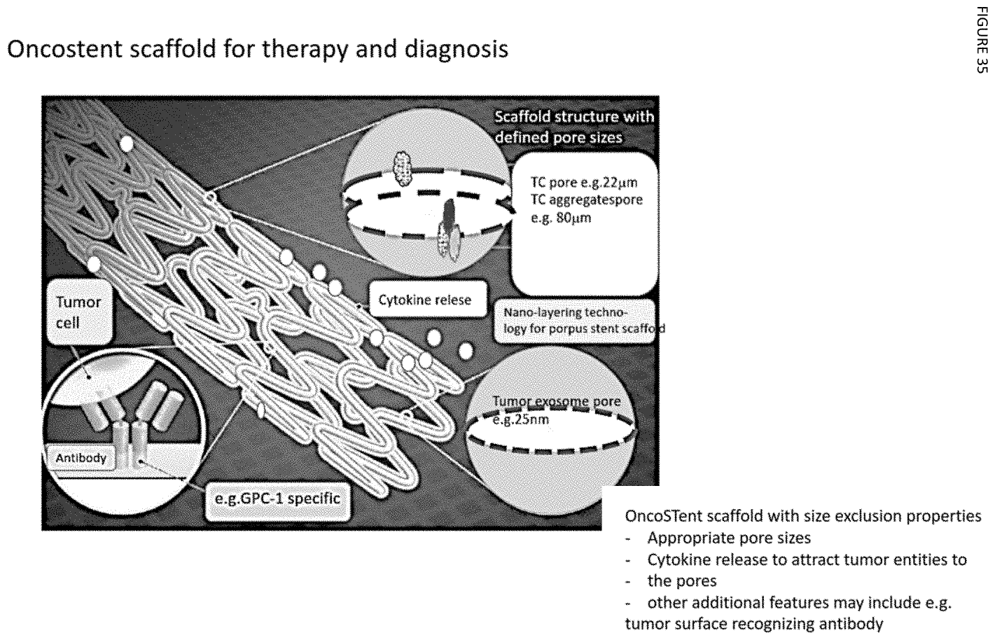

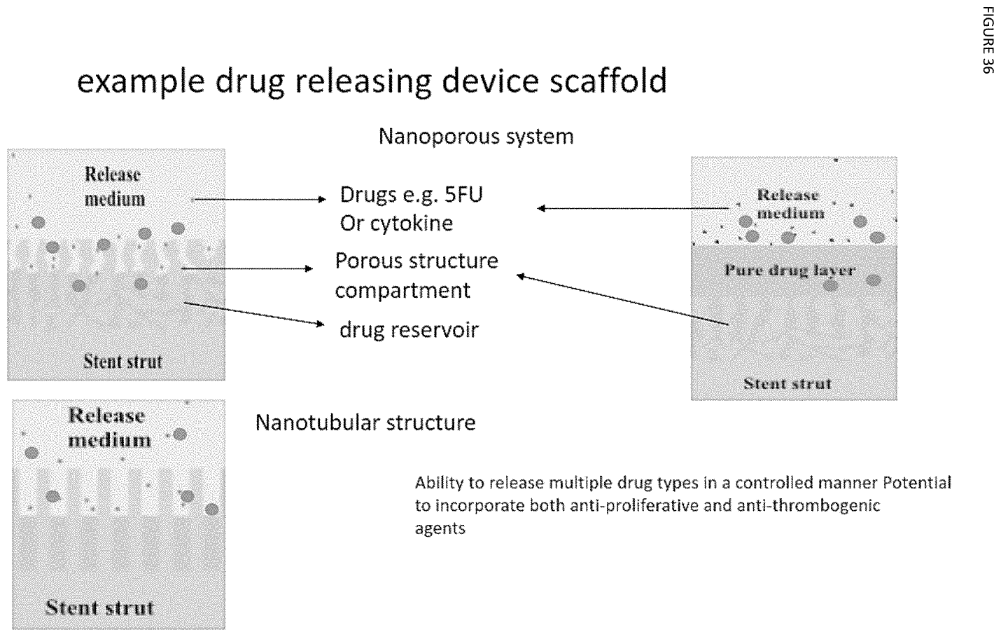

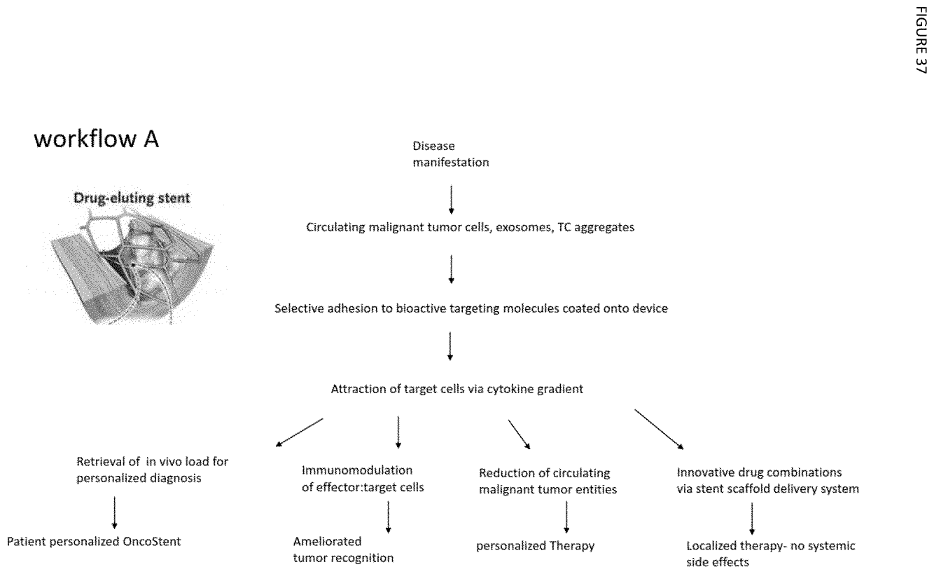

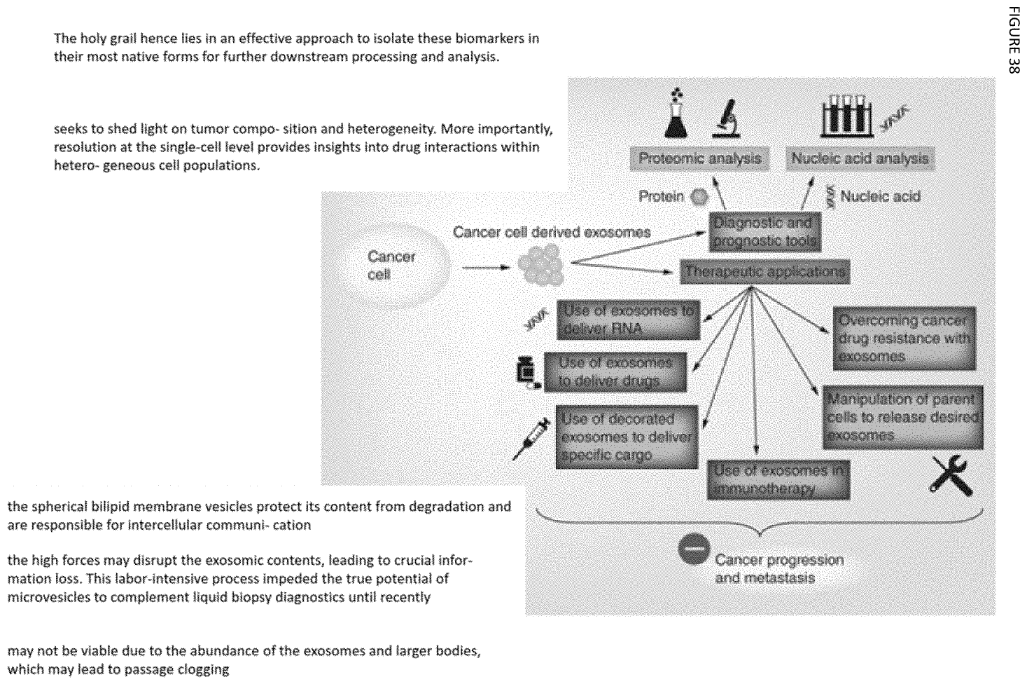

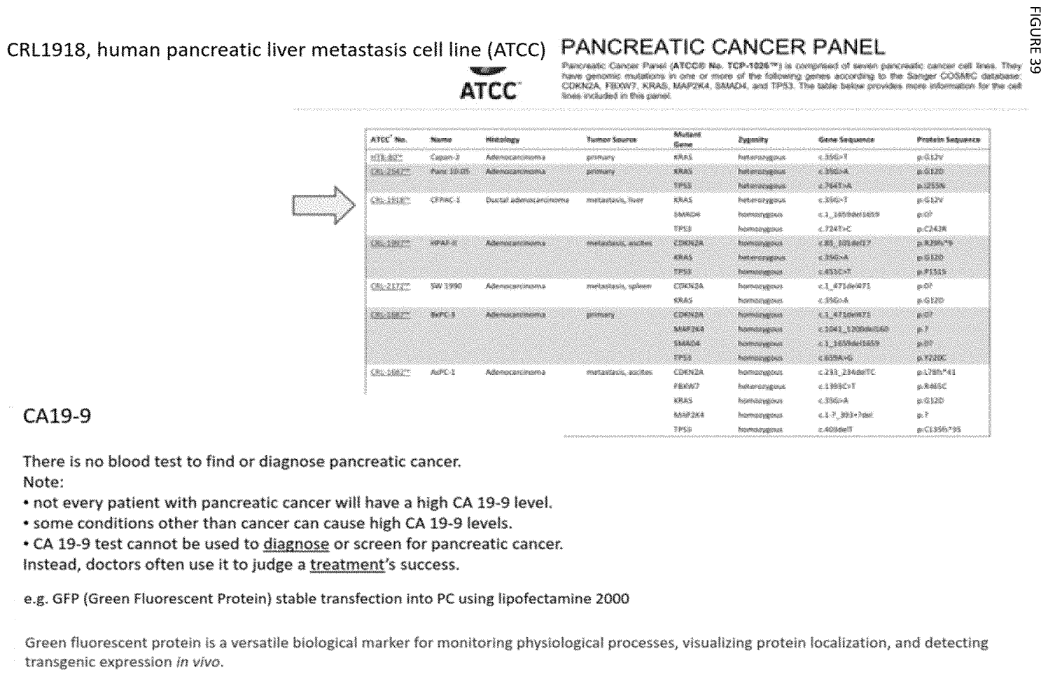

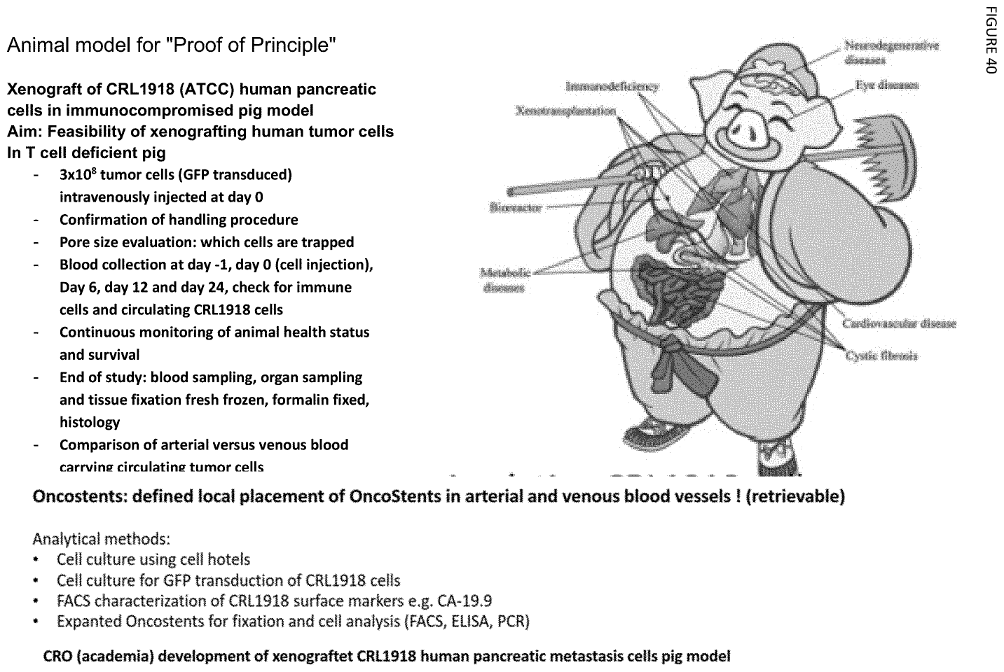

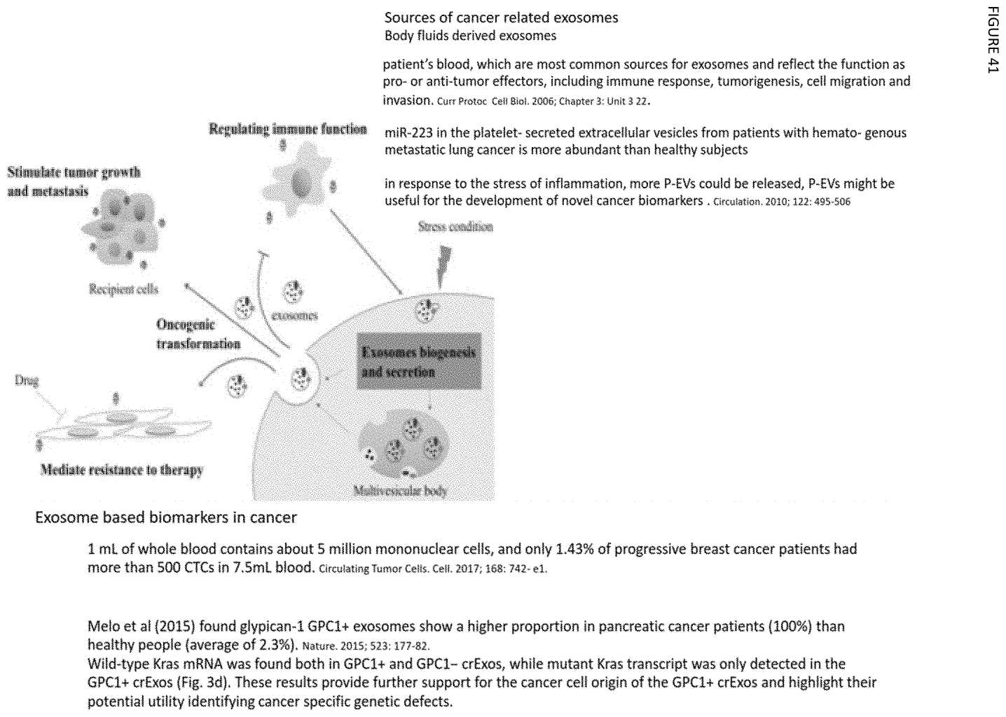

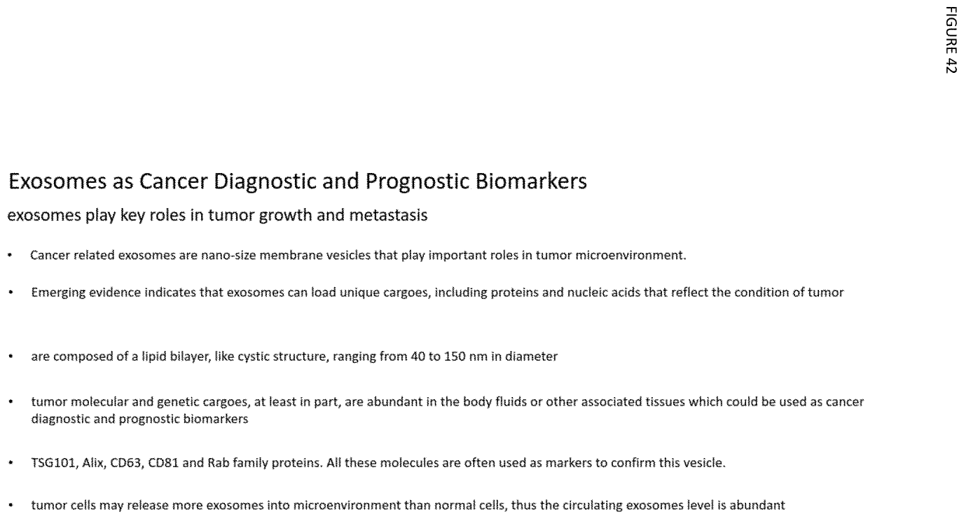

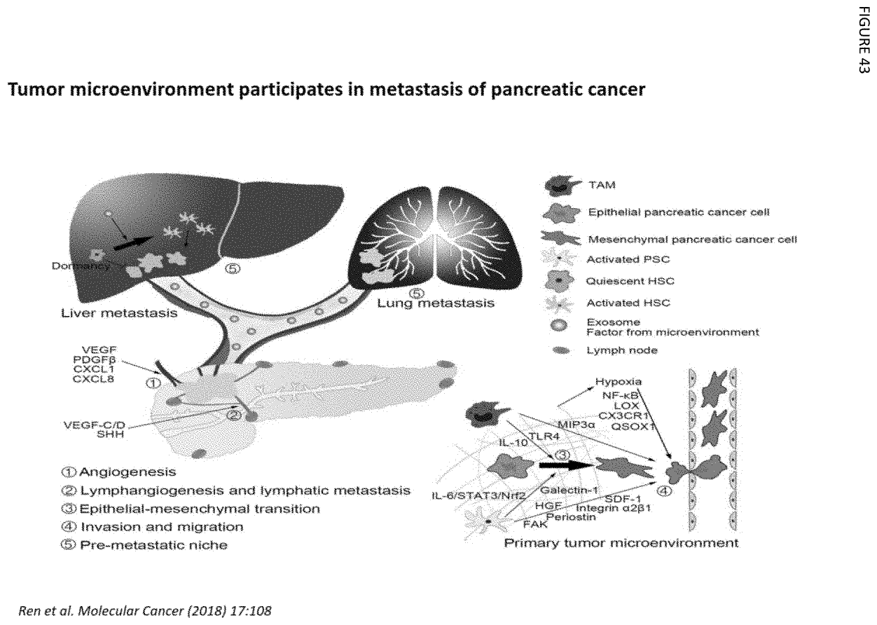

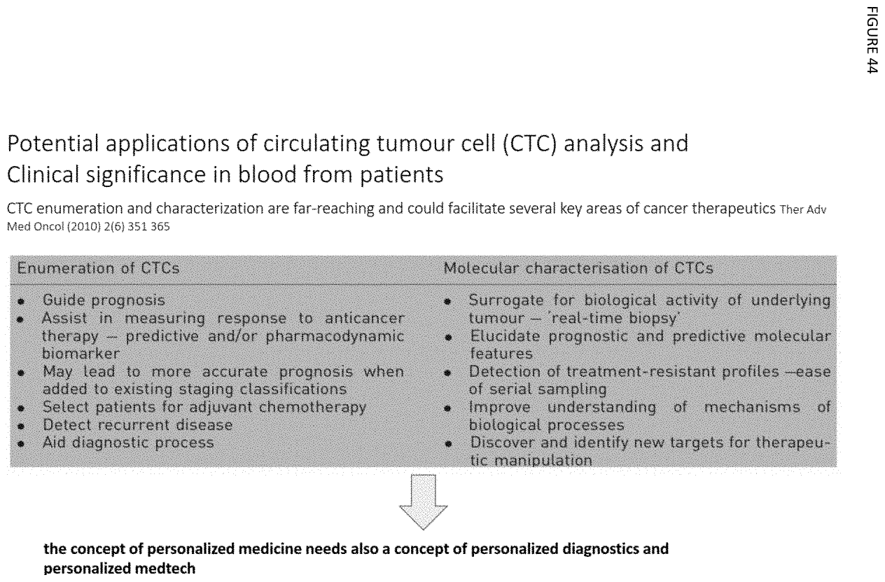

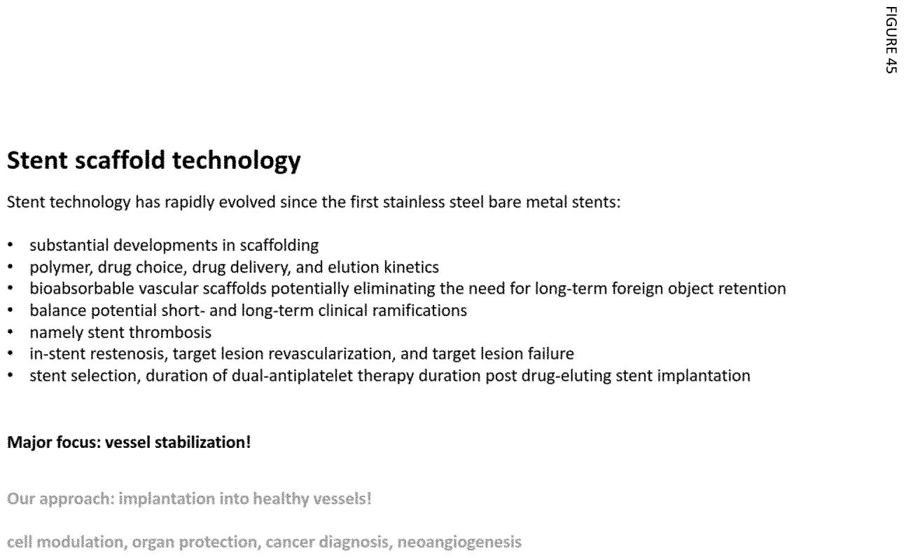

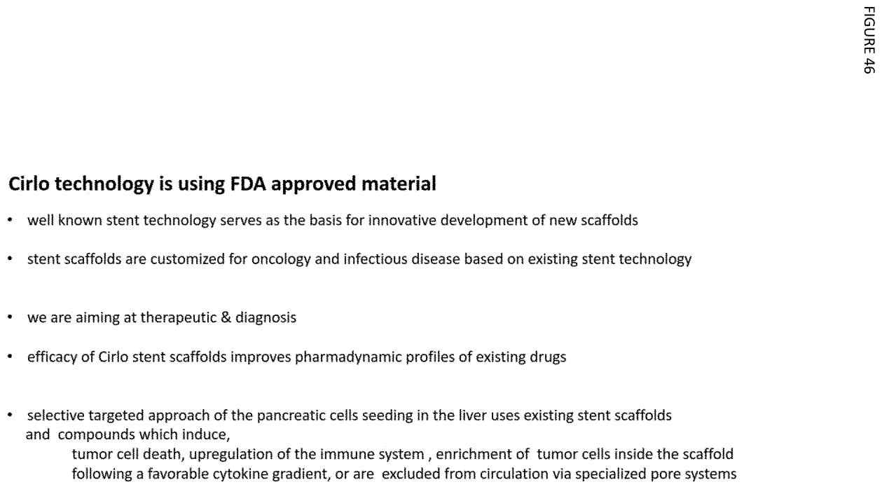

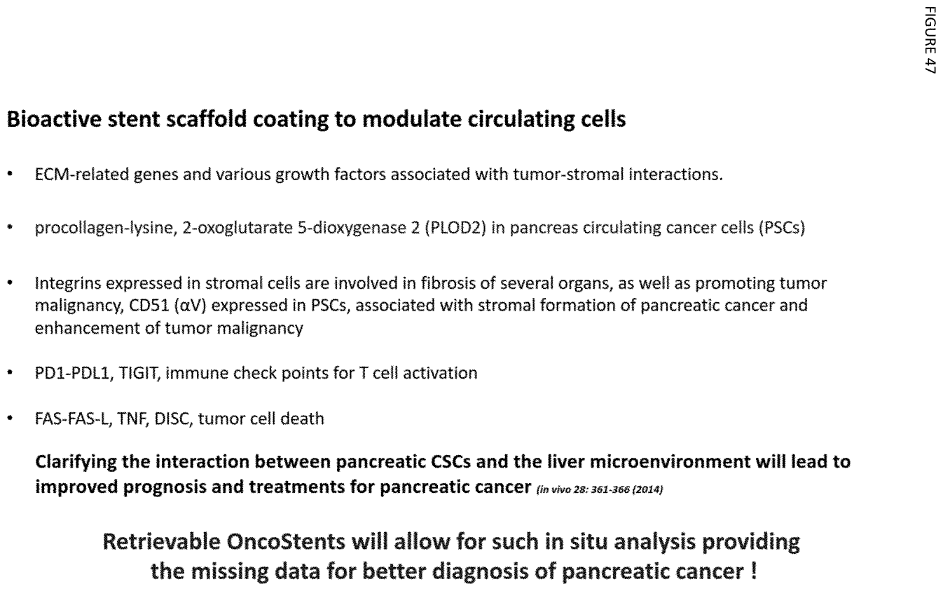

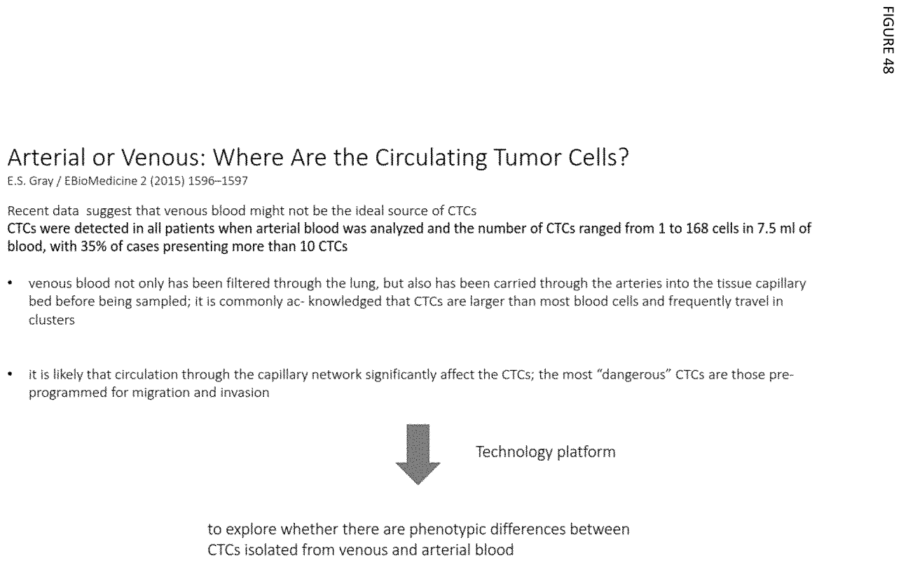

FIG. 62 shows an example of a flow directional catheter device with an interactive expandable segment (balloon) according to the invention.

FIGS. 63 to 65 provide sequence information for embodiments of the present invention.

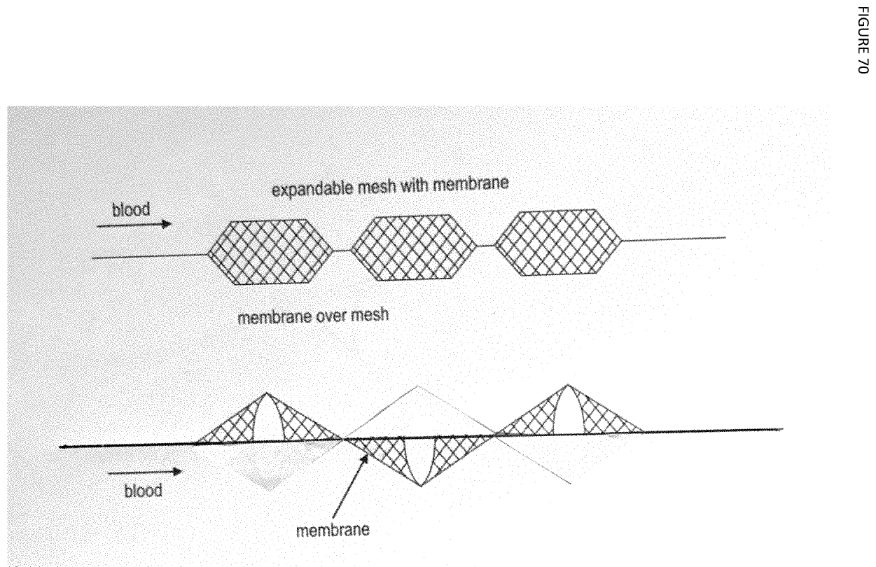

FIGS. 66 to 70 show further embodiments of the present invention.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in a first main aspect a tubular shaped elongated catheter device assembly comprising with a distal and a proximal end, comprising an intraluminal distal segment and an extraluminal proximal segment, comprising one or more chemical and/or biological agents, for interaction with bodily fluids of luminal organs, wherein the intraluminal segment comprises at least one expandable cross-sectional area which in its expanded state is smaller than the cross-sectional area of the luminal target site and wherein at least the expandable portion of the intraluminal segment is capable of interacting with at least one component of the bodily fluid via an interactive contact surface.

The term “tubular shaped elongated catheter device assembly” as used herein in the context of all main aspects, as well as related embodiments, relates to a device which is designed to be implanted into a mammalian body, preferably into a human body. The device comprises an intraluminal distal segment and an extraluminal proximal segment, wherein the intraluminal segment comprises at least one expandable cross-sectional area which in its expanded state is smaller than the cross-sectional area of the luminal target site. The term “smaller” as used in this context means about 10%, 20%, 30%, 40%, 50%, 60% or more than 60% reduced in size. The device advantageously further comprises an interactive function which is conveyed by one or more chemical and/or biological agents, i.e. it comprises agents designed for molecular or biochemical interaction with bodily fluids of luminal organs, in particular with components in said bodily fluids. The interaction is envisaged to lead to a recruiting or binding of said components of the bodily fluid to the device. The interaction takes place at least at the expandable portions of the intraluminal segment of the device via an interactive contact surface. Components of the bodily fluids such as circulating cells or parts thereof, e.g. exosomes or vesicles, are accordingly filtered or removed from the fluid, e.g. blood circulation. In a typical embodiment, the device is capable of filtering and thereby recruiting circulating tumor or circulating metastatic cell or motile parts of tumor cells, as well as tumor cell aggregates or tumor cell exosomes. The filtering and recruiting activity is designed to lead to a removal of circulating tumor and/or circulating metastatic cell and/or motile parts of tumor cells, as well as cell aggregates formed by tumor or metastatic cells, as well as exosomes or similar sub-cellular microvesicle, from bodily fluids of luminal organs. It is preferred that said removal is from the bloodstream or from lymphatic fluids. The present invention, in particular envisages the recruiting of cells, cell aggregates, as well as of motile parts of tumor cells such as circulating microvesicles which are small membrane-bound cell fragments (sizes between 30 and 1000 nm diameter) or exosomes, which have 30 to 100 nm in diameter. Circulating microvesicles and exosomes were recently shown to have roles in cell signaling and intercellular molecular communication. Circulating microvesicles are typically actively released into the extracellular space to interact with specific target cells and have been demonstrated to deliver bioactive molecules. In many tumors, circulating microvesicle levels increase. The level and biochemistry of exosomes and microvesicles may provide suitable indicators for tumor severity. Similarly, the level, amount and biochemistry of immune cells and cell aggregates, e.g. cell groups of 2 to 15 cells which are transported in the bodily fluid as an aggregate, are considered to constitute suitable indicators for tumor development and severity.

In specific embodiments in the context of all main aspects, the catheter device is a “flow directed balloon tipped vascular catheter”. This catheter form is flow-directed with a balloon tip that is inserted via an internal jugular or subclavian vein or the femoral vein. The catheter is subsequently guided by the bodily fluid, e.g. blood, flow into downstream areas such as the superior vena cava, the right atrium, right ventricle and then into the pulmonary artery. In this embodiment, the distal end of the intraluminal segment is comprised of a compliant balloon, wherein the balloon diameter is smaller than the surrounding vessel diameter. The term “smaller” as used in this context means about 5%, 10%, 20%, 30%, 40% or more than 40% reduced in size. Advantageously, the balloon is non-obstructive to surrounding fluid flow. Portions of the intraluminal segment are further expandable and interactive with elements of bodily fluids in the target area via chemical and/or biological agents as described herein.

In a specific embodiment the at least one expandable portion of the intraluminal segment is an inflatable balloon, wherein said expandable portion is designed to bind a tumor cell, a tumor cell exosome, and/or a tumor cell aggregate and/or an immune cell.

In preferred embodiments the cross-sectional area of the intraluminal segment is at least about 50% smaller than the cross-sectional area of the luminal target site. For example, the cross-sectional area is, for example, 55%, 60%, 65%, 70%, 75% or 80% smaller than the cross-sectional area of the luminal target site. Corresponding devices are is capable of allowing for a rapid and unobstructed flow of bodily fluids.

In preferred embodiments, the device comprises sections permitting free flow of bodily fluids without any obstructing structural elements at any given point along the longitudinal extension of the device. In further embodiments, the device comprises sections permitting at least 30%, preferably 50%, e.g. 55%, 60%, 65%, 70%, 75%, 80% or more cross-sectional unhindered flow of bodily fluids.

In further embodiments, the device comprises sections which permit a free flow of bodily fluids, which are interrupted by one or more sections permitting an interaction with components of the bodily fluids. In such embodiments, the interactive sections spare cross-sectional areas of free flow.

In typical embodiments, the interactive function is provided by intraluminal segments comprising at least one expandable portion. The term “expandable portion” as used herein relates to an increase in size towards the luminal borders or vessel walls. It is particularly preferred that the expandable portion is reversibly expandable, i.e. can be reduced in its size, e.g. when intending to replace the device or parts of it. The expandable portion is, in several embodiments of the invention, capable of increasing the interactive contact surface with the bodily fluid. Accordingly, cells, cell aggregates or exosomes are allowed to interact with the device in an efficient manner due to the increase surface of the expandable portions of the device of the present invention.

In order to avoid flow obstruction in the luminal target sites or organs, it is preferred that the expandable portion at maximal expansion comprises a cross-sectional area of equal or less than about two thirds of the cross-sectional area of the luminal target site. For example, the cross-sectional area may be about 65%, 60%, 50%, 45%, 40% or less of the cross-sectional area of the luminal target site.

In further embodiments, the expandable portions which only partially cover the cross-sectional area of the luminal target are arranged in a suitable manner. It has been found by the inventors that a sequential order along the longitudinal extension in a clockwise orientation is very well suited to maximize the interaction between bodily fluid components and device, while avoiding any flow obstruction at the luminal target site. It is particularly preferred to have a clockwise orientation with more than 90° differences between the sequential positions, e.g. 100°, 105°, 110°, 115°, 120° or more, or any value in between the mentioned values.

In further embodiments, the device comprises at least two localization markers. These markers should preferably be opposite to each other. The term “localization marker” as used herein in the context of all main aspects, as well as related embodiments, means that device comprises an element which can be detected from the outside, e.g. during surgery or when monitoring the device during operation. It is particularly preferred that the markers are located at the starting and end point of a segment comprising chemical and/or biological agents. This allows for a convenient detection of the interactive zones, e.g. for additional analysis, replacement etc.

It is further preferred that the localization marker is a radiopaque marker, an ultrasound marker, an MRT marker or a CT marker, preferably further comprising at least one sensor such as an optical sensor, an analyte detecting sensor, a thermal sensor or a flow sensor. The term “radiopaque marker” as used herein in the context of all main aspects, as well as related embodiments, relates to materials which block X-ray radiation. Examples of such markers include platinum, gold, tantalum, or stainless steel, or a radiopaque ink. It is particularly preferred that the radiopaque marker is included in the device design. For example, the marker may be located at the proximal and distal end, or on at least two opposite portions of the outermost structural element of the device body. Such a design can advantageously allow to judge radial expansion under medical imaging, e.g. online X-ray analysis, and thus improve device expansion and placing etc. An “ultrasound marker” as used herein relates to materials which can be detected by ultrasound techniques, e.g. IUVS imaging technologies. An “MRT marker” or a “CT marker” as used herein in the context of all main aspects, as well as related embodiments, refers to a marker which allows to image the device during MRT or CT imaging procedures. Additional sensors may further be placed to obtain a more comprehensive set of parameters for the in vivo environment of the device. Corresponding parameters may, in specific embodiments, be obtained via wireless transmission or direct sensing with apparatuses interacting with the device. For example, an integrated ohmmeter or electrical resistance sensor may record or sense the increase of electrical resistance when cells adhere to the surface of the stent.

The device according to the present invention may be provided, in preferred embodiments, with one or more suitable properties, which can be combined or mixed according to necessities or circumstances. In certain embodiments, all properties may be given in device. The device according to the invention is hence designed to comprise one or more of said properties, in particular of properties (i) to (v) as mentioned below.

These properties include: (i) it is freely floating in a target vessel. The term “freely floating” as used herein in the context of all main aspects, as well as related embodiments, means that the device is not fixed at the lumen of the vessel and can thus move within the bodily fluid. The device have, however, to be anchored, e.g. by floating wires or extraluminal fixation elements. In preferred embodiments, the proximal end of the extraluminal segment is connected to an extraluminal fixation element for temporary implantation and for fixation of the intraluminal device and prevention of intraluminal migration of the free floating device. The advantage of this approach is that the device can be used as free floating device without any continued circumferential vessel wall contact in the target luminal organ, thus avoiding vessel trauma and tissue growth. Independent fixation of the device in the target lumen needs antimigratory mechanical properties such as radial force against the vessel or e.g. hook-like fixation elements, all of which inflict trauma to the target vessel and incalculable risks, also with respect to retrieval of the device.

Furthermore, (ii) the device is freely positionable in a target vessel. The term “freely positionable” as used herein in the context of all main aspects, as well as related embodiments, means that the device can be placed at any position in a target vessel. Potential size differences between target vessels may be reflected by using differently sized devices, or by making use of mechanisms which allow to ad-just the size of the device to the target vessel, e.g. by further opening or closing the device. It is preferred that device is designed to be freely positionable in a minimal invasive surgery approach, e.g. with endoscopic technology including cameras and grapplers etc.

Furthermore, (iii) the device is retrievable. The term “retrievable” as used herein in the context of all main aspects, as well as related embodiments, means that the device can removed from its implantation site, e.g. by reducing its size or retracting extended elements, without leaving significant residues and without destroying or damaging the target vessel where is has been implanted. The way the device is retrieved can be any mechanism known to the skilled person. Preferably, the retrieval is performed by catheter means. It is further preferred that the retrieval be performed in a minimal invasive manner, e.g. making use of catheters, endoscopic technology etc. It is particularly preferred that least one docking element is present at the proximal end of the device. The term “docking element” as used herein relates to a structural component which allows for an interaction with an auxiliary tool such as a catheter or endoscope tool etc. The interaction may, in particular, be designed for a retrieval of the device, e.g. after a certain period of time. The docking may, for example, include a mechanical coupling between the device and an interaction tool such as a catheter. Alternatively, the device may provide a docking element in the form of a protrusion which is easily reachable and detectable, allowing for a grabbing or catching of the device, e.g. in analogy to vena cava filters.

A further property (iv) of the device according to the present invention is that it is anchorable in a target vessel. The term “anchorable” as used herein in the context of all main aspects, as well as related embodiments, means that the device cannot be moved or does not float within the target vessel where it has been implanted, but stays at the position of its implantation. This is preferably achieved by a contact between the expanded device and the surrounding tissue or vessel wall wherein the contact force is depending on the radial expansion force of the device or certain device elements, e.g. in analogy to self-expanding peripheral stents or vessel occluders. Also anchoring can be achieved by adding extensions of the device which seek contact or extrude into neighboring tissue or vessel walls as described for atrial occluders such as watchman occluders. It is preferred that the device is atraumatically anchorable in a target vessel.

Yet another property (v) of the device according to the present invention is that it is designed to fit into a permanent implant present in a target vessel. The device may, according to this property, be designed as a moveable and retrievable part of an implant. The implant may be present at a certain position in a target vessel. The device according to the present invention may be introducible into said implant and, e.g. after a certain period of time or after having reached the end of its working period (e.g. after 2, 3, 4, 5, 6, 7, 8, 9, 10, 11, 12, 15, 18, 20, 24 or more months), be removed therefrom. The device according to the invention may hence, in a preferred embodiment, be designed as a shuttle entity, which temporarily docks at a permanent implant present in a target vessel. The shuttle may, in further embodiments, be retrievable with a catheter, more preferably in a minimal invasive manner.

In further specific embodiment, the device may also be catheter based. The term “catheter based” as used herein in the context of all main aspects, as well as related embodiments, means that the device can be packed and/or provided to the patient inside of a catheter. The term “catheter” as used herein in the context of all main aspects, as well as related embodiments, relates to a thin tube made from medical grade materials that be inserted in the body to treat diseases or perform a surgical procedure. A catheter may be any suitable catheter known to the skilled person. Typical examples include polymers based catheters comprising material such as silicone rubber, nylon, polyurethane, polyethylene terephthalate (PET), latex, or thermoplastic elastomers. Also envisaged are polyimidine catheters. The catheter may, in certain embodiments, be connected to a deployment mechanism and may house a medical device that can be delivered over a guidewire. The catheter may include a guidewire lumen for over-the-wire guidance and may be used for delivering a device according to the invention to the target vessel. In certain embodiments, the catheter may have braided metal strands within the catheter wall to increase structural integrity. The structural elements of the catheter tip may further be bonded or laser welded to the braided strands of the catheter to improve the performance characteristics of the catheter tip. In specific embodiment, the device comprises a longitudinally extending catheter.

In further embodiments, the device comprises a through lumen housing of the expandable portions of the device. Also envisaged are devices characterized by at least one wire lumen within at least the intraluminal segment of the device. Also preferred is a reversibly expandable segment which is comprised of at least one expandable balloon surface. This balloon surface is designed to have a capability for interaction with at least one component of the bodily fluid. In this embodiment, the expandable balloon is preferably arranged with the distal end of the intraluminal portion of the device. The advantage of such a component is that the balloon can be used as an interactive and flow directing balloon simultaneously.

In certain embodiments the device according to the invention comprises a reversible expandable device body. The device may accordingly be expanded along the line from the proximal to the distal end of the intraluminal segment. The expansion may be directed along the line from the proximal to the distal end, or perpendicularly thereto. The expansion may, in specific embodiments, increase the volume of the device by 10, 20, 30, 40, 50, 60, 70, 80, 90, 100, 150, 200, 300, 400, 500, 600, 700, 800, 900, 1000% or more than 1000% or by any value in between the mentioned values.

The elongated tubular shape is preferably provided by a memory shaped wire, or memory shaped spiraling wire, which forms a tubular spiral. An example is a flexible nitinol wire. More preferably, the memory shaped spiraling wire may be modified into a single spiral-like wire structure, characterized by incomplete wire circles and an interdigiting structure.

It is further preferred that the extensions of the wire are provided in the form of circular loops or ellipsoids or non-straight longitudinal extensions.

In particularly preferred embodiments the expandable portions comprise a porous membranous surface. The term “porous membranous surface” as used herein in the context of all main aspects, as well as related embodiments, means that the device comprises pores, preferably of different sizes, which are arranged in a membrane structure. These structures are provided at the surface of the expand-able portions, allowing for a maximization of interaction with components of the bodily fluids.

In further embodiments the expandable portion comprises a filter membrane. Accordingly, the device as defined above comprises at least one filter membrane. The term “filter membrane” as used herein in the context of all main aspects, as well as related embodiments, relates to a selective barrier, mainly performing the function of a separator, e.g. by allowing for a filtering process as describe. The filter membrane may, accordingly, be designed to allow for partial separation of ingredients of bodily fluids such as blood or lymphatic fluid. Preferably, the filter membrane may be designed to allow for the passage of liquid portions, proteins or subcellular fragments in bodily fluids, e.g. blood. It is further preferred that blood cells like erythrocytes, thrombocytes can pass the membrane. In preferred embodiments, the filter membrane has at least one of the following properties: (i) the filter membrane is attached to and arranged with the expandable portion of the device between its proximal and distal end; and (ii) the filter membrane is a non-permanent, retrievable filter membrane. It is further particularly preferred that the filter membrane is incapable of allowing the passage of cellular entities such as circulating tumor cells, circulating metastatic cell, cell aggregates of tumor cells or metastatic cells. In certain specific embodiments also motile parts of tumor cells, e.g. exosomes originating from primary tumors may not pass the membrane. Advantageously, the device is thus capable of providing an increase, preferably maximized exposure of the membrane surface to the bodily fluid, e.g. the blood or lymph circulation, while at the same time avoiding stasis or thromboses. Accordingly, a maximized filter surface is provided allowing for a maximized bodily fluid, e.g. blood exposure.

In a further set of embodiments, the filter membrane comprises, essentially consists of, or consists of one or more microfilaments. For example, the filter membrane comprises, essentially consists of, or consists of a multitude of longitudinally extending microfilaments, each of which is coatable by bioactive agent, thus creating a large bioactive surface area and permitting blood flow. The microfilaments may, in certain embodiments, be arranged parallel and straight or parallel and spiraling or in other arrangements. Further details may also be derived from FIG. 57, which shows and illustrates the corresponding embodiment.

In preferred embodiments, the filter membrane may comprise pores. These pores or the pores of the porous membranous surface as mentioned above may have a pore diameter which ranges from about 25 nm to about 100 μm in diameter. In preferred embodiments, the pores have different size ranges includes the range of about 25 nm to 100 nm in diameter, the range of about 100 nm to 10 μm in diameter, the range of about 10 μm to 25 μm in diameter and the range of about 25 μm to 100 μm in diameter. These ranges, which may all be present in one device or only a sub-group thereof, are adapted to the bodily fluid component to the recruited by the device, i.e. exosomes and the like being of the size of 25 nm to 100 nm, small cells being of the size of 7 μm to 20 μm, circulating tumor cells being of the size of 10 μm to 25 μm, cell aggregates being of the size of 25 μm to 100 μm, and other cells or cell aggregates, e.g. cancer-associated fibroblasts (CAFs) being of the size of 10 μm to 15 μm, myeloid-derived suppressor cells (MDSc), and other antigen-presenting cells (APCs) being of the size of 10 μm to 20 μm. It is further preferred that the pores are provided in a differential manner such as comprising differential ranges of 25 nm to 100 nm, 100 nm to 10 μm, 10 μm to 25 μm, or 25 μm to 100 μm. In further embodiments, the pores may have a diameter of 25 nm, 40 nm, 50 nm, 75 nm, 100 nm, 200 nm, 500 nm, 750 nm, 1 μm, 5 μm, 10 μm, 15 μm, 20 μm, 50 μm, or 100 μm or any value in between the mentioned values. Different pores in one device or one filter membrane or surface may, in further embodiments, be provided with the same diameter, or with two or more different diameters. It is preferred that pores with different diameters be present.

In embodiments, in which more than one filter membrane is present in a device, said filter membranes may preferably have differential pore diameters and/or differential pore pattern. It is particularly preferred that the diameter ranges from about 25 nm to 100 nm, 100 nm to 10 μm, 10 μm to 25 μm, or 25 μm to 100 μm or any value in between the mentioned values. The term “differential pore pattern” as used herein in the context of all main aspects, as well as related embodiments, refers to the geometric arrangement of groups of pores on the filter membrane or surface, e.g. 4 to 100 pores, or groups of such basic groups pores etc. This arrangement may have any suitable form, e.g. be an equidistant pattern, or have a circular, rectangular, ellipsoid, linear, concentric or stellar form. The mentioned pattern may be different over the extension of one filter membrane or surface, e.g. a proximal circular pattern may be followed by a more distal rectangular pattern etc. In case more than one filter membrane is present in a device according to the present invention, the pattern between said more than one filter membranes may be different, e.g. one filter membrane may show a circular pattern, whereas the neighboring filter membrane or surface may provide a rectangular pattern etc.

The filter membrane may, in specific embodiments, be itself expandable. Accordingly, an expansion of the device may be followed or implemented by an expansion of the filter membrane. In certain embodiments, the filter membrane is attached to or arranged with the proximal and distal end of the device. In further embodiments, the filter membrane is designed as a non-permanent, retrievable filter membrane. According to these embodiments, the filter membrane may be separated from the remainder of the device and be removed therefrom, e.g. with a catheter or based on endoscopic techniques. The filter membrane may, in further embodiments, be designed as replaceable entity allowing for an exchange of a filter membrane after a certain period of time or after having reached the end of its working period (e.g. after 2, 3, 4, 5, 6, 7, 8, 9, 10, 11, 12, 15, 18, 20, 24 days; after 2, 3, 4, 5, 6, 7, 8, 9, 10, 11, 12, 15, 18, 20, 24 weeks; or after 2, 3, 4, 5, 6, 7, 8, 9, 10, 11, 12, 15, 18, 20, 24 or more months). It is preferred that the end of the working period does not surpass 24 months. In specific embodiments, the filter membrane may be retrievable as whole, or in parts. The partial retrievability may be implemented as segmented retrievability, e.g. via separable portions of the filter membrane, e.g. 10%, 20%, 30%, 40%, 50%, 60%, 70%, 80%, 90% etc., which can individually be retrieved, e.g. the distal part may be retrieved, whereas the proximal part is not retrieved etc. In further embodiments, any retrieval may followed by replacement of the retrieved filter membrane by a new filter membrane or the same filter membrane after washing and preparation for a secondary use. Also envisaged is the retrieval of more than one segment at a time.

The present invention also envisages that the filter membrane is self-expandable. In further embodiments, the entire filter device assembly implant is self-expandable. The term “self-expandable” as used herein in the context of all main aspects, as well as related embodiments, relates to the property of the filter membrane or device to be expandable within a target vessel once it has reached its envisaged destination. The self-expansion may, for example, be started when withdrawing auxiliary tools such as catheters, as defined herein above. The self-expansion of the filter membrane or entire device may, in preferred embodiments, be activated by balloon inflation. It is preferred that at least a portion of the device, or the entire device be activated by balloon inflation. The term “balloon inflation” as used herein in the context of all main aspects, as well as related embodiments, refers to the activity of expansion of a device by inflating a balloon element within the device. For example, a self-expanding device may comprise securing bands preventing the self-expansion of the device during the passage to the extended destination. Upon arrival, a balloon may be introduced into the device and be inflated within the device. Subsequently, said securing bands may be broken which leads to a self-expansion of the device. Subsequently, the balloon may be withdrawn, e.g. via catheter element.

It is further envisaged that the device according to the present invention comprises at least two filter membranes, each of which incompletely covers the cross-sectional area. These filter membranes may be arranged in any suitable form or shape. It is preferred that the filter membranes are arranged in tandem position along the longitudinal axis of the device body. Particularly preferred is a tandem arrangement along the longitudinal axis of the device body, wherein said filter membranes are opposite to each other within the circumference of the device body. Alternatively, they may be shifted in clockwise orientation. Such a shifting may advantageously be implemented for more than 2 filter membranes in tandem position, e.g. 3, 4, 5, 6, 7, 8, 9, 10 or more filter membranes. The device according to the present invention may, in further embodiments, comprise alternating non-completely covering filter membranes as defined herein

The present invention further envisages that the device is provided in any suitable form or architecture. It is particularly preferred that the device is provided in a tubular, onion like, pearl-chain-like, or a birds-nest like shape, or in any mixture of these shapes. Examples of corresponding forms and shapes can, for example, be derived from FIG. 24, 25, 26, 27, 28, 29, 30, or 31. The form or shape of the device may, in certain embodiments, be followed also be the form or shape of the filter membranes. For example, the device according to the present invention may comprise alternating non-completely covering filter membranes which are provided in a pearl-chain, onion type or birds-nest like shape. In other embodiments, the device comprises a multitude of longitudinally extending microfilaments, which may be arranged parallel or non-parallel to each other in straight configuration or tortuous e.g. spiralling configurations.

In another specific embodiment the device has a grid-net-like or scaffold-like shape. It preferably comprises one or more chemical and/or biological agents as defined herein. The device may be used for interaction with tumor cells in a liquid space in the vicinity of an organ or in an organ, e.g. in the space of disse. The device may be coated as described herein. It is further envisaged that the device is placed inside a tumor mass or close to a tumor masse. With wishing to be bound by theory it is assumed that the grid-net-like or scaffold-like device is able to modulate the tumor immune response by augmenting the target effector ratio inside the scaffold. A patient's immune system may accordingly be modulated via priming of the immune cells.

A grid-net-like or scaffold-like shaped device may preferable comprise biocoating molecules for coating of any scaffold for reduction of tumor mass such as an anti-CD44v6 antibody, an anti-alpha6beta1 antibody, an alpha6beta4 antibody, exosomal integrins and laminin receptor interaction and alphavbeta5, an antibody targeting fibronectin receptor and/or alpha-v exosomes in circulation. Also envisages is the presence of an anti-GMCSF antibody from tumor cells to block the recruitment of Ly6C+ and Ly6G+ myeloid cells into metastases. Further envisaged is the inhibition of S100A8, a proinflammatory factor from Kupffer cells of the liver, by antibody or peptide.

Without wishing to be bound by theory, it is assumed that tumor cells in circulation encounter Liver sinusoidal endothelial cells (LSECs) when they reach the liver. In order to escape the defence mechanism and cell death fate, the tumor cells are believed to escape while traveling along with other cells to form clusters which shield them from attacks and shear stress. Inflammatory mediators such as IL1beta, TNFalpha and IL18 are believed to initiate a cascade that facilitates the rapid exit of tumor cells and TC aggregates from the vasculature into less toxic microenvironment. The sinusoidal endothelium in the liver is assumed to participate actively in different phases of metastasis. Upregulation of adhesion molecules by LSECs, CAMs (e.g. E-selectin, VCAM-1, ICAM-1), is believed to enhance TC adhesion and transendothelial migration into the space of Disse. Inhibition of E-Selectin is further assumed to inhibit transendothelial T cell migration.

According to further embodiments, the tubular shaped elongated catheter device assembly, the grid-net-like or scaffold-like shaped device of the present invention, or the tubular shaped device assembly of the present invention may be placed near the site of metastasis in the liver. This placement is assumed to change the growth pattern within the space of Disse. It is invent particular envisaged to block Ang2 which is believed to inhibit the formation of intrametastatic vessels via the inhibition of the “co-opt” mechanism between the matrix of the space of Disse and LSEC and T-cells. It is further envisaged to block the NOTCH1 expression, which is assumed to lead to a reduced angiogenesis in micrometastases.

The present invention further envisages in certain embodiments, for devices having immuno-modulatory effects as described herein, to block IL10. This is assumed to reduce regulatory T cells at the site of tumor growth.

It is further preferred to provide a device as defined herein with a coating with CXCL9 and CXCL 10. It is assumed that this will favor macrophage class 1, leading to an antitumorigenic effect. It sis further envisaged to block the axis of CCR2/CCL2, which is hypothesised to reduce hepatic metastases and angiogenesis.

The present invention further envisages a minimally invasive implantation of a device comprising a bioactive coating membrane inside a tumor. The device may preferably be biodegradable and thus be resorbed by the body after a certain period of time. Without wishing to be bound by theory it is assumed that such an implantation will lead to an immunogenic tumor cell death, the reduction of tumor mass, and resensitization of T Cell for e.g. for Gemcitabine.

It is particularly preferred to place the device to be implanted around a sinusoidal lining epithelial cells in the liver sinusoids.

In a further preferred embodiment, a grid-net like or scaffold-like shaped device may deliver one or more toxins directly or together with therapeutic antibodies. These toxins or antibodies may be provided in a suitable reservoir zone or area. It is further envisaged to apply heat to a device according to the present invention which is assumed to accelerate tumor mass reduction and to lead to faster biomolecule release kinetics.

In certain additional embodiments a device according to the present invention may be placed in a vessel, e.g. a draining vessel, preferably of the pancreas to the liver. In certain embodiments it is preferred to functionally focus on the interface of the tumor cell and the liver, e.g. the space of Disse. Without wishing to be bound by theory, it is assumed that in this area extracellular vesicles, soluble-factors, and cell-cell contacts are provided by stellate cells that can mediate supportive or immunosuppressive effects. Through the release of trophic factors also tumor cells can be attracted and supported as reported for MSCs in other organs. If the stem cell character of stellate cells cannot be maintained, cell differentiation into epithelial cell lineages such as hepatocytes can be induced to promote liver tissue reconstitution. If appropriate signals from their surrounding environment are missing, stellate cells sustain their activated state and deposit ECM proteins leading to fibrosis and cirrhosis. In chronic diseases associated with fibrosis/cirrhosis, the perivascular niche of stellate cells is believed to be severely affected. This is assumed to explain impaired liver regeneration and prevention of metastasizing tumor cell homing in liver sinusoids. It further hypothesized that stellate cell engagement in niche formation critically depends on their activation, as non-activated stellate cells seem to support a quiescence-associated phenotype of pancreatic ductal adenocarcinoma cells via interleukin-8 release while this positive effect is lost when stellate cells activate and become myofibroblasts as investigated in vitro. The stem cell-friendly microenvironment in the space of Disse and the provision of niche elements by activated stellate cells in conjunction with their immunosuppressive functions is assumed to be the reason for the frequent homing of migrating tumor cells in the liver, predisposing this organ for metastasis. Alterations of this perivascular niche in chronic diseases may explain impaired homing of intrahepatic metastasis in liver cirrhosis.

The filter membrane as used in the context of device according to the present invention may be composed of any suitable material. Envisaged examples include elastic or foldable polymer materials. Particularly preferred is polyurethane. Also envisaged is the use of micromeshes. In preferred embodiments, these micromeshes may comprise ultrathin wires, metallic or polymeric materials. In particularly preferred embodiments, the filter membranes according to the present invention is at least partially coated with one or more chemical and/or biological agents as defined herein. The term “coated” as used herein in the context of all main aspects, as well as related embodiments, means that all or at least some sectors of the filter membrane material as defined herein is covered by said chemical and/or biological agent(s). The coverage may, in certain embodiments, be present at one or both sides of the filter membrane. The “side” of the filter membrane refers to the geometric from of said filter membrane which has the shape of a thin sheet, thus comprising two sides, while the edge is not counted as side. In preferred embodiments, the filter membrane may be fully or partially coated on its interior side only. The “interior side” as used herein in the context of all main aspects, as well as related embodiments, refers to the side of the filter membrane which is proximal to the lumen of the device or distal to the vessel wall, where the device is anchored. In further embodiments, the filter membrane may be fully or partially coated on its exterior side only. The “exterior side” as used herein in the context of all main aspects, as well as related embodiments, refers to the side of the filter membrane which is distal to the lumen of the device or proximal to the vessel wall, where the device is anchored. In yet another set of embodiments, the filter membrane may be fully or partially coated on both sides. A “partial coating” may be a coating of about less than 1% to about 99% of the area of a filter membrane, e.g. of one side of the filter membrane or both sides of the filter membrane as defined above. For example, a partial coating may comprise 1, 5, 10, 15, 20, 25, 30, 40, 50, 60, 70, 75, 80, 85, 90, 91, 92, 93, 94, 95, 96, 97, 98 or 99% or any value in between the mentioned values of the area of a filter membrane.

In certain embodiments, the coating may differ between different filter membranes of a device. For a device comprising 2, 3, 4, 5, 6, 7 or more different filter membranes, a number of 2, 3, 4, 5, 6, 7 or more coatings may be provided. Alternatively, different filter membranes may be provided with one coating only. In further embodiments, the coating may change along the axis of the device, e.g. from proximal to distal over one filter membrane or over various filter membranes. In further embodiments, biological agents may be provided as coating in different combinations, e.g. as combination of adhesion and death signals, or as a combination of adhesion and immune modulation function.

In further embodiments the filter membrane as defined herein covers at least one cross-sectional area of the body of a device according to the present invention, in particular of the body of a device having a tubular form. For example, the filter membrane may cover 1, 2, 3, 4, 5, 6, 7, 8, 9, 10 or more cross-sectional areas of said body of a device according to the present invention. It is particularly preferred that the plane of the filter membrane is arranged perpendicular to the direction of the longitudinal axis of the body of a device according to the present invention. Also envisaged are different angles, e.g. non-perpendicular angles, between the plane of the filter membrane and the direction of the longitudinal axis, e.g. 10°, 20°, 30°, 40°, 50°, 60°, 70°, 80° or 85° or any value in between said values.

The present invention also envisages that the filter membrane is disposed within or around an expandable body, is characterized by a memory shaped metallic or memory shaped polymer structure and/or comprises a detachment mechanism cooperating between the expandable portion and an intraluminal segment of the device. Also preferred are devices comprising longitudinally extending free floating microfilaments. In specific embodiments, the device comprises usually the device constraining, an outer sheath concentrically disposed about the device. In this embodiment, the outer sheath and the device are movable relative to one another, so that by withdrawal of the outer sheath, the device may unfold and positioned.

In a particularly preferred embodiment, the device as defined herein additionally comprises a downstream embolic debris filter, e.g. a retrievable downstream embolic filter. The term “embolic filter” as used herein in the context of all main aspects, as well as related embodiments, relates to a basket like structure at the distal end of the device, which is capable of catching debris or clots from the bodily fluid, e.g. from the blood, thereby preventing embolic events. The embolic filter has preferably a pore diameter of >100 μm, e.g. 110, 120, 130, 150, 200, 300, 400 or 500 μm or more or any value in between the mentioned values. Further details would be known to the skilled person or can be derived from suitable literature sources such as Shashni et al., Biol Pharm Bull, 2018; 41(4):487-503.

The device may be composed of, or be partially composed of, or comprise any suitable structural support material. According to certain embodiments, the structural support material may be metal. Preferred examples are stainless steel, gold, titanium, gold-titanium alloy, cobalt-chromium alloy, tantalum, tungsten, platinum-radium alloy, tantalum alloy, magnesium, and nickel-titanium alloy, e.g. nitinol, silver or copper. Alternatively or additionally, the support material may be plastic or polymeric material. Also envisaged is the use of memory shape materials, e.g. elastic memory shape meshwork material. Preferred examples include memory shape elastic wires. In yet another alternative, the structural support material may be a material readable by tomography or other imaging techniques, e.g. X-ray or MRI (e.g. tungsten). The present invention further envisages, in very specific embodiments that the device body is not biodegradable or composed of biomaterial or biodegradable material. The term “device body” as used herein in the context of all main aspects, as well as related embodiments, relates to all parts of the device besides the one or more chemical and/or biological agents as defined herein above. Typically, the device body relates to the structural components of the device which provide for suitable placement at the desired target vessel and its capability of resisting impacting surrounding forces such as blood flow, vessel contraction or the like.

In alternative embodiments, the device or the device body may be biodegradable or composed of biomaterial or of biodegradable material, e.g. the device is composed of, is partially composed of, or comprises structural support material selected from the group comprising biodegradable or bioresorbable material. In a group of embodiments, the device may be made of polylactic acid, which is a naturally dissolvable material that is used in medical implants such as dissolving sutures. In certain further embodiments, the flexible tube and the balloon as described herein may be removed after implanting the device. Accordingly, the present invention envisages a biodegradable device which provides a temporary mechanical scaffolding. Subsequently, it may be bioabsorbed within a reasonable period, leaving behind the healed and remodeled artery preventing possible complications with devices that have be recovered by surgery. It is particularly preferred that the material for the biodegradable device constitutes a reasonable combination of a technical suitable degradation rate and mechanical integrity. Accordingly, the degradation rate should initially be low to maintain mechanical support, e.g. for the artery to remodel and after complete remodeling, when there is a loss of mechanical integrity, the degradation rate increases rapidly so as to prevent the retention of degradation products near the implant site. An overall time period for degradation of up to 3 years, e.g. of 1 year, 2 years, 2.5 years, 3 years or any time value in between the mentioned values is envisaged. In further embodiments, the device may comprise pharmaceutical com-positions or compounds, e.g. one or more of those mentioned herein, which are released in accordance with the degree of disintegration or degradation of the device.

In accordance with certain specific embodiments of the present invention, the biodegradable device is manufactured with, or composed of at least two classes of materials: polymers and metals. For example, metals to be used in the context of biodegradable devices may be magnesium alloys, iron and iron alloys, or zinc and zinc alloys. Clinical studies have shown that magnesium is an excellent candidate for cardiovascular applications as Mg ions inhibit platelet activation, relax vascular smooth muscle cells, and prevent vasoconstriction and subsequent increase blood pressure by inhibiting hormones such as angiotensin and norepinephrine. Also magnesium, due to its high electronegativity, exhibits excellent hypothrombogenic property. Above all excess Mg from the human body is believed to be efficiently eliminated by the kidney. Certain Mg alloys such as Mg-2.2Nd-0.1Zn-0.4Zr (JDBM-2) show very high biocompatibility, mechanical integrity, and degradation kinetics and are preferred herein.

Further envisaged are nano polymeric foam blends in a solid. The present invention additionally envisages the use of a range of synthetic biodegradable polymers, based on PLA, PGA, or co-polymers (e.g., PLGA) thereof, which may be provided in several morphologies and architectures. Also envisaged are different bioactive ceramics, such as calcium phosphates, hydroxyapatite powders and bioactive glass fibers, and as well as highly porous biocompatible nanocomposites. With wishing to be bound by theory, it is believed that the rate of scaffold bioactivity can be controlled by the amount of bioactive filler incorporated in the polymer matrix.

Further envisaged polymeric materials are poly-L-lactic acid (PLLA), polyglycolic acid (PGA), poly(D,L-lactide/glycolide) copolymer (PDLA) and polycaprolactone (PCL). Examples of suitable and envisaged resorbable polymers include PL Poly(L-lactide), PC Poly(ε-caprolactone), PLC Poly(L-lactide/ε-caprolactone), PLG Poly(L-lactide/Glycolide), PDL Poly(DL-lactide), PL Poly(L-lactide), PC Poly(ε-caprolactone), PLC Poly(L-lactide/ε-caprolactone), PLG Poly(L-lactide/Glycolide), PDL Poly(DL-lactide), PLDL Poly(L-DL lactide), PG Poly(Glycolide), PLDL Poly(L-DL lactide) and PG Poly(Glycolide).

The filtering and recruiting activity of the device is mainly achieved by the presence of one or more chemical and/or biological agents which are comprised on said device and fulfil their activity in situ. An accessory effect improving the filtering or recruiting may be provided by the form and design of the device itself, e.g. by reducing the circulation velocity of the bodily fluid, preferably of the blood stream. Accordingly, the retention time of circulating tumor cell and/or circulating metastatic cell and/or motile parts of tumor cells at or on the device can advantageously be extended, allowing for molecular interactions between the circulating tumor cell and/or circulating metastatic cell and/or motile parts of tumor cells on the one hand, and the one or more chemical and/or biological agents located on the device on the other hand.

It is particularly preferred that the device as defined herein be designed according to current implant techniques and technologies (e.g. stent implantation, septal occluder implantation, vena cava filter implantation). It is further preferred that it comprises an inactive (unexpanded, folded or constraint state) and an active (expanded, unfolded, unconstraint) state. The device is introduced into the target organ in an unexpanded state and is released at the target position into the expanded and active state.

In a further aspect of the first main aspect the invention envisages a tubular shaped device assembly for intraluminal use with a distal and a proximal end, comprising an intraluminal segment which comprises one or more chemical and/or biological agents, for interaction with bodily fluids of luminal organs, wherein at least one portion of the intraluminal segment is expandable and capable of interacting with at least one component of the surrounding bodily fluids via an interactive expandable contact sur-face, wherein the maximal increase in interactive surface area is at least 3 fold, wherein the interactive segment in its expanded state leaves at least 50% of the surrounding continuous cross-sectional luminal plane void of any structural or interactive component of the expansive device at any level of the longitudinal extension of the interactive segment of the device. The device is, in certain embodiments, essentially tubular shaped, which means that it may comprise besides a tubular form also other forms, or that the tubular form is predominant, but is complemented by further different forms, which may vary in accordance with the required functions and applications.

The device accordingly may comprise an intraluminal distal segment and an extraluminal proximal segment, further comprising at least two channels within at least a portion of the longitudinal extension of the device, wherein at least one channel is a through channel for use as wire or infusion channel, and wherein at least one channel is a balloon inflation channel, wherein at least one balloon inflation channel is in fluid connection with at least one flow directable compliant expandable balloon, wherein the balloon is arranged around the distal end section of the intraluminal device, wherein expanded balloon diameters are smaller than the surrounding tubular organ diameter, and wherein at least the expandable portions of the intraluminal segment are interactive with elements of bodily fluids in the target area. Further information may be derived, for example, from FIG. 62.

The tubular shaped device assembly for intraluminal use comprises at least one expandable portion of the intraluminal segment which his an inflatable balloon. Said expandable portion is designed to bind a tumor cell, a tumor cell exosome, and/or a tumor cell aggregate, and/or a pathogen.

It is further envisaged that any expanded and interactive portion at any level of the longitudinal extension of the intraluminal segment spares at least about 50% of the surrounding cross-sectional area from any flow obstructive device components or interactive device elements in favor of more than 50% cross-sectional area void of any device elements adjacent or within the interactive site of the device.

Also preferred are embodiment which envisage that the intraluminal segment comprises at least two reversibly expandable interactive portions which are arranged in a tandem like order along the longitudinal axis of the intraluminal segment. They may also be arranged sequentially and eccentrically and be spaced to each other on the circumference of the tubular intraluminal segment, with an offset in a clockwise orientation with at least 90° differences or opposite to each other. The difference in circumferential position may preferably be 180°, or similar values such as about 170°, about 160°, 190°, 200° or any value in between the mentioned values.

Further envisaged is an embodiment, wherein at least one expandable portion of the intraluminal segment is extending over any length. The length may preferably range from about 10 mm to more than 50% of the intraluminal length of the device assembly, e.g. 20 mm, 50 mm, 100 mm, 200 mm, 300 mm, or more. The expandable portion is designed to be capable of increasing the interactive contact sur-face with the bodily fluid.

Also envisaged is a device assembly, wherein at least a portion of the intraluminal segment of the device provides structural elements as a reservoir for a drug or a biological agent or their compounds, wherein release of these agents is controlled by the release kinetics of the drug compound or is activated by expansive forces such as balloon or self expansion. The drug or biological agent may be, for example, a cytokine, a small molecule, a cytostatic, an anti-cancer drug, an anti-thrombotic drugs, a cardiovascular drug, a drug against a neurologic disease.

Further envisaged is a device assembly as defined herein which additionally comprising at least two localization markers on the intraluminal segment. The markers may identify the proximal and distal end of interactive sites. Such interactive sites may be localized throughout the device, preferably be localized eccentrically and opposite to each other on the circumference of the tubular shaped device. It is preferred that the markers are characterized by a different configuration and that they are visualizable by medical imaging. It is particularly preferred that the eccentric markers are designed to permit visualization of longitudinal and rotational positioning of the device. Said markers comprise contrast deposits visible in medical imaging. It is particularly preferred that said markers are MRI markers. Examples of suitable markers comprise Gadolinium and similar materials like tungsten.

Said contrast deposits may be arranged in any suitable form or on the basis of any suitable architecture. It is preferred that they are arranged within balloon like cavities, more preferably along a non-through lumen such as the inflation lumen.

Also envisaged are device assemblies, wherein the specific device design maintains continuous cross-sectional areas of more than about 60% void of any structural or interactive device components or zones, e.g. as defined herein, adjacent or along its interactive expandable sites at any given point along the longitudinal extension of the device assembly The continuous cross-sectional areas which are maintained void of structural or interactive device components may be, in certain embodiments, range from 50% to 90%.

In certain embodiments, the device assembly according to the present invention comprises in-active sections which permit a free flow of bodily fluids along or within the device, interrupted by one or more sections comprising interactive sites which are designed to permit an interaction with components of the bodily fluids, e.g. ad defined herein comprising certain coatings etc., preferably a filtering by means of a membrane like component. In preferred embodiments the interactive sites spare cross-sectional areas of more than 50%, e.g. 60%, 70%, 80%, 85%, 90%, of the cross-sectional surrounding area for free flow void of any device assembly elements.

In certain embodiments, the device assembly has one or more of the one or more of the properties as defined in the context of the tubular shaped elongated catheter device assembly described above. It is particularly preferred that the device assembly is atraumatically anchorable in a target vessel.

It is further envisaged that the device assembly as defined above is characterized by at least one wire lumen within at least the intraluminal segment of the device and at least one inflation lumen. For example it may comprise 1, 2, 3, 4, 5 or more wire lumen, and/or it may comprise 1, 2, 3, 4, 5 or more inflation lumen.

Also envisaged is a device assembly which is connected to an energy emanating and/or producing module, which allows to destroy target cells and/or target tissues, e.g. cancerous cells. The energy emanating and/or producing module may, for example, generate light, an electrical field, a magnetic field, heat or ultrasound, or any combination or sub-group of the above. The energy emanating module may preferably be situated outside of the subject's body and be connected therewith via a cable, endoscopic device, optical fiber, tube or any other suitable means. It is preferred that a device which is designed to make use of light energy comprises one or more optical fibers for light application. The term “optical fiber” as used herein relates to a flexible, transparent fiber made of silica or plastic for transmitting light between the two ends of the fiber widely used in fiber-optic communications. The optical fiber is hence meant to be a light transmitting element. In the context of the present embodiments it is envisaged to allow the application of light at one or more defined sites of the device and/or close to the device, preferably at a site where target cells are accumulated or are present, or where a target tissue is present. The term “light application” as used herein refers to any type of photo or light conveyed induction or usage in the context of the device as described herein. The device according to this aspect of the invention may accordingly be connected to a light emanating entity, which is preferably connected to an optical fiber. The light may be monochromatic light or a laser beam. The light application may be performed at different wavelengths, or ranges of wavelengths. Typically, a range between 350 nm to 750 nm is used. Particularly preferred wavelengths are 375-400 nm, 630 nm, 635 nm, 652 nm, 665 nm, 670 m, 689 nm, and 732 nm. Preferably, the light application is used for photodynamic therapy. This therapeutic approach, which is envisaged by the present invention, requires the presence of photosensitizer drugs, which may be placed on the device or associated to it. Suitable and envisaged examples of photosensitizer drugs are photofrin, verteporfin, foscan, levulan, metivix, benzvix, hexvix, purlytin, BOPP, photochlor, lutex, Pc4, or talaporfin. The application of light, e.g. of a wavelength as mentioned above, which is advantageously adapted or adaptable to the photosensitizer drug used activates the photosensitizer which lead to type 1 or type 2 reactions and ultimately transform or destroy tissue or cells, e.g. via reactive oxygen species or via radicals. It is particularly preferred that said light application is used in the context of cancerous or tumor tissues or cells, which can be recruited and subsequently be destroyed according to the principles of the present invention. In certain embodiments photosensitizer drugs may be provided independently form the outside at the site of the device, or they may be present in a reservoir structure of the device as defined herein and can be released deliberately.

Further details would be known to the skilled person or can be derived from suitable literature sources such as Dolmans et al., 2003, Nature Reviews, Cancer, 3, 380-387.

In further embodiments the device may be used in the context of an electrical field or a magnetic field. This field may preferably be provided from the outside of the subject via a suitable field generator. The field may preferably be focused to the site of the device and accordingly be used to destroy target cells or target tissue at said site. Also envisaged is the use of heat, which may be applied to the site of the device via focused infrared treatment. The use of ultrasound may accordingly be focused to the device and be implemented by a transducer which is placed at the skin of the subject in the vicinity of the device. Further details would be known to the skilled person or can be derived from suitable literature sources such as Sengupta and Balla, 2018, Journal of Advanced Research, 14, 97-111. Also envisaged are further probes for energy application or transmission of sensor activity.

Further envisaged is a device assembly, wherein at least one expandable interactive site comprises magnetically active components. Such components may be used for different activities including a scavenging magnetically marked antigen or antibody complexes, interacting with magnetically functionalized proteins or small molecules.

The present invention further envisages that the tubular shaped device assembly for intraluminal use with a distal and a proximal end comprises one or more or all of the features defined in the context of the tubular shaped elongated catheter device assembly of the first main context or of the devices as defined in the context of the second or third main context of the present invention.

In yet another aspect of the first main aspect the present invention relates to a multilumen tubular device, characterized by a multilumen design and by a flow-directable balloon design. The multi-lumen concept may implement any of the device forms defined herein above or below.

The term “multilumen” or “multilumen design” as used herein relates to a longitudinally extending tubular structure comprising more than one lumina, e.g. channel, extending within the device. Such channels may comprise elements of different function such as glass fibers, endoscopic instruments, electric or electronic tubes or equipment etc.

The multilumen device may accordingly, in certain embodiments, comprise reversibly expand-able interactive sites which are characterized by interactive surfaces for physical and biochemical or molecular interaction with elements of bodily fluids, wherein at least two interactive sites are arranged in a longitudinally extended tandem position and in a circumferentially clockwise offset position of at least about 90° around the tubular device, wherein a continuous cross-sectional area of at least >50% of total surrounding luminal cross-section is void of any device components at any point along the longitudinal extension of the device. In further embodiments, the device may further be designed for non permanent use as free floating intraluminal device with extracorporeal fixation unit. In further embodiments, at least the beginning and end of an interactive site of the device is marked by markers readable by any medical imaging technique, preferably MRI, and wherein eccentric markers of different configurations on the tubular device permit appreciation of rotational position by medical imaging techniques.

The present invention further envisages that multilumen device as mentioned comprises one or more or all of the features defined in the context of the tubular shaped elongated catheter device assembly of the first main context or of the devices as defined in the context of the second or third main context of the present invention.

In a central aspect of the present invention in the context of the first main aspect the one or more chemical and/or biological agents as described perform different chemical, biological and/or bio-chemical activities. Said activities may ultimately contribute and facilitate an effective recruiting of circulating tumor cells, circulating metastatic cell or motile parts of tumor cells, thus allowing for a removal of said cells, cell aggregates or parts of tumor cells or exosomes from the bodily fluid, e.g. from the blood and at the same time allow for a prolonged and safe use of the device within a subject, as well as the efficient and extensive removal of these cells and parts thereof from the subject, even after a prolonged time, e.g. after 2 to 30 days, 4 to 8 weeks, or 1 to 24 months. To achieve one or more, preferably all, of these functions, the one or more chemical and/or biological agents are provided as coating on the device, in particular on the filter membrane of the device as defined herein above.

Without wishing to be bound by theory, it is assumed that such tumorous cell recruiting and removal processes at the surface of the device implant are influenced by several factors, which may advantageously be used in the context of the present invention. One important factor, which is largely implemented by the physical form and design of the device is the feeding of bodily fluids, e.g. blood or lymph, towards sections of the device which comprise one or more chemical and/or biological agents. For example, the device may be designed such that filter membranes comprising said one or more chemical and/or biological agents are optimally exposed to bodily fluids, e.g. blood or lymph, comprising cells or parts thereof such as circulating tumor cells or exosomes, for circulating metastatic cell or for motile parts of tumor cells or for aggregates of cells. The exposure may further be improved by mechanical fluid manipulation, e.g. specific designs of the device such as depicted in FIGS. 24 to 31 or defined above. A further factor is the coating of the device with suitable chemical or biological agents. The coating may fulfil different functions which can differ along the length of the device or according to its intended use. For example, the provision of a passive coating with one or more polymeric materials as defined herein below fulfils inter alia an antithrombotic function and may also serve as a basis for further chemical and/or biological coating.

A further important factor is the presence of an ECM-like structure on the surface of the device, as defined herein below. The ECM-like structure may be provided in an equal manner throughout the device, or it may be provided in certain sectors or regions or the device only, or in certain sectors or regions of the device different forms of these structures may be present. The ECM-like structure essentially fulfils the function of a structural and/or biochemical support for surrounding cells, which is assumed to be of elevated importance once cells have settled down on or within the device. Furthermore, the ECM-like structure resembles the microenvironment of the tumor or metastases. This ECM-like structure which is typically composed of laminins, adhesion molecules or chemical entities serves as homing compartment within the device. Yet another important factor, which is considered relevant for the initial recruiting steps is the presence of a biological agent, which is capable of binding to a tumor marker on a cell or part thereof, in particular on a recruiting a circulating tumor or circulating metastatic cell or motile parts of tumor cells. These different factors may, in specific embodiments, present together at one area of the device, or they may fulfil their functions in different areas or the device. Also suitable mixture of any of these factors are envisaged. Furthermore, the present invention envisages different forms of devices which may comprise or implement either all or a sub-group of said factors. The overall shape, the intended use, the specific medical condition of a subject, the intended time of use and/or the tumor form may require an adjustment of the presence of above mentioned factors or functions. Additional factors which may also be taken into account are the tumor staging, i.e. the phase or stadium of the disease, and/or details on a pre-treatment, e.g. whether the subject has already been treated, how successful this treatment was and the time period since the treatment, as well as a possible chemotherapeutic resistance etc.

Accordingly, a device of the invention may, particularly preferred embodiments, comprise (i) a passive coating with one or more polymeric materials, (ii) as well as an ECM-like structure, and (iii) an active coating which is capable of binding to a tumor marker on a cell or part thereof. In further embodiments, at least one of these elements (i) to (iii) may not be present. The present invention envisages all combinations of (i), (ii) and (iii) as defined above.

In one group of embodiments of the invention the coating may thus be a passive coating. The term “passive coating” as used herein in the context of all main aspects, as well as related embodiments, means that the coated agent is chemically or biochemically inert. Such a passive coating is typically not involved in cell recruiting or removal processes. Passive coatings may advantageously exhibit antithrombotic properties. Passive coatings may also serve as a basis for further chemical and/or biological coating. Passive coatings may also permit to design release kinetics of chemicals/drugs. A further function of the passive coating is protection against corrosion and/or the provision of linkage or conjugation option for additional coating layers, e.g. biological agents as defined herein. The passive coating may be performed, for instance, with one or more polymeric materials or may comprise such materials. Preferred examples of such materials are ethylene vinyl acetate (EVA), latexes, urethanes, polyurethanes, polysiloxanes, styrene-ethylene/butylene styrene block copolymers (SEBS), polytetrafluoroethylene (PTFE) and linear aliphatic polyesters. The passive coating according to the present invention is connected to the device as defined above via adherence mechanisms. In preferred embodiments, the adherence is conveyed via adhesive layer between the surface of the device, comprising at the surface, for example, metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper; plastic or polymeric material; elastic memory shape meshwork material such as memory shape elastic wires or a material readable by tomography or other imaging techniques, e.g. X ray or MRI (e.g. tungsten); or any mixture thereof, and said coating. The adhesive layer may, for example, be composed or may comprise sugar, starch, polyvinyl-alcohol or degradable products thereof.

In a further group of embodiments, the chemical and/or biological agents composing the coating of the device constitute an extracellular matrix-like structure. The term “extracellular matrix like structure” or “ECM-like structure” as used herein in the context of all main aspects, as well as related embodiments, relates to a structure which simulates the three-dimensional network of extracellular macromolecules of an ECM and thereby provides structural and/or biochemical support for surrounding cells. The ECM-like structure accordingly provides, at least partially, important functionalities for a recruiting and removal from circulation of circulating tumor cells, circulating metastatic cell or motile parts of tumor cells. The ECM-like structure, for example, is capable of slowing down cells, or may increase their interaction competence for binding interactions, e.g. with biological agents as defined herein. To provide an extracellular-matrix-like structure, the present invention envisages the provision of extracellular molecules typically present in the extracellular matrix, in particular in the extracellular matrix of a mammal, more preferably in the extracellular matrix of a human being. The identity of the ECM constituting macromolecules may be adapted to the subject in which the device is to be used. For example, for human beings the typical composition of human ECMs may be used. For animals, e.g. cats, dogs, horses, cattle etc. corresponding ECM compositions may be employed.

In specific embodiments, the macromolecules used to provide an ECM like structure, i.e. the macromolecules which are provided as coating for the device according to the present invention, are proteoglycans, non-proteoglycan-polysaccharides, elastin; fibronectin or laminin. Particularly preferred are mixtures of these macromolecules. Preferred examples of suitable proteoglycans include heparan sulfate, chondroitin sulfate and/or keratin sulfate. Preferred examples of suitable non-proteoglycan-polysaccharides include hyaluronic acid. The present invention further envisages the employment of ECM-like structures in the form of protein mixtures. Such mixtures are, for examples, secreted by Engelbreth-Holm-Swarm (EHS) mouse sarcoma cells. Also preferred are Matrigel, BioCoat or GelTrex mixtures. It is particularly preferred that protein mixtures with human proteins are used.

According to the present invention, the device comprising a coating as defined herein provides an environment for circulating tumor cells, for circulating metastatic cell or for motile parts of tumor cells, for exosomes, for cell aggregates, and normal cells such as immune cells. The environment may, for example, allow metastatic cells to settle down in or on the device and thus leave the bodily fluid circulation, e.g. lymph or blood. Similarly, tumor cells which are non-metastatic but circulate through the body may be stimulated to settle down in or on the device and thus leave the bodily fluid circulation, e.g. lymph or blood. This function is preferably implemented by the use of, or presence of a biological agent in or on the device, in particular as part of the coating, wherein said biological agent is capable of binding to a tumor marker on a cell or part thereof, in particular on a recruiting a circulating tumor or circulating metastatic cell or motile parts of tumor cells. In further embodiments, the present invention also envisages a binding to immune cells or corresponding markers on immune cells, i.e. immune cell markers. This type of biological agent which is capable of directly interacting with a tumor marker or immune cell marker is referred to by the present invention as “active coating” or part of an active coating.

The term “tumor marker” as used herein in the context of all main aspects, as well as related embodiments, is understood as a protein such as, for example, an enzyme, a structural protein, a receptor protein, or a hormone, a fragment of a protein, a conjugated protein, a peptide or a carbohydrate, which is present on the surface of a tumor cell or motile parts of tumor cells and/or is produced by these cells or parts thereof. Such tumor markers may be indicative for any type of tumor which leads to circulating tumor cells or to metastatic cells. Also envisaged are synthetic tumor markers, e.g. peptide based tumor marker based on artificial sequences known or assumed to relevant as tumor marker. It is preferred that the tumor markers are tumor markers specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma. In further preferred embodiments, the tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphav beta5, P-Selectin, L-Selectin, Integrin alphav beta5, Integrin alpha4 beta7, Integrin alpha2 beta1, Integrin alpha2 beta3, Integrin alphav beta3, Galectin-3, N-CAM, L-Selectin, LPAM-I (alpha4 beta2), CTLA, Integrin alpha4 beta1, Integrin alphaE beta7, CCR10, Axl/Mer, Anxa2-R or Desmoglein I (DG I). Further envisaged are E-selectin ligands such as HCELL, PSGL1, MUC1, as well as LGALS3BP. These markers are known to the skilled person. Further information may be derived from suitable literature references such as Aceto et al., Cell, 2014, 158, 5, 1110-1122 or databases such as Genecards (https://www.genecards.org; last visited on Jun. 26, 2019), or Genbank at NCBI (https://www.ncbi.nlm.nih.gov/genbank; last visited on Jun. 26, 2019).

The term “capable of binding to a tumor marker” as used herein in the context of all main aspects, as well as related embodiments, means that the biological agent performs a molecular, non-covalent binding interaction with said tumor marker and/or further components on a cell or part thereof, which allows for an at least temporary fixation of the cell carrying the tumor marker at the place of the biological agent. Without wishing to be bound by theory, it is assumed that such a binding is mediated by binding capacity depending on signals inside of the passing cells. These signals typically result from the interaction with the specific biological agents as defined herein. They may include, for example, cell death signals, or immune modulation signals. The present invention envisages several components which are capable of binding to a tumor marker. Preferred examples are a tumor-marker specific antibody or a fragment thereof, the protein CD133 or a fragment or domain thereof, the protein VEGFR-1 or a fragment or domain thereof, a homing factor or a fragment or domain thereof; and a tumor-marker specific lectin or a fragment or domain thereof.

Examples of tumor-marker specific antibodies envisaged by the present invention include panitumab, matuzumab, nimotuzumab or derivatives thereof. Further envisaged are additional antibodies against EGFR family members, or ErbB2/HER2 family members. Further details would be known to the skilled person or can be derived from suitable literature sources such as Huang and Buchsbaum, Immunotherapy, 2009; 1(2): 223-239.

CD133 is pentaspan transmembrane glycoprotein. The protein typically localizes to membrane protrusions and is often expressed on adult stem cells, where it is thought to function in maintaining stem cell properties by suppressing differentiation. The protein consists of five transmembrane segments, with the first and second segments and the third and fourth segments connected by intracellular loops while the second and third as well as fourth and fifth transmembrane segments are connected by extracellular loops. Further information may be derived from suitable literature sources such as Glumac and LeBeau, Clin Trans Med, 2018, 7, 18.

VEGFR-1 relates to vascular endothelial growth factor receptor 1 which is encoded by the FLT1 gene in humans. The protein has been shown to interact with PLCG1 and vascular endothelial growth factor B (VEGF-B).

The term “homing factor” as used herein in the context of all main aspects, as well as related embodiments, relates to cell adhesion molecules which typically interact with corresponding or cognate interactors such as adresssins on target tissues. For example, a hepatic microenvironment essentially determines tumor cell dormancy and metastatic outgrowth of pancreatic ductal adenocarcinoma. Further information may be derived from suitable literature sources such as Lenk et al., Oncolmmunology, 2018, 7, 1.

The present invention, in preferred embodiments, envisages the use of one or more of the following homing factors: Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

The term “immune cell” as used herein in the context of all main aspects, as well as related embodiments, refers to a CD8⁺ cell, a dendritic cell, a T cell, an engineered T cell, a B cell, an NK cell, a HSC, a MPP, a CLP, a CMP, a MEP, a GMP, a monocyte, a macrophage, a neutrophil, an eosinophil, a basophil, a mast cell, a megakaryocyte, or a platelet. The term “immune cell marker” relates to a marker present on an immune cell as defined above, e.g. any one of a CD8⁺ cell, a dendritic cell, a T cell, an engineered T cell, a B cell, an NK cell, a HSC, a MPP, a CLP, a CMP, a MEP, a GMP, a monocyte, a macrophage, a neutrophil, an eosinophil, a basophil, a mast cell, a megakaryocyte, or a platelet. In specific embodiments, the immune cell marker may be one or more of CD34, CD49, CD90/Thy1, CD10, CD45RA, CD38, CD135, CD123, NKp46, CD56, CD94, CD3, CD16, CD18, CD19, CD14, CD11b, CD11c, HLA-DR, CD68, CD163, CD32, CD44, CD55, CD45, CD125, CD193, F4/80, Siglec-8, CD235a, CD33, CD117, CD203c, FIERI, CD22, CD41b, CD42a, CD42b, CD61.

In specific embodiments any combination of the above mentioned elements, e.g. homing factors, CD133, VEGFR-1 or antibody may be used. Also envisaged is the use of one type of element, e.g. one specific homing factor, in a specific area of the device, followed by a different type of element in the neighbouring area etc.

The present invention further envisages that a biological agent as defined herein, e.g. a homing factor, an antibody etc. is linked to the passive coating of the device via a spacer element. Also envisaged is the linkage to the structural support material of the device, e.g. stainless steel, gold, titanium, gold-titanium alloy, cobalt-chromium alloy, tantalum, platinum-radium alloy, tantalum alloy, magnesium, nickel-titanium alloy, e.g. nitinol, silver or copper; plastic or polymeric material; elastic memory shape meshwork material such as memory shape elastic wires or a material readable by tomography or other imaging techniques, e.g. X ray; or any mixture thereof, which may be present on the surface of said filter device assembly implant. The term “linked” as used herein refers to a persistent connection between said biological agent and the device. The linkage may either be performed with the structural support material as defined above. In such an embodiment, the linkage may be implemented as binding to a metal ion resin, preferably ion-NTA or ion-agarose. Alternatively, the linkage may be performed with the passive coating of the device. In corresponding embodiments, the linkage may be implemented as covalent binding between parts of the passive coating and the biological agent or the spacer element attached to it.

The term “spacer element” as used herein in the context of all main aspects, as well as related embodiments, relates to a distance piece which is capable of spatially separating the biological agent form the surface of the device. This separating allows for a sterically unhindered interaction of the biological agent with a component on a circulating tumor cells or the like. In preferred embodiments the spacer elements are composed or partially composed of a peptide or a polypeptide. It is particularly preferred that the Fc part of an antibody or multi-histidine tag be used. Also envisaged is the employment of a nucleic acid; a modified nucleic acid; or a polymer such as PEG, PLA, PVA, polyethylene or polypropylene. The spacer element may have any suitable length. In a preferred embodiment, the spacer element has a length of about 1 to 20 nm, e.g. 1, 2, 3, 4, 5, 6, 7, 8, 9, 10, 11, 12, 13, 14, 15, 16, 17, 18, 19 or 20 nm or any value in between the mentioned values. Also envisaged are shorter spacer elements in a length of 7 to 25 amino acids, e.g. 7, 8, 9, 10, 11, 12, 13, 14, 15, 16, 17, 18, 19, 20, 21, 22, 23, 24, 25 amino acids, or equivalents thereof.

In addition to its function as spatial separator between the biological agent and the surface of the device, the spacer element further fulfils the function of a separator between biological agents as defined herein. The present invention accordingly envisages the provision of biological agents in a density which allows for an efficient binding of the biological agent to a tumor cell or part thereof. According to preferred embodiments, the spacer elements may be provided in a density of 2 to 500 per μm2 on the surface of the device. For example, a density of 2, 5, 10, 15, 20, 30, 40, 50, 60, 70, 80, 90, 100, 150, 200, 250, 300, 350, 400, 450 or 500 per μm2 on the surface of the device may be used.

The present invention further relates to biological agents—as part of the device—which comprise, essentially consists of, or consists of a binding domain capable of binding to a tumor marker. The term “binding domain capable of binding to a tumor marker” as used herein relates to an amino acid sequence which comprises a non-covalent binding functionality for a tumor marker as mentioned herein above. Preferably, the binding domain is capable of binding to any of CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphav beta5, P-Selectin, L-Selectin, Integrin alphav beta5, Integrin alpha4 beta7, Integrin alpha2 beta1, Integrin alpha2 beta3, Integrin alphav beta3, Galectin-3, N-CAM, L-Selectin, LPAM-I (alpha4 beta2), CTLA, Integrin alpha4 beta1, Integrin alphaE beta7, CCR10, Axl/Mer, Anxa2-R or Desmoglein I (DG I). The present invention also envisages binding domains for further tumor marker including those which have not yet been identified.

In preferred embodiments, the binding domain is a peptide or polypeptide molecule. The do-main may have any suitable length. It is preferred that it has length of about 20 to about 250 amino acids. More preferably, the binding domain has a length of about 20 to about 120 amino acids. In further preferred embodiments, the binding domain has a length of about 20, 25, 30, 35, 40, 45, 50, 55, 60, 65, 70, 75, 80, 85, 90, 95, 100, 105, 110, 115 or 120 amino acids, or any value in between the mentioned values.

Information on suitable binding domains capable of binding to a tumor marker can be derived from literature resources or databases, e.g. from Partyka et al., Proteomics, 2012, 12(13), 2212-2220.

The binding domain capable of binding to a tumor marker according to the present invention may be provided in any suitable form. For example, the binding domain may be provided as part of a larger protein structure, wherein said larger protein structure may fulfil presenting or structural functions, thus improving the expose of the binding domain. Further envisaged is combination of more than one binding domain per biological agent unit. The term “biological agent unit” as used herein means that a biological agent may comprise one or more functions, but is attached to the device in a single manner, e.g. via a linker or spacer element as defined herein. Accordingly, a biological agent unit may comprise 2, 3, 4, 5 or more binding domains and/or structural protein components. Also envisaged is the presence of intervening peptide elements within the unit between the domains. Also envisaged is the additional use a linker element between the biological agent and said spacer. The additional linker may have a length of 1, 2, 3, 4, 5, 6, 7, 8, 9, 10, 11, 12, 13, 14, 15, 16, 17, 18, 19 or 20 amino acids. The linkage between the biological agent and the spacer may further be implemented as covalent or non-covalent connection.

In further embodiments, the binding domain capable of binding to a tumor marker according to the present invention may be combined with one or more additional functional domains. Preferably, such a further functional domain is or comprises, partially comprises or consists of an apoptosis inducing factor or a functional domain of an apoptosis inducing factor capable of inducing apoptosis. Preferred examples of suitable apoptosis inducing factors are FasL/CD95L, TNF-alpha, APO3L and APO2L/TRAIL. The presence of such apoptosis inducing factors advantageously allows the device of the present invention to not only attract tumor cells or metastatic cells, but also to subsequently induce a killing of these cells via apoptotic processes. The present invention further envisages the use of additional killing approaches based on molecular interactions between the surface of a cancer cell or metastatic cell and an interactor.

In specific embodiments of the present invention the domain capable of binding to a tumor marker and the domain capable of inducing apoptosis are provided as fused domains. Also envisaged is the alternative that the domains are linked via a linker element of about 1 to 20 amino acids length. For example, the linker element may be a peptide having a length of 1, 2, 3, 4, 5, 6, 7, 8, 9, 10, 11, 12, 13, 14, 15, 16, 17, 18, 19 or 20 amino acids.

In further specific embodiments of the present invention the biological agent is provided as linear or circular element or as an element composed of linear and circular parts. It is particularly preferred to provide the biological agent as a linear or circular or partially linear or circular peptide or poly-peptide. Typical embodiments envisage that the circular biological agent has or is part of a structure comprising a loop or a loop and a stem. Also envisaged are linear structures, which are linked to a spacer element as defined herein. The loop structure or linear structure may comprise said preferably comprise the biological agent, e.g. the tumor binding domain or the apoptosis inducing domain, at an exposed position. Said exposition allows for an efficient interaction, e.g. leading to the binding to a tumor marker or a tumor cell, or the induction of apoptosis.

In a further preferred embodiment the peptide as used in the context of the present invention has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined conjugated with stabilizing components such as PEG or lipids, preferably forming lipoglycopeptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, comprises, essentially consists of, or consists of D-amino acids and/or L-amino acids; (xi) it partially comprises, comprises, essentially consists of, or consists of homo-amino acids such as beta-homo-amino acids, N-methyl amino acids, or alpha-methyl-amino acids; (xii) it partially comprises, comprises, essentially consists of, or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide. Particularly preferred is the use of cell penetrating functions. Also envisages is any combination of these peptides with components of the device as mentioned above. Further information on specific sequences of peptides envisaged by the present invention may be derived from FIG. 63, 64 or 65.

In further specific embodiments a biological agent as defined herein above comprises or is linked to one or more additional elements. For example, the biological agent may be linked to a sugar, to branched or unbranched multiple sugar structures, to alkynes or azides, to streptavidin, biotin, amines, carboxylic acids, active esters, epoxides or to aziridines.

In yet another specific group of embodiments, the device according to the present invention comprises a pharmaceutical agent. For example the pharmaceutical agent may be provided as part of a coating as defined herein. The pharmaceutical agent may, in certain embodiments, improve or support one or more functions of the device, e.g. the recruiting or removal function for tumor cells or metastatic cells. For example, the pharmaceutical agent may be a cytotoxic compound which kills recruited tumor cells or metastatic cells. In particularly preferred embodiment, the pharmaceutical agent is an antiproliferative agent or an anticoagulant. The provision of an antiproliferative agent on or in the device allows for a reduction of cell growth, e.g. tumorous growth or metastatic growth in the device. Furthermore, the antiproliferative agent is useful to reduce the growth of endothelial cells in the neighborhood of the device, thus preventing an overgrowth or occlusion of the device, or an embolism. The provision of an anti-coagulant agent on or in the device allows for the prevention of blood clotting inside the device and also an occlusion of the device or an embolism. Preferred examples of suitable antiproliferative agents are paclitaxel or sirolimus. Preferred examples of suitable anticoagulants are reteplase or heparin.

The pharmaceutical agent may be provided in an amount which is adjusted to the intended period of use. In further embodiments, the pharmaceutical agent may be released in a time-controlled manner, e.g. in a steady concentration over the entire intended period of use. Suitable controlled release technologies are known to the skilled person or can be derived from suitable literature references such as Senst B, Basit H, Borger J. Drug Eluting Stent (DES) Compounds. In: StatPearls [Internet]. Treasure Island (FL): StatPearls Publishing; 2019 January (available at https://www.ncbi.nlm.nih.gov/books/NBK537349/)

In a further aspect the present invention relates to device as defined herein for use in treating cancer and/or metastasis, or for use in preventing cancer and/or metastasis in a subject.

The term “subject” or “patient” used herein in the context of all main aspects, as well as related embodiments, refers to a mammal. “Mammal” as used herein in the context of all main aspects, as well as related embodiments, is intended to have the same meaning as commonly understood by one of ordinary skill in the art. Preferred mammals are primates, cows, sheep, goats, horses, dogs, cats, rabbits, rats, mice and the like. In particularly preferred embodiments, the subject is a human being. The term includes as treatment target group persons affected by a pathological condition. Also envisaged as treatment target group are healthy subjects or subjects showing no symptoms of a disease, preferably showing no symptoms of cancer or metastasis. Further envisaged as treatment target group are subjects being at risk of developing cancer and/or metastasis.

The term “administered” as used herein in the context of all main aspects, as well as related embodiments, relates to the provision and possible maintenance of a therapeutically effective form of the device at any suitable place in the subject's body. By “therapeutically effective form” is meant a dose or number of biological agents on or in the device that produces the effects for which it is administered. The exact dose or number of biological agents will depend on the purpose of the treatment, and will be ascertainable by one skilled in the art using known techniques. As is known in the art and described herein, adjustments for localized usage, age, body weight, general health, sex, diet, time of administration/use, drug interaction and the severity of the condition may be necessary, and will be ascertainable with routine experimentation by those skilled in the art.

The device of the present invention may be used in both human therapy and veterinary therapy, preferably in human therapy.

The device described herein may be used or administered alone or in combination with other treatments. Combination treatments are envisioned for cancer immunotherapy, for example via co-ad-ministration of checkpoint inhibitors such as anti-CTLA-4 and anti-PD1 antibodies, or for chemotherapy, for example by co-administration of alkylating agents or DNA and RNA polymerase inhibitors. Further combination approaches may involve targeting cancer cells by mitochondrial mediated apoptosis, targeting cancer cells by ROS mediated apoptosis, targeting cancer cells by death receptor mediated apoptosis, targeting cancer cells by cell cycle mediated apoptosis, targeting cancer cells by regulating multiple sig-nailing pathways and transcription factors. Also envisaged are additional targeting approaches for the STAT3 Pathway, the PI3K/AKT/mTOR Pathway, the MAPK/ERK (Ras-Raf-MEK-ERK) Pathway, the Wnt/13-Catenin Pathway, for Hypoxia-inducible Factor-1α (HIF-1α), for the COX-2/PGE2 pathway, and combinations with cytokines and chemotherapy. In specific embodiments the death receptor peptides to be used in this context may be of the following type: Apo-3L/TWEAK and DR3 APo-3; Apo-2L/TRAIL and DR4/5; TNFalpha and TNF-R2 and R1; FasL or Fas/CD95.

The terms “treat” or “treatment”, as used in the context of all main aspects, as well as related embodiments, unless otherwise indicated by context, refer to therapeutic treatment and/or prophylactic measures to prevent the outbreak or relapse of a cancer disease, wherein the objective is to inhibit or slow down (lessen) an undesired physiological condition. For purposes of this invention, beneficial or desired clinical results include, but are not limited to, alleviation of symptoms, diminishment of extent of a cancer disease, stabilized (i.e., not worsening) state of a cancer disease, delay or slowing of a cancer disease progression, amelioration or palliation of the cancer disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder. The term “prevention” as used herein relates to prophylactic measures in order to impede or avoid the affection by cancer or cancerous metastasis of a subject. The prevention may, in certain embodiments, include quantitative or almost quantitative capturing of cancer cells or metastatic cells and a subsequent destruction or removal of these cells from the circulation.

The treatment or prevention may further, in specific embodiments, involve a single administration or use of the device as defined above, or multiple administrations or uses. A corresponding administration or usage scheme may be adjusted to the sex or weight of the patient, the disease, the general health status of the subject etc. For example, the administration or use scheme may contemplate a usage within the subject's body for one week, two weeks, 3, 4, 5, 6, 7, 8, 10, 11, 12 weeks, 4, 5, 6, 7, 8, 9, 10, 11, 12, 18, 24 or more months, or any time period in between the mentioned periods. These usage schemes can of course be adjusted or changed by the medical practitioner in accordance with the subject's reaction to the treatment/prevention and/or the course of the pathological condition.

The device as defined above may accordingly be administered to the subject in as a controlled active, controlled release and toxic entities (tumor cells or metastatic cells) accumulating agent.

The administration or implantation of the device may preferably be performed during and/or after the treatment of a subject with a therapeutic agent. The treatment of a subject as mentioned here is typically an anti-cancer treatment. The administration or implantation of the device may, in further embodiments, be preferably performed during and/or after surgery removing a tumor load. It is particularly preferred that the administration scheme foresees an implantation immediately after said surgery or, in the alternative, as ultimate step of the surgery to capturing not removed cancer cells.

The term “cancer” or “tumor” as used herein in the context of all main aspects, as well as related embodiments, relates to a pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms typically show partial or complete lack of structural organization and functional coordination with the normal tissue and most invade surrounding tissues, metastasize to several sites, and are likely to recur after attempted removal and to cause the death of the patient unless adequately treated. As used herein in the context of all main aspects, as well as related embodiments, the term “neoplasia” is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma skin cancer, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma. Also envisaged are further cancer forms known to the skilled person or derivable from suitable literature sources such as Pavlopoulou et al., 2015, Oncol Rep., 33, 1, 3-18. The cancer may, in certain embodiments, be a refractory cancer. A cancer or tumor may be assumed to be residually present if a subject has undergone surgery as treatment for the cancer or tumor.

The term “metastasis” as used herein in the context of all main aspects, as well as related embodiments, relates to a tumor's spread from an initial or primary site to a different or secondary site within the subject's body. A metastasis may be provoked by or circulating metastatic cells, which are have typically acquired the ability to penetrate the walls of lymphatic or blood vessels, after which they are able to circulate through the bloodstream to other sites and tissues in the body, re-penetrate the vessel or walls and continue to multiply, eventually forming another clinically detectable tumor. A metastasis is hence a secondary tumor form based on circulating cells.

It is particularly preferred that the cancer to be treated or prevented is a colon cancer, breast cancer, lung cancer, melanoma, esophageal cancer, prostate cancer, pancreatic cancer, ovarian cancer, myeloma, or a lymphoma such as ALL, CLL, or AML. It is further particularly preferred that the metastasis to be treated or prevented is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarial tumor or nephroblastom such as Wilms tumor. Due to its shape and, in particular, its functionality as defined herein above, the device of the present invention is capable of quantitatively or almost quantitatively capturing circulating tumor cells or metastatic cells or motile parts of tumor cells in a subject's body. Due to its shape and, in particular, its functionality as defined herein above the device of the present invention is accordingly capable of preventing downstream organs or tissues from being reached by tumor cells or metastatic cells or motile parts of tumor cells which are circulating in a subject's body. It is particularly preferred to treat tumor or tumor cells in or close to the space of Disse.

In further specific embodiments, the device of the present invention is specifically designed to be implanted into a luminal organ, preferably a blood vessel. Examples of envisaged blood vessels are an artery, an elastic artery, a distributing artery, an arteriole, a capillary, a venule or a vein. It is further envisaged to design the device for an implantation into a heart chamber or an elastic artery. The device may accordingly be designed as free floating device with connecting wires to maintain its position. This concept also allows for retrieval. In further specific embodiments, the device of the present invention is specifically designed to be implanted into a lymphatic vessel. Particularly preferred is vena cava, or any other vessel wherein said device can suitably be used free floating. Further envisaged and preferred is a transvascular implantation. In certain embodiments, the device is designed to be transvascularly advanced and implanted at a target site. The device may accordingly be designed to have adequate dimensions for an advancement in the target tissue or region. It may, in further embodiments, be catheter based and preferably steerable. It is particularly preferred that the device is steerable is by its torque stable structure or by comprising a steerable wire. Also envisaged in the present invention is a percutaneous implantation, a minimal invasive implantation, an endoscopically guided implantation, e.g. via ultrasound or magnetic resonance devices, or a laparoscopic implantation. The implantation may, in preferred embodiments, be performed directly in a tumor or tumorous tissue or in the close vicinity of a tumor or tumorous tissue, or in any other target organ or tissue, preferably into luminal or tubular parts of an organ. Also envisaged are corresponding device designs and their use.

In certain specific embodiments a device to the implanted via a percutaneous implantation, a minimal invasive implantation, an endoscopically guided implantation, e.g. via ultrasound or magnetic resonance devices, a laparoscopic implantation, or a transcutaneous implantation may have a specific form or design, e.g. may not be composed of filters as defined herein. Preferably, the device may have or comprise a porous solid architecture or design, or may have the form of porous solid scaffold.

It is particularly preferred that the device of the present invention is used by implantation in a blood or lymphatic vessel downstream of an existing cancer site in a subject. If such a positioning downstream of an existing cancer is envisaged, it is preferred that the device is used by implantation in close proximity to said existing cancer site.

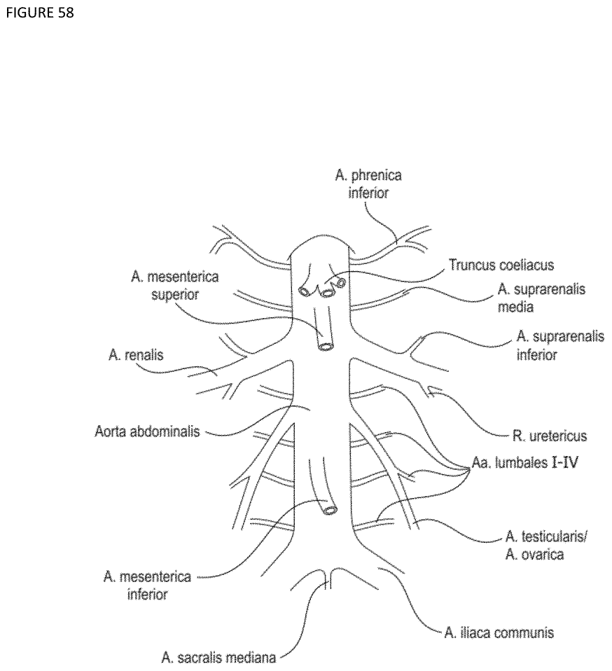

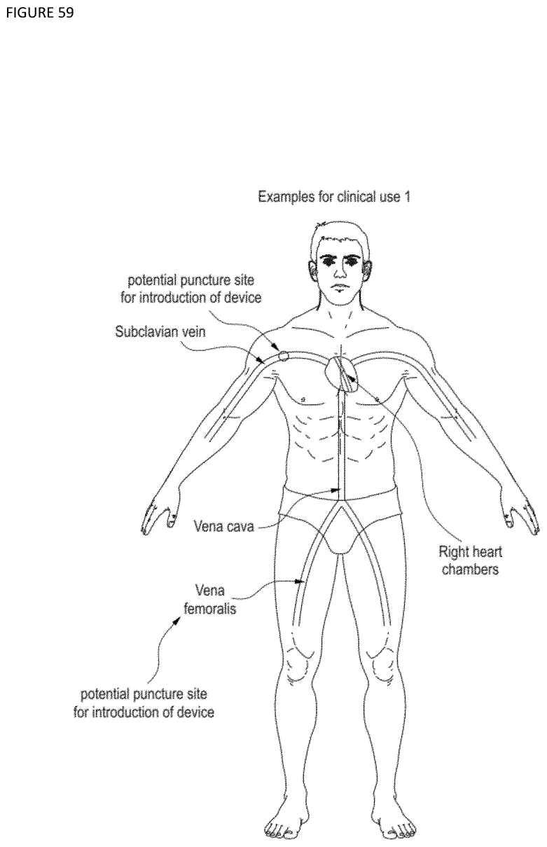

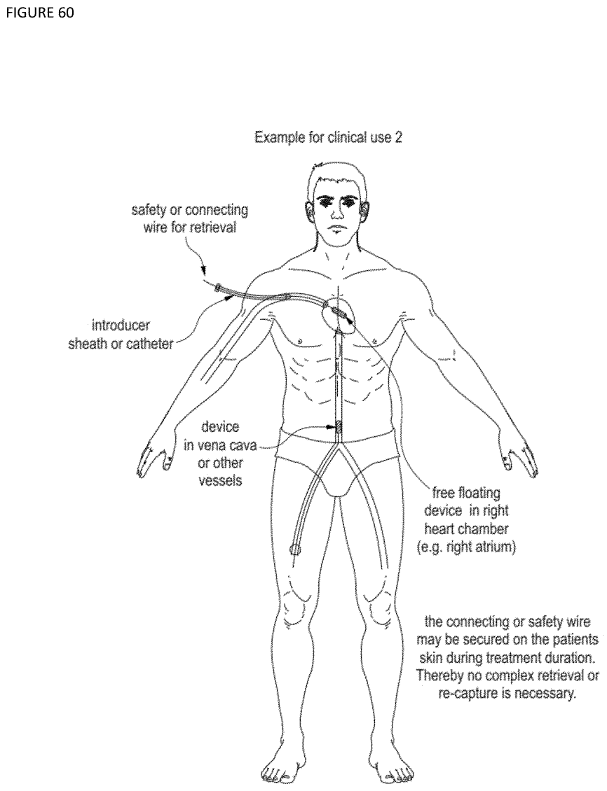

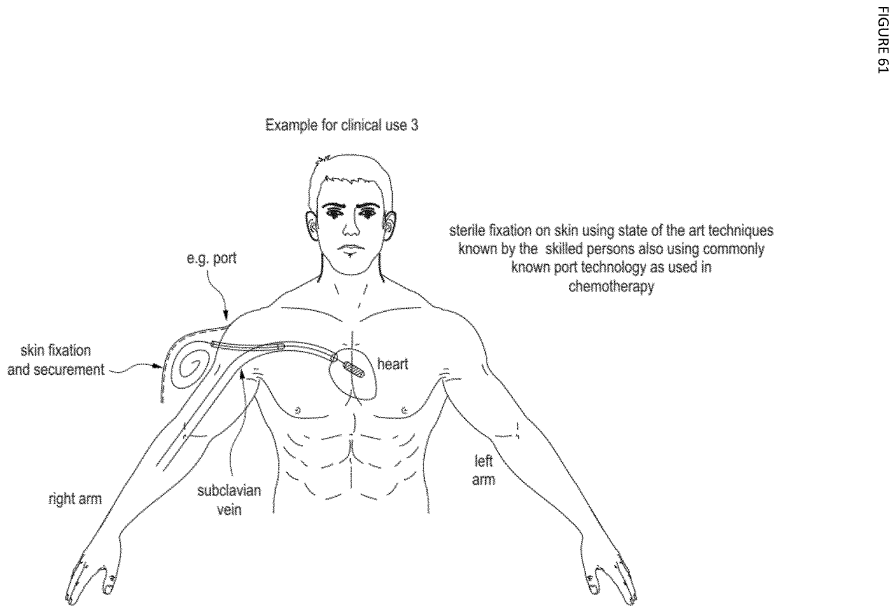

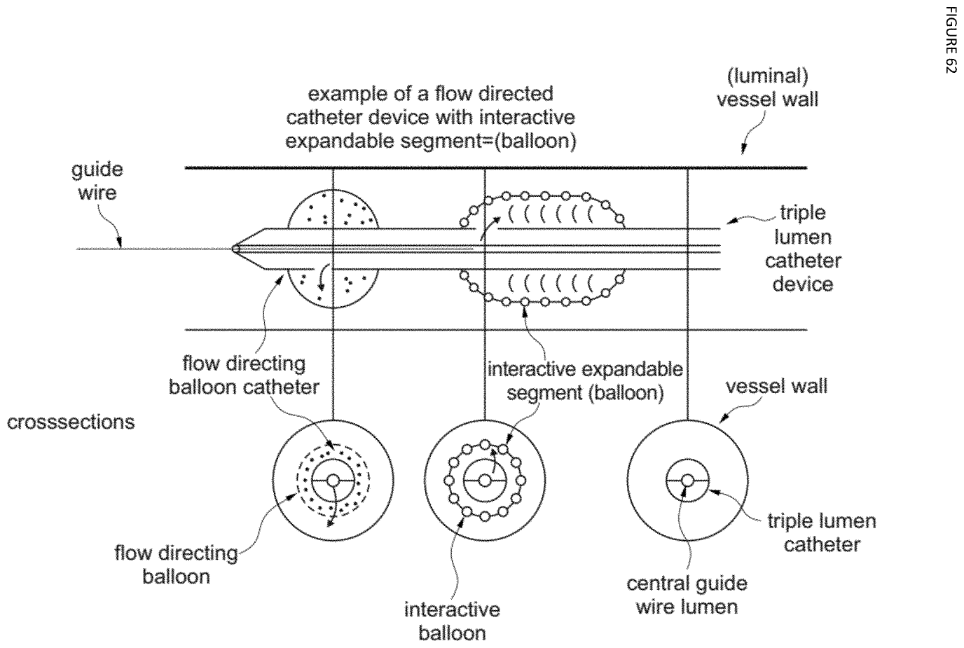

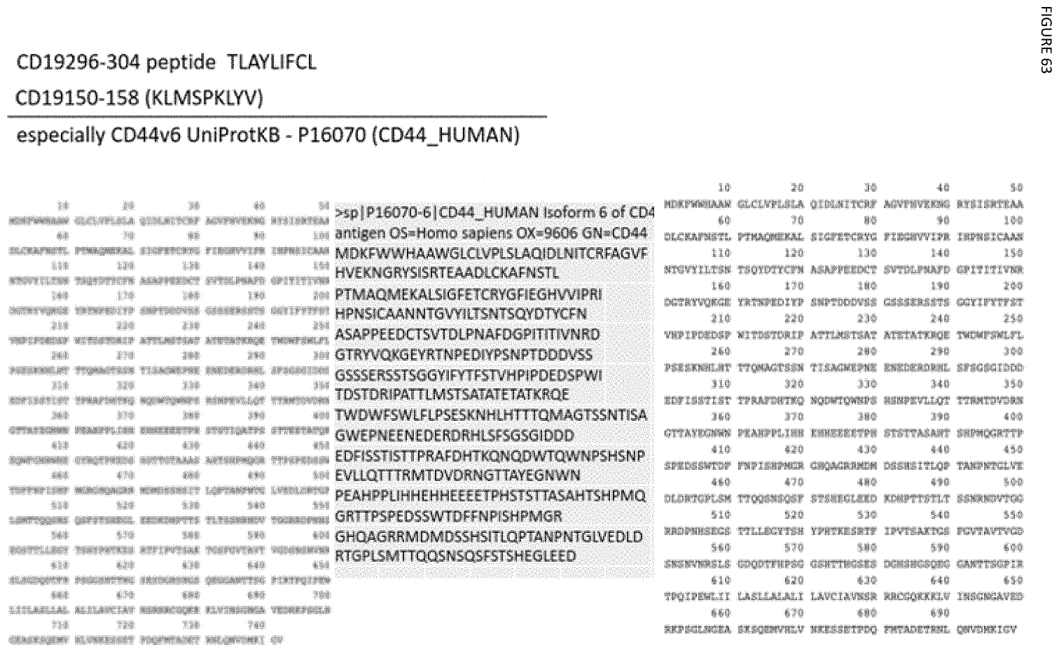

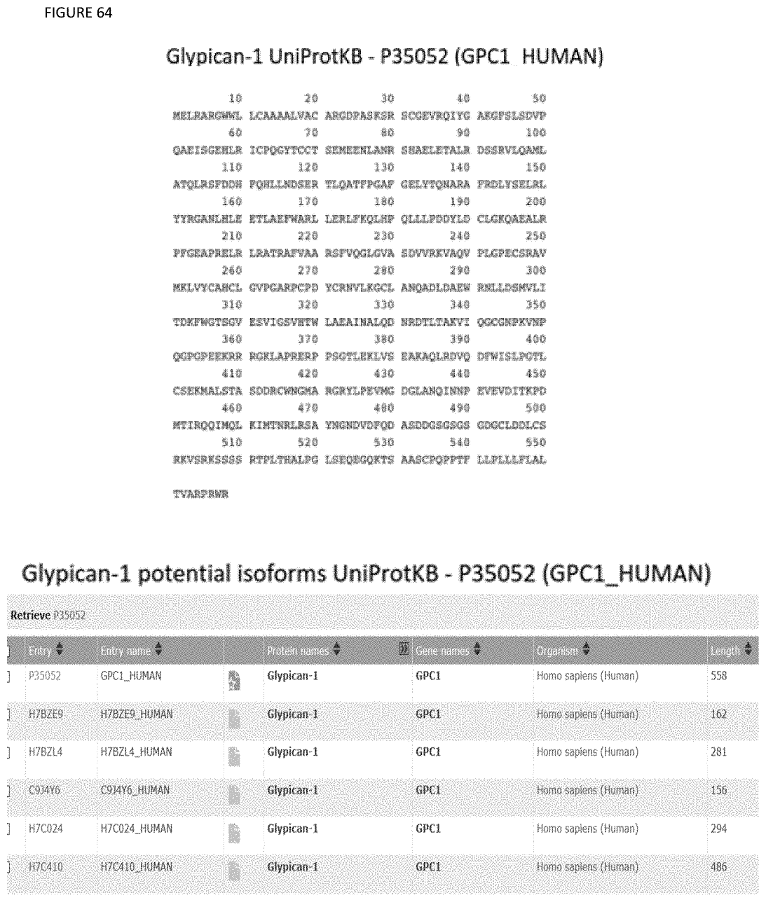

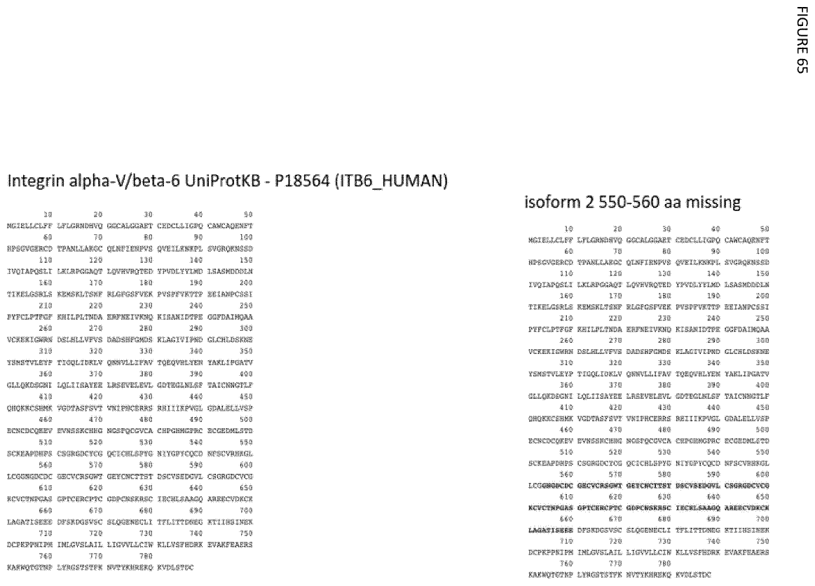

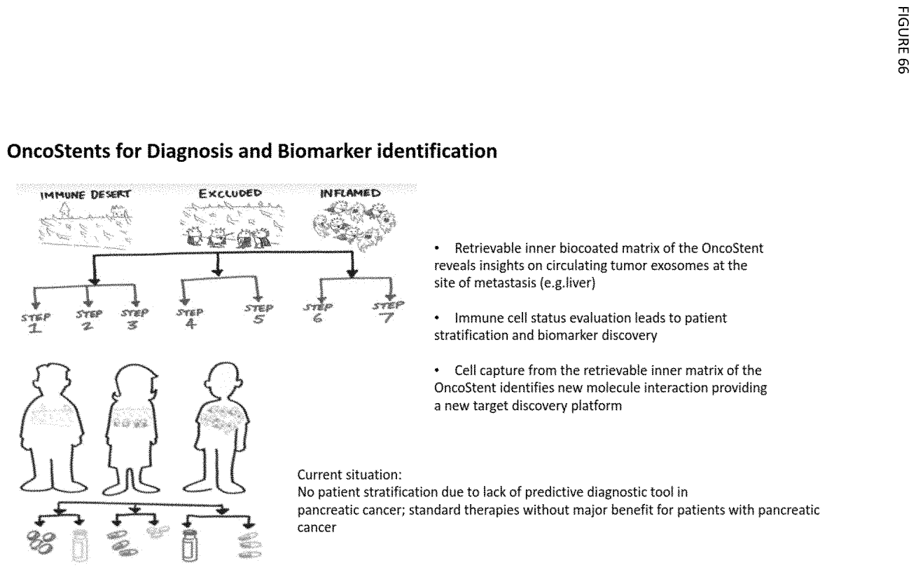

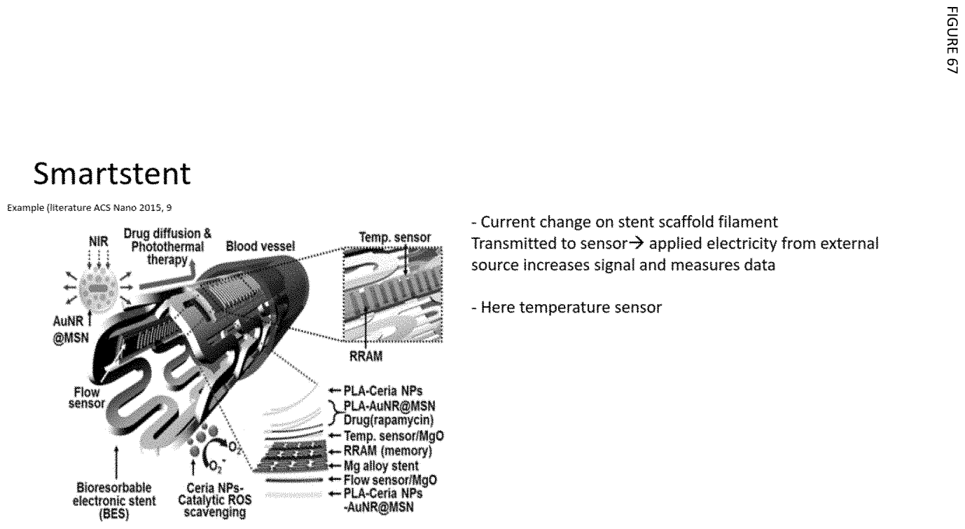

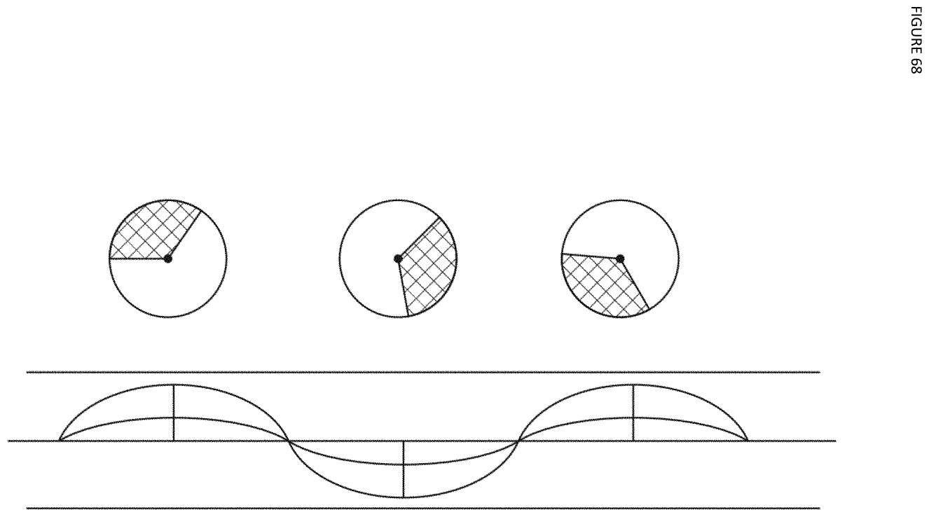

In further preferred embodiments that the device of the present invention is used by implantation in a blood or lymphatic vessel upstream of a tissue with a high risk of developing metastasis. Further information may be derived, for example, from FIG. 58, which shows relevant blood vessels and mentions the connected tissues.

In yet another aspect the present invention relates to a method of treating cancer and/or metastasis, comprising implanting a device according to the invention into a subject in need thereof. Also envisaged is a method of preventing cancer and/or metastasis, comprising implanting a device according to the invention into a healthy subject or a subject being at risk of developing cancer and/or metastasis.

In a further aspect the present invention also envisages a method of manufacturing a device as defined herein. The method preferably comprises the step of providing a biological agent by expressing said biological agent as polypeptide in a suitable host cell. Furthermore, the biological agent, e.g. a poly-peptide, may be modified, e.g. by adding one or more of a sugar, a branched or unbranched multiple sugar structure, an alkyne, an azide, a streptavidin, a biotin, an amine, a carboxylic acid, an active ester, a epoxide or an aziridine. The method may further comprise coating steps which cover or at least partially cover the device with a passive coating and/or an ECM-like structure and/or a biological agent or active coating.

The present invention concerns in a second main aspect a method for recovering cells, aggregates of cells and/or tumor cell derived exosomes and/or proteins and/or nucleic acids and/or recruited immune cells from a tubular shaped elongated catheter device assembly comprising one or more chemical and/or biological agents wherein said device, which is capable of recruiting a circulating tumor and/or circulating metastatic cell and/or motile parts of tumor cells and/or an aggregate of tumor cells and/or a tumor cell derived exosome and thereby removes said cell or motile part thereof from circulation, was implanted in a blood or lymphatic vessel downstream of an existing cancer site or close to a site of potential metastasis formation in a subject, wherein said device is retrievable or partially retrievable, preferably by catheter means and/or in a minimal invasive manner. The tubular shaped elongated catheter device assembly is preferably a tubular shaped elongated catheter device assembly as defined herein above in the context of the first main aspect. In an alternative embodiment, the present invention relates to a method for recovering cells, aggregates of cells and/or tumor cell derived exosomes and/or proteins and/or nucleic acids and/or recruited immune cells from a tubular shaped device assembly for intraluminal use as defined herein above in the context of the first main aspect. In yet another alternative embodiment, the present invention relates to a method for recovering cells, aggregates of cells and/or tumor cell derived exosomes and/or proteins and/or nucleic acids and/or recruited immune cells from a multilumen tubular device as defined herein above in the context of the first main aspect.

The term “recovering cells aggregates of cells and/or tumor cell derived exosomes and/or proteins and/or nucleic acids and/or recruited immune cells” as used herein in the context of the second main aspect refers to an activity of obtaining, ex vivo, cells or parts of cells as mentioned from a tubular shaped elongated catheter device assembly in a living or non-living condition. It is preferred that the cells are recovered as living cells. Cell portions or components of cells such as nucleic acids or proteins are preferably recovered in a complete manner. It is further envisaged that the recovery process lead, in specific embodiments, to a cultivation procedure which is implemented in a suitable cell culture environment or the like. In certain embodiments, the cell culture approach aims at the cultivation of cell consortia found on the previously implanted device. In a preferred embodiment, the present method includes the step of cultivation of recovered cell(s).

The recovery process further includes obtaining any molecular, chemical, histological and/or physical information about the found cells or cell parts known to the skilled person. Accordingly, the present invention envisages in a preferred embodiment the performance of molecular, chemical, histological and/or physical analysis of the recovered cell(s) or parts of it. For example, the cells can be analysed microscopically, or with be exposed to light or electric stimuli. Also envisaged is the use of Raman spectroscopy or luminescence analysis.

Also envisaged is the analysis of nucleic acid sequences, the analysis of proteins or peptides, or of sugars or lipids of the cells or cell parts, or exosomes, the analysis of surface markers, a biochemical analysis of the cells, the identification of known cells, in particular immune cells etc. All obtained information, as well as samples of the recovered cells, are preferably stores, e.g. in tissue or cellular depositories or databases. Both are preferable linked and, in further embodiments, provided on server or in cloud system for centralized data management or the like. The recovery process may, in specific embodiment, further include a direct cooling or freezing of the cells or cell parts, e.g. for subsequent analysis or for transports to different locations, e.g. laboratories, hospitals etc.

In specific embodiments, the catheter device is a flow directed balloon tipped vascular catheter as described herein.

In a specific embodiment the at least one expandable portion of the intraluminal segment is an inflatable balloon, wherein said expandable portion is designed to bind a tumor cell, a tumor cell exosome, and/or a tumor cell aggregate and/or an immune cell.

In preferred embodiments the cross-sectional area of the intraluminal segment is at least about 50% smaller than the cross-sectional area of the luminal target site. For example, the cross-sectional area is, for example, 55%, 60%, 65%, 70%, 75% or 80% smaller than the cross-sectional area of the luminal target site. Corresponding devices are is capable of allowing for a rapid and unobstructed flow of bodily fluids.

In preferred embodiments, the device comprises sections permitting free flow of bodily fluids without any obstructing structural elements at any given point along the longitudinal extension of the device. In further embodiments, the device comprises sections permitting at least 30%, preferably 50%, e.g. 55%, 60%, 65%, 70%, 75%, 80% or more cross-sectional unhindered flow of bodily fluids.

In further embodiments, the device comprises sections which permit a free flow of bodily fluids, which are interrupted by one or more sections permitting an interaction with components of the bodily fluids. In such embodiments, the interactive sections spare cross-sectional areas of free flow.

In typical embodiments, the interactive function is provided by intraluminal segments comprising at least one expandable portion. The term “expandable portion” as used herein relates to an increase in size towards the luminal borders or vessel walls. It is particularly preferred that the expandable portion is reversibly expandable, i.e. can be reduced in its size, e.g. when intending to replace the device or parts of it. The expandable portion is, in several embodiments of the invention, capable of increasing the interactive contact surface with the bodily fluid. Accordingly, cells, cell aggregates or exosomes are allowed to interact with the device in an efficient manner due to the increase surface of the expandable portions of the device of the present invention.

In order to avoid flow obstruction in the luminal target sites or organs, it is prefer that the expandable portion at maximal expansion comprises a cross-sectional area of equal or less than about two thirds of the cross-sectional area of the luminal target site. For example, the cross-sectional area may be about 65%, 60%, 50%, 45%, 40% or less of the cross-sectional area of the luminal target site.

In further embodiments, the expandable portions which only partially cover the cross-sectional area of the luminal target are arranged in a suitable manner. It has been found by the inventors that a sequential order along the longitudinal extension in a clockwise orientation is very well suited to maximize the interaction between bodily fluid components and device, while avoiding any flow obstruction at the luminal target site. It is particularly preferred to have a clockwise orientation with more than 90° differences between the sequential positions, e.g. 100°, 105°, 110°, 115°, 120° or more, or any value in between the mentioned values.

In further embodiments, the device comprises at least two localization markers. These markers should preferably be opposite to each other.

It further preferred that the localization marker is a radiopaque marker, an ultrasound marker, an MRT marker or a CT marker, preferably further comprising at least one sensor such as an optical sensor, an analyte detecting sensor, a thermal sensor or a flow sensor as described herein.

The device according to the present invention may be provided, in preferred embodiments, with one or more suitable properties, which can be combined or mixed according to necessities or circumstances. In certain embodiments, all properties may be given in device. The device according to the invention is hence designed to comprise one or more of said properties, in particular of properties (i) to (v) as mentioned below.

These properties include: (i) it is freely floating in a target vessel, as defined herein. Further-more, (ii) the device is freely positionable in a target vessel, as defined herein. Furthermore, (iii) the device is retrievable, as defined herein. A further property (iv) of the device according to the present invention is that it is anchorable in a target vessel, as defined herein. It is preferred that the device is atraumatically anchorable in a target vessel.

Yet another property (v) of the device according to the present invention is that it is designed to fit into a permanent implant present in a target vessel. The device may, according to this property, be designed as a moveable and retrievable part of an implant. The implant may be present at a certain position in a target vessel. The device according to the present invention may be introducible into said implant and, e.g. after a certain period of time or after having reached the end of its working period (e.g. after 2, 3, 4, 5, 6, 7, 8, 9, 10, 11, 12, 15, 18, 20, 24 or more months), be removed therefrom. The device according to the invention may hence, in a preferred embodiment, be designed as a shuttle entity, which temporarily docks at a permanent implant present in a target vessel. The shuttle may, in further embodiments, be retrievable with a catheter, more preferably in a minimal invasive manner.

In further specific embodiment, the device may also be catheter based, as described herein.

In further embodiments, the device comprises a through lumen housing of the expandable portions of the device. Also envisaged are devices characterized by at least one wire lumen within at least the intraluminal segment of the device. Also preferred is a reversibly expandable segment which is comprised of at least one expandable balloon surface. This balloon surface is designed to have a capability for interaction with at least one component of the bodily fluid. In this embodiment, the expandable balloon is preferably arranged with the distal end of the intraluminal portion of the device. The advantage of such a component is that the device can be expanded towards the lumen via operation of the balloon.

In certain embodiments the device according to the invention comprises a reversible expandable device body. The device may accordingly be expanded along the line from the proximal to the distal end. The expansion may be directed along the line from the proximal to the distal end, or perpendicularly thereto. The expansion may, in specific embodiments, increase the volume of the device by 10, 20, 30, 40, 50, 60, 70, 80, 90, 100, 150, 200, 300, 400, 500, 600, 700, 800, 900, 1000% or more than 1000% or by any value in between the mentioned values.

The elongated tubular shape is preferably provided by a memory shaped wire, or memory shaped spiraling wire, which forms a tubular spiral. An example is a flexible nitinol wire. More preferably, the memory shaped spiraling wire may be modified into a single spiral-like wire structure, characterized by incomplete wire circles and an interdigiting structure.

It is further preferred that the extensions of the wire are provided in the form of circular loops or ellipsoids or non-straight longitudinal extensions.

In particularly preferred embodiments the expandable portions comprise a porous membranous surface, as described herein.

In further embodiments the expandable portion comprises a filter membrane. Accordingly, the device as defined above comprises at least one filter membrane, as described herein.

In a further set of embodiments, the filter membrane comprises, essentially consists of, or consists of one or more microfilaments. For example, the filter membrane comprises, essentially consists of, or consists of a multitude of longitudinally extending microfilaments, each of which is coatable by bioactive agent, thus creating a large bioactive surface area and permitting blood flow. The microfilaments may, in certain embodiments, be arranged parallel and straight or parallel and spiraling or in other arrangements. Further details may also be derived from FIG. 51, which shows and illustrates the corresponding embodiment.

In preferred embodiments, the filter membrane may comprise pores. These pores or the pores of the porous membranous surface as mentioned above may have a pore diameter which ranges from about 25 nm to about 100 μm in diameter. In preferred embodiments, the pores have different size ranges includes the range of about 25 nm to 100 nm in diameter, the range of about 100 nm to 10 urn in diameter, the range of about 10 μm to 25 μm in diameter and the range of about 25 μm to 100 μm in diameter. These ranges, which may all be present in one device or only a sub-group thereof, are adapted to the bodily fluid component to the recruited by the device, i.e. exosomes and the like being of the size of 25 nm to 100 nm, small cells being of the size of 100 nm to 10 μm, circulating tumor cells being of the size of 10 μm to 25 μm, and cell aggregates being of the size of 25 μm to 100 μm. Other cells or cell aggregates, e.g. cancer-associated fibroblasts (CAFs) typically have a size of 10 μm to 15 μm, myeloid-derived suppressor cells (MDSc), and other antigen-presenting cells (APCs) have a size of 10 μm to 20 μm. It is further preferred that the pores are provided in a differential manner such as comprising differential ranges of 25 nm to 100 nm, 100 nm to 10 μm, 10 μm to 25 μm, or 25 μm to 100 μm. In further embodiments, the pores may have a diameter of 25 nm, 40 nm, 50 nm, 75 nm, 100 nm, 200 nm, 500 nm, 750 nm, 1 μm, 5 μm, 10 μm, 15 μm, 20 μm, 50 μm, or 100 μm or any value in between the mentioned values. Different pores in one device or one filter membrane or surface may, in further embodiments, be provided with the same diameter, or with two or more different diameters. It is preferred that pores with different diameters be present.

In embodiments, in which more than one filter membrane is present in a device, said filter membranes may preferably have differential pore diameters and/or differential pore pattern as defined herein. It is particularly preferred that the diameter ranges from about 25 nm to 100 nm, 100 nm to 10 μm, 10 μm to 25 μm, or 25 μm to 100 μm or any value in between the mentioned values.

The filter membrane may, in specific embodiments, be itself expandable. Accordingly, an expansion of the device may be followed or implemented by an expansion of the filter membrane. In certain embodiments, the filter membrane is attached to or arranged with the proximal and distal end of the device. In further embodiments, the filter membrane is designed as a non-permanent, retrievable filter membrane. According to these embodiments, the filter membrane may be separated from the remainder of the device and be removed therefrom, e.g. with a catheter or based on endoscopic techniques. The filter membrane may, in further embodiments, be designed as replaceable entity allowing for an exchange of a filter membrane after a certain period of time or after having reached the end of its working period (e.g. after 2, 3, 4, 5, 6, 7, 8, 9, 10, 11, 12, 15, 18, 20, 24 days; after 2, 3, 4, 5, 6, 7, 8, 9, 10, 11, 12, 15, 18, 20, 24 weeks; or after 2, 3, 4, 5, 6, 7, 8, 9, 10, 11, 12, 15, 18, 20, 24 or more months). It is preferred that the end of the working period does not surpass 24 months. In specific embodiments, the filter membrane may be retrievable as whole, or in parts. The partial retrievability may be implemented as segmented retrievability, e.g. via separable portions of the filter membrane, e.g. 10%, 20%, 30%, 40%, 50%, 60%, 70%, 80%, 90% etc., which can individually be retrieved, e.g. the distal part may be retrieved, whereas the proximal part is not retrieved etc. In further embodiments, any retrieval may followed by replacement of the retrieved filter membrane by a new filter membrane or the same filter membrane after washing and preparation for a secondary use. Also envisaged is the retrieval of more than one segment at a time.

The present invention also envisages that the filter membrane is self-expandable. In further embodiments, the entire filter device assembly implant is self-expandable, as described herein.

It is further envisaged that the device according to the present invention comprises at least two filter membranes, each of which incompletely covers the cross-sectional area. These filter membranes may be arranged in any suitable form or shape. It is preferred that the filter membranes are arranged in tandem position along the longitudinal axis of the device body. Particularly preferred is a tandem arrangement along the longitudinal axis of the device body, wherein said filter membranes are opposite to each other within the circumference of the device body. Alternatively, they may be shifted in clockwise orientation. Such a shifting may advantageously be implemented for more than 2 filter membranes in tandem position, e.g. 3, 4, 5, 6, 7, 8, 9, 10 or more filter membranes. The device according to the present invention may, in further embodiments, comprise alternating non-completely covering filter membranes as defined herein

The present invention further envisages that the device is provided in any suitable form or architecture. It is particularly preferred that the device is provided in a tubular, onion like, pearl-chain-like, or a birds-nest like shape, or in any mixture of these shapes. Examples of corresponding forms and shapes can, for example, be derived from FIG. 24, 25, 26, 27, 28, 29, 30, or 31. The form or shape of the device may, in certain embodiments, be followed also be the form or shape of the filter membranes. For example, the device according to the present invention may comprise alternating non-completely covering filter membranes which are provided in a pearl-chain, onion type or birds-nest like shape. In other embodiments, the device comprises a multitude of longitudinally extending microfilaments, which may be arranged parallel or non parallel to each other in straight configuration or tortuous e.g. spiraling configurations.

The filter membrane may be composed of any suitable material. Envisaged examples include elastic or foldable polymer materials. Particularly preferred is polyurethane. Also envisaged is the use of micromeshes. In preferred embodiments, these micromeshes may comprise ultrathin wires, metallic or polymeric materials. In particularly preferred embodiments, the filter membranes according to the present invention is at least partially coated with one or more chemical and/or biological agents as defined herein above or below.

In certain embodiments, the coating may differ between different filter membranes of a device. For a device comprising 2, 3, 4, 5, 6, 7 or more different filter membranes, a number of 2, 3, 4, 5, 6, 7 or more coatings may be provided. Alternatively, different filter membranes may be provided with one coating only. In further embodiments, the coating may change along the axis of the device, e.g. from proximal to distal over one filter membrane or over various filter membranes. In further embodiments, biological agents may be provided as coating in different combinations, e.g. as combination of adhesion and death signals, or as a combination of adhesion and immune modulation function.

In further embodiments the filter membrane as defined herein covers at least one cross-sectional area of the body of a device according to the present invention, in particular of the body of a device having a tubular form. For example, the filter membrane may cover 1, 2, 3, 4, 5, 6, 7, 8, 9, 10 or more cross-sectional areas of said body of a device according to the present invention. It is particularly preferred that the plane of the filter membrane is arranged perpendicular to the direction of the longitudinal axis of the body of a device according to the present invention. Also envisaged are different angles, e.g. non-perpendicular angles, between the plane of the filter membrane and the direction of the longitudinal axis, e.g. 10°, 20°, 30°, 40°, 50°, 60°, 70°, 80° or 85° or any value in between said values.

The present invention also envisages that the filter membrane is disposed within or around an expandable body, is characterized by a memory shaped metallic or memory shaped polymer structure and/or comprises a detachment mechanism cooperating between the expandable portion and an intraluminal segment of the device. Also preferred are devices comprising longitudinally extending free floating microfilaments. In specific embodiments, the device comprises an outer sheath concentrically disposed about the device. In this embodiment, the outer sheath and the device are movable relative to one another.

In a particularly preferred embodiment, the device as defined herein additionally comprises a downstream embolic debris filter, e.g. a retrievable downstream embolic filter.

The device may be composed of, or be partially composed of, or comprise any suitable structural support material. According to certain embodiments, the structural support material may be metal. Preferred examples are stainless steel, gold, titanium, gold-titanium alloy, cobalt-chromium alloy, tantalum, tungsten, platinum-radium alloy, tantalum alloy, magnesium, nickel-titanium alloy, e.g. nitinol, silver or copper. Alternatively or additionally, the support material may be plastic or polymeric material. Also envisaged is the use of memory shape materials, e.g. elastic memory shape meshwork material. Preferred examples include memory shape elastic wires. In yet another alternative, the structural support material may be a material readable by tomography or other imaging techniques, e.g. X ray. The present invention further envisages, in very specific embodiments, that the device body is not biodegradable or composed of biomaterial or biodegradable material. In alternative embodiments, the device or the device body may be biodegradable or composed of biomaterial or of biodegradable material, e.g. the device is composed of, is partially composed of, or comprises structural support material selected from the group comprising biodegradable or bioresorbable material, preferably as defined herein above in the context of the first main aspect. The filtering and recruiting activity of the device is mainly achieved by the presence of one or more chemical and/or biological agents which are comprised on said device and fulfil their activity in situ. An accessory effect improving the filtering or recruiting may be provided by the form and design of the device itself, e.g. by reducing the circulation velocity of the bodily fluid, preferably of the blood stream. Accordingly, the retention time of circulating tumor cell and/or circulating metastatic cell and/or motile parts of tumor cells at or on the device can advantageously be extended, allowing for molecular interactions between the circulating tumor cell and/or circulating metastatic cell and/or motile parts of tumor cells on the one hand, and the one or more chemical and/or biological agents located on the device on the other hand.

It is particularly preferred that the device as defined herein be designed according to current implant techniques and technologies (e.g. stent implantation, septal occluder implantation, vena cava filter implantation). It is further preferred that it comprises an inactive (unexpanded, folded or constraint state) and an active (expanded, unfolded, unconstraint) state. The device is introduced into the target organ in an unexpanded state and is released at the target position into the expanded and active state

In a central aspect of the second main aspect of the present invention the one ore more chemical and/or biological agents as mentioned above perform different chemical, biological and/or biochemical activities. Said activities may ultimately contribute and facilitate an effective recruiting of circulating tumor cells, circulating metastatic cell or motile parts of tumor cells, thus allowing for a removal of said cells, cell aggregates or parts of tumor cells or exosomes from the bodily fluid, e.g. from the blood and at the same time allow for a prolonged and safe use of the device within a subject, as well as the efficient and extensive removal of these cells and parts thereof from the subject, even after a prolonged time, e.g. after 2 to 30 days, 4 to 8 weeks, or 1 to 24 months. To achieve one or more, preferably all, of these functions, the one ore more chemical and/or biological agents are provided as coating on the device, in particular on the filter membrane of the device as defined herein above.

A further important factor is the presence of an ECM-like structure on the surface of the device, as defined herein below. The ECM-like structure may be provided in an equal manner throughout the device, or it may be provided in certain sectors or regions or the device only, or in certain sectors or regions of the device different forms of these structures may be present. The ECM-like structure essentially fulfils the function of a structural and/or biochemical support for surrounding cells, which is assumed to be of elevated importance once cells have settled down on or within the device. Furthermore, the ECM-like structure resembles the microenvironment of the tumor or metastases. This ECM-like structure which is typically composed of laminins, adhesion molecules or chemical entities serves as homing compartment within the device. Yet another important factor, which is considered relevant for the initial recruiting steps is the presence of a biological agent, which is capable of binding to a tumor marker on a cell or part thereof, in particular on a recruiting a circulating tumor or circulating metastatic cell or motile parts of tumor cells. These different factors may, in specific embodiments, present together at one area of the device, or they may fulfil their functions in different areas or the device. Also suitable mixture of any of these factors are envisaged. Furthermore, the present invention envisages different forms of devices which may comprise or implement either all or a sub-group of said factors. The overall shape, the intended use, the specific medical condition of a subject, the intended time of use and/or the tumor form may require an adjustment of the presence of above mentioned factors or functions. Additional factors which may also be taken into account are the tumor staging, i.e. the phase or stadium of the disease, and/or details on a pretreatment, e.g. whether the subject has already been treated, how successful this treatment was and the time period since the treatment, as well as a possible chemotherapeutic resistance etc.

Accordingly, a method of the present invention in the context of the second main aspect relates to the use of device which, in preferred embodiments, comprises (i) a passive coating with one or more polymeric materials, (ii) as well as an ECM-like structure, and (iii) an active coating which is capable of binding to a tumor marker on a cell or part thereof. In further embodiments, at least one of these elements (i) to (iii) may not be present. The present invention envisages all combinations of (i), (ii) and (iii) as defined above.

In one group of embodiments of the invention the coating may thus be a passive coating.

In a further group of embodiments, the chemical and/or biological agents composing the coating of the device constitute an extracellular matrix-like structure, as defined herein.

In specific embodiments, the macromolecules used to provide an ECM like structure, i.e. the macromolecules which are provided as coating for the device according to the present invention, are proteoglycans, non-proteoglycan-polysaccharides, elastin; fibronectin or laminin. Particularly preferred are mixtures of these macromolecules. Preferred examples of suitable proteoglycans include heparan sulfate, chondroitin sulfate and/or keratin sulfate. Preferred examples of suitable non-proteoglycan-polysaccharides include hyaluronic acid. The present invention further envisages the employment of ECM-like structures in the form of protein mixtures. Such mixtures are, for examples, secreted by Engelbreth-Holm-Swarm (EHS) mouse sarcoma cells. Also preferred are Matrigel, BioCoat or GelTrex mixtures. It is particularly preferred that protein mixtures with substitutions of human proteins are used.

According to the present invention, the method in the context of the second main aspect makes use of a device as defined herein above in the context of the first or second main aspect comprising a coating as defined herein, which provides an environment for circulating tumor cells, for circulating metastatic cell or for motile parts of tumor cells, for exosomes, for cell aggregates and for immune cells. The environment may, for example, allow metastatic cells to settle down in or on the device and thus leave the bodily fluid circulation, e.g. lymph or blood and/or it allows for immune cells to interact with cells being present on or in the device. Similarly, tumor cells which are non-metastatic but circulate through the body may be stimulated to settle down in or on the device and thus leave the bodily fluid circulation, e.g. lymph or blood. This function is preferably implemented by the use of, or presence of a biological agent in or on the device, in particular as part of the coating, wherein said biological agent is capable of binding to a tumor marker on a cell or part thereof, in particular on a recruiting a circulating tumor or circulating metastatic cell or motile parts of tumor cells. In further embodiments, the present invention also envisages a binding to immune cells or corresponding markers on immune cells, i.e. immune cell markers.

Examples of tumor-marker specific antibodies envisaged by the present invention include panitumab, matuzumab, nimotuzumab or derivatives thereof. Further envisaged are additional antibodies against EGFR family members, or ErbB2/HER2 family members. Further details would be known to the skilled person or can be derived from suitable literature sources such as Huang and Buchsbaum, Immunotherapy, 2009; 1(2): 223-239.

The present invention, in preferred embodiments, envisages the use of one or more homing factors as defined above.

In specific embodiments any combination of the above mentioned elements, e.g. homing factors, CD133, VEGFR-1 or antibody may be used. Also envisaged is the use of one type of element, e.g. one specific homing factor, in a specific area of the device, followed by a different type of element in the neighbouring area etc.

The present invention further envisages that a biological agent as defined herein, e.g. a homing factor, an antibody etc. is linked to the passive coating of the device via a spacer element. Also envisaged is the linkage to the structural support material of the device, e.g. stainless steel, gold, titanium, gold-titanium alloy, cobalt-chromium alloy, tantalum, platinum-radium alloy, tantalum alloy, magnesium, nickel-titanium alloy, e.g. nitinol, silver or copper; plastic or polymeric material; elastic memory shape meshwork material such as memory shape elastic wires or a material readable by tomography or other imaging techniques, e.g. X ray; or any mixture thereof, which may be present on the surface of said filter device assembly implant.

In addition to its function as spatial separator between the biological agent and the surface of the device, the spacer element further fulfils the function of a separator between biological agents as defined herein. The present invention accordingly envisages the provision of biological agents in a density which allows for an efficient binding of the biological agent to a tumor cell or part thereof. According to one embodiments, the spacer elements may be provided in a density of 2 to 500 per μm2 on the surface of the device. For example, a density of 2, 5, 10, 15, 20, 30, 40, 50, 60, 70, 80, 90, 100, 150, 200, 250, 300, 350, 400, 450 or 500 per μm2 on the surface of the device may be used. According to a preferred embodiment, the spacer elements are provided in a density of 1 mg per cm2 on the surface of the device.

The present invention further relates to biological agents—as part of the device—which comprise, essentially consists of, or consists of a binding domain capable of binding to a tumor marker as defined herein.

In preferred embodiments, the binding domain is a peptide or polypeptide molecule. The do-main may have any suitable length. It is preferred that it has length of about 20 to about 250 amino acids. More preferably, the binding domain has a length of about 20 to about 120 amino acids. In further preferred embodiments, the binding domain has a length of about 20, 25, 30, 35, 40, 45, 50, 55, 60, 65, 70, 75, 80, 85, 90, 95, 100, 105, 110, 115 or 120 amino acids, or any value in between the mentioned values.

The binding domain capable of binding to a tumor marker according to the present invention may be provided in any suitable form. For example, the binding domain may be provided as part of a larger protein structure, wherein said larger protein structure may fulfil presenting or structural functions, thus improving the expose of the binding domain. Further envisaged is combination of more than one binding domain per biological agent unit as defined herein.

In further embodiments, the binding domain capable of binding to a tumor marker according to the present invention may be combined with one or more additional functional domains.

Also envisaged is that the domains are linked via a linker element of about 1 to 20 amino acids length. For example, the linker element may be a peptide having a length of 1, 2, 3, 4, 5, 6, 7, 8, 9, 10, 11, 12, 13, 14, 15, 16, 17, 18, 19 or 20 amino acids.

In further specific embodiments of the present invention the biological agent is provided as linear or circular element or as an element composed of linear and circular parts. It is particularly preferred to provide the biological agent as a linear or circular or partially linear or circular peptide or poly-peptide. Typical embodiments envisage that the circular biological agent has or is part of a structure comprising a loop or a loop and a stem. Also envisaged are linear structures, which are linked to a spacer element as defined herein. The loop structure or linear structure may comprise said preferably comprise the biological agent, e.g. the tumor binding domain, at an exposed position. Said exposition allows for an efficient interaction, e.g. leading to the binding to a tumor marker or a tumor cell.

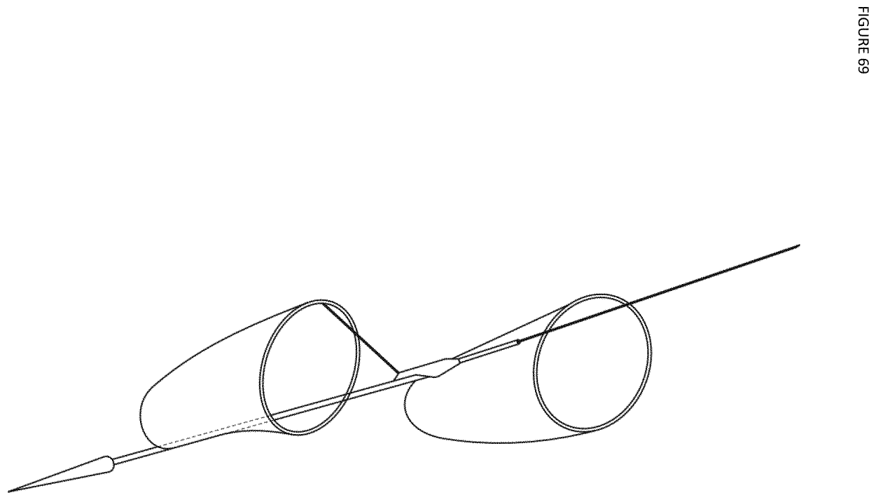

In a further preferred embodiment the peptide as used in the context of the present invention has one or more of the properties: (i) to (xxii) as defined herein above. Also envisaged is any combination of these peptides with components of the device as mentioned above. Further information on specific sequences of peptides envisaged by the present invention may be derived from FIG. 61, 62 or 63.

In further specific embodiments a biological agent as defined herein above comprises or is linked to one or more additional elements. For example, the biological agent may be linked to a sugar, to branched or unbranched multiple sugar structures, to alkynes or azides, to streptavidin, biotin, amines, carboxylic acids, active esters, epoxides or to aziridines.

In preferred embodiments of the method in the context of the second main aspect the recovering of cells and/or proteins from the device is performed ex vivo after 1, 2, 3, 4, 5, 6, 7 days, 2, 3, 4 weeks or 2, 3, 4, 5, 6, 7, 8, 9, 10, 11, 12 months of implantation time. The time point of recovery may be adjusted or determined according to previous experience with the same device type, or a similar health condition of the patient. The recovery may, in specific embodiment, be performed only with parts of the device, e.g. retrievable components such as the expandable portions etc. Accordingly, a retrieval of such a component may also be used for a replacement of the component by a fresh element. Thereby, the overall implantation time of the stationary or non-retrieval part of the device may be prolonged 1, 2, 3, 4, 5, 6, 7, 8, 9, 10 times or more. The time point of recovery may further be determined according to secondary events such as a planed surgery, the presence of certain diagnostic parameters for the patient, e.g. certain tumor or inflammation marker obtained alternatively etc. According to further embodiments, the time point may be derived according to the analysis of the cancer-, metastasis-, and immune-status of the patient.

In a further embodiment, the time point of recovery may be determined in accordance with a signal obtainable from the device. For example, the device may be designed to send a filling status signal to an outside station, or the filling status may be observable or detectable form the outside of the subject's body, e.g. by ultrasound measurement or the like. Accordingly should a signal indicate a filling state at or beyond a predefined threshold or should the measured filling status indicate that such a threshold is reached or passed, the device or parts of it may be recovered.

In a further embodiment, recovering cells and/or proteins is performed subsequent to a tumor treatment of a patient. It is particularly preferred that subsequent to the tumor treatment any circulating tumor and/or circulating metastatic cell and/or motile part of tumor cells and/or aggregates of tumor cells and/or tumor cell derived exosomes which are still present in the circulation are recovered. At the same time, immune cells present in the bodily fluid can be recovered.

In a further aspect in the context of the second main aspect the present invention relates to cell, a cell aggregate or an exosome or part of any of the before mentioned obtained from the method as described above. In a preferred embodiment, the cells are tumor cells or immune cells, or subcellular vesicles such as exosomes which have been obtained or recovered from the device as described herein. The present invention particularly envisages cells or cell fragments or exosomes which have a molecular identify that has not been described before, e.g. show new genomic sequence. This can easily be verified by sequence determination of the cells.

Similarly, the present invention, in a different aspect in the context of the second main aspect, relates to a protein or nucleic acid obtained from the method of as defined above. The present invention particularly envisages proteins or nucleic acid which have a molecular identify that has not been described before, e.g. show new genomic sequence.

In a further aspect in the context of the second main aspect the present invention relates to a method of diagnosing cancer and/or metastasis, or of determining an increased likelihood for developing cancer and/or metastasis and/or of determining the subject's metastatic immune status, preferably near a metastatic site. This method comprises implanting a device as defined herein into a subject in need thereof and detecting the presence of circulating tumor and/or circulating metastatic cell and/or motile part of tumor cells and/or aggregates of tumor cells and/or tumor cell derived exosomes and/or immune cells captured by the device subsequent to the recovery of said device. The term “immune status” as used herein relates to the presence, identity and amount of immune cells determined. The status preferably determined in the vicinity of a metastatic site.

In a further aspect in the context of the second main aspect the present invention relates to a method of diagnosing cancer and/or metastasis, or of determining an increased likelihood for developing cancer and/or metastasis, comprising implanting a device as defined herein into a healthy subject, a subject showing no symptoms of a disease, preferably symptoms of cancer or metastasis, or a subject being at risk of developing cancer and/or metastasis. The method accordingly aims at detecting the presence of circulating tumor and/or circulating metastatic cell and/or motile part of tumor cells and/or aggregates of tumor cells and/or tumor cell derived exosomes captured by the device in the mentioned subjects. Such an event triggers a treatment of the subject and/or further diagnostic steps.

In yet another aspect in the context of the second main aspect the present invention relates to a method of monitoring the effect of a disease treatment, preferably cancer treatment, comprising implanting a device as defined herein into a patient currently being treated for a disease, preferably cancer and/or metastasis, or a patient who has finished his disease treatment, preferably cancer and/or metastasis treatment. The implantation is preferably performed within a time period of about 1 week to 2 years and subsequently recovering said device. The subsequent recovery may be performed as describe herein, e.g. after a further period of time, e.g. between days or years.

In specific embodiments, the device as described herein in the context of the second main aspect is specifically designed to be implanted into a luminal organ, preferably a blood vessel. Examples of envisaged blood vessels are an artery, an elastic artery, a distributing artery, an arteriole, a capillary, a venule or a vein. Further envisaged are devices for an implantation into a heart chamber or an elastic artery. The device may accordingly be designed as free floating device with connecting wires to maintain its position. This concept also allows for retrieval. In further specific embodiments, the device as described herein is specifically designed to be implanted into a lymphatic vessel. Particularly preferred is vena cava, or any other vessel wherein said device can suitably be used free floating. Further envisaged and preferred is a transvascular implantation. In certain embodiments, the device is designed to be transvascularly advanced and implanted at a target site. The device may accordingly be designed to have adequate dimensions for an advancement in the target tissue or region. It may, in further embodiments, be catheter based and preferably steerable. It is particularly preferred that the device is steerable is by its torque stable structure or by comprising a steerable wire. Also envisaged in the present invention is a percutaneous implantation, a minimal invasive implantation, an endoscopically guided implantation, e.g. via ultrasound or magnetic resonance devices, or a laparoscopic implantation. The implantation may, in preferred embodiments, be performed directly in a tumor or tumorous tissue or in the close vicinity of a tumor or tumorous tissue, or in any other target organ or tissue, as well as corresponding device designs and their use.

In certain specific embodiments a device to the implanted via a percutaneous implantation, a minimal invasive implantation, an endoscopically guided implantation, e.g. via ultrasound or magnetic resonance devices, a laparoscopic implantation, or a transcutaneous implantation may have a specific form or design, e.g. may not be composed of filters as defined herein. Preferably, the device may have or comprise a porous solid architecture or design, or may have the form of porous solid scaffold.

It is particularly preferred that the device as described herein in the context of the second main aspect is used by implantation in a blood or lymphatic vessel downstream of an existing cancer site in a subject. If such a positioning downstream of an existing cancer is envisaged, it is preferred that the device is used by implantation in close proximity to said existing cancer site.

In further preferred embodiments the device in the context of the second main aspect of the present invention is used by implantation in a blood or lymphatic vessel upstream of a tissue with a high risk of developing metastasis. Further information may be derived, for example, from FIG. 56, which shows relevant blood vessels and mentions the connected tissues.

It is particularly preferred that the cancer or tumor to be treated is a colon cancer, breast cancer, lung cancer, melanoma, esophageal cancer, prostate cancer, pancreatic cancer, ovarian cancer, myeloma, or a lymphoma such as ALL, CLL, or AML. It is further particularly preferred that the metastasis to be treated or prevented is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarial tumor or nephroblastom such as Wilms tumor.

In yet another aspect in the context of the second main aspect the present invention relates to a method of data collection comprising (i) monitoring a device as described herein implanted in a subject via ultrasound scanning, tomography, optical, or by determining the device environment for changes indicative of a disease; and (ii) collecting data over time for recognizing changes of the monitored values. This method is based on the employment of remote recognition options provided by ultrasound or tomography approaches. Also optical signals may be obtained from the device, e.g. via cables. The data collection may be performed over any suitable period of time. The collected data may, for example, be employed for the determination whether a replacement of the device is necessary.

In yet another aspect in the context of the second main aspect the present invention relates to a method of identifying a target cell, a target protein or a target nucleic acid. This method comprises analyzing a cell, a cell aggregate or an exosome or part of any of the before, or a protein or nucleic acid obtained or recovered from a device as described herein. Also the analysis of correspondingly obtained immune cells or cells with immune markers is envisaged.

The analysis of obtained elements, e.g. cells etc. can advantageously lead to the generation of personalized tailored device, or help to provide personalized therapy and diagnosis, e.g. with a medical device. Furthermore, a metastasis technology platform for new target discovery can accordingly be obtained.

In yet another embodiment in the context of the second main aspect, the approach can provide a suitable source for material and knowhow for a personalized tumor vaccination treatment.

The recovering of cells, aggregates of cells and/or tumor cell derived exosomes and/or proteins and/or nucleic acids from the device may be performed after any suitable amount of time, e.g. after a time period of about 1 day to 2 years, e.g. after 1 week, 2 weeks, 3 weeks, 4 weeks, 2 months, 4, months, 6 months, 8 months, 12, months, 1.5 years, 2 years etc.

It is further preferred that said recovered cells are sequenced and/or biochemically analyzed and/or compared with previous data or database values on recovered cells to provide a disease status profile. In certain embodiments, the present invention envisages the construction of databases, which are preferably reachable via internet or intranet communication.

In a further embodiment in the context of the second main aspect the method comprises a step of prognosticating the length and/or outcome of the treatment.

It is also preferred that said method comprises a step of adjusting a disease treatment strategy to the diagnostic or target identification values obtained. The skilled person can modify the type and/or amount of pharmaceutical agent to be administered, e.g. in accordance with the cells or exosomes etc. obtained, the point in time when the cells or exosomes were detected, or the amount detected.

The present invention concerns in a third main aspect a tubular shaped elongated catheter device assembly comprising one or more chemical and/or biological agents wherein the device is capable of interacting with a circulating tumor cell and/or a circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein within bodily fluids of luminal organs, wherein said device is designed to allow a read out and/or monitoring of the device with respect to the recruiting of or interaction with a circulating tumor and/or circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein. The tubular shaped elongated catheter device assembly is preferably a tubular shaped elongated catheter device assembly as defined herein above in the context of the first main aspect. In an alternative embodiment, the present invention relates to a tubular shaped device assembly for intraluminal use as defined herein above in the context of the first main aspect, comprising one or more chemical and/or biological agents wherein the device is capable of interacting with a circulating tumor cell and/or a circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein within bodily fluids of luminal organs, wherein said device is designed to allow a read out and/or monitoring of the device with respect to the recruiting of or interaction with a circulating tumor and/or circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein. In yet another alternative embodiment, the present invention relates to a multilumen tubular device as defined herein above in the context of the first main aspect comprising one or more chemical and/or biological agents wherein the device is capable of interacting with a circulating tumor cell and/or a circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein within bodily fluids of luminal organs, wherein said device is designed to allow a read out and/or monitoring of the device with respect to the recruiting of or interaction with a circulating tumor and/or circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein. In yet another alternative embodiment, the present invention relates to a method for recovering cells, aggregates of cells and/or tumor cell derived exosome and/or an immunologic cell such as a T cell, B cell or dendritic cell, and/or an antibody complex such as an autoantibody complex, and/or a neurologic se-rum marker protein.

One of the main functions of the device in the context of the third main aspect as defined above is to allow a read out and/or monitoring of the device with respect to the recruiting of or interaction with a circulating tumor and/or circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein. This readout or monitoring function is provided, in a preferred embodiment, by ultrasound scanning, tomography, optical analysis, and/or by electrochemical measurements. The device is accordingly designed as a smart device which is capable of generating signals which can be detected outside of the device, typically outside of the patient's body, where the device resides. An “ultrasound scanning” as used herein in the context of the third main aspect may for example be implemented via ultrasound techniques such as IUVS imaging technologies. “Tomography” based monitoring may, for example, be implemented via MRT or CT techniques. An “optical analysis” may be implemented via chemo-luminescence or fluorescence, which can be detected, e.g. via direct connection to an outside receiving station. Particularly preferred is the monitoring of the device via electrochemical measurements, as will be explained in more detail herein below. In a further preferred embodiment the monitoring aims at the environment of the device, which may be scrutinized for changes indicative of a disease. These changes may include several parameters such as pH, temperature, pressure, light emission, or ion concentrations.

In a typical embodiment, the device is capable of filtering and thereby recruiting circulating tumor or circulating metastatic cell or motile parts of tumor cells, as well as tumor cell aggregates or tumor cell exosomes and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein. The filtering and recruiting activity is designed to lead to a removal of circulating tumor and/or circulating metastatic cell and/or motile parts of tumor cells, as well as cell aggregates formed by tumor or metastatic cells, as well as exosomes or similar sub-cellular microvesicle, and/or of immunologic cells such as a T cells, B cells or dendritic cells, and/or an antibody complex such as an autoantibody complex, and/or a neurologic serum marker protein from bodily fluids of luminal organs. It is preferred that said removal is from the bloodstream or from lymphatic fluids. The present invention, in particular envisages the recruiting of cells, cell aggregates, as well as of motile parts of tumor cells such as circulating microvesicles which are small membrane-bound cell fragments (sizes between 30 and 1000 nm diameter) or exosomes, which have 30 to 100 nm in diameter. Circulating microvesicles and exosomes were recently shown to have roles in cell signaling and intercellular molecular communication. Circulating microvesicles are typically actively released into the extracellular space to interact with specific target cells and have been demonstrated to deliver bioactive molecules. In many tumors, circulating microvesicle levels increase. The level and biochemistry of exosomes and microvesicles may provide suitable indicators for tumor severity. Similarly, the level, amount and biochemistry of cell aggregates, e.g. cell groups of 2 to 15 cells which are transported in the bodily fluid as an aggregate, are considered to constitute suitable indicators for tumor development and severity. Another particularly preferred group of component to be recruited is immunologic cells such as a T cell, B cell or dendritic cell. Also particularly preferred is the recruiting of antibody complexes. A prominent and envisaged example of such complexes are autoantibody complexes, which typically occur during some neurologic diseases.

In specific embodiments, the catheter device in the context of the third main aspect is a flow directed balloon tipped vascular catheter as described herein.

In a specific embodiment the at least one expandable portion of the intraluminal segment is an inflatable balloon, wherein said expandable portion is designed to bind a tumor cell, a tumor cell exosome, and/or a tumor cell aggregate and/or an immune cell, and/or an antibody complex such as an autoantibody complex, and/or a neurologic serum marker protein.

In preferred embodiments the cross-sectional area of the intraluminal segment is at least about 50% smaller than the cross-sectional area of the luminal target site. For example, the cross-sectional area is, for example, 55%, 60%, 65%, 70%, 75% or 80% smaller than the cross-sectional area of the luminal target site. Corresponding devices are capable of allowing for a rapid and unobstructed flow of bodily fluids.

In preferred embodiments, the device in the context of the third main aspect comprises sections permitting free flow of bodily fluids without any obstructing structural elements at any given point along the longitudinal extension of the device. In further embodiments, the device comprises sections permit-ting at least 30%, preferably 50%, e.g. 55%, 60%, 65%, 70%, 75%, 80% or more cross-sectional unhindered flow of bodily fluids.

In further embodiments, the device comprises sections which permit a free flow of bodily fluids, which are interrupted by one or more sections permitting an interaction with components of the bodily fluids. In such embodiments, the interactive sections spare cross-sectional areas of free flow.

In typical embodiments, the interactive function is provided by intraluminal segments comprising at least one expandable portion. The term “expandable portion” as used herein relates to an increase in size towards the luminal borders or vessel walls. It is particularly preferred that the expandable portion is reversibly expandable, i.e. can be reduced in its size, e.g. when intending to replace the device or parts of it. The expandable portion is, in several embodiments of the invention, capable of increasing the interactive contact surface with the bodily fluid. Accordingly, cells, cell aggregates or exosomes are allowed to interact with the device in an efficient manner due to the increase surface of the expandable portions of the device of the present invention.

In order to avoid flow obstruction in the luminal target sites or organs, it is prefer that the expandable portion at maximal expansion comprises a cross-sectional area of equal or less than about two thirds of the cross-sectional area of the luminal target site. For example, the cross-sectional area may be about 65%, 60%, 50%, 45%, 40% or less of the cross-sectional area of the luminal target site.

In further embodiments, the expandable portions which only partially cover the cross-sectional area of the luminal target are arranged in a suitable manner. It has been found by the inventors that a sequential order along the longitudinal extension in a clockwise orientation is very well suited to maximize the interaction between bodily fluid components and device, while avoiding any flow obstruction at the luminal target site. It is particularly preferred to have a clockwise orientation with more than 90° differences between the sequential positions, e.g. 100°, 105°, 110°, 115°, 120° or more, or any value in between the mentioned values.

In further embodiments, the device comprises at least two localization markers. These markers should preferably be opposite to each other.

It further preferred that the localization marker is a radiopaque marker, an ultrasound marker, an MRT marker or a CT marker, preferably further comprising at least one sensor such as an optical sensor, an analyte detecting sensor, a thermal sensor or a flow sensor.

The device according to the present invention may be provided, in preferred embodiments, with one or more suitable properties, which can be combined or mixed according to necessities or circumstances. In certain embodiments, all properties may be given in a device. The device according to the invention is hence designed to comprise one or more of said properties, in particular of properties (i) to (v) as mentioned below.

These properties include: (i) it is freely floating in a target vessel, as defined herein. Further-more, (ii) the device is freely positionable in a target vessel, as defined herein. Furthermore, (iii) the device is retrievable, das defined herein. A further property (iv) of the device according to the present invention is that it is anchorable in a target vessel, as defined herein.

Yet another property (v) of the device according to the present invention is that it is designed to fit into a permanent implant present in a target vessel. The device may, according to this property, be designed as a moveable and retrievable part of an implant. The implant may be present at a certain position in a target vessel. The device according to the present invention may be introducible into said implant and, e.g. after a certain period of time or after having reached the end of its working period (e.g. after 2, 3, 4, 5, 6, 7, 8, 9, 10, 11, 12, 15, 18, 20, 24 or more months), be removed therefrom. The device according to the invention may hence, in a preferred embodiment, be designed as a shuttle entity, which temporarily docks at a permanent implant present in a target vessel. The shuttle may, in further embodiments, be retrievable with a catheter, more preferably in a minimal invasive manner.

In further specific embodiment, the device may also be catheter based, as described herein.

In further embodiments, the device comprises a through lumen housing of the expandable portions of the device. Also envisaged are devices characterized by at least one wire lumen within at least the interluminal segment of the device. Also preferred is a reversibly expandable segment which is comprised of at least one expandable balloon surface. This balloon surface is designed to have a capability for interaction with at least one component of the bodily fluid. In this embodiment, the expandable balloon is preferably arranged with the distal end of the intraluminal portion of the device. The advantage of such a component is that the device can be expanded towards the lumen via operation of the balloon.

In certain embodiments the device according to the invention comprises a reversible expandable device body. The device may accordingly be expanded along the line from the proximal to the distal end. The expansion may be directed along the line from the proximal to the distal end, or perpendicularly thereto. The expansion may, in specific embodiments, increase the volume of the device by 10, 20, 30, 40, 50, 60, 70, 80, 90, 100, 150, 200, 300, 400, 500, 600, 700, 800, 900, 1000% or more than 1000% or by any value in between the mentioned values.

The elongated tubular shape is preferably provided by a memory shaped wire, or memory shaped spiraling wire, which forms a tubular spiral. An example is a flexible nitinol wire. More preferably, the memory shaped spiraling wire may be modified into a single spiral-like wire structure, characterized by incomplete wire circles and an interdigiting structure.

It is further preferred that the extensions of the wire are provided in the form of circular loops or ellipsoids or non-straight longitudinal extensions.

In particularly preferred embodiments the expandable portions comprise a porous membranous surface, as described herein.

In further embodiments the expandable portion comprises a filter membrane. Accordingly, the device as defined above comprises at least one filter membrane, as described herein.

In a further set of embodiments, the filter membrane comprises, essentially consists of, or consists of one or more microfilaments. For example, the filter membrane comprises, essentially consists of, or consists of a multitude of longitudinally extending microfilaments, each of which is coatable by bioactive agent, thus creating a large bioactive surface area and permitting blood flow. The microfilaments may, in certain embodiments, be arranged parallel and straight or parallel and spiraling or in other arrangements. Further details may also be derived from FIG. 55, which shows and illustrates the corresponding embodiment.

In preferred embodiments, the filter membrane may comprise pores. These pores or the pores of the porous membranous surface as mentioned above may have a pore diameter which ranges from about 25 nm to about 100 μm in diameter. In preferred embodiments, the pores have different size ranges includes the range of about 25 nm to 100 nm in diameter, the range of about 100 nm to 10 μm in diameter, the range of about 10 μm to 25 μm in diameter and the range of about 25 μm to 100 μm in diameter. These ranges, which may all be present in one device or only a sub-group thereof, are adapted to the bodily fluid component to the recruited by the device, i.e. exosomes and the like being of the size of 25 nm to 100 nm, small cells being of the size of 100 nm to 10 μm, circulating tumor cells being of the size of 10 μm to 25 μm, and cell aggregates being of the size of 25 μm to 100 μm. It is further preferred that the pores are provided in a differential manner such as comprising differential ranges of 25 nm to 100 nm, 100 nm to 10 μm, 10 μm to 25 μm, or 25 μm to 100 μm. In further embodiments, the pores may have a diameter of 25 nm, 40 nm, 50 nm, 75 nm, 100 nm, 200 nm, 500 nm, 750 nm, 1 μm, 5 μm, 10 μm, 15 μm, 20 μm, 50 μm, or 100 μm or any value in between the mentioned values. Different pores in one device or one filter membrane or surface may, in further embodiments, be provided with the same diameter, or with two or more different diameters. It is preferred that pores with different diameters be present. It is also envisaged that pores and their edges and inner lumen (sacks) are loaded with cytokines, e.g. IL12 and antibodies or peptides which tightly bind the trapped tumor entity.

In embodiments, in which more than one filter membrane is present in a device, said filter membranes may preferably have differential pore diameters and/or differential pore pattern as defined herein. It is particularly preferred that the diameter ranges from about 25 nm to 100 nm, 100 nm to 10 μm, 10 μm to 25 μm, or 25 μm to 100 μm or any value in between the mentioned values.

The filter membrane may, in specific embodiments, be itself expandable. Accordingly, an expansion of the device may be followed or implemented by an expansion of the filter membrane. In certain embodiments, the filter membrane is attached to or arranged with the proximal and distal end of the device. In further embodiments, the filter membrane is designed as a non-permanent, retrievable filter membrane. According to these embodiments, the filter membrane may be separated from the remainder of the device and be removed therefrom, e.g. with a catheter or based on endoscopic techniques. The filter membrane may, in further embodiments, be designed as replaceable entity allowing for an exchange of a filter membrane after a certain period of time or after having reached the end of its working period (e.g. after 2, 3, 4, 5, 6, 7, 8, 9, 10, 11, 12, 15, 18, 20, 24 days; after 2, 3, 4, 5, 6, 7, 8, 9, 10, 11, 12, 15, 18, 20, 24 weeks; or after 2, 3, 4, 5, 6, 7, 8, 9, 10, 11, 12, 15, 18, 20, 24 or more months). It is preferred that the end of the working period does not surpass 24 months. In specific embodiments, the filter membrane may be retrievable as whole, or in parts. The partial retrievability may be implemented as segmented retrievability, e.g. via separable portions of the filter membrane, e.g. 10%, 20%, 30%, 40%, 50%, 60%, 70%, 80%, 90% etc., which can individually be retrieved, e.g. the distal part may be retrieved, whereas the proximal part is not retrieved etc. In further embodiments, any retrieval may be followed by replacement of the retrieved filter membrane by a new filter membrane or the same filter membrane after washing and preparation for a secondary use. Also envisaged is the retrieval of more than one segment at a time.

The present invention also envisages that the filter membrane is self-expandable. In further embodiments, the entire filter device assembly implant is self-expandable, as described herein.

It is further envisaged that the device according to the present invention comprises at least two filter membranes, each of which incompletely covers the cross-sectional area. These filter membranes may be arranged in any suitable form or shape. It is preferred that the filter membranes are arranged in tandem position along the longitudinal axis of the device body. Particularly preferred is a tandem arrangement along the longitudinal axis of the device body, wherein said filter membranes are opposite to each other within the circumference of the device body. Alternatively, they may be shifted in clockwise orientation. Such a shifting may advantageously be implemented for more than 2 filter membranes in tandem position, e.g. 3, 4, 5, 6, 7, 8, 9, 10 or more filter membranes. The device according to the present invention may, in further embodiments, comprise alternating non-completely covering filter membranes as defined herein

The present invention further envisages that the device is provided in any suitable form or architecture. It is particularly preferred that the device is provided in a tubular, onion like, pearl-chain-like, or a birds-nest like shape, or in any mixture of these shapes. Examples of corresponding forms and shapes can, for example, be derived from FIG. 24, 25, 26, 27, 28, 29, 30, or 31. The form or shape of the device may, in certain embodiments, be followed also be the form or shape of the filter membranes. For example, the device according to the present invention may comprise alternating non-completely covering filter membranes which are provided in a pearl-chain, onion type or birds-nest like shape. In other embodiments, the device comprises a multitude of longitudinally extending microfilaments, which may be arranged parallel or non-parallel to each other in straight configuration or tortuous e.g. spiralling configurations.

The device may be composed of, or be partially composed of, or comprise any suitable structural support material. According to certain embodiments, the structural support material may be metal. Preferred examples are stainless steel, gold, titanium, gold-titanium alloy, cobalt-chromium alloy, tantalum, tungsten, platinum-radium alloy, tantalum alloy, magnesium, nickel-titanium alloy, e.g. nitinol, silver or copper. Alternatively or additionally, the support material may be plastic or polymeric material. Also envisaged is the use of memory shape materials, e.g. elastic memory shape meshwork material. Preferred examples include memory shape elastic wires. In yet another alternative, the structural support material may be a material readable by tomography or other imaging techniques, e.g. X-ray. The present invention further envisages, in very specific embodiments, that the device body is not biodegradable or composed of biomaterial or biodegradable material. In alternative embodiments, the device or the device body may be biodegradable or composed of biomaterial or of biodegradable material, e.g. the device is composed of, is partially composed of, or comprises structural support material selected from the group comprising biodegradable or bioresorbable material, preferably as defined herein above in the context of the first main aspect.

The filter membrane may be composed of any suitable material. Envisaged examples include elastic or foldable polymer materials. Particularly preferred is polyurethane. Also envisaged is the use of micromeshes. In preferred embodiments, these micromeshes may comprise ultrathin wires, metallic or polymeric materials. In particularly preferred embodiments, the filter membranes according to the present invention is at least partially coated with one or more chemical and/or biological agents as defined herein.

In certain embodiments, the coating may differ between different filter membranes of a device. For a device comprising 2, 3, 4, 5, 6, 7 or more different filter membranes, a number of 2, 3, 4, 5, 6, 7 or more coatings may be provided. Alternatively, different filter membranes may be provided with one coating only. In further embodiments, the coating may change along the axis of the device, e.g. from proximal to distal over one filter membrane or over various filter membranes. In further embodiments, biological agents may be provided as coating in different combinations, e.g. as combination of adhesion and death signals, or as a combination of adhesion and immune modulation function.

In further embodiments the filter membrane as defined herein covers at least one cross-sectional area of the body of a device according to the present invention, in particular of the body of a device having a tubular form. For example, the filter membrane may cover 1, 2, 3, 4, 5, 6, 7, 8, 9, 10 or more cross-sectional areas of said body of a device according to the present invention. It is particularly preferred that the plane of the filter membrane is arranged perpendicular to the direction of the longitudinal axis of the body of a device according to the present invention. Also envisaged are different angles, e.g. non-perpendicular angles, between the plane of the filter membrane and the direction of the longitudinal axis, e.g. 10°, 20°, 30°, 40°, 50°, 60°, 70°, 80° or 85° or any value in between said values.

The present invention also envisages that the filter membrane is disposed within or around an expandable body, is characterized by a memory shaped metallic or memory shaped polymer structure and/or comprises a detachment mechanism cooperating between the expandable portion and an intraluminal segment of the device. Also preferred are devices comprising longitudinally extending free floating microfilaments. In specific embodiments, the device comprises an outer sheath concentrically disposed about the device. In this embodiment, the outer sheath and the device are movable relative to one another.

In a particularly preferred embodiment, the device as defined herein additionally comprises a downstream embolic debris filter, e.g. a retrievable downstream embolic filter.

In central embodiments of the invention the coating as mentioned above is a coating with one or more conductive materials. The term “conductive material” as used herein in the context of the third main aspect refers to organic polymers that conduct charge. Such polymers may have metallic conductivity or can be semiconductors. In another embodiment, the conductive material is a partial coating. In a preferred embodiment, the coating may cover between 5% and 95% of the surface of the device, e.g. 5, 10, 15, 20, 25, 30, 35, 40, 45, 50, 55, 60, 65, 70, 80, 90 or 95% of the surface of the device or, alternatively, of the filter membranes. In a further embodiment, the coating is provided in the form of symmetrically distributed areas on the surface of the device. It is particularly preferred that said symmetrical coating is provided within pores, e.g. as defined herein above.

In a preferred embodiment, the conductive materials are electro-active polymeric (EAP) materials. The term “electro-active polymeric material” refers to a class of organic materials that allow the direct delivery of electrical, electrochemical and electromechanical stimulation to cells. The family of electro-active polymeric materials may include conductive polymers, electrets, piezoelectric and photovoltaic materials. EAP materials may typically be classified in linear and aromatic (homo- and heteroaromatics) conjugated polymers. In one embodiment, the EAP materials may comprise poly(acetylene) (PAc), poly (p-vinylene) (PPV), poly (p-phenylene) (PPP), poly (γ-phenylene sulphide) (PPS), polypyrrole (PPy), polyaniline (PANI), polythiophene (PTh), poly (3,4-ethylenedioxythiophene) (PEDOT), emeraldine base polyaniline (EB-PANI), polypyrrole/graphene (PYG), poly(3,4-ethylenedioxythiophene) poly(styrenesulfo-nate) (PEDOT:PSS) and/or poly(isothianaphtene) (PITN).

In a further embodiment, the EAP material coating comprises one or more areas of insulator, semi-conductive and/or or purely conductive EAP materials. The term “insulator EAP materials” refers to an EAP material whose internal electric charges do not flow freely, whereas “purely conductive EAP materials” allow the flow of charge or electrical current freely in one or more directions. The electrical conductivity of “semi-conductive EAP materials” falls between that of purely conductive EAP materials and insulator EAP materials. Electrical conductivity, i.e. the ability of a material to pass current as described, may be measured in siemens per meter (S m⁻¹) or Siemens per centimeter (S cm⁻¹). In one embodiment, the value of electrical conductivity of insulator EAP materials may be between about 10⁻⁹ to 10⁻¹² S cm⁻¹, more preferably about 10 to 10⁻¹¹ S cm¹, most preferably about 10 to 10⁻¹² S cm⁻¹. The value of electrical conductivity of semi-conductive EAP materials may be between about 10 to 10 S cm⁻¹, about 10 to 10 S cm⁻¹, about 10 to 10 S cm⁻¹, about 10 to 10⁻⁶ S cm⁻¹, about 10 to 10 S cm⁻¹, about 10 to 10⁻⁴ S cm⁻¹, about 10 to 10⁻⁴ S cm⁻¹, about 10 to 10 S cm⁻¹, or about 10 to 10⁻² S cm⁻¹. It is preferred that the value of electrical conductivity of purely conductive EAP materials is between about 10⁶ to 10 S cm⁻¹, about 10⁵ to 10 S cm⁻¹, about 10⁴ to 10 S cm⁻¹, about 10³ to 10 S cm⁻¹, or about 10² to 10 S cm⁻¹.

According to the present invention, the EAP materials may, in certain embodiments, be doped or non-doped. The terms “doped”/“doping” or “non-doped” relates to the condition of EAP materials wherein their electrical conductivity is increased or decreased, respectively. In this context, the terms may also relate to the presence or absence of a chemical agent that is introduced for direct interaction with the polymer chain, which increases or decreases the electrical conductivity of EAP materials. For example, the extraction of electrons from the valence band (p-doping) generates positive charged holes in the electronic structure, while adding electrons to the conduction band (n-doping) generates a negative charge. In both cases, a possible charge mobility of electrons is created, so the conductivity is enhanced. The present invention also envisages doping processes such as chemical doping, electrochemical, photo-doping, charge injection doping, non-redox doping and secondary doping. Doped EAPs with increased conductivity are, for example, poly(acytylene) (Pac), poly(p-vinylene) (PPV), poly(p-phenylene) (PPP), poly(p-phenylene suplphide) (PPS), polypyrrole (PPy), polyaniline (PANI), polythiphene (PTh), poly(3,4-ethylenedioxythiophene), and poly(isothianaphtene) (PITN) but are not limited thereto.

The coating may further be provided on porous filter material. In this context the materials typically comprise additional co-porogenes. The term “porogen” refers to an agent for creating pores in organic materials. For example, NaCl crystals, crystallized sodium chloride particles of various sizes, and polyethylene glycol (PEG) powder may be used in the method of porogen generation to prepare porous scaffolds. It is preferred that a combination of NaCl crystals and PEG powder be used.

In further embodiments the device in the context of the third main aspect additionally comprises one or more substrate electrodes. The term “electrode” as used herein refers to a solid electric conductor, usually metal, used as either of the two terminals of an electrically conducting material; it conducts current into and out of the material. Herein envisaged is the use of cathode electrodes, i.e. of an electrode from which electrons emerge and which is designated as negative, or of anode electrodes which receives electrons and is designated as positive. It is particularly preferred that the electrodes are composed of platinum, glassy carbon, gold, SnO₂, metallized plastics, carbon fibers or TiO₂.

The activity of the device, such as filtering and recruiting, is mainly achieved by the presence of one or more chemical and/or biological agents which are comprised on said device and fulfil their activity in situ. An accessory effect improving the filtering or recruiting may be provided by the form and design of the device itself, e.g. by reducing the circulation velocity of the bodily fluid, preferably of the blood stream. Accordingly, the retention time of circulating tumor cell and/or circulating metastatic cell and/or motile parts of tumor cells or of immunologic cell such as a T cell, B cell or dendritic cell and/or an antibody complex such as an autoantibody complex, and/or a neurologic serum marker protein at or on the device can advantageously be extended, allowing for molecular interactions between the circulating tumor cell and/or circulating metastatic cell and/or motile parts of tumor cells and/or immunologic cell such as a T cell, B cell or dendritic cell and/or an antibody complex such as an autoantibody complex, and/or a neurologic serum marker protein on the one hand, and the one or more chemical and/or biological agents located on the device on the other hand.

It is particularly preferred that the device as defined herein be designed according to current implant techniques and technologies (e.g. stent implantation, septal occluder implantation, vena cava filter implantation). It is further preferred that it comprises an inactive (unexpanded, folded or constraint state) and an active (expanded, unfolded, unconstraint) state. The device is introduced into the target organ in an unexpanded state and is released at the target position into the expanded and active state

In a central aspect of the present invention the one or more chemical and/or biological agents as mentioned above perform different chemical, biological and/or biochemical activities. Said activities may ultimately contribute and facilitate an effective recruiting of circulating tumor cells, circulating metastatic cell or motile parts of tumor cells, thus allowing for a removal of said cells, cell aggregates or parts of tumor cells or exosomes and/or immunologic cell such as a T cell, B cell or dendritic cell and/or an antibody complex such as an autoantibody complex, and/or a neurologic serum marker protein from the bodily fluid, e.g. from the blood and at the same time allow for a prolonged and safe use of the device within a subject, as well as the efficient and extensive removal of these cells and parts thereof or antibody complexes or neurologic serum marker proteins from the subject, even after a prolonged time, e.g. after 2 to 30 days, 4 to 8 weeks, or 1 to 24 months. To achieve one or more, preferably all, of these functions, the one or more chemical and/or biological agents are provided as coating on the device, in particular on the filter membrane of the device as defined herein above.

A further important factor is the presence of an ECM-like structure on the surface of the device, as defined herein below. The ECM-like structure may be provided in an equal manner throughout the device, or it may be provided in certain sectors or regions or the device only, or in certain sectors or regions of the device different forms of these structures may be present. The ECM-like structure essentially fulfils the function of a structural and/or biochemical support for surrounding cells, which is assumed to be of elevated importance once cells have settled down on or within the device. Furthermore, the ECM-like structure resembles the microenvironment of the tumor or metastases. This ECM-like structure which is typically composed of laminins, adhesion molecules or chemical entities serves as homing compartment within the device. Yet another important factor, which is considered relevant for the initial recruiting steps is the presence of a biological agent, which is capable of binding to a tumor marker on a cell or part thereof, in particular on a recruiting a circulating tumor or circulating metastatic cell or motile parts of tumor cells. These different factors may, in specific embodiments, present together at one area of the device, or they may fulfil their functions in different areas or the device. Also suitable mixture of any of these factors are envisaged. Furthermore, the present invention envisages different forms of devices which may comprise or implement either all or a sub-group of said factors. The overall shape, the intended use, the specific medical condition of a subject, the intended time of use and/or the tumor form may require an adjustment of the presence of above mentioned factors or functions. Additional factors which may also be taken into account are the tumor staging, i.e. the phase or stadium of the disease, and/or details on a pretreatment, e.g. whether the subject has already been treated, how successful this treatment was and the time period since the treatment, as well as a possible chemotherapeutic resistance etc., or the immunologic sate of the patient, or the stage or severity of a disease such as an neurologic disease involving certain amounts of antibody complexes or the like.

Accordingly, a device of the invention may, in particularly preferred embodiments, besides the conductive materials, e.g. EAP materials mentioned above, further comprise (i) a passive coating with one or more polymeric materials, (ii) as well as an ECM-like structure, and (iii) an active coating which is capable of binding to a tumor marker, to an immune cell marker on a cell or part thereof or to an antibody complex. In further embodiments, at least one of these elements (i) to (iii) may not be present. The present invention envisages all combinations of (i), (ii) and (iii) as defined above.

In one group of embodiments of the invention the coating may thus be a passive coating, as defined herein.

In a further group of embodiments, the chemical and/or biological agents composing the coating of the device constitute an extracellular matrix-like structure, as defined herein.

In specific embodiments, the macromolecules used to provide an ECM like structure, i.e. the macromolecules which are provided as coating for the device according to the present invention, are proteoglycans, non-proteoglycan-polysaccharides, elastin; fibronectin or laminin. Particularly preferred are mixtures of these macromolecules. Preferred examples of suitable proteoglycans include heparan sulfate, chondroitin sulfate and/or keratin sulfate. Preferred examples of suitable non-proteoglycan-polysaccharides include hyaluronic acid. The present invention further envisages the employment of ECM-like structures in the form of protein mixtures. Such mixtures are, for examples, secreted by Engelbreth-Holm-Swarm (EHS) mouse sarcoma cells. Also preferred are Matrigel, BioCoat or GelTrex mixtures. It is particularly preferred that protein mixtures with substitutions of human proteins are used.

According to the present invention, the device comprising a coating as defined herein provides an environment for circulating tumor cells, for circulating metastatic cell or for motile parts of tumor cells, for exosomes, for cell aggregates, as well as for immunologic cell such as a T cell, B cell or dendritic cell and/or an antibody complex such as an autoantibody complex, and/or a neurologic serum marker protein. The environment may, for example, allow metastatic cells to settle down in or on the device and thus leave the bodily fluid circulation, e.g. lymph or blood. Similarly, tumor cells which are non-metastatic but circulate through the body may be stimulated to settle down in or on the device and thus leave the bodily fluid circulation, e.g. lymph or blood. This function is preferably implemented by the use of, or presence of a biological agent in or on the device, in particular as part of the coating, wherein said biological agent is capable of binding to a tumor marker on a cell or part thereof, in particular on a recruiting a circulating tumor or circulating metastatic cell or motile parts of tumor cells. In further embodiments, the present invention also envisages a binding to immune cells or corresponding markers on immune cells, i.e. immune cell markers. In yet another set of embodiments, the present invention also envisages a binding to an antibody complex such as an autoantibody complex. Also envisaged are neurologic serum marker proteins, e.g. associated with certain neurologic diseases such as Parkinson's.

Examples of tumor-marker specific antibodies envisaged by the present invention include panitumab, matuzumab, nimotuzumab or derivatives thereof. Further envisaged are additional antibodies against EGFR family members, or ErbB2/HER2 family members. Further details would be known to the skilled person or can be derived from suitable literature sources such as Huang and Buchsbaum, Immunotherapy, 2009; 1(2): 223-239.

The present invention, in preferred embodiments in the context of the third main aspect, envisages the use of one or more of the following homing factors: Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

The term “antibody complex” as used herein in the context of the third main aspect which is sometimes also called “immune complex” relates to a molecule formed from the integral binding of an antibody to a soluble antigen. These complexes may themselves cause illness when they are deposited in organs, for example, in certain forms of vasculitis. In preferred embodiment, the antibody complexes are autoantibody complexes formed by a mammalian body itself. An “autoantibody” is generally understood as an antibody produced by the immune system that is directed against one or more of the individual's own proteins. Several autoimmune diseases are caused by such autoantibodies. Autoimmunity typically involves the loss of normal immune homeostasis such that the organism produces an abnormal response to its own self tissue. The body accordingly generates autoantibodies that attack normal cells by mistake. At the same time regulatory T cells fail to keep the immune system in line, which results in a misguided attack on the own body, leading to autoimmune diseases. Nearly any body part can be involved, including heart, brain, nerves, muscles, skin, eyes, joints, lungs, kidneys, glands, the digestive tract, and blood vessels. Examples of such diseases, which are also envisaged by the present invention, include lupus erythematosus, alopecia areata, celiac disease, diabetes mellitus type 1, Graves' disease, inflammatory bowel disease, multiple sclerosis, psoriasis and rheumatoid arthritis.

The term “neurologic serum marker protein” as used herein in the context of the third main aspect refers to a protein or biomarker which can be detected in a bodily fluid, in particular when the subject is affected by a neurologic disease. In the context of the present invention the serum marker protein is thus directly associated with a neurologic disease. It is in a specific embodiment associated with Parkinson's disease. Examples of corresponding serum marker proteins include alpha-synuclein, UCH-L1, beta-glucocerebrosidase, Abeta42, Tau protein, NFL, or BDNF.

In specific embodiments any combination of the above mentioned elements, e.g. homing factors, CD133, VEGFR-1 or antibody etc. may be used. Also envisaged is the use of one type of element, e.g. one specific homing factor, in a specific area of the device, followed by a different type of element in the neighbouring area etc.

The present invention further envisages that a biological agent as defined herein, e.g. a homing factor, an antibody etc. is linked to the passive coating of the device via a spacer element. Also envisaged is the linkage to the structural support material of the device, e.g. stainless steel, gold, titanium, gold-titanium alloy, cobalt-chromium alloy, tantalum, tungsten, platinum-radium alloy, tantalum alloy, magnesium, nickel-titanium alloy, e.g. nitinol, silver or copper; plastic or polymeric material; elastic memory shape meshwork material such as memory shape elastic wires or a material readable by tomography or other imaging techniques, e.g. X ray; or any mixture thereof, which may be present on the surface of said filter device assembly implant.

In addition to its function as spatial separator between the biological agent and the surface of the device, the spacer element further fulfils the function of a separator between biological agents as defined herein. The present invention accordingly envisages the provision of biological agents in a density which allows for an efficient binding of the biological agent to a tumor cell or part thereof. According to preferred embodiments, the spacer elements may be provided in a density of 1 mg per cm² on the surface of the device. In further embodiments, spacer elements may be provided in a density of 2 to 500 per μm2 on the surface of the device. For example, a density of 2, 5, 10, 15, 20, 30, 40, 50, 60, 70, 80, 90, 100, 150, 200, 250, 300, 350, 400, 450 or 500 per μm2 on the surface of the device may be used.

The present invention further relates to biological agents—as part of the device—which comprise, essentially consists of, or consists of a binding domain capable of binding to a tumor marker as defined herein.

In preferred embodiments, the binding domain is a peptide or polypeptide molecule. The do-main may have any suitable length. It is preferred that it has length of about 20 to about 250 amino acids. More preferably, the binding domain has a length of about 20 to about 120 amino acids. In further preferred embodiments, the binding domain has a length of about 20, 25, 30, 35, 40, 45, 50, 55, 60, 65, 70, 75, 80, 85, 90, 95, 100, 105, 110, 115 or 120 amino acids, or any value in between the mentioned values.

The binding domain capable of binding to a tumor marker according to the present invention may be provided in any suitable form. For example, the binding domain may be provided as part of a larger protein structure, wherein said larger protein structure may fulfil presenting or structural functions, thus improving the expose of the binding domain. Further envisaged is combination of more than one binding domain per biological agent unit as defined herein.

In further embodiments, the binding domain capable of binding to a tumor marker according to the present invention may be combined with one or more additional functional domains. Preferably, such a further functional domain is or comprises, partially comprises or consists of an apoptosis inducing factor or a functional domain of an apoptosis inducing factor capable of inducing apoptosis. Preferred examples of suitable apoptosis inducing factors are FasL/CD95L, TNF-alpha, APO3L and APO2L/TRAIL. The presence of such apoptosis inducing factors advantageously allows the device of the present invention to not only attract tumor cells or metastatic cells, but also to subsequently induce a killing of these cells via apoptotic processes. The present invention further envisages the use of additional killing approaches based on molecular interactions between the surface of a cancer cell or metastatic cell and an interactor.

In specific embodiments of the present invention the domain capable of binding to a tumor marker and the domain capable of inducing apoptosis are provided as fused domains. Also envisaged is the alternative that the domains are linked via a linker element of about 1 to 20 amino acids length. For example, the linker element may be a peptide having a length of 1, 2, 3, 4, 5, 6, 7, 8, 9, 10, 11, 12, 13, 14, 15, 16, 17, 18, 19 or 20 amino acids.

In further specific embodiments of the present invention the biological agent is provided as linear or circular element or as an element composed of linear and circular parts. It is particularly preferred to provide the biological agent as a linear or circular or partially linear or circular peptide or poly-peptide. Typical embodiments envisage that the circular biological agent has or is part of a structure comprising a loop or a loop and a stem. Also envisaged are linear structures, which are linked to a spacer element as defined herein. The loop structure or linear structure may comprise said preferably comprise the biological agent, e.g. the tumor binding domain or the apoptosis inducing domain, at an exposed position. Said exposition allows for an efficient interaction, e.g. leading to the binding to a tumor marker or a tumor cell, or the induction of apoptosis.

In a further preferred embodiment the peptide as used in the context of the present invention in the context of the third main aspect has one or more of the properties (i) to (xxii) as defined herein above. Also envisaged is any combination of these peptides with components of the device as mentioned above. Further information on specific sequences of peptides envisaged by the present invention may be derived from FIG. 65, 66 or 67.

In further specific embodiments the loop or linear structure as mentioned above comprises additional elements selected from the group comprising a sensor unit or an interaction unit such as an external interaction unit. The term “sensor unit” relates to a molecular unit which detects a physiological or other condition within the subject's body, converts the sensed value to an electrical signal, and then transmits the electrical signal to a receiving or monitor or external interaction unit, preferably in continuous wireless or wire-based communication with the sensor unit. In one embodiment, the sensor unit may comprise one or more sensors. Further details would be known to the skilled person or can be derived from suitable literature sources such as Son et al., AcsNano, 2015, 9, 6, 5937-5946.

In a preferred embodiment, the sensor unit is a molecular conformation sensor. This sensor is capable of changing its conformation upon binding of e.g. a ligand to the biological agent. Subsequently, it conveys this conformation change to a receiving unit, which may be close to the spacer element. Alternatively, it may convey the information to a part of the extracellular matrix-like structure, to the coating of the device and/or on the surface of the implant. A further option is the emission of light or heat upon said conformational change, which may be implemented by the use of suitable dyes or heat producing agents. Both, light and heat, can be detected, e.g. in situ and be signaled to a receiving unit, e.g. outside of the subject or residing on the device. In a particularly preferred embodiment the conformational change is converted into a sensed signal which is transmitted from the device to the environment, e.g. to a receiving unit in the environment.

An “interaction unit” means any unit or module which is capable of interaction with the sensor. Such a unit may be external to the device and operate as an electron or signal receiving unit.

In further specific embodiments a biological agent as defined herein above comprises or is linked to one or more additional elements. For example, the biological agent may be linked to a sugar, to branched or unbranched multiple sugar structures, to alkynes or azides, to streptavidin, biotin, amines, carboxylic acids, active esters, epoxides or to aziridines.

In a further embodiment the device is partially composed of or comprises material selected from the group comprising material readable by tomography or suitable for electrochemical or electronic readouts. Such materials include platinum, gold, or tantalum; or electro-active polymeric (EAP) materials as defined herein.

In a further embodiment the device as defined in the context of the third main aspect comprises, is combined with or is designed to be combinable with one or more elements selected from (i) an electrical electrode, e.g. as defined above (ii) a microprocessor or circuit component, (iii) a read-out module, (iv) a digital domain, (v) a controller component and (vi) a communication module.

The read-out module may determine a signal strength of the information transmitted, e.g. from the sensor unit to the read-out module; information that is shown in electronic form, for example on a computer screen, for example a read-out of device parameter or environmental parameters.

The read-out module may also include a warning device, an audio communications device such as an audio transceiver, and other features. For example, the warning signal may be generated responsive to the information transmitted from the sensor unit.

In a preferred embodiment, the read-out module is composed of an analog front-end (AFE) and an analog-to-digital converter (ADC), capable of capturing and processing the sensed signal and converting it to the digital domain. The term “analog front-end” refers to a set of analog signal conditioning circuitry that uses sensitive analog amplifiers, often operational amplifiers, filters and integrated circuits for sensors, radio receivers, and other circuits to provide a configurable and flexible electronics functional block, needed to interface a variety of sensors to an antenna, analog to digital converter or a microcontroller.

In particularly preferred embodiment the sensed signal is processed by a digital signal processor (DSP). A “DSP” is a specialized microprocessor (or a SIP block) chip, with its architecture optimized for the operational needs of digital signal processing. DSPs are fabricated on MOS integrated circuit chips. The goal of a DSP is usually to measure, filter or compress continuous analog signals. DSPs often use special memory architectures that are able to fetch multiple data or instructions at the same time. DSPs as envisaged herein also implement data compression technology, with the discrete cosine transform (DCT) in particular being a widely used compression technology in DSPs.

In a further preferred embodiment, the read-out module is capable of registering certain device parameters. Such parameters include oxygen content, sugar content, temperature, ion concentration, impedance, conductivity, pH, pressure, colors, color changes or color pattern, fluorescence, or bioluminescence. The read-out module may accordingly comprise sensors or detectors for any of the mentioned parameters. Suitable sensors and their implementation would be known to the skilled person. For example, oxygen content may be detected by an oxygen sensor capable of measuring the proportion of oxygen in the liquid being analyzed, i.e. blood. Further examples include blood glucose sensors, bimetal temperature sensors, potentiometric ion sensors for determining ion concentration, conductivity sensors, pH meters, pressure sensors, and light detecting sensor for determination of colors or luminescence etc.

The digital domain typically collects and stores information from one or more of the mentioned modules. It may serve as documentation center for the device during operation. The digital domain may further be closely connected to the communication module and provide information to be sent out to a receiving unit.

The controller unit is envisaged as control instance for all sensory and operational procedures of the device. It is typically connected to the digital domain and the read-out unit and controls communication via the communication module. It comprises one or more microprocessors and optionally data storage capabilities.

The communication module allows for wireless communication with a remote receiving station. This communication module is, in certain embodiments, based on high-speed wireless communication standards such as LTE (long-term evolution), or GSM/EDGE or UMTS/HSPA technologies, or any other suitable high-speed wireless communication technology or standard, e.g. also technologies which will be developed in the future, or are not yet commercially available such as 5G or successors thereof. It is preferred that the communication module allows for real-time communication with a remote receiving station. The communication may preferably collect and transmit data from the modules present in the device to a receiving module. The communication module may, in further embodiments, also be equipped with a second or further communication module, e.g. a WiFi or WLAN module for local data transfer in a surrounding which provides suitable receiving possibilities. In alternative embodiments, the communication module may be capable, or may additionally be capable of transferring data with further protocols such as NarrowBand IOT (NB-IoT). NarrowBand IoT (NB-IoT) is a Low Power Wide Area Network (LPWAN) radio technology standard developed to enable a wide range of devices and services to be connected using cellular telecommunications bands. NB-IoT is a narrowband radio technology typically designed for the Internet of Things (IoT) and is one of a range of Mobile IoT (MIoT) technologies standardized by the 3rd Generation Partnership Project (3GPP). The present invention further envisages the use of similar technologies such as eMTC (enhanced Machine-Type Communication) and EC-GSM-IoT. In further embodiments, the communication module may further be capable of receiving information form an (outside) receiving module, e.g. with respect to device operations such as release of drugs etc.

Further envisaged is the use of RF (radio frequency) or RFID technology. The RFID technology uses electromagnetic or electrostatic coupling in the RF portion of the electromagnetic spectrum to transmit signals. RFIDs may generally be classified as active or passive. Active RFID systems typically have 3 components: (a) a reader, transceiver or interrogator, (b) antenna, and (c) a transponder or IC programmed with information. Active RFID tags typically possess a microchip circuit (transponder or integrated circuit (IC)) and an internal power source, e.g. a battery, and when operably connected to an antenna, the active RFID tag transmits a signal from the microchip circuit through the power obtained from the internal battery. Typically, active RFID tags such as transponders and beacons are used. In one example, a system may use an active transponder. In this scenario, the reader sends a signal and when the antenna and tag are operably connected, the tag will send a signal back, e.g. with the relevant information programmed to the transponder. In a different scenario, an active beacon is used wherein the beacon sends out a signal on a periodic basis and it thus does not rely on the reader's signal. In contrast to active systems, passive RFID systems comprise (a) a reader, transceiver or interrogator, (b) antenna, and (c) a tag programmed with information. A passive RFID tag typically includes a microchip or integrated circuit (IC), and it may contain the antenna as an integral component of the tag or as a separate device. In passive systems, the tag typically does not include a power source. In one example, the antenna can be an internal component of the tag, i.e., the antenna and IC can be contained in a single device. However, until operably connected in the device, the antenna and IC may not interact. Alternatively, the antenna and IC may be provided on separate components. Typically, passive tags wait for a signal from an RFID reader. The reader thus sends energy to an antenna which converts that energy into an RF wave which is transmitted into the read zone. Once the tag is read within the read zone, the RFID tags internal antenna is typically powered via RF waves. Accordingly, the tags antenna fuel the IC with energy which generates a signal back to the RF system. Such process of change in the electromagnetic or RF wave, can advantageously be detected by the reader (e.g. via the antenna), which may in turn interpret the information. Accordingly, passive RFID tags have typically no internal power source and normally comprise an IC and an internal antenna. The tag may, in specific embodiments, comprise an electronic product code (EPC) or a similar code, which is a 96-bit string of data. Also envisaged are alternative codes, which allow to identify a product or element. The RFID tags may be used at different frequencies, e.g. at a low frequency (LF) of 125-134 kHz, at a high frequency (HF) of 5-7 MHz, at a HF and Near-Field Communication (NFC) frequency of 13.56 MHz, at an ultra-high frequency (UHF) of 433 MHz, 865-868 MHz, 902-928 MHz, or in the Giga Hertz band of 2.45 to 5.8 GHz. It is preferred to make use of a frequency at or around 13.56 MHz.

Also envisaged is the employment of electric resistance measurements. The term “electric resistance measurement” means that an electric signal which is provided to the device, e.g. from the outside, or alternatively also from an internal site in the device, which, in turn, elicits a signal in the device in dependence on the electrical current conduction and resistance encountered in the device. This electric current or resistance is provided in dependence on the presence of bound cells, cell aggregates, exosomes, antibody complex or proteins etc. Typically, an increased resistance is indicative for the presence of bound cells, cell aggregates, exosomes or parts thereof. This simple measurement approach allows for an online and rapid alerting upon cellular interaction and a fast detection of molecular events, which is considered to be diagnostically valuable.

Further envisaged is the use of optic fibers or a cable connections. For example, an optical signal may be transmitted from the inside of the subject's body to an external read-out device or receiving unit when the chemical and/or biological agents of the device interact with a circulating tumor cell and/or metastatic cell and/or motile parts of tumor cells and/or an aggregate of tumor cells and/or a tumor cell derived exosome and/or an immunologic cell such as a T cell, B cell or dendritic cell and/or antibody complexes or serum markers within the bodily fluids of luminal organs. Similarly, electric signals generated in the device may be transmitted to the outside via “cable connections”, i.e. metal cables.

An outside receiving module, as mentioned herein may, according to certain embodiments of the invention be, or be comprised or integrated in, or be associated with: a mobile phone, a computer, a tablet, a handheld device, or a network server, or a network cloud computing device. It is particularly preferred to provide the outside receiving module as a hand-held radio transmission and receiving device. The “network server” is meant comprise a computer system or computer program, which is used as the central repository of data and various programs that are shared by users in a network. In the context of the electric resistance measurement as defined herein above, a hand-held physiological signal measurement device is preferred.

The hand-held physiological signal measurement device or hand-held radio transmission and receiving device may, in specific embodiments in the context of the third main aspect, comprises one or more of the following: (i) a housing, (ii) a plurality of electrodes attached to the surface of the housing, (iii) a contact with the skin surface of the subject, obtaining from the user a physiological signal's; (iv) a front-end circuit, preferably located inside the housing and connected to the plurality of electrodes to receive the physiological signal; (v) an analog/digital conversion circuit, preferably located inside the housing and connected to the front end circuit; (vi) a wireless transceiver interface located inside the housing; (vii) a processing unit preferably located in the inner housing, which is connected to the analog/digital conversion circuit and a transceiver.

In further embodiments in the context of the third main aspect, the receiving module is connected with a data processing system. Said data processing system preferably comprises a program capable of collecting data sent by said communication module over time and/or of analyzing or processing said data. In a further preferred embodiment, the program is additionally capable of representing said data graphically and/or comparing said data with one or more reference values and/or wherein said program is capable of comparing a threshold value with a measured electrical current value. These steps thus allow monitoring clinically relevant events and transforming them into a signal which is being read from the external signal receiving module. The threshold may be defined according to previous experiences, or be preferably derived from background noise.

In a further central embodiment in the context of the third main aspect, the device as defined herein additionally comprises a drug release module. Said release module is preferably controllable by the communication module as defined herein. The “drug release module” as used herein relates to one or more reservoirs containing a pharmaceutically active compound and a control means for selectively releasing an effective amount of the pharmaceutically active compound from the reservoir(s). Also comprised are one or more electrodes or sensors for monitoring, stimulation, or both; and a microcontroller for controlling operational interaction of the drug release module and additional modules or units. It is preferred that the release module is controllable by heat, electrical, magnetic or ultrasound stimuli.

The one or more pharmaceutically active compounds comprised in said drug release module may be any suitable pharmaceutically active compound known the skilled person. Preferably, the pharmaceutically active compound is an anti-cancer drug, an anti-thrombotic drugs, a cardiovascular drug, or drug against an neurologic disease, e.g. Alzheimer's disease, Parkinson', or a drug against an autoimmune disease as mentioned herein.

In a further aspect the present invention relates to device as defined herein in the context of the third main aspect for use in treating cancer and/or metastasis, or for use diagnosing and/or monitoring a disease, preferably cancer, tumor metastases, a cardiovascular disease, or for determining an increased likelihood for developing a disease, preferably cancer, metastases, a cardiovascular disease, or a neurologic disease such as Alzheimer's disease in a subject.

The device of the present invention may be used in both human diagnosis and veterinary diagnosis, preferably in human diagnostic approaches.

In a further aspect in the context of the third main aspect the present invention relates to a tubular shaped elongated catheter device assembly as defined herein or another device as defined herein in the context of the third main aspect and comprising one or more features of the third main aspect for use in preventing a disease, preferably cancer, metastases, cardiovascular diseases, neurologic diseases. In yet another aspect the present invention relates to a tubular shaped elongated catheter device assembly as defined herein for use in treating a disease, preferably cancer, metastases, a cardiovascular disease, or a neurologic disease.

The treatment or prevention may further, in specific embodiments, involve a single administration or use of the device as defined above, or multiple administrations or uses. Such uses include, for example, drug releases as defined herein above. A corresponding administration or usage scheme may be adjusted to the sex or weight of the patient, the disease, the general health status of the subject etc. For example, the administration or use scheme may contemplate a usage within the subject's body for one week, two weeks, 3, 4, 5, 6, 7, 8, 10, 11, 12 weeks, 4, 5, 6, 7, 8, 9, 10, 11, 12, 18, 24 or more months, or any time period in between the mentioned periods. These usage schemes can of course be adjusted or changed by the medical practitioner in accordance with the subject's reaction to the treatment/prevention and/or the course of the pathological condition.

The device as defined above may accordingly be administered to the subject in as a controlled active, controlled release and toxic entities (tumor cells or metastatic cells) accumulating agent.

The administration or implantation of the device may preferably be performed during and/or after the treatment of a subject with a therapeutic agent. The treatment of a subject as mentioned here is typically an anti-cancer treatment. The administration or implantation of the device may, in further embodiments, be preferably performed during and/or after surgery removing a tumor load. It is particularly preferred that the administration scheme foresees an implantation immediately after said surgery or, in the alternative, as ultimate step of the surgery to capturing not removed cancer cells.

It is particularly preferred that the cancer to be treated or prevented is a colon cancer, breast cancer, lung cancer, melanoma, esophageal cancer, prostate cancer, pancreatic cancer, ovarian cancer, myeloma, or a lymphoma such as ALL, CLL, or AML. It is further particularly preferred that the metastasis to be treated or prevented is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarial tumor or nephroblastom such as Wilms tumor. Due to its shape and, in particular, its functionality as defined herein above, the device of the present invention is capable of quantitatively or almost quantitatively capturing circulating tumor cells or metastatic cells or motile parts of tumor cells in a subject's body. Due to its shape and, in particular, its functionality as defined herein above the device of the present invention is accordingly capable of preventing downstream organs or tissues to be reached by tumor cells or metastatic cells or motile parts of tumor cells which are circulating in a subject's body.

In further specific embodiments, the device of the present invention is specifically designed to be implanted into a luminal organ, preferably a blood vessel. Examples of envisaged blood vessels are an artery, an elastic artery, a distributing artery, an arteriole, a capillary, a venule or a vein. It is further envisaged to design the device for an implantation into a heart chamber or an elastic artery. The device may accordingly be designed as free floating device with connecting wires to maintain its position. This concept also allows for retrieval. In further specific embodiments, the device of the present invention is specifically designed to be implanted into a lymphatic vessel. Particularly preferred is vena cava, or any other vessel wherein said device can suitably be used free floating. Further envisaged and preferred is a transvascular implantation. In certain embodiments, the device is designed to be transvascularly advanced and implanted at a target site. The device may accordingly be designed to have adequate dimensions for an advancement in the target tissue or region. It may, in further embodiments, be catheter based and preferably steerable. It is particularly preferred that the device is steerable is by its torque stable structure or by comprising a steerable wire. Also envisaged in the present invention is a percutaneous implantation, a minimal invasive implantation, an endoscopically guided implantation, e.g. via ultrasound or magnetic resonance devices, or a laparoscopic implantation. The implantation may, in preferred embodiments, be performed directly in a tumor or tumorous tissue or in the close vicinity of a tumor or tumorous tissue, or in any other target organ or tissue, as well as corresponding device designs and their use.

In certain specific embodiments a device to the implanted via a percutaneous implantation, a minimal invasive implantation, an endoscopically guided implantation, e.g. via ultrasound or magnetic resonance devices, a laparoscopic implantation, or a transcutaneous implantation may have a specific form or design, e.g. may not be composed of filters as defined herein. Preferably, the device may have or comprise a porous solid architecture or design, or may have the form of porous solid scaffold.

It is particularly preferred that the device of the present invention is used by implantation in a blood or lymphatic vessel downstream of an existing cancer site in a subject. If such a positioning downstream of an existing cancer is envisaged, it is preferred that the device is used by implantation in close proximity to said existing cancer site.

In further preferred embodiments that the device of the present invention is used by implantation in a blood or lymphatic vessel upstream of a tissue with a high risk of developing metastasis. Further information may be derived, for example, from FIG. 60, which shows relevant blood vessels and mentions the connected tissues.

In yet another aspect the present invention relates to a method of treating cancer and/or metastasis, comprising implanting a device according to the invention into a subject in need thereof. Also envisaged is a method of preventing cancer and/or metastasis, comprising implanting a device according to the invention into a healthy subject or a subject being at risk of developing cancer and/or metastasis.

In yet another aspect of the third main aspect the present invention relates to a method of diagnosing or monitoring a disease such as cancer and/or metastasis and/or a cardiovascular disease, or of determining an increased likelihood for developing a disease such as cancer and/or metastasis or a cardiovascular disease comprising implanting a tubular shaped elongated catheter device assembly as defined herein into a subject in need thereof and detecting the presence of cancer and/or metastatic cells captured by the implant.

In yet another aspect of the third main aspect the present invention relates to a method of diagnosing or monitoring a disease such as cancer and/or metastasis and/or a cardiovascular disease, or of determining an increased likelihood for developing a disease such as cancer and/or metastasis and/or a cardiovascular disease comprising implanting a tubular shaped elongated catheter device assembly as defined herein into a healthy subject, a subject showing no symptoms of a disease, preferably symptoms of cancer or metastasis or a cardiovascular disease, or a subject being at risk of developing a disease such as cancer and/or metastasis or a cardiovascular disease and detecting the presence of cancer and/or metastatic cells captured by the device.

In yet another aspect of the third main aspect the present invention relate to a method of monitoring the effect of disease treatment, comprising implanting a tubular shaped elongated catheter device assembly as defined herein into a patient currently being treated for a disease such as cancer and/or metastasis and or a cardiovascular disease, or a patients who has finished his disease treatment such as cancer and/or metastasis or cardiovascular disease or neurologic disease treatment within a time period of about 1 day to 2 years.

Also envisaged is a method of data collection comprising (i) monitoring a tubular shaped elongated catheter device assembly as defined herein implanted in a subject via ultrasound scanning, tomography, optical, or electrochemical measurements, or by determining the tubular shaped elongated catheter device assembly environment for changes indicative a disease electrochemical measurements; and (ii) collecting data over time for recognizing changes of the monitored values.

The present invention further envisages in the context of the third main aspect a method of manufacturing a device as defined herein in the context of the third main aspect. The method preferably comprises the step of providing a biological agent by expressing said biological agent as polypeptide in a suitable host cell. Furthermore, the biological agent, e.g. a polypeptide, may be modified, e.g. by adding one or more of a sugar, a branched or unbranched multiple sugar structure, an alkyne, an azide, a streptavidin, a biotin, an amine, a carboxylic acid, an active ester, a epoxide or an aziridine. The method may further comprise coating steps which cover or at least partially cover the device with a passive coating and/or an ECM-like structure and/or a biological agent or active coating. Also included are steps of coating with EAPs and/or the additional of sensors or electronic modules as described herein.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

Human pancreatic cancer cells were purchased from ATCC CFPAC-1 ATCC® CRL-1918™ pancreas; derived from metastatic: liver; human origin. LOT:64094719. The human pancreatic cancer cell line was purchased from American Type Culture Collection (Manassas, Va.). Cells were thawed washed 2-3 times (1200 rpm, 3′, RT) and subsequently cultured in MDM (Life Technologies) supplemented with 10% FBS (Life Technologies) and PenStrep at 37° C. and 5% CO2 in a humidified incubator.

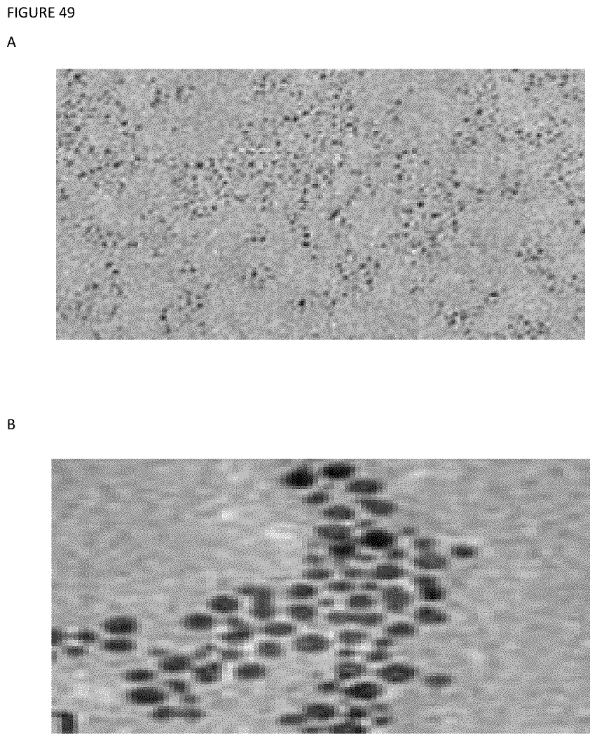

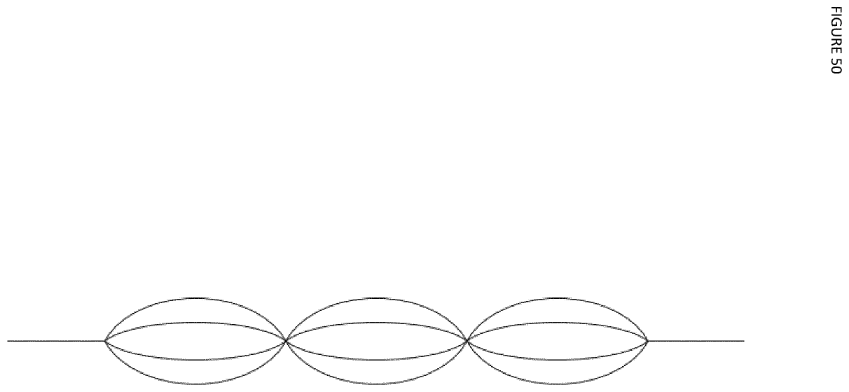

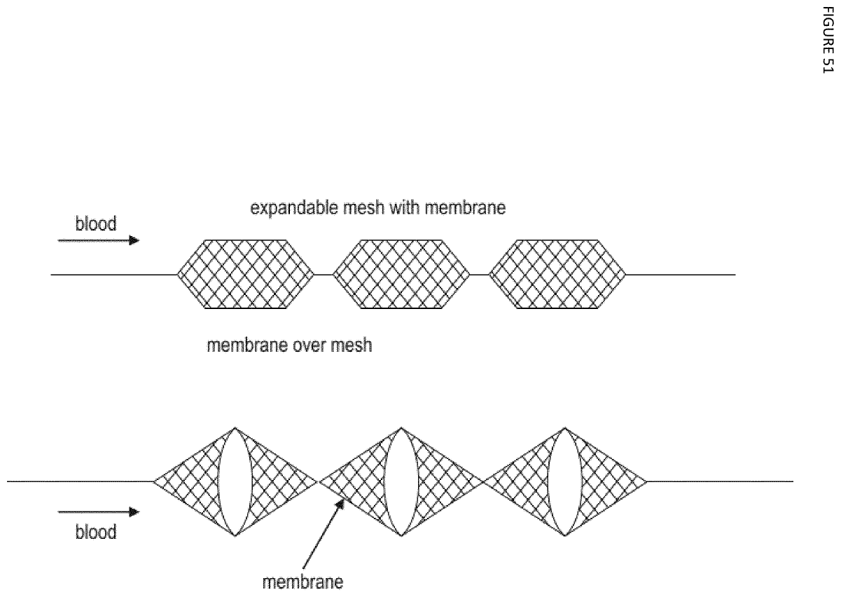

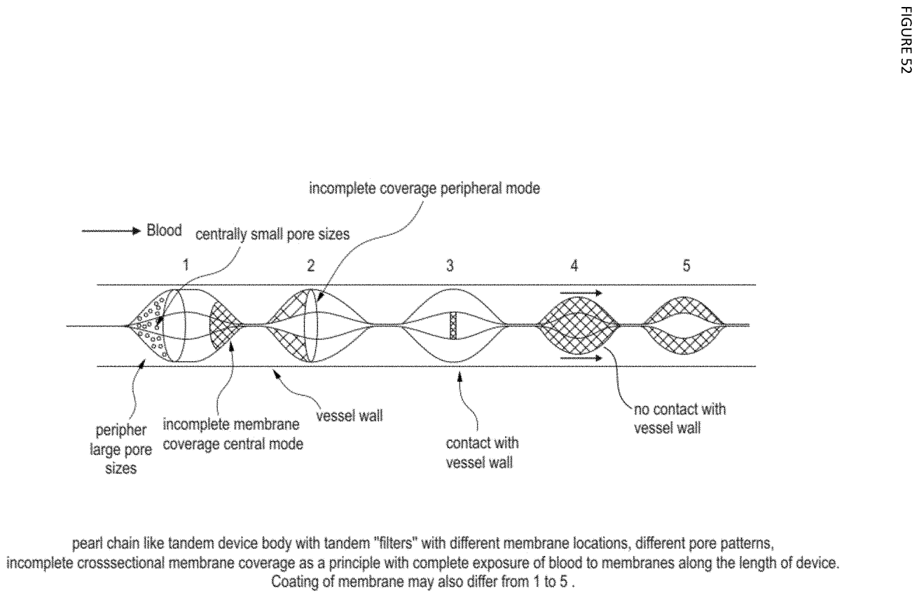

All of the experiments were performed with late passage cells. Cells grew in 3D-spheroids after 6-8 passages. After cell spit 1:10 cells were incubated in fresh medium and transferred to 12 well plate (see below). At day 5 spheroids were counted using 25× amplification under a microscope to establish an average number of spheroids. At day 5, CFPAC-1 cells and spheroids were incubated with Taxus Liberté, Paclitaxel-eluting coronary stent system Monorail (size 2.5 mm×20 mm) for another 10 days. At day 15 spheroids were counted under a microscope (see FIG. 49).

The coated stent was placed inside a well of a 12 well plate as mentioned above. Under normal conditions the splitted cells, with a cell scraper, immediately started to build 3D structures. These structures looked like branches, which turned into spheroid structures upon further culturing. Under these conditions approximately n=30 3D structures were counted at day 15 under a microscope. In the well with the coated stent, spheroid structures were markedly reduced at day 15 (see FIG. 49). There was still a monolayer of cells in the bottom of the well but the number of spheroids were less compared to the well without the stent. Under the microscope about 10-15 3D structures were counted. Many of those structures demonstrated tree- or finger-like branches, some spheroids were present, n=12. It is assumed that Paclitaxel slowed down growth capacity of CFPC-1 cells to generate 3-D spheroid structures.

In addition, RNA was prepared (RNeasy Plus Mini Kit, Qiagen) in order to investigate the transcription profile of cells +/− Paclitaxel treatment. RNA was stored at −80° C. Transcription profile investigation is conducted at state of spheroid generation in vitro.

### Example 2

In a separate experiment (data not shown) CFPC-1 cells were labeled with Cell Tracker Orange Fluorescent Probe from Lonza. Cell labeling was performed according to manufacturer's protocol, Cat. No. PA-3012. Tumor cells express CA19-9 antigen. Using an antibody CA19-9 monoclonal antibody (121SLE), Thermo Fisher, tumor cells were labeled. Preoperative elevated CA-19-9 levels in patients with stage I pancreatic carcinoma decrease to normal values following surgery. When used serially, CA19-9 can predict recurrence of disease prior to radiographic or clinical findings. Antibody working solution was prepared according to manufactures protocol. SEFAR MEDIF AB 03-125/39 70-100 M 102 cm, SEFaR MEDIF AB 03-170/54 102 cm and SEFAR PETEX 07-6/5 115 cm and SEFAR PETEX 07-5/1 115 cm were submerged in anti CA19-9 antibody working solution. The membranes were first autoclaved and then incubated in solution for 1 hour at RT. The membranes were cut in app. 1 cm² and put in a tube (plastic straw).

The artificial device was submerged into a 12 well media solution containing the tumor cells. Using a 1 ml Pipette cells were flushed through the device 10-12 times within 10 hours. The membranes without the antibody “coating” did not retain as many tumor cells labeled with fCell Tracker Orange, compared to controls. Controls were membranes without antibody coating. The intensity of fluorescence was counted in a fluorescence microscope, Zeiss.

All of the experiments were performed with late passage cells. Cells grew in 3D-spheroids after 6-8 passages.

### Example 3

### In Vitro Experiments: Chandler Loop

The below described in vitro system is a ready to use device system for a loop with a maximum diameter of 200 mm consisting of a rotation unit, a removable inox loop cradle for polymer tubing loops tube connectors or accurate closing lap joints and a temperature-controlled water basin (see also British Journal of Pharmacology (2008) 153, 124-131). The chandler loop system enables simulation of extracorporal blood circulation (ECC). The Chandler loop method provides a reliable technique for examining the effect of compounds or devices on rtPA-induced lysis in vitro. It is a reliable method to evaluate potential alterations in rtPA-induced lysis at clinically relevant concentrations of OncoStent scaffold coated bioactive molecules. This evaluation is performed for toxicological evaluation of our medical devices prior to in vivo studies).

Further in vitro tests applied for our device are being performed according to EMA Doc. Ref. EMEA/CHMP/EWP/110540/2007. Non-clinical testing requirements for the drug eluting stents, non-clinical pharmacokinetic testing. The Medical Devices Directive and its corresponding Guidelines state that in the case of implantable devices, active implantable devices and devices of Class III, evidence of the clinical performance and safety of a medical device is provided by means of clinical data.

In vitro cell culture experiments are performed using CRL1918 human pancreatic cancer cells of a liver metastasis. In cell culture binding experiments are performed to select the most bioactive composition for a coating of the device according to the invention, e.g. CXCR2, CXCR4, PDL1, TRAIL, RXR activation compounds and Mg²⁺. Co-cultures are performed using human immune cells and whole blood.

### Example 4

### In Vivo Experiments: T Cell Deficient Pig Model (Oncology)

A particular pathogen free animal model is used for in vivo experiments. The device for pancreatic cancer is developed considering the aspect of clinical relevance (EMA Doc. Ref. EMEA/CHMP/EWP/110540/2007). The devices according to the invention show equivalence with regard to anticipated clinical studies:

Clinically: used for the same clinical condition or purpose; used at the same site in the body; used in similar population (including age, anatomy, physiology); have similar relevant critical performance according to expected clinical effect for specific intended use.

Technically: used under similar conditions of use; have similar specifications and properties; viscosity, surface characteristics; be of similar design; use similar deployment methods (if relevant); have similar principles of operation (same implant material, same bioactive coating material), same size.

Biologically: use of same materials in contact with the same human tissues or body fluids.

3-month old T cell deficient pigs are injected intravenously with 300 Mio CRL-1918 human cells. Time points for acceptable pathological evaluation depend upon the specifics of DES (i.e., polymer and medicinal substance characteristics, elution kinetics, etc). The pig receives three the devices according to the invention at the time of xenografting. The devices are placed into different vessels according to major pathways of tumor cell dissemination.

### Placement of Devices:

Truncus coeliacus (common port for three arteries coming from the Aorta adbominalis; Arteria splenica, Arteria gastroduodenalis) and Arteria mesenterica superior
Vena cava inferior
Venae pancreaticae, Vena splenica
Arteria lienalis

Further details can be derived from FIG. 58.

CRL-1918 human pancreatic cancer cells are transduced (Lipofectamine, non-viral transfection system) in order to allow for convenient and rapid analysis of circulating tumor cells in vivo.

### Experimental Design:

Day 1: human CRL1918-GFP tumor cell inoculation, n=3×10⁸ cells, iv

Day 2: device bioactive scaffold implantation and device control implantation (non-active)

Day 0, 4, 8, 16, 30, 60: blood sampling, for biological characterization (expression of CA19-9 tumor marker, exosome formation, quantitative analysis and clinical chemistry)

Day 4, 8, 16, 30 and 60: ultrasound examination of the device and vessel lumen (no thrombus formation)

Day 60: end of study, sacrifice, organ collection (liver, spleen, lung, brain, lymph nodes, intestine)

Day 0, 20, 40, 60: glucose tolerance test for pancreas functionality testing (IGTT)

### Biological Characterization:

Tumor cell analysis after blood collection is performed via FACS and ELISA to evaluate tumor cell surface marker expression and metastatic potential. TUNEL tests are performed to evaluate cell apoptosis potential. ADCC for immunological potential screen. Formation of exosomes (samples of days 30 and 60).

### Histology:

Staining Ki67 cell proliferation in liver tissue, CA19-9 staining in sampled organ tissue to evaluate metastatic spread. GFP measuring using fluorescence microscopy.

For evaluation of disease free survival non-active and active devices are being placed in separate animals.

### Device Specifications Used:

The following specifications are used for the experiments:

Size exclusion filter, pore sizes up to 100 nm diameter; device to scavenge circulating tumor exosomes; material e.g. nitinol; no bioactive coating.

Size exclusion filter, pore sizes up to 100 nm diameter; devices to scavenge circulating tumor exosomes; material e.g. nitinol; with bioactive coating.

Size exclusion filter, pore sizes range from 70-100 nm diameter; devices to scavenge circulating tumor exosomes; material e.g. nitinol; no bioactive coating.

Devices of different matrices regarding design and material (e.g. carbon) and e.g. onion-shaped membranes as scaffolds.

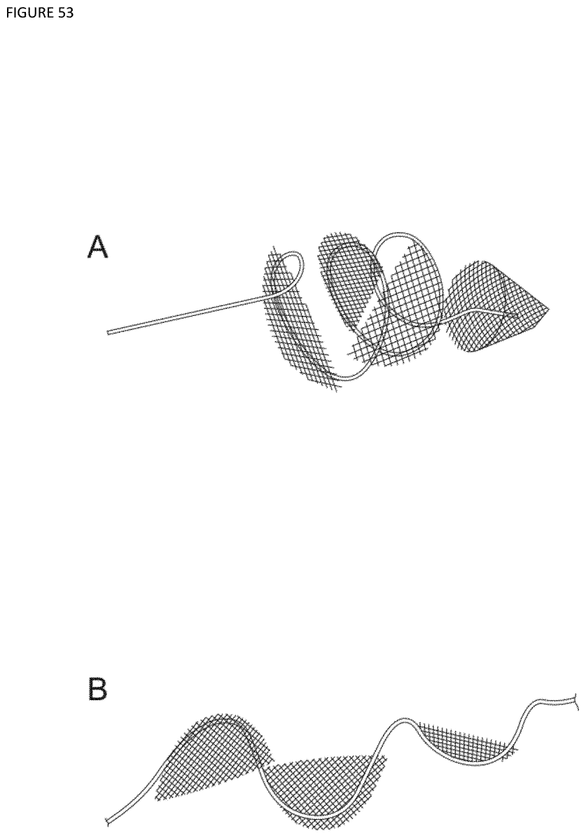

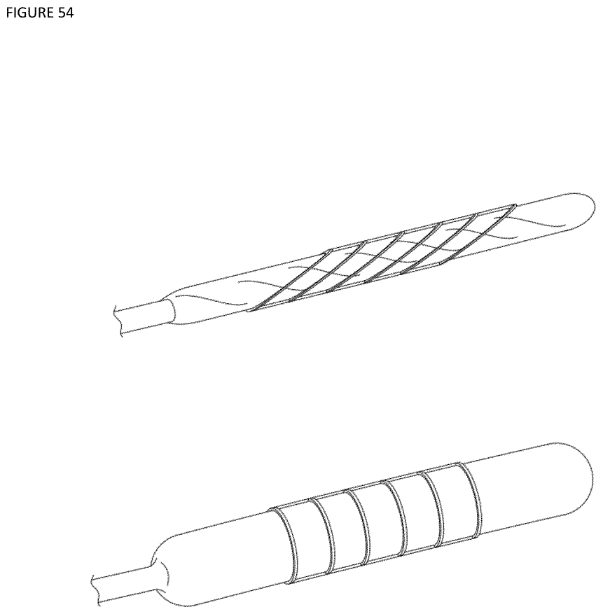

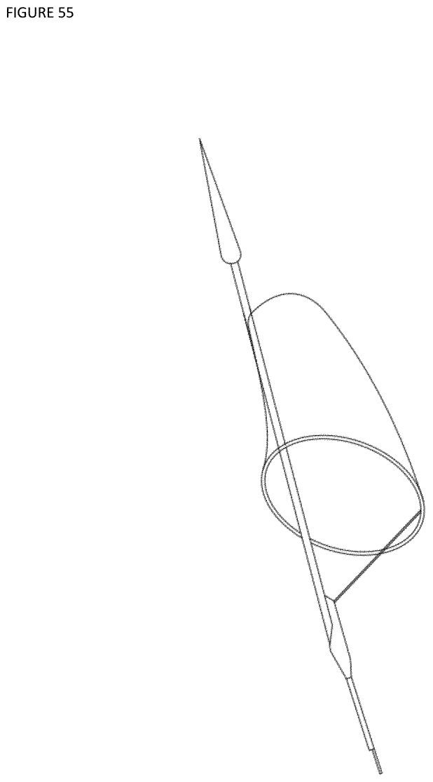

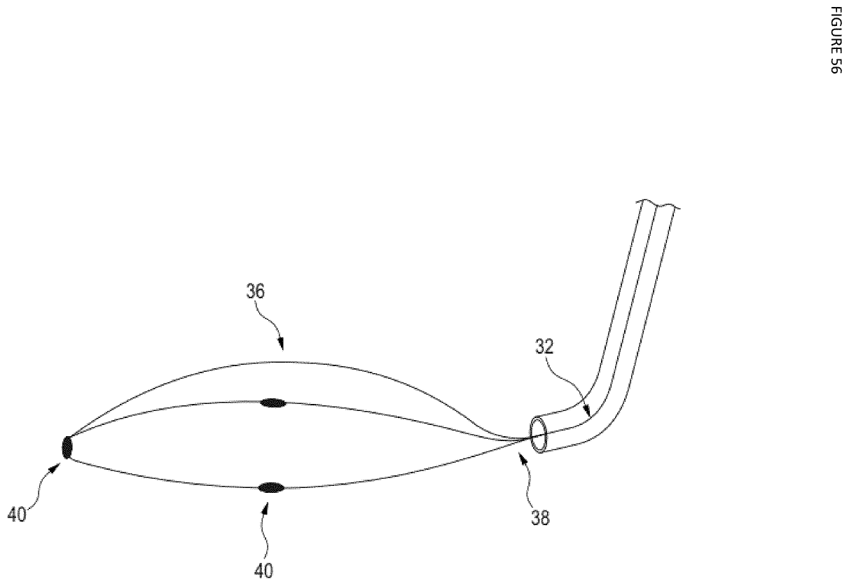

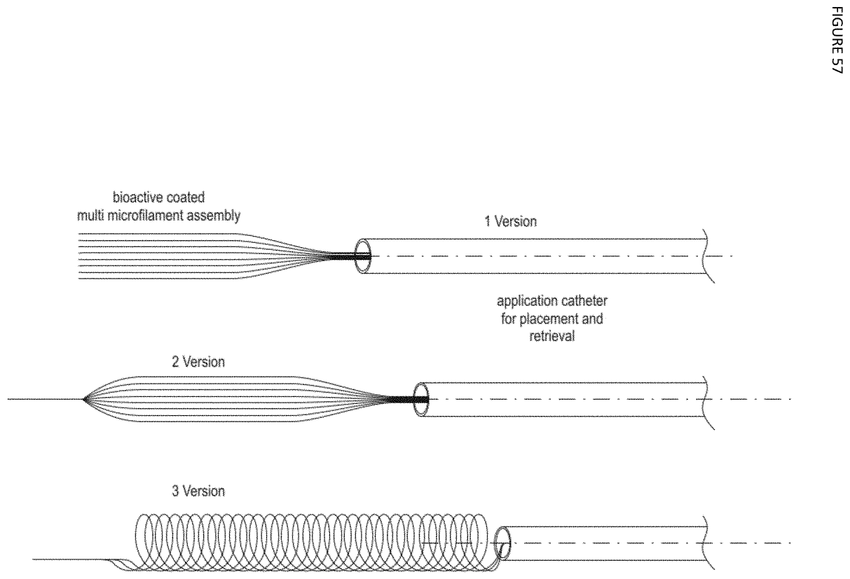

Preferably, devices as described in FIGS. 52 to 64 and explained in the corresponding passages of the description, above, are used.

Combination of up to three different devices with respect to size exclusion in addition to bioactive molecule selection and coating of the matrix

Combination of up to three different devices with respect to size exclusion in addition to bioactive molecule selection and coating of the matrix and drug elution e.g. Mg²⁺.

Devices with bioactive immune-molecule coating to modify circulating tumor cells (Il-10, PDL1 etc.).

Devices with bioactive proapoptotic molecule coating (FAS, FASL etc.).

Devices with cell coating to allow for specific viable interaction with circulating tumor cells.

Devices with bioactive smart peptide coating to tag circulating tumor cells.

Devices with cytokine reservoir to attract circulating tumor cells.

Devices with combination of bioactive coating of cells, cytokine gradient and peptide tags.

### Filter Device Assembly Implant Specifications Used:

The following specifications are used for the experiments:

Size exclusion filter, pore sizes up to 100 nm diameter; filter device assembly implants to scavenge circulating tumor exosomes; material e.g. nitinol; no bioactive coating.

Size exclusion filter, pore sizes up to 100 nm diameter; filter device assembly implants to scavenge circulating tumor exosomes; material e.g. nitinol; with bioactive coating.

Size exclusion filter, pore sizes range from 70-100 nm diameter; filter device assembly implants to scavenge circulating tumor exosomes; material e.g. nitinol; no bioactive coating.

Filter device assembly implants of different matrices regarding design and material (e.g. carbon) and e.g. onion-shaped membranes as scaffolds.

Preferably, filter device assembly implants as described in FIGS. 24 to 31 and explained in the corresponding passages of the description, above, are used.

Combination of up to three different filter device assembly implants with respect to size exclusion in addition to bioactive molecule selection and coating of the matrix

Combination of up to three different filter device assembly implants with respect to size exclusion in addition to bioactive molecule selection and coating of the matrix and drug elution e.g. Mg2+.

Filter device assembly implants with bioactive immune-molecule coating to modify circulating tumor cells (Il-10, PDL1 etc.).

Filter device assembly implants with bioactive proapoptotic molecule coating (FAS, FASL etc.).

Filter device assembly implants with cell coating to allow for specific viable interaction with circulating tumor cells.

Filter device assembly implants with bioactive smart peptide coating to tag circulating tumor cells.

Filter device assembly implants with cytokine reservoir to attract circulating tumor cells.

Filter device assembly implants with combination of bioactive coating of cells, cytokine gradient and peptide tags.

1. A tubular shaped elongated catheter device assembly with a distal and a proximal end, comprising an intraluminal distal segment and an extraluminal proximal segment, comprising one or more chemical and/or biological agents, for interaction with bodily fluids of luminal organs, wherein the intraluminal segment comprises at least one expandable cross-sectional area which in its expanded state is smaller than the cross-sectional area of the luminal target site and wherein at least the expandable portion of the intraluminal segment is capable of interacting with at least one component of the bodily fluid via an interactive contact surface.

2. The tubular shaped elongated catheter device assembly of claim 1, wherein the catheter device is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

3. The tubular shaped elongated catheter device assembly of claim 1, wherein the at least one expandable portion of the intraluminal segment is an inflatable balloon, wherein said expandable portion is designed to bind a tumor cell, a tumor cell exosome, and/or a tumor cell aggregate and/or a pathogen.

4. The tubular shaped elongated catheter device assembly of any one of claims 1 to 3, wherein the cross-sectional area of the intraluminal segment is at least about 50% smaller than the cross-sectional area of the luminal target site.

5. The tubular shaped elongated catheter device assembly of any one of claims 1 to 4, wherein the intraluminal segment comprises at least one expandable portion, preferably a reversibly expandable portion.

6. The tubular shaped elongated catheter device assembly of claim 5, wherein the expandable portion is capable of increasing the interactive contact surface with the bodily fluid.

7. The tubular shaped elongated catheter device assembly of any one of claims 1 to 6, wherein the expandable portion at maximal expansion comprises a cross-sectional area of equal or less than about two thirds of the cross-sectional area of the luminal target site.

8. The tubular shaped elongated catheter device assembly of any one of claims 1 to 7, wherein the expandable portions only partially covering the cross-sectional area of the luminal target are arranged sequentially along the longitudinal extension in a clockwise orientation, preferably with more than 90° differences between the sequential positions.

9. The tubular shaped elongated catheter device assembly of any one of claims 1 to 8, additionally comprising at least two localization markers, preferably opposite to each other.

10. The tubular shaped elongated catheter device assembly of claim 9, wherein said localization markers are located at the starting and end point of a segment comprising chemical and/or biological agents.

11. The tubular shaped elongated catheter device assembly of claim 9 or 10, wherein said localization marker is a radiopaque marker, an ultrasound marker, an MRT marker or a CT marker, preferably further comprising at least one sensor such as an optical sensor, an analyte detecting sensor, a thermal sensor or a flow sensor.

12. The tubular shaped elongated catheter device assembly of any one of claims 1 to 11, wherein the device comprises sections permitting free flow of bodily fluids without any obstructing structural elements at any given point along the longitudinal extension of the device.

13. The tubular shaped elongated catheter device assembly of claim 12, wherein the device comprises sections permitting at least 30%, preferably 50% cross-sectional unhindered flow of bodily fluids.

14. The tubular shaped elongated catheter device assembly of any one of claims 1 to 13, wherein the device comprises sections which permit a free flow of bodily fluids, interrupted by one or more sections permitting an interaction with components of the bodily fluids, preferably a filtering by means of a membrane like component, wherein preferably the interactive sections spare cross-sectional areas of free flow.

15. The tubular shaped elongated catheter device assembly of any one of claims 1 to 14, wherein the tubular shaped elongated catheter device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is anchorable in a target vessel; (v) it is designed to fit into and be connected to a permanent device present in a target vessel as a shuttle docking to a receiving site.

16. The tubular shaped elongated catheter device assembly of any one of claims 1 to 15, wherein the proximal end of the extraluminal segment is connected to an extraluminal fixation element for temporary implantation and for fixation of the intraluminal device and prevention of intraluminal migration of the free floating device.

17. The tubular shaped elongated catheter device assembly of any one of claims 1 to 16, comprising a through lumen housing of the expandable portions of the device.

18. The tubular shaped elongated catheter device assembly of any one of claims 1 to 17, wherein said device is characterized by at least one wire lumen within at least the interluminal segment of the device.

19. The tubular shaped elongated catheter device assembly of any one of claims 1 to 18, wherein the reversibly expandable segment is comprised of at least one expandable balloon surface having a capability for interaction with at least one component of the bodily fluid, wherein the expandable balloon is preferably arranged with the distal end of the intraluminal portion of the device.

20. The tubular shaped elongated catheter device assembly of any one of claims 1 to 19, wherein the elongated tubular shape is provided by a memory shaped wire, preferably a flexible nitinol wire.

21. The tubular shaped elongated catheter device assembly of any one of claims 1 to 20, comprising extensions of the wire in the form of circular loops or ellipsoids or non-straight longitudinal extensions.

22. The tubular shaped elongated catheter device assembly of any one of claims 1 to 21, wherein the expandable portion comprises a porous membranous surface.

23. The tubular shaped elongated catheter device assembly of any one of claims 1 to 22, wherein the expandable portion comprises a filter membrane.

24. The tubular shaped elongated catheter device assembly of claim 23, wherein the filter membrane has one or more of the following properties: (i) it comprises pores, (ii) it is expandable; (iii) it is retrievable; (iv) it is disposed within or around an expandable body; (v) it is characterized by a memory shaped metallic or memory shaped polymer structure; (vi) it comprises a detachment mechanism cooperating between the expandable portion and an intraluminal segment of the device or (vi) it is self-expandable.

25. The tubular shaped elongated catheter device assembly of any one of claims 1 to 24, wherein the device additionally comprises a longitudinally extending free floating microfilaments; or comprises an outer sheath concentrically disposed about the device, wherein the outer sheath and the device are movable relative to one another.

26. The tubular shaped elongated catheter device assembly of any one of claims 1 to 25, wherein the device additionally comprises a downstream embolic debris filter, wherein the filter is preferably retrievable and/or has a pore diameter of >100 μm.

27. The tubular shaped elongated catheter device assembly of any one of claims 1 to 26, wherein the device comprises a longitudinally extending catheter.

28. The tubular shaped elongated catheter device assembly of any one of claims 23 to 27, wherein said filter membrane has at least one of following properties: (i) the filter membrane is attached to and arranged with the expandable portion of the device between its proximal and distal end; (ii) the filter membrane is a non permanent, retrievable filter membrane.

29. The tubular shaped elongated catheter device assembly of any one of claims 24 to 28, wherein said pores have a pore diameter which ranges from about 25 nm to about 100 μm, preferably in a differential manner such as comprising differential ranges of 25 nm to 100 nm, 100 nm to 10 μm, 10 μm to 25 μm, or 25 μm to 100 μm.

30. The tubular shaped elongated catheter device assembly of any one of claims 5 to 29, wherein said expandable portions are at least partially coated with said one or more chemical and/or biological agents, wherein preferably said agents are permanently fixed or releasable.

31. The tubular shaped elongated catheter device assembly of any one of claims 1 to 30, wherein said device is provided in a tubular, onion like, pearl-chain-like, or a birds-nest like shape, or in any mixture of these shapes.

32. The tubular shaped elongated catheter device assembly claim 31, wherein said tubular shape is provided by a memory shaped spiraling wire, which forms a tubular spiral.

33. The tubular shaped elongated catheter device assembly of claim 32, wherein the memory shaped spiraling wire is modified into a single spiral-like wire structure, characterized by incomplete wire circles and an interdigiting structure.

34. The tubular shaped elongated catheter device assembly of any one of claims 31 to 33, wherein said onion like or pearl-chain-like shape is provided by an elastic memory shape meshwork.

35. A tubular shaped device assembly for intraluminal use with a distal and a proximal end, comprising an intraluminal segment which comprises one or more chemical and/or biological agents, for interaction with bodily fluids of luminal organs, wherein at least one portion of the intraluminal segment is expandable and capable of interacting with at least one component of the surrounding bodily fluids via an interactive expandable contact surface, wherein the maximal increase in interactive surface area is at least 3 fold, wherein the interactive segment in its expanded state leaves at least 50% of the surrounding continuous cross-sectional luminal plane void of any structural or interactive component of the expansive device at any level of the longitudinal extension of the interactive segment of the device.

36. The device assembly of claim 35, wherein said device comprises an intraluminal distal segment and an extraluminal proximal segment, further comprising at least two channels within at least a portion of the longitudinal extension of the device, wherein at least one channel is a through channel for use as wire or infusion channel, and wherein at least one channel is a balloon inflation channel, wherein at least one balloon inflation channel is in fluid connection with at least one flow directable compliant expandable balloon, wherein the balloon is arranged around the distal end section of the intraluminal device, wherein expanded balloon diameters are smaller than the surrounding tubular organ diameter, and wherein at least the expandable portions of the intraluminal segment are interactive with elements of bodily fluids in the target area.

37. The device assembly of claim 35 or 36, wherein the at least one expandable portion of the intraluminal segment is an inflatable balloon, wherein said expandable portion is designed to bind a tumor cell, a tumor cell exosome, and/or a tumor cell aggregate, and/or a pathogen.

38. The device assembly of any one of claims 35 to 37, wherein any expanded and interactive portion at any level of the longitudinal extension of the intraluminal segment spares at least about 50% of the surrounding cross-sectional area from any flow obstructive device components or interactive device elements in favor of more than 50% cross-sectional area void of any device elements adjacent or within the interactive site of the device.

39. The device assembly of any one of claims 35 to 38, wherein the intraluminal segment comprises at least two reversibly expandable interactive portions arranged in a tandem like order along the longitudinal axis of the intraluminal segment and sequentially arranged eccentrically and spaced to each other on the circumference of the tubular intraluminal segment, with an offset in a clockwise orientation with at least 90° differences or opposite to each other, preferably with 180° difference in circumferential position.

40. The device assembly of claim 39, wherein at least one expandable portion of the intraluminal segment is extending over any length ranging from about 10 mm to more than 50% of the intraluminal length of the device assembly and is capable of increasing the interactive contact surface with the bodily fluid.

41. The device assembly of any one of claims 35 to 40, wherein at least a portion of the intraluminal segment of the device provides structural elements as reservoirs for drug or biological agents or their compounds, wherein release of these agents is controlled by the release kinetics of the drug compound or is activated by expansive forces such as balloon or self expansion.

42. The device assembly of any one of claims 35 to 41, additionally comprising at least two localization markers on the intraluminal segment, identifying the proximal and distal end of interactive sites, localized eccentrically and opposite to each other on the circumference of the tubular shaped device, the markers being characterized by different configuration as visualizable by medical imaging, wherein the eccentric markers are designed to permit visualization of longitudinal and rotational positioning.

43. The device assembly of claim 42, wherein said localization markers for medical imaging comprise contrast deposits visible in medical imaging.

44. The device assembly of claim 43, wherein said contrast deposits visible in medical imaging are MRI visible, preferably comprising Gadolinium.

45. The device assembly of claim 42 or 43, and wherein said contrast deposits are arranged within balloon like cavities along a non-through lumen such as the inflation lumen.

46. The device assembly of any one of claims 35 to 45, wherein the specific device design maintains continuous cross-sectional areas of more than 60% void of any structural or interactive device components adjacent or along its interactive expandable sites at any given point along the longitudinal extension of the device assembly.

47. The device assembly of any one of claims 35 to 46, wherein the device assembly comprises inactive sections which permit a free flow of bodily fluids along or within the device, interrupted by one or more sections comprising interactive sites which are designed to permit an interaction with components of the bodily fluids, preferably a filtering by means of a membrane like component, wherein preferably the interactive sites spare cross-sectional areas of more than 50% of the cross-sectional surrounding area for free flow void of any device assembly elements.

48. The device assembly of any one of claims 35 to 47, wherein device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is atraumatically anchorable in a target vessel; (v) it is designed to fit into and be connected to a permanent device present in a target vessel as a shuttle docking to a receiving site.

49. The device assembly of any one of claims 35 to 48, wherein said device assembly is characterized by at least one wire lumen within at least the intraluminal segment of the device and at least one inflation lumen.

50. The device assembly of any one of claims 35 to 49, wherein a through lumen within the device assembly houses an optical fiber for light application or a probe for energy application or transmission of sensor activity, preferably an ultrasound probe.

51. The device assembly of any one of claims 35 to 50, wherein at least one expandable interactive site comprises magnetically active components for scavenging magnetically marked antigen/antibody complexes

52. The device assembly of any one of claims 35 to 51, further comprising one or more of the features of any one of claims 2 to 34.

53. A multilumen tubular device, characterized by a multilumen design and by a flow-directable balloon design, comprising reversibly expandable interactive sites which are characterized by interactive surfaces for physical and biochemical or molecular interaction with elements of bodily fluids, wherein at least two interactive sites are arranged in a longitudinally extended tandem position and in a circumferentially clockwise offset position of at least 90° around the tubular device, wherein a continuous cross-sectional area of at least >50% of total surrounding luminal crossection is void of any device components at any point along the longitudinal extension of the device, wherein the device is designed for non permanent use as free floating intraluminal device with extracorporeal fixation unit, wherein at least the beginning and end of an interactive site of the device is marked by markers readable by any medical imaging technique and wherein eccentric markers of different configurations on the tubular device permit appreciation of rotational position by medical imaging techniques.

54. The multilumen tubular device of claim 53, further comprising one or more of the features of any one of claims 2 to 34 or 36 to 52.

55. The tubular shaped elongated catheter device assembly of any one of claims 1 to 34, the device assembly of any one of claims 35 to 52 or the multilumen tubular device of claim 53 or 54, wherein said device is composed of, is partially composed of, or comprises structural support material selected from the group comprising (i) metal, such as stainless steel, gold, titanium, gold-titanium alloy, cobalt-chromium alloy, tantalum, tungsten, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques such as X ray.

56. The tubular shaped elongated catheter device assembly of any one of claims 1 to 34 and 55, the device assembly of any one of claims 35 to 52 and 55 or the multilumen tubular device of any one of claims 53 to 55, wherein said device is self-expandable.

57. The tubular shaped elongated catheter device assembly, the device assembly or the multilumen tubular device of claim 56, wherein at least a portion of the device can be activated by balloon inflation.

58. The tubular shaped elongated catheter device assembly of any one of claims 1 to 34 and 55 to 57, the device assembly of any one of claims 35 to 52 and 55 to 57, or the multilumen tubular device of any one of claims 53 to 57, wherein said device comprises at least one docking element at least one end, preferably at the proximal end, for retrieval.

59. The tubular shaped elongated catheter device assembly of any one of claims 5 to 34 and 55 to 58, the device assembly of any one of claims 35 to 52 and 55 to 58, or the multilumen tubular device of any one of claims 53 to 58, wherein said expandable portion is composed of elastic, foldable polymer material such as polyurethane, or of micromeshes comprising ultrathin wires, metallic or polymeric material.

60. The tubular shaped elongated catheter device assembly of any one of claims 23 to 34 and 55 to 59, the device assembly of any one of claims 35 to 52 and 55 to 59, or the multilumen tubular device of any one of claims 53 to 59, wherein at least one cross-sectional area of the tubular device body is at least partially covered by the filter membrane.

61. The tubular shaped elongated catheter device assembly of any one of claims 23 to 34 and 55 to 60, the device assembly of any one of claims 35 to 52 and 55 to 60, or the multilumen tubular device of any one of claims 53 to 60, wherein the plane of the filter membrane is arranged perpendicular to the direction of the longitudinal axis of the device body.

62. The tubular shaped elongated catheter device assembly of any one of claims 5 to 34 and 55 to 61, the device assembly of any one of claims 35 to 52 and 55 to 61, or the multilumen tubular device of any one of claims 53 to 61, wherein the plane of the filter membrane is in an angle which is non perpendicular to the longitudinal axis of the device body.

63. The tubular shaped elongated catheter device assembly of any one of claims 23 to 34 and 55 to 62, the device assembly of any one of claims 35 to 52 and 55 to 62, or the multilumen tubular device of any one of claims 53 to 62, wherein said device comprises at least two filter membranes, each of which incompletely covers the cross-sectional area, and which are arranged in tandem position along the longitudinal axis of the device body, preferably opposite to each other within the circumference of the device body or shifted in clockwise orientation in case of more than two filter membranes in tandem position.

64. The tubular shaped elongated catheter device assembly of any one of claims 5 to 34 and 55 to 63, the device assembly of any one of claims 35 to 52 and 55 to 63, or the multilumen tubular device of any one of claims 53 to 63, wherein said device comprises alternating non-completely covering filter membranes, preferably in association with a tubular scaffold like and/or in a pearl-chain, onion type, birds-nest like or bi- or trifoil like shape.

65. The tubular shaped elongated catheter device assembly of any one of claims 23 to 34 and 55 to 64, the device assembly of any one of claims 35 to 52 and 55 to 64, or the multilumen tubular device of any one of claims 53 to 64, wherein said filter membranes have differential pore diameters and/or differential pattern, preferably ranging from about 25 nm to about 100 μm, or wherein two or more filter membranes have differential pore diameters and/or differential pattern, preferably ranging from about 25 nm to about 100 μm such as comprising differential pore diameter ranges of 25 nm to 100 nm, 100 nm to 10 μm, 10 μm to 25 μm, or 25 μm to 100 μm.

66. The tubular shaped elongated catheter device assembly of any one of claims 23 to 34 and 55 to 65, the device assembly of any one of claims 35 to 52 and 55 to 65, or the multilumen tubular device of any one of claims 53 to 65, wherein said at least one filter membrane is fully or partially coated on its interior side; or on its exterior side; or on both sides with said one or more chemical and/or biological agents; or wherein said coating differs between different filter membranes.

67. The tubular shaped elongated catheter device assembly, the device assembly or the multilumen tubular device of claim 66, wherein said coating is a passive coating with one or more polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) or linear aliphatic polyesters.

68. The tubular shaped elongated catheter device assembly, the device assembly or the multilumen tubular device of claim 67, wherein said passive coating adheres to the structural support material via an adhesive layer, preferably of sugar, starch, polyvinylalcohol or degradable products of these materials.

69. The tubular shaped elongated catheter device assembly any one of claims 1 to 34 and 55 to 68, the device assembly of any one of claims 35 to 52 and 55 to 68, or the multilumen tubular device of any one of claims 53 to 68, wherein said one or more chemical and/or biological agents constitute an extracellular matrix-like structure.

70. The tubular shaped elongated catheter device assembly, the device assembly or the multilumen tubular device of claim 69, wherein said extracellular matrix-like structure is covalently or non-covalently bound to the passive coating and/or the device material.

71. The tubular shaped elongated catheter device assembly, the device assembly or the multilumen tubular device of claim 69 or 70, wherein said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof; preferably a protein mixture secreted by Engelbreth-Holm-Swarm (EHS) mouse sarcoma cells, Matrigel, BioCoat or GelTrex, more preferably protein mixtures with human proteins.

72. The tubular shaped elongated catheter device assembly of any one of claims 1 to 34 and 55 to 71, the device assembly of any one of claims 35 to 52 and 55 to 71, or the multilumen tubular device of any one of claims 53 to 71, wherein said device provides an environment for circulating metastatic cells.

73. The tubular shaped elongated catheter device assembly of any one of claims 1 to 34 and 55 to 72, the device assembly of any one of claims 35 to 52 and 55 to 72, or the multilumen tubular device of any one of claims 53 to 72, wherein said device comprises a biological agent as an active coating, which is capable of binding to a tumor marker; or to a cell of the immune system or a corresponding immune cell marker.

74. The tubular shaped elongated catheter device assembly, the device assembly or the multilumen tubular device of claim 73, wherein said tumor marker is specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma.

75. The tubular shaped elongated catheter device assembly, the device assembly or the multilumen tubular device of claim 73 or 74, wherein said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₇, CCR10, Axl/Mer, Anxa2-R or Desmoglein I (DG I).

76. The tubular shaped elongated catheter device assembly the device assembly or the multilumen tubular device of claim 73, wherein said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

77. The tubular shaped elongated catheter device assembly, the device assembly or the multilumen tubular device of claim 76, wherein said homing factor is Osteopontin, Hyaluronate, CXCL12, CCL21, Dipeptidyl Dipeptidase IV, PECAM-1, Bone Sialoprotein, Peripheral Node Addressin (CD34), MAD-CAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

78. The tubular shaped elongated catheter device assembly, the device assembly or the multilumen tubular device of claim 73, wherein said cell of the immune system is a CD8⁺ cell, a dendritic cell, a T cell, an engineered T cell, a B cell or an NK cell.

79. The tubular shaped elongated catheter device assembly, the device assembly or the multilumen tubular device of any one of claims 73 to 78, wherein said biological agent is linked to the passive coating of the device or to the structural support material via a spacer element.

80. The tubular shaped elongated catheter device assembly, the device assembly or the multilumen tubular device of claim 79, wherein said linkage to the structural support material is binding to a metal ion resin, such as ion-NTA or ion-agarose.

81. The tubular shaped elongated catheter device assembly, the device assembly or the multilumen tubular device of claim 79 or 80, wherein said spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

82. The tubular shaped elongated catheter device assembly, the device assembly or the multilumen tubular device of any one of claims 79 to 81, wherein said spacer has a length of about 1 to 20 nm.

83. The tubular shaped elongated catheter device assembly, the device assembly or the multilumen tubular device of any one of claims 79 to 82, wherein said spacer elements are provided in a density of 2 to 500 per μm² on the surface of the device.

84. The tubular shaped elongated catheter device assembly, the device assembly or the multilumen tubular device of any one of claims 73 to 83, wherein said biological agent comprises, essentially consists of, or consists of a binding domain capable of binding to a tumor marker.

85. The tubular shaped elongated catheter device assembly, the device assembly or the multilumen tubular device of claim 84, wherein said binding domain is peptide or polypeptide molecule having a length of about 20 to about 250 amino acids, preferably of about 20 to about 120 amino acids.

86. The tubular shaped elongated catheter device assembly, the device assembly or the multilumen tubular device of claim 85, wherein said binding domain has a length of 20, 25, 30, 35, 40, 45, 50, 55, 60, 65, 70, 75, 80, 85, 90, 95, 100, 105, 110, 115 or 120 amino acids.

87. The tubular shaped elongated catheter device assembly, the device assembly or the multilumen tubular device of any one of claims 84 to 86, wherein said biological agent additionally comprises one or more functional domains.

88. The tubular shaped elongated catheter device assembly, the device assembly or the multilumen tubular device of claim 87, wherein said further functional domain is or comprises, partially comprises or consists of an apoptosis inducing factor or a functional domain of an apoptosis inducing factor capable of inducing apoptosis, or a domain capable of binding to a cell of the immune system.

89. The tubular shaped elongated catheter device assembly, the device assembly or the multilumen tubular device of claim 88, wherein said apoptosis inducing factor is FasL/CD95L, TNF-alpha, APO3L or APO2L/TRAIL.

90. The tubular shaped elongated catheter device assembly, the device assembly or the multilumen tubular device of claim 88 or 89, wherein said domain is capable of binding to a tumor marker and said domain capable of inducing apoptosis are provided as fused domains or are linked via a linker element of about 1 to 20 amino acids length.

91. The tubular shaped elongated catheter device assembly, the device assembly or the multilumen tubular device of any one of claims 79 to 90, wherein said biological agent is covalently or non-covalently connected to said spacer.

92. The tubular shaped elongated catheter device assembly, the device assembly or the multilumen tubular device of claim 91, wherein said connection is a linker element.

93. The tubular shaped elongated catheter device assembly, the device assembly or the multilumen tubular device of claim 92, wherein said linker element is a peptide, preferably having a length of 1, 2, 3, 4, 5, 6, 7, 8, 9, 10, 11, 12, 13, 14, 15, 16, 17, 18, 19 or 20 amino acids.

94. The tubular shaped elongated catheter device assembly, the device assembly or the multilumen tubular device of any one of claims 73 to 93, wherein said biological agent is provided as linear or circular element or as an element composed of linear and circular parts, preferably as a linear or circular or partially linear/circular peptide or polypeptide, or a protein with secondary or tertiary structure conformation.

95. The tubular shaped elongated catheter device assembly, the device assembly or the multilumen tubular device of claim 94, wherein said circular biological agent has or is part of a structure comprising a loop or a loop and a stem, such as a human Fc part; or of a linear structure, which is linked to said spacer element.

96. The tubular shaped elongated catheter device assembly, the device assembly or the multilumen tubular device of claim 95, wherein said loop structure or linear structure comprises said biological agent at an exposed position allowing for the binding to a tumor marker or a tumor cell, a tumor cell exosome, and/or a tumor cell aggregate.

97. The tubular shaped elongated catheter device assembly, the device assembly or the multilumen tubular device of any one of claims 81 to 83 or 93 to 96, wherein said peptide has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined conjugated with stabilizing components such as PEG or lipids, preferably forming lipoglycopeptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, comprises, essentially consists of, or consists of D-amino acids and/or L-amino acids; (xi) it partially comprises, comprises, essentially consists of, or consists of homo-amino acids such as beta-homo-amino acids, N-methyl amino acids, or alpha-methyl-amino acids; (xii) it partially comprises, comprises, essentially consists of, or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide.

98. The tubular shaped elongated catheter device assembly, the device assembly or the multilumen tubular device of any one of claims 73 to 97, wherein said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

99. The tubular shaped elongated catheter device assembly of any one of claims 1 to 34 and 55 to 98, the device assembly of any one of claims 35 to 52 and 55 to 98, or the multilumen tubular device of any one of claims 53 to 98, wherein said device further comprises a pharmaceutical agent, preferably selected from the group comprising an antiproliferative agent and an anticoagulant.

100. The tubular shaped elongated catheter device assembly, the device assembly or the multilumen tubular device of claim 99, wherein said antiproliferative agent is paclitaxel, sirolimus, or an analogue thereof.

101. The tubular shaped elongated catheter device assembly, the device assembly or the multilumen tubular device of claim 99, wherein said anticoagulant is reteplase, heparin, or a peptide such as a bifunctional peptide preventing coagulation.

102. A tubular shaped elongated catheter device assembly as defined in any one of claims 1 to 34 and 55 to 101, the device assembly of any one of claims 35 to 52 and 55 to 101, or the multilumen tubular device of any one of claims 53 to 101 for use in treating cancer and/or metastasis, preferably in luminal organs such as blood vessels.

103. A tubular shaped elongated catheter device assembly as defined in any one of claims 1 to 34 and 55 to 101, the device assembly of any one of claims 35 to 52 and 55 to 101, or the multilumen tubular device of any one of claims 53 to 101 for use in preventing cancer and/or metastasis, preferably in luminal organs such as blood vessels.

104. The tubular shaped elongated catheter device assembly for use, the device assembly for use or the multilumen tubular device for use of claim 102 or 103, wherein said device is capable of quantitatively capturing circulating tumor and/or circulating metastatic cell and/or motile parts of tumor cells and/or an aggregate of tumor cells and/or a tumor cell derived exosome, preferably circulating metastatic cells, in a subject's body.

105. The tubular shaped elongated catheter device assembly for use, the device assembly for use or the multilumen tubular device for use of claim 104, wherein said device is capable of preventing downstream organs or tissues to be reached by circulating tumor and/or circulating metastatic cell and/or motile parts of tumor cells and/or an aggregate of tumor cells and/or a tumor cell derived exosome, preferably metastatic cells, which are circulating in a subject's body.

106. The tubular shaped elongated catheter device assembly for use, the device assembly for use or the multilumen tubular device for use of any one of claims 102 to 105, wherein said cancer is colon cancer, breast cancer, lung cancer, melanoma, esophageal cancer, prostate cancer, pancreatic cancer, ovarian cancer, myeloma, lymphoma such as ALL, CLL, AML.

107. The tubular shaped elongated catheter device assembly for use, the device assembly for use or the multilumen tubular device for use of any one of claims 102 to 106 wherein said metastasis is derived from a colon tumor, breast tumor, lung tumor, e.g. small cell lung tumors, squamous cell carcinoma melanoma, prostate tumor, pancreas tumor, lymphoma, T-cell tumor such as Mycosis fungoides, neuroblastoma, sarcoma, fibrosarcoma, ovarian tumor or nephroblastoma such as Wilms tumor.

108. The tubular shaped elongated catheter device assembly of any one of claims 1 to 34 and 55 to 91, the device assembly of any one of claims 35 to 52 and 55 to 91, or the multilumen tubular device of any one of claims 53 to 91, or the tubular shaped elongated catheter device assembly for use, the device assembly for use or the multilumen tubular device for use of any one of claims 102 to 107, wherein said device is designed to be implanted into a luminal organ, preferably a blood vessel such as an artery, an elastic artery, a distributing artery, an arteriole, a capillary, a venule or a vein, preferably into vena cava, or wherein said device is designed to be free floating, or wherein said device is designed to be implanted percutaneously, via a minimal invasive implantation, endoscopically, laparoscopically, or transcutaneously.

109. The tubular shaped elongated catheter device assembly for use, the device assembly for use or the multilumen tubular device for use of any one of claims 102 to 108, wherein said device is implanted in a blood or lymphatic vessel downstream of an existing cancer site in a subject.

110. The tubular shaped elongated catheter device assembly for use, the device assembly for use or the multilumen tubular device for use of claim 109, wherein said device is implanted in close proximity to said existing cancer site.

111. The tubular shaped elongated catheter device assembly for use, the device assembly for use or the multilumen tubular device for use of any one of claims 102 to 108, wherein said device is implanted in a blood vessel upstream of a tissue with a high risk of developing metastasis.

112. The tubular shaped elongated catheter device assembly for use, the device assembly for use or the multilumen tubular device for use of any one of claims 102 to 110, wherein said device is implanted during and/or after the treatment of a subject with a therapeutic agent or during and/or after surgery removing a tumor load.

113. The tubular shaped elongated catheter device assembly for use, the device assembly for use or the multilumen tubular device for use of claim 112, wherein said treatment is an anti-cancer therapy.

114. The tubular shaped elongated catheter device assembly for use, the device assembly for use or the multilumen tubular device for use of claim 103, wherein said device is implanted into a healthy subject or a subject showing no symptoms of a disease, preferably symptoms of cancer or metastasis.

115. The tubular shaped elongated catheter device assembly for use, the device assembly for use or the multilumen tubular device for use of claim 103, wherein said device is implanted into a subject being at risk of developing cancer and/or metastasis.

116. A method of treating cancer and/or metastasis, comprising implanting a tubular shaped elongated catheter device assembly as defined in any one of claims 1 to 34 and 55 to 101, a device assembly as defined in any one of claims 35 to 52 and 55 to 101, or a multilumen tubular device as defined in any one of claims 53 to 101 into a subject in need thereof.

117. A method of preventing cancer and/or metastasis, comprising implanting a tubular shaped elongated catheter device assembly as defined in any one of claims 1 to 34 and 55 to 101, a device assembly as defined in any one of claims 35 to 52 and 55 to 101, or a multilumen tubular device as defined in any one of claims 53 to 101 into a healthy subject or a subject being at risk of developing cancer and/or metastasis.

118. A method of manufacturing a tubular shaped elongated catheter device assembly as defined in any one of claims 1 to 34 and 55 to 101, a device assembly as defined in any one of claims 35 to 52 and 55 to 101, or a multilumen tubular device as defined in any one of claims 53 to 101.

119. The method of claim 118, comprising the step of providing a biological agent as defined in any one of claims 73 to 98 by expressing said biological agent as polypeptide in a suitable host cell, optionally further modifying the polypeptide by adding one or more elements as defined in claim 98.

120. A method for recovering cells, aggregates of cells and/or tumor cell derived exosomes and/or proteins and/or nucleic acids from a tubular shaped elongated catheter device assembly comprising one or more chemical and/or biological agents wherein said device, which is capable of recruiting a circulating tumor and/or circulating metastatic cell and/or motile parts of tumor cells and/or an aggregate of tumor cells and/or a tumor cell derived exosome and/or an immune cell and thereby removes said cell or motile part thereof from circulation, was implanted in a blood or lymphatic vessel downstream of an existing cancer site or close to a site of potential metastasis formation in a subject, wherein said device is retrievable or partially retrievable, preferably by catheter means and/or in a minimal invasive manner.

121. The method of claim 120, wherein the device is an elongated tubular catheter based device assembly, with an intraluminal segment carrying the distal tip of the device and an extraluminal segment carrying the proximal end of the device, comprising a flow directed balloon tipped vascular catheter, wherein the distal end of the intraluminal segment is comprised of a compliant balloon, the balloon being in fluid connection with the proximal end of the extraluminal segment, wherein portions of the intraluminal segment are expandable and interactive with elements of bodily fluids in the target area.

122. The method of claim 120, wherein the at least one expandable portion of the intraluminal segment is an inflatable balloon, wherein said expandable portion is designed to bind a tumor cell, a tumor cell exosome, and/or a tumor cell aggregate and/or an immune cell.

123. The method of any one of claims 120 to 122, wherein the cross-sectional area of the intraluminal segment is at least about 50% smaller than the cross-sectional area of the luminal target site.

124. The method of any one of claims 120 to 123, wherein the intraluminal segment comprises at least one expandable portion, preferably a reversibly expandable portion.

125. The method of claim 124, wherein the expandable portion is capable of increasing the interactive contact surface with the bodily fluid.

126. The method of any one of claims 120 to 125, wherein the expandable portion at maximal expansion comprises a cross-sectional area of equal or less than about two thirds of the cross-sectional area of the luminal target site.

127. The method of any one of claims 120 to 126, wherein the expandable portions only partially covering the cross-sectional area of the luminal target are arranged sequentially along the longitudinal extension in a clockwise orientation, preferably with more than 90° differences between the sequential positions.

128. The method of any one of claims 120 to 127, wherein the device additionally comprises at least two localization markers, preferably opposite to each other.

129. The method of claim 128, wherein said localization markers are located at the starting and end point of a segment comprising chemical and/or biological agents.

130. The method of any one of claims claim 128 or 129, wherein said localization marker is a radiopaque marker, an ultrasound marker, an MRT marker or a CT marker, preferably further comprising at least one sensor such as an optical sensor, an analyte detecting sensor, a thermal sensor or a flow sensor.

131. The method of any one of claims 120 to 130, wherein the device comprises sections permitting free flow of bodily fluids without any obstructing structural elements at any given point along the longitudinal extension of the device.

132. The tubular shaped elongated catheter device assembly of any one of claim 131, wherein the device comprises sections permitting at least 30%, preferably 50% cross-sectional unhindered flow of bodily fluids.

133. The tubular shaped elongated catheter device assembly of any one of claims 120 to 132, wherein the device comprises sections which permit a free flow of bodily fluids, interrupted by one or more sections permitting an interaction with components of the bodily fluids, preferably a filtering by means of a membrane like component, wherein preferably the interactive sections spare >30% of cross-sectional area of the cross-section of the surrounding luminal organ such as blood vessel, permitting to maintain free fluid flow along or through the device.

134. The method of claim 120, wherein said tubular shaped elongated catheter device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is anchorable in a target vessel; (v) it is designed to fit into and be connected to a permanent device present in a target vessel as a shuttle docking to a receiving site.

135. The method of any one of claims 120 to 134, wherein the proximal end of the extraluminal segment is connected to an extraluminal fixation element for temporary implantation and for fixation of the intraluminal device and prevention of intraluminal migration of the free floating device.

136. The method of any one of claims 120 to 135, wherein said device comprises a through lumen housing of the expandable portions of the device.

137. The method of any one of claims 120 to 136, wherein said device is characterized by at least one wire lumen within at least the intraluminal segment of the device.

138. The method of any one of claims 120 to 137, wherein the reversibly expandable segment is comprised of at least one expandable balloon surface having a capability for interaction with at least one component of the bodily fluid, wherein the expandable balloon is preferably arranged with the distal end section of the intraluminal portion of the device.

139. The method of any one of claims 120 to 138, wherein the elongated tubular shape is provided by a memory shaped wire, preferably a flexible nitinol wire.

140. The method of any one of claims 120 to 139, comprising extensions of the wire in the form of circular loops or ellipsoids or non-straight longitudinal extensions.

141. The method of any one of claims 120 to 140, wherein the expandable portion comprises a porous membranous surface.

142. The method of any one of claims 120 to 141, wherein the expandable portion comprises a filter membrane.

143. The method of claim 142, wherein the filter membrane has one or more of the following properties: (i) it comprises pores, (ii) it is expandable, (iii) it is retrievable; (iv) it is disposed within or around an expandable body; (v) it is characterized by a memory shaped metallic or memory shaped polymer structure; (vi) it comprises a detachment mechanism cooperating between the expandable portion and an intraluminal segment of the device or (vi) it is self-expandable.

144. The method of any one of claims 120 to 143, wherein the device additionally comprises a longitudinally extending free floating microfilaments; or comprises an outer sheath concentrically disposed about the device, wherein the outer sheath and the device are movable relative to one another.

145. The method of any one of claims 120 to 144, wherein the device additionally comprises a downstream embolic debris filter, wherein the filter is preferably retrievable and/or has a pore diameter of about >100 μm.

146. The method of any one of claims 120 to 145, wherein the device comprises a longitudinally extending catheter.

147. The method of any one of claims 142 to 146, wherein said filter membrane has at least one of following properties: (i) the filter membrane is attached to and arranged with the expandable portion of the device between its proximal and distal end; (ii) the filter membrane is a non permanent, retrievable filter membrane.

148. The method of any one of claims 143 to 147, wherein said pores have a pore diameter which ranges from about 25 nm to about 100 μm, preferably in a differential manner such as comprising differential ranges of 25 nm to 100 nm, 100 nm to 10 μm, 10 μm to 25 μm, or 25 μm to 100 μm.

149. The method of any one of claims 124 to 148, wherein said expandable portions are at least partially coated with said one or more chemical and/or biological agents.

150. The method of any one of claims 120 to 149, wherein said device is provided in a tubular, onion like, pearl-chain-like, or a birds-nest like shape, or in any mixture of these shapes.

151. The method of claim 150, wherein said tubular shape is provided by a memory shaped spiraling wire, which forms a tubular spiral.

152. The method of claim 151, wherein the memory shaped spiraling wire is modified into a single spiral-like wire structure, characterized by incomplete wire circles and an interdigiting structure.

153. The method of any one of claims 150 to 152, wherein said onion like or pearl-chain-like shape is provided by an elastic memory shape meshwork.

154. The method of any one of claims 120 to 153, wherein the device is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques such as X ray.

155. The method of any one of claims 120 to 154, wherein the device is self-expandable.

156. The method of claim 155, wherein at least a portion of the device can be activated by balloon inflation.

157. The method of any one of claims 120 to 156, wherein the device comprises at least one docking element at the proximal end for retrieval.

158. The method of any one of claims 124 to 157, wherein said expandable portion is composed of elastic, foldable polymer material such as polyurethane, or of micromeshes comprising ultrathin wires, metallic or polymeric material.

159. The method of any one of claims 142 to 158, wherein at least one cross-sectional area of the tubular device body is at least partially covered by the filter membrane.

160. The method of claims 142 to 159, wherein the plane of the filter membrane is arranged perpendicular to the direction of the longitudinal axis of the device body.

161. The method of any one of claims 124 to 160, wherein the plane of the filter membrane is in an angle which is non perpendicular to the longitudinal axis of the device body.

162. The method of any one of claims 144 to 161, wherein the device comprises at least two filter membranes, each of which incompletely covers the cross-sectional area, and which are arranged in tandem position along the longitudinal axis of the device body, preferably opposite to each other within the circumference of the device body or shifted in clockwise orientation in case of more than 2 filter membranes in tandem position.

163. The method of any one of claims 124 to 164, wherein the device comprises alternating non-completely covering filter membranes, preferably in a pearl-chain, onion type or birds-nest like shape.

164. The method of any one of claims 142 to 163, wherein the filter membranes have differential pore diameters and/or differential pattern, preferably ranging from about 25 nm to about 100 μm, or wherein two or more filter membranes have differential pore diameters and/or differential pattern, preferably ranging from about 25 nm to about 100 μm such as comprising differential pore diameter ranges of 25 nm to 100 nm, 100 nm to 10 μm, 10 μm to 25 μm, or 25 μm to 100 μm.

165. The method of claims 142 to 165, wherein said at least one filter membrane is fully or partially coated on its interior side; or on its exterior side; or on both sides with said one or more chemical and/or biological agents; or wherein said coating differs between different filter membranes.

166. The method of claim 165, wherein said coating is a passive coating with one or more polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) or linear aliphatic polyesters.

167. The method of claim 166, wherein said passive coating adheres to the structural support material via an adhesive layer, preferably of sugar, starch, polyvinylalcohol or degradable products of these materials.

168. The method of any one of claims 120 to 167, wherein said one or more chemical and/or biological agents constitute an extracellular matrix-like structure.

169. The method of claim 168, wherein said extracellular matrix-like structure is covalently or non-covalently bound to the passive coating and/or the device material.

170. The method of claim 168 or 169, wherein said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof; preferably a protein mixture secreted by Engelbreth-Holm-Swarm (EHS) mouse sarcoma cells, Matrigel, BioCoat or GelTrex, more preferably protein mixtures with human proteins.

171. The method of any one of claims 120 to 170, wherein the device provides an environment for circulating metastatic cells.

172. The method of any one of claims 120 to 171, wherein the device comprises a biological agent as an active coating, which is capable of binding to a tumor marker; or to a cell of the immune system or a corresponding immune cell marker.

173. The method of claim 172, wherein said tumor marker is specific for breast tumors, prostate tumors, pancreas tumors, colon tumors, small cell lung tumors, lymphoma, multiple lymphoma, T-cell tumors, Mycosis fungoides, Melanoma, neuroblastoma, sarcoma, fibrosarcoma, Wilms tumor or Squamous cell carcinoma.

174. The method of claim 172 or 173, wherein said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Galectin-3, N-CAM, L-Selectin, LPAM-I (alpha₄ beta₂), CTLA, Integrin alpha₄ beta₁, Integrin alpha_{E} beta₂, CCR10, Axl/Mer, Anxa2-R or Desmoglein I (DG I).

175. The method of claim 172, wherein said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

176. The method of claim 175, wherein said homing factor is Osteopontin, Hyaluronate, CXCL12, CCL21, Dipeptidyl Dipeptidase IV, PECAM-1, Bone Sialoprotein, Peripheral Node Addressin (CD34), MAD-CAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

177. The method of claim 172, wherein said cell of the immune system is a CD8⁺ cell, a dendritic cell, a T cell, an engineered T cell, a B cell or an NK cell.

178. The method of any one of claims 172 to 177, wherein said biological agent is linked to the passive coating of the device or to the structural support material via a spacer element.

179. The method of claim 178, wherein said linkage to the structural support material is binding to a metal ion resin, such as ion-NTA or ion-agarose.

180. The method of claim 178 or 179, wherein said spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

181. The method of any one of claims 178 to 180, wherein said spacer has a length of about 1 to 20 nm.

182. The method of any one of claims 178 to 181, wherein said spacer elements are provided in a density of 1 mg per cm² on the surface of the device.

183. The method of any one of claims 172 to 182, wherein said biological agent comprises, essentially consists of, or consists of a binding domain capable of binding to a tumor marker.

184. The method of claim 183, wherein said binding domain is peptide or polypeptide molecule having a length of about 20 to about 250 amino acids, preferably of about 20 to about 120 amino acids.

185. The method of claim 184, wherein said binding domain has a length of 20, 25, 30, 35, 40, 45, 50, 55, 60, 65, 70, 75, 80, 85, 90, 95, 100, 105, 110, 115 or 120 amino acids.

186. The method of any one of claims 183 to 185, wherein said biological agent additionally comprises one or more functional domains.

187. The method of any one of claims 178 to 186, wherein said biological agent is covalently or non-covalently connected to said spacer.

188. The method of claim 187 wherein said connection is a linker element.

189. The method of claim 188, wherein said linker element is a peptide, preferably having a length of 1, 2, 3, 4, 5, 6, 7, 8, 9, 10, 11, 12, 13, 14, 15, 16, 17, 18, 19 or 20 amino acids.

190. The method of any one of claims 172 to 189, wherein said biological agent is provided as linear or circular element or as an element composed of linear and circular parts, preferably as a linear or circular or partially linear/circular peptide or polypeptide, or a protein with secondary or tertiary structure conformation.

191. The method of claim 190, wherein said circular biological agent has or is part of a structure comprising a loop or a loop and a stem, such as a human Fc part; or of a linear structure, which is linked to said spacer element.

192. The method of claim 191, wherein said loop structure or linear structure comprises said biological agent at an exposed position allowing for the binding to a tumor marker or a tumor cell, a tumor cell exosome, and/or a tumor cell aggregate.

193. The method of any one of claims 180 to 182 or 189 to 192, wherein said peptide has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined conjugated with stabilizing components such as PEG or lipids, preferably forming lipoglycopeptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, comprises, essentially consists of, or consists of D-amino acids and/or L-amino acids; (xi) it partially comprises, comprises, essentially consists of, or consists of homo-amino acids such as beta-homo-amino acids, N-methyl amino acids, or alpha-methyl-amino acids; (xii) it partially comprises, comprises, essentially consists of, or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide.

194. The method of any one of claims 172 to 193, wherein said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

195. The method of any one of claims 120 to 194, wherein the part of the device which is retrievable comprises the filter membrane or part of it, or an embolic filter or part of it.

196. The method of any one of claims 120 to 195 wherein the recovering of cells and/or proteins from the device is performed ex vivo after 1, 2, 3, 4, 5, 6, 7 days, 2, 3, 4 weeks or 2, 3, 4, 5, 6, 7, 8, 9, 10, 11, 12 months of implantation time, or after a signal indicating a filling state at or beyond a predefined threshold is received or measured.

197. The method of any one of claims 120 to 196, wherein the recovering cells and/or proteins is performed subsequent to a tumor treatment, preferably for recovering any circulating tumor and/or circulating metastatic cell and/or motile part of tumor cells and/or aggregates of tumor cells and/or tumor cell derived exosomes which are still present in the circulation.

198. The method of any one of claims 120 to 197, comprising the cultivation of recovered cell(s).

199. The method any one of claims 120 to 198, comprising the molecular, chemical, histological and/or physical analysis of the recovered cell(s).

200. The method of any one of claims 120 to 199, comprising the analysis of proteins and/or nucleic acids present in or on the recovered cell(s).

201. A cell, a cell aggregate or an exosome or part of any of the before mentioned, obtained from the method of any one of claims 120 to 198.

202. A protein or nucleic acid obtained from the method of any one of claims 120 to 198.

203. A method of diagnosing cancer and/or metastasis, or of determining an increased likelihood for developing cancer and/or metastasis and/or of determining the subject's metastatic immune status, preferably near a metastatic site, comprising implanting a device as defined in any one of claims 120 to 195 into a subject in need thereof and detecting the presence of circulating tumor and/or circulating metastatic cell and/or motile part of tumor cells and/or aggregates of tumor cells and/or tumor cell derived exosomes and or immune cells captured by the device subsequent to the recovery of said device.

204. A method of diagnosing cancer and/or metastasis, or of determining an increased likelihood for developing cancer and/or metastasis, comprising implanting a device as defined in any one of claims 120 to 195 into a healthy subject, a subject showing no symptoms of a disease, preferably symptoms of cancer or metastasis, or a subject being at risk of developing cancer and/or metastasis and detecting the presence of circulating tumor and/or circulating metastatic cell and/or motile part of tumor cells and/or aggregates of tumor cells and/or tumor cell derived exosomes captured by the device.

205. A method of monitoring the effect of a disease treatment, preferably cancer treatment, comprising implanting a device as defined in any one of claims 120 to 195 into a patient currently being treated for a disease, preferably cancer and/or metastasis, or a patients who has finished his disease treatment, preferably cancer and/or metastasis treatment, within a time period of about 1 week to 2 years and subsequently recovering said device.

206. A method of data collection comprising
(i) monitoring a device as defined in any one of claims 120 to 195 implanted in a subject via ultrasound scanning, tomography, optical, or by determining the device environment for changes indicative of a disease; and
(ii) collecting data over time for recognizing changes of the monitored values.

207. A method of identifying a target cell, target protein or target nucleic acid, comprising analyzing a cell, a cell aggregate or an exosome or part of any of the before mentioned as defined in claim 201, or a protein or nucleic acid as defined in claim 202.

208. The method of claim 205, wherein the method comprises a recovering of cells, aggregates of cells and/or tumor cell derived exosomes and/or proteins and/or nucleic acids from the device after a time period of about 1 day to 2 years.

209. The method of claim 208, wherein said recovered cells are sequenced and/or biochemically analyzed and/or compared with previous data or database values on recovered cells to provide a disease status profile.

210. The method of claim 208 or 209, wherein said method comprises a step of prognosticating the length and/or outcome of the treatment.

211. The method of any one of claims 203 to 210, wherein said method comprises a step of adjusting a disease treatment strategy to the diagnostic or target identification values obtained, preferably by modifying the type and/or amount of pharmaceutical agent to be administered.

212. A tubular shaped elongated catheter device assembly comprising one or more chemical and/or biological agents wherein the device is capable of interacting with a circulating tumor cell and/or a circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex and/or a neurologic serum marker protein within bodily fluids of luminal organs, wherein said device is designed to allow a read out and/or monitoring of the device with respect to the recruiting of or interaction with a circulating tumor and/or circulating metastatic cell and/or motile parts of tumor cells and/or an aggregate of tumor cells and/or a tumor cell derived exosome and/or an immunologic cell such as a T cell, B cell or dendritic cell, and/or an antibody complex such as an autoantibody complex, and/or a neurologic serum marker protein.

213. The tubular shaped elongated catheter device assembly of claim 212, wherein said read out and/or monitoring is performed via ultrasound scanning, tomography, optical analysis, and/or by electrochemical measurements, and/or by determining the implant environment for changes indicative of a disease.

214. The tubular shaped elongated catheter device assembly of claim 212 or 213, wherein the device has a distal and a proximal end and comprises an intraluminal distal segment and an extraluminal proximal segment, wherein the intraluminal segment comprises at least one expandable cross-sectional area which in its expanded state is smaller than the cross-sectional area of the luminal target site and wherein at least the expandable portion of the intraluminal segment is capable of interacting with at least one component of the bodily fluid via an interactive contact surface.

215. The tubular shaped elongated catheter device assembly of any one of claims 212 to 214, wherein the catheter device is a flow directed balloon tipped vascular catheter, wherein the distal end of the intraluminal segment is comprised of a compliant balloon, wherein the balloon diameter is smaller than the surrounding vessel diameter, the balloon being non-obstructive to surrounding fluid flow, wherein portions of the intraluminal segment are expandable and interactive with elements of bodily fluids in the target area.

216. The tubular shaped elongated catheter device assembly of claim 214 or 215, wherein the at least one expandable portion of the intraluminal segment is an inflatable balloon, wherein said expand-able portion is designed to bind a tumor cell, a tumor cell exosome, and/or a tumor cell aggregate, and/or an immunologic cell such as a T cell, B cell or dendritic cell and/or an antibody complex such as an autoantibody complex, and/or a neurologic serum marker protein.

217. The tubular shaped elongated catheter device assembly of any one of claims 214 to 216, wherein the cross-sectional area of the intraluminal segment is at least about 50% smaller than the cross-sectional area of the luminal target site.

218. The tubular shaped elongated catheter device assembly of any one of claims 214 to 217, wherein the intraluminal segment comprises at least one expandable portion, preferably a reversibly expandable portion.

219. The tubular shaped elongated catheter device assembly of claim 218, wherein the expandable portion is capable of increasing the interactive contact surface with the bodily fluid.

220. The tubular shaped elongated catheter device assembly of any one of claims 214 to 219, wherein the expandable portion at maximal expansion comprises a cross-sectional area of equal or less than about two thirds of the cross-sectional area of the luminal target site.

221. The tubular shaped elongated catheter device assembly of any one of claims 214 to 220, wherein the expandable portions only partially covering the cross-sectional area of the luminal target are arranged sequentially along the longitudinal extension in a clockwise orientation, preferably with more than 90° differences between the sequential positions.

222. The tubular shaped elongated catheter device assembly of any one of claims 214 to 221, additionally comprising at least two localization markers, preferably opposite to each other.

223. The tubular shaped elongated catheter device assembly of claim 222, wherein said localization markers are located at the starting and end point of a segment comprising chemical and/or biological agents.

224. The tubular shaped elongated catheter device assembly of claim 222 or 223, wherein said localization marker is a radiopaque marker, an ultrasound marker, an MRT marker or a CT marker.

225. The tubular shaped elongated catheter device assembly of any one of claims 212 to 224, wherein the device comprises sections permitting free flow of bodily fluids without any obstructing structural elements at any given point along the longitudinal extension of the device.

226. The tubular shaped elongated catheter device assembly of claim 225, wherein the device comprises sections permitting at least 30%, preferably 50% cross-sectional unhindered flow of bodily fluids.

227. The tubular shaped elongated catheter device assembly of any one of claims 212 to 224, wherein the device comprises sections which permit a free flow of bodily fluids, interrupted by one or more sections permitting an interaction with components of the bodily fluids, preferably a filtering by means of a membrane like component, wherein preferably the interactive sections spare cross-sectional areas of free flow.

228. The tubular shaped elongated catheter device assembly of any one of claims 212 to 227, wherein the tubular shaped elongated catheter device assembly has one or more of the following properties: (i) it is freely floating in a target vessel; (ii) it is freely positionable in a target vessel, preferably in a minimal invasive manner; (iii) it is retrievable, preferably by catheter means and/or in a minimal invasive manner; (iv) it is anchorable in a target vessel; (v) it is designed to fit into and be connected to a permanent device present in a target vessel as a shuttle docking to a receiving site.

229. The tubular shaped elongated catheter device assembly of any one of claims 214 to 228, wherein the proximal end of the extraluminal segment is connected to an extraluminal fixation element for temporary implantation and for fixation of the intraluminal device and prevention of intraluminal migration of the free floating device.

230. The tubular shaped elongated catheter device assembly of any one of claims 214 to 229, comprising a through lumen housing of the expandable portions of the device.

231. The tubular shaped elongated catheter device assembly of any one of claims 214 to 230, wherein said device is characterized by at least one wire lumen within at least the interluminal segment of the device.

232. The tubular shaped elongated catheter device assembly of any one of claims 214 to 231, wherein the reversibly expandable segment is comprised of at least one interactive expandable balloon sur-face having a capability for interaction with at least one component of the bodily fluid, wherein the expandable balloon is preferably arranged with the distal end of the intraluminal portion of the device and wherein the interactive balloon may preferably also serve as a flow directing balloon.

233. The tubular shaped elongated catheter device assembly of any one of claims 212 to 222, wherein the elongated tubular shape is provided by a memory shaped wire, preferably a flexible nitinol wire.

234. The tubular shaped elongated catheter device assembly of any one of claims 212 to 233, comprising extensions of the wire in the form of circular loops or ellipsoids or non-straight longitudinal extensions.

235. The tubular shaped elongated catheter device assembly of any one of claims 214 to 234, wherein the expandable portion comprises a porous membranous surface.

236. The tubular shaped elongated catheter device assembly of any one of claims 214 to 235, wherein the expandable portion comprises a filter membrane.

237. The tubular shaped elongated catheter device assembly of claim 236, wherein the filter membrane has one or more of the following properties: (i) it comprises pores, (ii) it is expandable; (iii) it is retrievable; (iv) it is disposed within or around an expandable body; (v) it is characterized by a memory shaped metallic or memory shaped polymer structure; (vi) it comprises a detachment mechanism cooperating between the expandable portion and an intraluminal segment of the device or (vi) it is self-expandable.

238. The tubular shaped elongated catheter device assembly of any one of claims 212 to 237, wherein the device additionally comprises longitudinally extending free floating microfilaments; or comprises an outer sheath concentrically disposed about the device, wherein the outer sheath and the device are movable relative to one another.

239. The tubular shaped elongated catheter device assembly of any one of claims 212 to 238, wherein the device additionally comprises a downstream embolic debris filter, wherein the filter is preferably retrievable and/or has a pore diameter of >100 μm.

240. The tubular shaped elongated catheter device assembly of any one of claims 212 to 239, wherein the device comprises a longitudinally extending catheter.

241. The tubular shaped elongated catheter device assembly of any one of claims 236 to 240, wherein said filter membrane has at least one of following properties: (i) the filter membrane is attached to and arranged with the expandable portion of the device between its proximal and distal end; (ii) the filter membrane is a non permanent, retrievable filter membrane.

242. The tubular shaped elongated catheter device assembly of any one of claims 237 to 241, wherein said pores have a pore diameter which ranges from about 25 nm to about 100 μm, preferably in a differential manner such as comprising differential ranges of 25 nm to 100 nm, 100 nm to 10 μm, 10 μm to 25 μm, or 25 μm to 100 μm.

243. The tubular shaped elongated catheter device assembly of any one of claims 214 to 242, wherein said wherein said expandable portions are at least partially coated with said one or more chemical and/or biological agents.

244. The tubular shaped elongated catheter device assembly of any one of claims 212 to 243, wherein said device is provided in a tubular, onion like, pearl-chain-like, or a birds-nest like shape, or in any mixture of these shapes.

245. The tubular shaped elongated catheter device assembly of claim 244, wherein said tubular shape is provided by a memory shaped spiraling wire, which forms a tubular spiral.

246. The tubular shaped elongated catheter device assembly of claim 219, wherein the memory shaped spiraling wire is modified into a single spiral-like wire structure, characterized by incomplete wire circles and an interdigiting structure.

247. The tubular shaped elongated catheter device assembly of claim 244, wherein said onion like or pearl-chain-like shape is provided by an elastic memory shape meshwork.

248. The tubular shaped elongated catheter device assembly of any one of claims 212 to 247, wherein said implant is composed of, is partially composed of, or comprises structural support material selected from the group comprising (i) metal, such as stainless steel, gold, titanium, gold-titanium alloy, cobalt-chromium alloy, tantalum, platinum-radium alloy, tantalum alloy, magnesium, nickel-titanium alloy, e.g. nitinol, silver or copper, (ii) plastic or polymeric material, (iii) elastic memory shape meshwork material such as memory shape elastic wires or (iv) a material readable by tomography or other imaging techniques such as X ray and wherein the device body is not biodegradable or composed of biomaterial or biodegradable material.

249. The tubular shaped elongated catheter device assembly of any one of claims 212 to 248, wherein said device is self-expandable.

250. The tubular shaped elongated catheter device assembly of claim 249, wherein at least a portion of the implant can be activated by balloon inflation.

251. The tubular shaped elongated catheter device assembly of any one of claims 212 to 250, wherein said device comprises at least one docking element at the proximal end for retrieval.

252. The tubular shaped elongated catheter device assembly of any one of claims 214 to 251, said expandable portion is composed of elastic, foldable polymer material such as polyurethane, or of micromeshes comprising ultrathin wires, metallic or polymeric material.

253. The tubular shaped elongated catheter device assembly of any one of claims 236 to 252, wherein at least one cross-sectional area of the tubular device body is at least partially covered by the filter membrane.

254. The tubular shaped elongated catheter device assembly of any one of claims 236 to 253, wherein the plane of the filter membrane is arranged perpendicular to the direction of the longitudinal axis of the device body.

255. The tubular shaped elongated catheter device assembly of any one of claims 236 to 254, wherein the plane of the filter membrane is in an angle which is non perpendicular to the longitudinal axis of the device body.

256. The tubular shaped elongated catheter device assembly of any one of claims 236 to 255, wherein the device comprises at least two membranes, each of which incompletely covers the cross-sectional area, and which are arranged in tandem position along the longitudinal axis of the device body, preferably opposite to each other within the circumference of the device body or shifted in clockwise orientation in case of more than 2 membranes in tandem position.

257. The tubular shaped elongated catheter device assembly of any one of claims 236 to 256, wherein said device comprises alternating non-completely covering filter membranes, preferably in a pearl-chain, onion type or birds-nest like shape.

258. The tubular shaped elongated catheter device assembly of any one of claims 236 to 246, wherein said filter membranes have differential pore diameters and/or differential pattern, preferably ranging from about 25 nm to about 100 μm, or wherein two or more filter membranes have differential pore diameters and/or differential pattern, preferably ranging from about 25 nm to about 100 μm such as comprising differential pore diameter ranges of 25 nm to 100 nm, 100 nm to 10 μm, 10 μm to 25 μm, or 25 μm to 100 μm.

259. The tubular shaped elongated catheter device assembly of any one of claims 236 to 257, wherein said at least one filter membrane is fully or partially coated on its interior side; or on its exterior side; or on both sides with said one or more chemical and/or biological agents; or wherein said coating differs between different filter membranes.

260. The tubular shaped elongated catheter device assembly of claim 259, wherein said coating is a coating with one or more conductive materials.

261. The tubular shaped elongated catheter device assembly of claim 260, wherein said conductive materials are electro-active polymeric (EAP) materials.

262. The tubular shaped elongated catheter device assembly of claim 261 or 262, wherein said coating with conductive materials is a partial coating, preferably a coating covering between 5% and 95% of the surface of the device.

263. The tubular shaped elongated catheter device assembly of claim 262, wherein said coating is provided in the form of symmetrically distributed areas on the surface of the device, preferably within pores.

264. The tubular shaped elongated catheter device assembly of any one of claims 261 to 263, wherein said EAP material coating comprises one or more areas of insulator (about 10⁻⁹ to 10⁻¹²5 cm⁻¹), semi-conductive (about 10 to 10⁻⁹ S cm⁻¹) and/or or purely conductive (about 10⁶ to 105 cm⁻¹) EAP materials.

265. The tubular shaped elongated catheter device assembly of any one of claims 261 to 264, wherein said EAP materials comprise poly(acetylene) (PAc), poly (p-vinylene) (PPV), poly (p-phenylene) (PPP), poly (γ-phenylene sulphide) (PPS), polypyrrole (PPy), polyaniline (PANI), polythiophene (PTh), poly (3,4-ethylenedioxythiophene) (PEDOT), emeraldine base polyaniline (EB-PANI), polypyrrole/graphene (PYG), poly(3,4-ethylenedioxythiophene) poly(styrenesulfo-nate) (PEDOT:PSS) and/or poly(isothianaphtene) (PITN).

266. The tubular shaped elongated catheter device assembly of claim 264 or 265, wherein said EAP materials are doped or non-doped.

267. The tubular shaped elongated catheter device assembly of any one of claims 260 to 266, wherein the coating is provided on porous filter material, additionally comprising co-porogenes such as NaCl crystals and PEG powder.

268. The tubular shaped elongated catheter device assembly of any one of claims 260 to 267, wherein said device additionally comprises one or more substrate electrodes, preferably composed of platinum, glassy carbon, gold, SnO₂, metallized plastics, carbon fibers or TiO₂.

269. The tubular shaped elongated catheter device assembly of any one of claims 260 to 268, wherein the device comprises, at least in some areas, additionally a passive coating with one or more non electroactive polymeric materials such as ethylene vinyl acetate (EVA), latexes, urethanes, polyurethanes, polysiloxanes, styrene-ethylene/butylene styrene block copolymers (SEBS), polytetrafluoroethylene (PTFE) am linear aliphatic polyesters.

270. The tubular shaped elongated catheter device assembly of claim 269 wherein said passive coating adheres to the structural support material via an adhesive layer, preferably of sugar, starch, polyethylene or degradable products of these materials.

271. The tubular shaped elongated catheter device assembly of any one of claims 261 to 270, wherein said one or more chemical and/or biological agents constitute an extracellular matrix-like structure layer, wherein said extracellular matrix-like structure layer is preferably located above or in juxtaposition to said EPA materials or said layer composed of said EPA materials.

272. The tubular shaped elongated catheter device assembly of claim 271, wherein said extracellular matrix-like structure is covalently or non-covalently bound to the coating and/or the device material, wherein preferably a layer composed of EPA materials is pervaded by elements implementing the covalent or non-covalent binding to the coating and/or the device.

273. The tubular shaped elongated catheter device assembly of claim 271 or 272, wherein said chemical and/or biological agents constituting an extracellular matrix-like structure are selected from the group comprising proteoglycans, such as heparan sulfate, chondroitin sulfate and/or keratin sulfate; non-proteoglycan-polysaccharides such as hyaluronic acid; collagen; elastin; fibronectin and laminin, or a mixture thereof, or integrins; preferably a protein mixture secreted by Engelbreth-Holm-Swarm (EHS) mouse sarcoma cells, Matrigel, BioCoat or GelTrex, more preferably protein mixtures with substitutions of human proteins.

274. The tubular shaped elongated catheter device assembly of any one of claims 212 to 273, wherein said device comprises a biological agent as an active coating, which is capable of binding to a tumor marker and/or a biological agent as an active coating, which is capable of binding to an immunologic receptor or interactor or to a cell of the immune system.

275. The tubular shaped elongated catheter device assembly of claim 274, wherein said tumor marker is specific for breast tumors, prostate tumors, pancreas tumors, colon tumors, small cell lung tumors, lymphoma, multiple lymphoma, T-cell tumors, Mycosis fungoides, melanoma, neuroblastoma, sarcoma, fibrosarcoma, Wilms tumor or Squamous cell carcinoma.

276. The tubular shaped elongated catheter device assembly of claim 274 or 275, wherein said tumor marker is CCR4, CCR6, CCR7, IGF, LFA-1, VLA-4, VLA-5, CD44, CD44 v4-v7, CD44 v6-v7, CD44 D3 (v6-v7), CD44-R (v8-v10), CD44 v10, CD-44R1, CXCR3, CXCR4, CXCR5, CXCR6, CXCR7, Surface Fibronectin, PECAM-1 (CD31), CAM 120/180, Integrin alphaᵥ beta₅, P-Selectin, L-Selectin, Integrin alphaᵥ beta₅, Integrin alpha₄ beta₇, Integrin alpha₂ beta₁, Integrin alpha₂ beta₃, Integrin alphaᵥ beta₃, Ga-lectin-3, N-CAM, L-Selectin, LPAM-I (alpha₄ beta₂), CTLA, Integrin alpha₄ beta₁, Integrin alpha_{E} beta₂, CCR10, Axl/Mer, Anxa2-R or Desmoglein I (DG I).

277. The tubular shaped elongated catheter device assembly of claim 274, wherein said cell of the immune system is a CD8⁺ cell, a dendritic cell, a T cell, an engineered T cell, a B cell or an NK cell.

278. The tubular shaped elongated catheter device assembly of claim 274, wherein said biological agent which is capable of binding to a tumor marker is selected from one or more of the group comprising a tumor-marker specific antibody or a fragment thereof, CD133 or a fragment or domain thereof, VEGFR-1 or a fragment or domain thereof, a homing factor or a fragment or domain thereof; and a tumor-marker specific lectin or a fragment or domain thereof.

279. The tubular shaped elongated catheter device assembly of claim 278 wherein said homing factor is Osteopontin, Hyaluronate, CXCL12, CCL21, Dipeptidyl Dipeptidase IV, PECAM-1, Bone Sialoprotein, Peripheral Node Addressin (CD34), MADCAM-1, VCAM-1, Collagen type I, Fibronectin, Osteonectin, N-CAM, FGF Receptor, GlyCAM-1, ICAM-1, ICAM-2, ICAM-3, E-Selectin, E-Cadherin, HECA-452, CCL27, CXCL9 (Mig), SDF-1, CXCL16, GAS-6, Anxa2, T140 or CXCL10 (IP10).

280. The tubular shaped elongated catheter device assembly of any one of claims 274 to 279, wherein said biological agent is linked to the coating of the device or to the structural support material via a spacer element.

281. The tubular shaped elongated catheter device assembly of claim 280, wherein said linkage to the structural support material is binding to a metal ion resin, such as ion-NTA or ion-agarose.

282. The tubular shaped elongated catheter device assembly of claim 280 or 281, wherein said spacer element is composed or partially composed of a peptide or polypeptide, preferably the Fc part of an antibody or multi-histidine tag; a nucleic acid; a modified nucleic acid; or a polymer such as PEG, PLA, PVA, polyethylene or polypropylene.

283. The tubular shaped elongated catheter device assembly of any one of claims 280 to 282, wherein said spacer has a length of about 1 to 20 nm.

284. The tubular shaped elongated catheter device assembly of any one of claims 280 to 283, wherein said spacer elements are provided in a density of 1 mg per cm² on the surface of the device.

285. The tubular shaped elongated catheter device assembly of any one of claims 284 to 284, wherein said biological agent comprises, essentially consists of, or consists of a binding domain capable of binding to a tumor marker or a binding domain capable of binding to an immunologic receptor or interactor or to a cell of the immune system.

286. The tubular shaped elongated catheter device assembly of claim 285, wherein said binding domain is a peptide or polypeptide molecule having a length of about 20 to about 250 amino acids, preferably of about 20 to about 120 amino acids.

287. The tubular shaped elongated catheter device assembly of claim 286 wherein said binding domain has a length of 20, 25, 30, 35, 40, 45, 50, 55, 60, 65, 70, 75, 80, 85, 90, 95, 100, 105, 110, 115 or 120 amino acids.

288. The tubular shaped elongated catheter device assembly of any one of claims 285 to 287, wherein said biological agent additionally comprises one or more functional domains.

289. The tubular shaped elongated catheter device assembly of any one of claims 274 to 280, wherein said biological agent is covalently or non-covalently connected to said spacer.

290. The tubular shaped elongated catheter device assembly of claim 289, wherein said connection is a linker element.

291. The tubular shaped elongated catheter device assembly of claim 290, wherein said linker element is a peptide having a length of 1, 2, 3, 4, 5, 6, 7, 8, 9, 10, 11, 12, 13, 14, 15, 16, 17, 18, 19 or 20 amino acids.

292. The tubular shaped elongated catheter device assembly of any one of claims 274 to 291, wherein said biological agent is provided as linear or circular element or as an element composed of linear and circular parts, preferably as a linear or circular or partially linear/circular peptide or polypeptide.

293. The tubular shaped elongated catheter device assembly of claim 292, wherein said circular biological agent has or is part of a structure comprising a loop or a loop and a stem; or of a linear structure, which is linked to said spacer element.

294. The tubular shaped elongated catheter device assembly of claim 293, wherein said loop structure or linear structure comprises said biological agent at an exposed position allowing for the binding to a tumor marker or a tumor cell, a tumor cell exosome, and/or a tumor cell aggregate or for the binding to an immunologic receptor or interactor or a cell of the immune system and/or for the binding of an antibody complex such as an autoantibody complex, and/or a neurologic serum marker protein.

295. The tubular shaped elongated catheter device assembly of any one of claims 282 to 284 or 291 to 294, wherein said peptide has one or more of the following properties: (i) it recognizes a linear or conformational (discontinuous) epitope; (ii) it is capable of recognizing a tumor cell, a tumor cell exosome, and/or a tumor cell aggregate; (iii) it is capable of recognizing an immune cell such as a CD8⁺ cell, a dendritic cell, a T cell, an engineered T cell, a B cell or an NK cell; (iv) it operates as antagonistic peptide for a target receptor; (v) it operates as agonistic peptide for a target receptor; (vi) it is provided with a density of >1 mg/cm²; (vii) it is combined or conjugated with stabilizing components such as PEG or lipids, preferably forming lipoglycopeptides; (viii) it is composed of natural and/or synthetic (non-natural) amino acids; (ix) it comprises stabilized alpha-helices, beta-sheets or beta-turns, preferably via the presence of non-natural amino acids; (x) it partially comprises, comprises, essentially consists of, or consists of D-amino acids and/or L-amino acids; (xi) it partially comprises, comprises, essentially consists of, or consists of homo-amino acids such as beta-homo-amino acids, N-methyl amino acids, or alpha-methyl-amino acids; (xii) it partially comprises, comprises, essentially consists of, or consists of citrulline, hydroxyproline, norleucin, 3-ntirotyrosine, nitroarginine, ornithine, napthylalanine, Abu, DAB, methionine sulfoxide and/or methionine sulfone; (xiii) it is bound to or covalently linked to a nanoparticle such as a mesoporous silicaparticle, (xiv) it is provided in a cyclized form, preferably via Cys-Cys cylization, backbone cyclization, thio-ester cyclization, or CLIPS cyclization, (xv) it is prenylated; (xvi) it comprises one or more additional spacers of varying polarity or length, preferably via amide linkage; (xvii) it comprises a radioactive isotope or a metal ion; (xviii) it comprises a biotin tag or an epitope tag such as HA tag, His tag, Myc tag; (xix) it comprises a stable, non-radioactive isotope such as a heavy C, N or H isotope; (xx) it has a cell penetrating function or comprises a protein transduction domain (PTD), preferably having the HIV-Tat sequence, Transportan sequence, KLA sequence, AGR sequence, LyP2 sequence, REA sequence, LSD sequence, HN-1 sequence, CTP sequence, HAP1 sequence, Penetratin sequence, or 293P-1 sequence; (xxi) it comprises a fluorophore, bioluminescence dye or chromophore; (xxii) it comprises more than one biological function, preferably being a bi-functional or tri-functional peptide.

296. The tubular shaped elongated catheter device assembly of claim any one of claims 293 to 295, wherein said loop or linear structure comprises additional elements selected from the group comprising a sensor unit or an interaction unit, preferably an external interaction unit.

297. The tubular shaped elongated catheter device assembly of claim 295, wherein said sensor unit is a conformation sensor capable of changing its conformation upon binding of a ligand to the biological agent and of (i) conveying this conformation change to a receiving unit, which is close to the spacer element, the extracellular matrix-like structure, the coating of the device and/or on the surface of the implant, or of (ii) emitting light or heat upon said conformational change.

298. The tubular shaped elongated catheter device assembly of claim 297, wherein said conformational change is converted into a sensed signal which is transmitted from the device to the environment.

299. The tubular shaped elongated catheter device assembly of any one of claims 274 to 297, wherein said biological agent comprises or is linked to one or more additional elements selected from the group comprising sugar, branched or unbranched multiple sugar structures, alkynes, azides, streptavidin, biotin, amines, carboxylic acids, active esters, epoxides and aziridines.

300. The tubular shaped elongated catheter device assembly of any one of claims 212 to 299, wherein said device is partially composed of or comprises material selected from the group comprising material readable by tomography or suitable for electrochemical or electronic readouts.

301. The tubular shaped elongated catheter device assembly of any one of claims 212 to 300, wherein said device further comprises, is combined with or is designed to be combinable with one or more elements selected from (i) an electrical electrode, (ii) a microprocessor or circuit component, (iii) a read-out module, (iv) a digital domain, (v) a controller component and (vi) a communication module.

302. The tubular shaped elongated catheter device assembly of claim 301, wherein said read-out module is composed of an analog front-end (AFE) and an analog-to-digital converter (ADC), capable of capturing and processing the sensed signal and converting it to the digital domain.

303. The tubular shaped elongated catheter device assembly of claim 302, wherein said sensed signal is processed by a digital signal processor (DSP).

304. The tubular shaped elongated catheter device assembly of any one of claims 301 to 303, wherein the communication module provides communication between the device and an outside receiving module.

305. The tubular shaped elongated catheter device assembly of claim 304, wherein said communication module operates with a wireless transceiver, preferably Bluetooth, a radiofrequency (RF) component, a WLAN or WiFi component, or with an optic fiber or a cable connection, or on the basis of electric resistance measurements.

306. The tubular shaped elongated catheter device assembly of claim 305, wherein said electric resistance measurement is based on an electric signal which is provided to the device, preferably from the outside, and which elicits a signal in dependence on the electrical current conduction and resistance in the device, wherein an increased resistance is indicative for the presence of bound cells, cell aggregates, exosomes or parts thereof.

307. The tubular shaped elongated catheter device assembly of any one of claims 212 to 306, wherein said device is connected to an optic fiber or a cable allowing for a read-out from outside of a subject's body.

308. The tubular shaped elongated catheter device assembly of any one of claims 301 to 307, wherein said read-out module is capable of registering device parameters with regard to one or more properties selected from the group comprising oxygen content, sugar content, temperature, ion concentration, impedance, conductivity, pH, pressure, colors, color changes or color pattern, fluorescence, or bioluminescence.

309. The tubular shaped elongated catheter device assembly of claim 308 wherein said outside receiving module is, or is comprised or integrated in, or associated with: a mobile phone, a computer, a tablet, a handheld device, or a network server, or a network cloud computing device, preferably a hand-held physiological signal measurement device or hand-held radio transmission and receiving device.

310. The tubular shaped elongated catheter device assembly of claim 309, wherein said hand-held physiological signal measurement device or hand-held radio transmission and receiving device comprises one or more of the following: (i) a housing, (ii) a plurality of electrodes attached to the surface of the housing, (iii) a contact with the skin surface of the subject, obtaining from the user a physiological signal's; (iv) a front-end circuit, preferably located inside the housing and connected to the plurality of electrodes to receive the physiological signal; (v) an analog/digital conversion circuit, preferably located inside the housing and connected to the front end circuit; (vi) a wireless transceiver interface located inside the housing; (vii) a processing unit preferably located in the inner housing, which is connected to the analog/digital conversion circuit and a transceiver.

311. The tubular shaped elongated catheter device assembly of any one of claims 308 to 310, wherein said receiving module is connected with a data processing system.

312. The tubular shaped elongated catheter device assembly of claim 311, wherein said data processing system comprises a program capable of collecting data sent by said communication module over time and/or of analyzing or processing said data.

313. The tubular shaped elongated catheter device assembly of claim 312, wherein said program is additionally capable of representing said data graphically and/or comparing said data with one or more reference values and/or wherein said program is capable of comparing a threshold value, preferably derived from background noise, with a measured electrical current value, thereby monitoring clinically relevant events and transforming them into a signal which is being read from the external signal receiving module.

314. The tubular shaped elongated catheter device assembly of any one of claims 212 to 313, wherein said device additionally comprises a drug release module.

315. The tubular shaped elongated catheter device assembly of claim 314, wherein said release module is controllable by the communication module.

316. The tubular shaped elongated catheter device assembly of claim 314, wherein said drug release module is controllable by heat, electrical, magnetic or ultrasound stimuli.

317. The tubular shaped elongated catheter device assembly of any one of claims 314 to 316, wherein said drug release module comprises one or more pharmaceutically active compounds such as an anti-cancer drug, an anti-thrombotic drugs, a cardiovascular drug, a drug against an neurologic disease, preferably Alzheimer's disease.

318. A tubular shaped elongated catheter device assembly as defined in any one of claims 212 to 317 for use in diagnosing and/or monitoring a disease, preferably cancer, tumor metastases, a cardiovascular disease, or for determining an increased likelihood for developing a disease, preferably cancer, metastases, a cardiovascular disease, or a neurologic disease such as Alzheimer's disease in a subject.

319. A tubular shaped elongated catheter device assembly as defined in any one of claims 212 to 317 for use in preventing a disease, preferably cancer, metastases, cardiovascular diseases, neurologic diseases.

320. A tubular shaped elongated catheter device assembly as defined in any one of claims 314 to 317 for use in treating a disease, preferably cancer, metastases, a cardiovascular disease, or a neurologic disease.

321. The tubular shaped elongated catheter device assembly for use of claims 318 to 320 wherein said cancer is colon cancer, breast cancer, lung cancer, melanoma, esophageal cancer, prostate cancer, myeloma, lymphoma such as ALL, CLL, AML, cervix carcinoma, renal carcinoma, urinary bladder carcinoma or brain tumors.

322. The tubular shaped elongated catheter device assembly for use of claims 318 to 320, wherein said metastasis is derived from a colon cancer, breast cancer, lung cancer, or melanoma

323. The tubular shaped elongated catheter device assembly of any one of claims 212 to 317, or tubular shaped elongated catheter device assembly for use of claims 318 to 322, wherein said device is designed to be implanted into a blood vessel such as an artery, an elastic artery, a distributing artery, an arteriole, a capillary, a venule or a vein or wherein said device is designed to be implanted percutaneously, via a minimal invasive implantation, endoscopically, laparoscopically, or transcutaneously.

324. The tubular shaped elongated catheter device assembly for use of any one of claims 318 to 322, wherein said device is implanted in a blood or lymphatic vessel downstream of an existing cancer site in a subject.

325. The tubular shaped elongated catheter device assembly for use of claim 324, wherein said device is implanted in close proximity to said existing cancer site

326. The tubular shaped elongated catheter device assembly for use of any one of claims 318 to 322, wherein said device is implanted in a blood vessel upstream of a tissue with a high risk of developing metastasis.

327. The tubular shaped elongated catheter device assembly for use of any one of claims 318 to 325, wherein said device is implanted during and/or after the treatment of a subject with a therapeutic agent or during and/or after surgery which removes a tumor load.

328. The tubular shaped elongated catheter device assembly for use of claim 327, wherein said treatment is an anti-cancer therapy.

329. The tubular shaped elongated catheter device assembly for use of claim 323, wherein said device is implanted into a healthy subject or a subject showing no symptoms of a disease, preferably symptoms of cancer or metastasis.

330. The tubular shaped elongated catheter device assembly for use of claim 323, wherein said device is implanted into a subject being at risk of developing cancer and/or metastasis.

331. A method of diagnosing or monitoring a disease such as cancer and/or metastasis and/or a cardiovascular disease, or of determining an increased likelihood for developing a disease such as cancer and/or metastasis or a cardiovascular disease comprising implanting a tubular shaped elongated catheter device assembly as defined in any one of claims 212 to 317 into a subject in need thereof and detecting the presence of cancer and/or metastatic cells captured by the implant.

332. A method of diagnosing or monitoring a disease such as cancer and/or metastasis and/or a cardiovascular disease, or of determining an increased likelihood for developing a disease such as cancer and/or metastasis and/or a cardiovascular disease comprising implanting a tubular shaped elongated catheter device assembly as defined in any one of claims 212 to 317 into a healthy subject, a subject showing no symptoms of a disease, preferably symptoms of cancer or metastasis or a cardiovascular disease, or a subject being at risk of developing a disease such as cancer and/or metastasis or a cardiovascular disease and detecting the presence of cancer and/or metastatic cells captured by the device.

333. A method of monitoring the effect of disease treatment, comprising implanting a tubular shaped elongated catheter device assembly as defined in any one of claims 312 to 317 into a patient currently being treated for a disease such as cancer and/or metastasis and or a cardiovascular disease, or a patients who has finished his disease treatment such as cancer and/or metastasis or cardiovascular disease or neurologic disease treatment within a time period of about 1 day to 2 years.

334. A method of data collection comprising
(i) monitoring a tubular shaped elongated catheter device assembly as defined in any one of claims 212 to 317 implanted in a subject via ultrasound scanning, tomography, optical, or electrochemical measurements, or by determining the tubular shaped elongated catheter device assembly environment for changes indicative a disease electrochemical measurements; and
(ii) collecting data over time for recognizing changes of the monitored values.
